SEQUENCE LISTING (11 sequences)

SEQ ID NO: 1 - protein, 124 aa, Artificial Sequence
  Polypeptide
EVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSS

SEQ ID NO: 2 - protein, 138 aa, Artificial Sequence
  Polypeptide
  MOD_RES (125)..(125): X can be an unnatural amino acid preferably a 4-azidophenylalanine residue
EVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSXENLYFQGHHHHHH

SEQ ID NO: 3 - protein, 125 aa, Artificial Sequence
  Polypeptide
  MOD_RES (125)..(125): X can be an unnatural amino acid preferably a 4-azidophenylalanine residue
EVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSX

SEQ ID NO: 4 - protein, 156 aa, Artificial Sequence
  Polypeptide
GGSHHHHHHGMASMTGGQQMGRDLYENLYFQGEVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSS

SEQ ID NO: 5 - protein, 157 aa, Artificial Sequence
  Polypeptide
  MOD_RES (157)..(157): X can be an unnatural amino acid preferably a 4-azidophenylalanine residue
GGSHHHHHHGMASMTGGQQMGRDLYENLYFQGEVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSX

SEQ ID NO: 6 - protein, 145 aa, Artificial Sequence
  Polypeptide
  MISC_FEATURE (125)..(144): Any one or all of amino acids 125-144 can either be present or absent
  MISC_FEATURE (125)..(144): X can be any amino acid
EVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSXXXXXXXXXXXXXXXXXXXXC

SEQ ID NO: 7 - protein, 144 aa, Artificial Sequence
  Polypeptide
  MISC_FEATURE (1)..(20): X can be any amino acid
  MISC_FEATURE (1)..(20): Any one or all of amino acids 1-20 can either be present or absent
XXXXXXXXXXXXXXXXXXXXEVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSS

SEQ ID NO: 8 - protein, 165 aa, Artificial Sequence
  Polypeptide
  MISC_FEATURE (125)..(144): X can be any amino acid
  MISC_FEATURE (125)..(144): Any one or all of amino acids 125-144 can either be present or absent
  MISC_FEATURE (146)..(165): X can be any amino acid
  MISC_FEATURE (146)..(165): Any one or all of amino acids 146-165 can either be present or absent
EVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSXXXXXXXXXXXXXXXXXXXXCXXXXXXXXXXXXXXXXXXXX

SEQ ID NO: 9 - protein, 165 aa, Artificial Sequence
  Polypeptide
  MISC_FEATURE (1)..(20): X can be any amino acid
  MISC_FEATURE (1)..(20): Any one or all of amino acids 1-20 can either be present or absent
  MISC_FEATURE (22)..(41): X can be any amino acid
  MISC_FEATURE (22)..(41): Any one or all of amino acids 22-41 can either be present or absent
XXXXXXXXXXXXXXXXXXXXCXXXXXXXXXXXXXXXXXXXXEVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSS

SEQ ID NO: 10 - protein, 146 aa, Artificial Sequence
  Polypeptide
EVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSLGTLCTPSRENLYFQGHHHHHH

SEQ ID NO: 11 - protein, 145 aa, Artificial Sequence
  Polypeptide
  MISC_FEATURE (2)..(21): X can be any amino acid
  MISC_FEATURE (2)..(21): Any one or all of amino acids 2-21 can either be present or absent
CXXXXXXXXXXXXXXXXXXXXEVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSS

(19) United States
(12) Patent Application Publication
(10) Pub. No.: US 2024/0293588 A1
(43) Pub. Date: Sep. 5, 2024

(54) THERAPEUTIC CONJUGATES

(71) Applicant: Starpharma Pty Ltd, Abbotsford, Victoria (AU)

(72) Inventors: Sudhir Ramnathrao Shengule, Abbotsford, Victoria (AU); David Owen, Abbotsford, Victoria (AU); Richard Hufton, Abbotsford, Victoria (AU); Kris Thurecht, Abbotsford, Victoria (AU)

(21) Appl. No.: 17/928,306

(22) PCT Filed: Jun. 3, 2021
(86) PCT No.: PCT/AU2021/050554

§ 371 Date: Nov. 29, 2022

(30) Foreign Application Priority Data
Jun. 3, 2020 (AU) 2020901833

(51) Int. Cl. A61K 51/10; A61K 51/06

(52) U.S. Cl. CPC A61K 51/1096; A61K 51/065; A61K 51/1045

(57) ABSTRACT

Provided herein is dendrimer-targeting agent conjugate comprising: (a) a dendrimer comprising i) a core unit (C); and ii) building units (BU), wherein the dendrimer has from two to six generations of building units; and wherein the core unit is covalently attached to at least two building units; and the dendrimer further comprising: b) a targeting agent which is covalently linked to the dendrimer by a spacer group; c) one or more first terminal groups attached to an outermost building unit of the dendrimer, wherein the first terminal group comprises a complexation group for complexing a radionuclide; and d) one or more second terminal groups attached to an outermost building unit of the dendrimer, wherein the second terminal group comprises a pharmacokinetic-modifying moiety, or a salt thereof. Also provided are compositions comprising the dendrimer-targeting agent conjugates, and methods of using the dendrimer-targeting agent conjugates and compositions comprising them in therapeutic and imaging applications.

### CROSS REFERENCE TO RELATED APPLICATIONS

This application is a U.S. National Stage Application filed under 35 U.S.C. § 371, based on International Patent Application No. PCT/AU2021/050554, filed on Jun. 3, 2021, which claims priority to Australian Patent Application No. 2020901833, filed Jun. 3, 2020. The entire contents of each of the above-referenced applications are incorporated herein by reference.

### FIELD

The present disclosure relates to targeting agent-dendrimer conjugates for therapy and imaging. The conjugates find use in therapeutic applications, for example in the treatment of tumours. The present disclosure also relates to pharmaceutical compositions comprising the conjugates, and methods of treatment using the conjugates.

### SEQUENCE LISTING

The instant application contains a Sequence Listing which has been filed electronically in ASCII format and is hereby incorporated by reference in its entirety. Said ASCII copy, created on Jun. 16, 2023, is named 136080-00701_ST25.txt and is 17,142 bytes in size.

### BACKGROUND

According to the WHO, cancer is the second leading cause of death worldwide, being responsible for an estimated 9.6 million deaths in 2018. The most prevalent cancers include those that affect lung, breast, colorectal, prostate, skin and stomach tissues.

There have been significant efforts into the research and development of new and efficacious oncology therapies. However, modern cancer therapy has thus far proven only partially successful in treating and prolonging the lives of patients with many common types of cancer. This limited success is due, in a large part, to the relative lack of specificity seen among many of the primary classes of anticancer agents and cytotoxic technologies. Indeed, a majority of the oncology therapies that are available today function on the premise of simply destroying any cells that show uncontrolled growth. Such a focus on nonspecific cell division results in treatments that are nonselective in that they inadvertently damage rapidly dividing non-tumorous cells (e.g., cells residing in the gut). As a result, administration of the oncology therapy often leads to irreversible damage to a patient's healthy cells, resulting in a myriad of side-effects that are detrimental to a patient's quality of life.

Such nonspecific oncology therapies include radionuclide therapy. Radionuclide therapy is a systemic treatment that uses a molecule labelled with a radionuclide to deliver a high level of radiation to tumorous cells to treat some cancers. The therapy uses ionizing radiation to kill cancer cells and shrink tumours by damaging the cells' DNA, thereby preventing these cells from continuing to grow and divide. Existing methods of delivering radiotherapy to the desired site includes mimetics, such as Xifigo (Ra223, Bayer) radioactive beads such as sirspheres (Y-90Sirtex), and targeted therapies such as Lutathera (AAA/Novartis).

While the radionuclide may be effective in reducing the growth and spread of cancer cells, a patient's healthy tissues may also be inadvertently damaged. A significant challenge remains in providing safe radionuclide therapies that achieve and maintain therapeutically relevant levels at the target site (e.g., tumorous cells) for a sustained period of time such that the radionuclide is efficacious. This challenge of a safe and long-lasting radionuclide therapy is compounded by the nature of the radionuclides themselves, which inadvertently damage healthy cells (e.g., blood cells and other cells of the immune system) that are exposed to the radionuclide for a prolonged period of time. Such undesirable exposure to healthy cells often manifests as intolerable side-effects in a cancer patient, which can further limit the effectiveness of the therapy. Essentially, at least for some radionuclide therapies, the pharmacokinetic/pharmacodynamic properties and/or side-effect profile of the radionuclide therapy is suboptimal.

Accordingly, there remains a need to develop safe and efficacious radionuclide therapies, wherein the pharmacokinetic/pharmacodynamic properties provide for potent, selective, long-lasting, and overall controlled delivery of radionuclides, all the while resulting in fewer side-effects to the patient.

### SUMMARY

In a first aspect, there is provided a dendrimer-targeting agent conjugate comprising:
a) a dendrimer comprising
i) a core unit (C); and
ii) building units (BU), each building unit being a lysine residue or an analogue thereof, wherein the dendrimer has from two to six generations of building units; and wherein the core unit is covalently attached to at least two building units;
b) a targeting agent which is covalently linked to the dendrimer by a spacer group;
c) one or more first terminal groups attached to an outermost building unit of the dendrimer, wherein the first terminal group comprises a complexation group for complexing a radionuclide; and
d) one or more second terminal groups attached to an outermost building unit of the dendrimer, wherein the second terminal group comprises a pharmacokinetic-modifying moiety;
or a salt thereof.

The dendrimer-targeting agent conjugate of the first aspect may have a radionuclide complexed with the complexation group to form a dendrimer-targeting agent therapeutic conjugate.

In an aspect, there is provided a dendrimer-targeting agent therapeutic conjugate comprising:
a) a dendrimer comprising
i) a core unit (C); and
ii) building units (BU), each building unit being a lysine residue or an analogue thereof, wherein the dendrimer has from two to six generations of building units; and wherein the core unit is covalently attached to at least two building units;
b) a targeting agent which is covalently linked to the dendrimer by a spacer group;
c) one or more first terminal groups attached to an outermost building unit of the dendrimer, wherein the first terminal group comprises a complexation group complexed with a radionuclide; and
d) one or more second terminal groups attached to an outermost building unit of the dendrimer, wherein the second terminal group comprises a pharmacokinetic-modifying moiety;
or a salt thereof.

In some embodiments, the targeting agent is a peptidic moiety having a molecular weight of up to about 150 kDa, or up to about 110 KDa, or up to about 80 KDa, or up to about 55 KDa, or up to about 20 kDa or up to about 16 kDa, and comprising an antigen-binding site.

In some embodiments, the targeting agent is a peptidic moiety having a molecular weight of up to about 80 kDa and comprising an antigen binding site.

In some embodiments, the targeting agent is selected from: an antibody, a heavy chain antibody, ScFV-Fc, Fab, Fab2, Fv, scFv or a single domain antibody. In some embodiments, the targeting agent comprises or consists of a heavy chain variable (V_{H}) domain. In some embodiments, the targeting agent comprises or consists of a light chain variable (V_{L}) domain. In some embodiments, the targeting agent has a molecular weight of about 5 kDa to about 30 kDa. In some embodiments, the targeting agent has a molecular weight of about 5 kDa to about 20 kDa.

In some embodiments, the targeting agent comprises fewer than 120 amino acid residues.

In some embodiments, the targeting agent is a HER2 targeting agent or an EGFR targeting agent.

In some embodiments, the targeting agent comprises or consists of any of the targeting agent amino acid sequences as defined herein.

In some embodiments, the targeting agent is a small molecule.

In some embodiments, the targeting agent is a small molecule that binds PSMA.

In some embodiments, the targeting agent is a DUPA analogue.

In some embodiments, targeting agent is one which binds to FAP.

In some embodiments, a covalent linkage between the targeting agent and the spacer group has been formed by reaction between complementary reactive functional groups present on an intermediate comprising the targeting agent and an intermediate comprising the dendrimer.

In some embodiments, the intermediate comprising the targeting agent comprises an unnatural amino acid residue, the unnatural amino acid residue having a side-chain including a reactive functional group. In some embodiments, the unnatural amino acid residue is a 4-azidophenylalanine residue.

In some embodiments, the spacer group comprises a PEG group.

In some embodiments, the targeting agent is covalently linked to the spacer group at or near the C-terminus of the peptidic moiety.

In some embodiments, the intermediate comprising the dendrimer comprises a reactive functional group which is an alkyne group. In some embodiments, the alkyne group is a dibenzocyclooctyne-amine group.

In some embodiments, as discussed above, the first terminal group comprises a complexation group complexed with a radionuclide. This may be considered to form a radionuclide-complexation moiety. In some embodiments, the complexation group is a DOTA, benzyl-DOTA, NOTA, DTPA, macropa, sarcophagine, DFO, EDTA or PEPA group.

In some embodiments, the radionuclide in the radionuclide-complexation moiety is a lutetium, gadolinium, gallium, zirconium, actinium, bismuth, astatine, technetium, lead, yttrium or copper radionuclide. In some embodiments, the radionuclide is a gadolinium, gallium, zirconium, lead, or lutetium radionuclide. In some embodiments, the radionuclide is an α-emitter. In some embodiments, the radionuclide is a β-emitter

In some embodiments, the pharmacokinetic-modifying moiety is a polyethylene glycol (PEG) group, or a polyethyloxazoline (PEOX) group, or a poly-(2) methyl-(2)-oxazolamine (POZ), or a poly(2-hydroxypropyl)methacrylamide (pHlPMA) group or a polysarcosine. In some embodiments, the pharmacokinetic-modifying moiety is a polyethylene glycol (PEG) group. In some embodiments, the pharmacokinetic-modifying moiety is a PEG group having an average molecular weight in the range of from 400 to 2400 Daltons or from 400 to 2200 Daltons or from 400 to 1400 Daltons.

In some embodiments, the dendrimer has four generations of building units. In some embodiments, the generations of building units are complete generations.

In some embodiments, the core unit is:

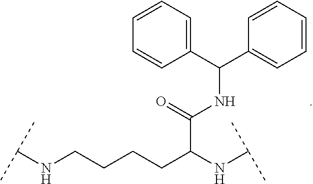

In some embodiments, the core unit comprises the structure:

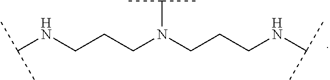

In some embodiments, the core unit is:

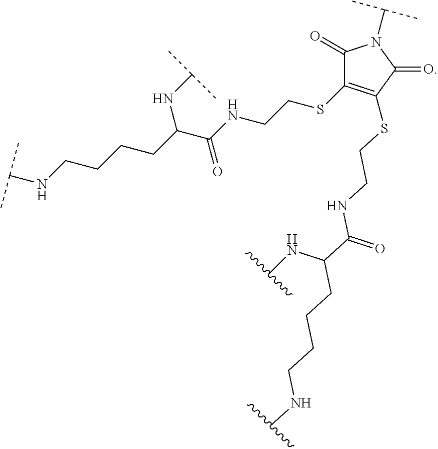

In some embodiments, the building units are lysine residues or an analogue thereof. In some embodiments, the building units are each:

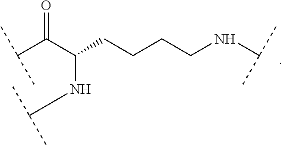

In some embodiments, from 1 to 3 of the nitrogen atoms present in the surface building units are attached to a first terminal group. In some embodiments, at least one third of the nitrogen atoms present in the surface building units are attached to a second terminal group. In some embodiments, at least one third of the nitrogen atoms present in the surface building units are attached to a third terminal group.

In some embodiments, the dendrimer comprises surface building units which contain a nitrogen atom which is capped with an acetyl group.

In some embodiments, the dendrimer is any of the example conjugates.

In a further aspect, there is provided a composition comprising a plurality of conjugates as defined herein.

In a further aspect, there is provided a pharmaceutical composition comprising:
i) a conjugate as defined herein; and
ii) a pharmaceutically acceptable excipient.

In some embodiments, the conjugate or pharmaceutical composition is for use in therapy. In some embodiments, the conjugate or pharmaceutical composition is for use in treating cancer. In such therapeutic embodiments, the dendrimer-targeting agent conjugate may have been complexed with a radionuclide to become a dendrimer-targeting agent therapeutic conjugate to be dispensed to a subject in need of such treatment. References herein to ‘conjugate’ or ‘dendrimer conjugate’ may include both the dendrimer-targeting agent conjugate and dendrimer-targeting agent therapeutic conjugate.

In a further aspect, there is provided the use of a conjugate or pharmaceutical composition as defined herein, in the manufacture of a medicament for the treatment of cancer.

In a further aspect, there is provided a method of treating cancer in a subject, comprising administering a therapeutically effective amount of a conjugate or a pharmaceutical composition as defined herein, to the subject. In some embodiments, the cancer is prostate cancer, pancreatic cancer, breast cancer or brain cancer. In some embodiments, the cancer is a brain cancer of a glioblastoma, meningioma, pituitary, nerve sheath, astrocytoma, oligodendroglioma, ependymoma, medulloblastoma, or craniopharyngioma.

In a further aspect, there is provided a method, use, or conjugate or composition for use as defined herein, wherein the conjugate is administered in combination with a further active agent.

In a further aspect, there is provided a kit for producing a therapeutic conjugate as defined herein, comprising:
a) a conjugate of the first aspect, as defined herein; and
b) a radionuclide.

In a further aspect, there is provided a process for producing a therapeutic conjugate as described herein, comprising contacting a conjugate of the first aspect as defined herein with a radionuclide, thereby producing the therapeutic conjugate.

### BRIEF DESCRIPTION OF THE DRAWINGS

FIG. 1a shows a dendrimer-nanobody conjugation reaction (lane Reaction mix) which generates a mixture of product dendrimers linked to 1, 2, 3, 4, or more nanobodies, as visualised by SDSPAGE and fluorescent imaging. SDSPAGE size markers (blue) represent approximate kilodalton masses.

FIG. 1b shows a dendrimer-nanobody conjugation reaction (lane Reaction mix) which generates a mixture of product dendrimers linked to 1, 2, 3, 4, or more nanobodies, as visualised by SDSPAGE and fluorescent imaging. SDSPAGE size markers (blue) represent approximate kilodalton masses.

FIG. 1c shows a dendrimer-nanobody conjugation reaction (lane Reaction mix) which generates a mixture of product dendrimers linked to 1, 2, 3, 4, or more nanobodies, as visualised by SDSPAGE and fluorescent imaging. SDSPAGE size markers (blue) represent approximate kilodalton masses.

FIG. 1d shows a dendrimer-nanobody conjugation reaction (lane M) which generates a mixture of product dendrimers linked to 1, 2, 3, 4, or more nanobodies, as visualised by SDSPAGE and fluorescent imaging. SDSPAGE size markers (blue) represent approximate kilodalton masses.

FIG. 1e shows a dendrimer-nanobody conjugation reaction (lane M) which generates a mixture of product dendrimers linked to 1, 2, 3, 4, or more nanobodies, as visualised by SDSPAGE and fluorescent imaging. SDSPAGE size markers (blue) represent approximate kilodalton masses.

FIG. 1f shows a dendrimer-nanobody conjugation reaction (lane M) which generates a mixture of product dendrimers linked to 1, 2, 3, 4, or more nanobodies, as visualised by SDSPAGE and fluorescent imaging. SDSPAGE size markers (blue) represent approximate kilodalton masses.

FIG. 1g shows a dendrimer-nanobody conjugation reaction (lane M) which generates a mixture of product dendrimers linked to 1, 2, 3, 4, or more nanobodies, as visualised by SDSPAGE and fluorescent imaging. SDSPAGE size markers (blue) represent approximate kilodalton masses.

FIG. 1h shows SDS-PAGE of fractions obtained from size exclusion. SDS-PAGE marker (blue) represents approximate kilodalton masses, red represents Cy5 fluorescence emitted from the dendrimers. Expected molecular weight of nanobody-dendrimer (˜25 kDa).

FIG. 1i shows SDS-PAGE of fractions obtained from size exclusion. SDS-PAGE marker (blue) represents approximate kilodalton masses, red represents Cy5 fluorescence emitted from the dendrimers. Expected molecular weight of nanobody-dendrimer (˜25 kDa).

FIG. 2 shows mean fluorescence intensity value of Compound 71 (control) and Compound 123 (targeted) with MDA-MB-231, MDA-MB-231/HER2 and SKOV-3 cells over 24 h. At least 10,000 cells were counted per measurement. Values are mean±standard deviation (SD; n=3).

FIG. 3 shows flow cytometry analysis of dendrimers incubated with HER2 positive cell (MDA-MB-231/HER2) and HER2 negative cell (MDA-MB-231), at 3.33 nM over 24 h incubation at 37° C. At least 10,000 cells were counted per measurement. Values are mean±standard deviation (SD; n=3).

FIG. 4 shows confocal microscopy images of MDA-MB-231 cells treated with a) Compound 71 (control) or b) Compound 123 (targeted) at a concentration of 3.33 nM for 24 h. Green, blue, and red fluorescence represent cell membrane stained with AF-488-WGA, nucleus stained with DAPI, and dendrimer labelled with Cy5, respectively. Scale bar=50 μm.

FIG. 5 shows confocal microscopy images of MDA-MB-231/HER2 cells treated with a) Compound 71 (control) or b) Compound 123 (targeted) at a concentration of 3.33 nM for 24 h. Green, blue, and red fluorescence represent cell membrane stained with AF-488-WGA, nucleus stained with DAPI, and dendrimer labelled with Cy5, respectively. Scale bar=50 μm.

FIG. 6 shows confocal microscopy images of SKOV-3 cells treated with a) Compound 71 (control) or b) Compound 123 (targeted) at a concentration of 3.33 nM for 24 h. Green, blue, and red fluorescence represent cell membrane stained with AF-488-WGA, nucleus stained with DAPI, and dendrimer labelled with Cy5, respectively. Scale bar=50 μm.

FIG. 7 shows tumour and blood distribution data of 3H-labelled Compound 72 and Compound 127 dendrimers after sacrifice at 48 h. All data was normalised by tissue mass. All data represents the mean±SEM (n=5); (NS=not significant; * =p-value<0.05).

FIG. 8 shows representative ex-vivo tumour distribution of Compound 71 and Compound 123 after sacrifice at 48 h. Data represents a typical field of view for (a) untargeted dendrimer (Compound 71) and (b) targeted dendrimer (Compound 123).

FIG. 9 shows images showing that targeted dendrimer (Compound 123) was uptaken into the core and peripheral regions of a tumour, and showing that control Compound 71 was not.

FIG. 10 shows a plot of mean tumour volume over time for mice inoculated with SKOV3 cells following treatment with vehicle, control Compound 71, targeted Compound 123, Kadcyla®, or Herceptin®.

FIG. 11 shows percentage survival over time for mice inoculated with SKOV3 cells following treatment with vehicle, control Compound 71, targeted Compound 123, Kadcyla®, or Herceptin®.

FIG. 12 shows mean % weight change over time for mice inoculated with SKOV3 cells following treatment with vehicle, control Compound 71, targeted Compound 123, Kadcyla®, or Herceptin®.

FIG. 13 shows internalisation kinetics of Generation 4 dendrimer with single-conjugated (Compound 91; MFI Single) or multiple-conjugated (Compound 92; MFI Multi) anti-HER2 nanobodies.

FIG. 14 shows confocal microscopy images of SKOV-3 cells after incubation with Compound 92 (multiple 2D3-dendrimer conjugate) at 37° C. for a) 1 h, b) 3 h, c) 6 h, or d) 24 h. Compound 92 is labelled with Cy5 (magenta), cell membrane is stained with AF488-WGA (green), and the nucleus is labelled with Hoechst 33342 (blue). Scale bar=30 μM.

FIG. 15 shows confocal microscopy images of SKOV-3 cells after incubation with Compound 91 (single 2D3-dendrimer conjugate) at 37° C. for a) 1 h, b) 3 h, c) 6 h, or d) 24 h. Compound 91 is labelled with Cy5 (magenta), cell membrane is stained with AF488-WGA (green), and the nucleus is labelled with Hoechst 33342 (blue). Scale bar=30 μM.

FIG. 16 is a radio TLC image for Compound 73 showing that ⁸⁹Zr was bound to the dendrimer.

FIG. 17 shows graphs of percentage injected zirconium dose per gram in (a) kidney, (b) liver, and (c) tumor over 9 days for Compounds 89, 91, and 93.

FIG. 18 shows representative maximum intensity projections of radiolabelled conjugates PET images of animals at 4 hours to 9 days. Data is represented in Becquerel per voxel (cm³) and have been thresholded to highlight tumour uptake.

FIG. 19 shows a plot of % change in tumour volume over time for balb/c nude mice inoculated with BT474 cells following treatment with vehicle, and test articles (Trastuzumab KY-3-310, HER2 nanobody targeted SRS-2-304, and untargeted RH-3-160) delivering 15 MBq ¹⁷⁷Lu.

FIG. 20 shows a plot of % change in tumour volume over time for balb/c nude mice inoculated with BT474 cells following treatment with vehicle, Trastuzumab KY-3-310, or HER2 nanobody targeted SRS-2-304, with varying doses of ¹⁷⁷Lu.

FIG. 21 shows SDS page gel of KY-2a, reduced and unreduced.

FIG. 22 shows SDS page gel of dendrimer conjugates SRS-15, SRS-16, SRS-17 (and corresponding starting materials).

FIG. 23 shows SDS page gel of dendrimer conjugates SRS-20, SRS-21 and SRS-22 (and corresponding starting materials).

Key to the sequence listing
SEQ ID NO: 1 single domain antibody is 2D3 amino acid sequence.
SEQ ID NO: 2 example single domain antibody amino acid sequence.
SEQ ID NO: 3 example single domain antibody amino acid sequence.
SEQ ID NO:4 example single domain antibody amino acid sequence.
SEQ ID NO: 5 example single domain antibody amino acid sequence.
SEQ ID NO: 6 example single domain antibody amino acid sequence.
SEQ ID NO: 7 example single domain antibody amino acid sequence.
SEQ ID NO: 8 example single domain antibody amino acid sequence.
SEQ ID NO: 9 example single domain antibody amino acid sequence.
SEQ ID NO: 10 example single domain antibody amino acid sequence.

### DESCRIPTION

### General Definitions

Unless specifically defined otherwise, all technical and scientific terms used herein shall be taken to have the same meaning as commonly understood by one of ordinary skill in the art (e.g., chemistry, biochemistry, medicinal chemistry, polymer chemistry, and the like).

As used herein, the term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−20%, more preferably +/−10%, of the designated value.

As used herein, the terms “a”, “an” and “the” include both singular and plural aspects, unless the context clearly indicates otherwise.

Unless otherwise indicated, terms such as “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

As used herein, the term “subject” refers to any organism that is susceptible to a disease or condition. For example, the subject can be an animal, a mammal, a primate, a livestock animal (e.g., sheep, cow, horse, pig), a companion animal (e.g., dog, cat), or a laboratory animal (e.g., mouse, rabbit, rat, guinea pig, hamster). In one example, the subject is a mammal. In one embodiment, the subject is human. In one embodiment, the subject is a non-human animal.

As used herein, the term “treating” includes alleviation of symptoms associated with a specific disorder or condition. For example, as used herein, the term “treating cancer” includes alleviating symptoms associated with cancer. In one embodiment, the term “treating cancer” refers to a reduction in cancerous tumour size. In one embodiment, the term “treating cancer” refers to an increase in progression-free survival. As used herein, the term “progression-free survival” refers to the length of time during and after the treatment of cancer that a patient lives with the disease, i.e., cancer, but does not have a recurrence or increase in symptoms of the disease.

As used herein, the term “prevention” includes prophylaxis of the specific disorder or condition. For example, as used herein, the term “preventing cancer” refers to preventing the onset or duration of the symptoms associated with cancer. In one embodiment, the term “preventing cancer” refers to slowing or halting the progression of the cancer. In one embodiment, the term “preventing cancer” refers to slowing or preventing metastasis.

The term “therapeutically effective amount”, as used herein, refers to a dendrimer being administered in an amount sufficient to alleviate or prevent to some extent one or more of the symptoms of the disorder or condition being treated. The result can be the reduction and/or alleviation of the signs, symptoms, or causes of a disease or condition, or any other desired alteration of a biological system. In one embodiment, the term “therapeutically effective amount” refers to a dendrimer being administered in an amount sufficient to result in a reduction in cancerous tumour size. In one embodiment, the term “therapeutically effective amount” refers to a dendrimer being administered in an amount sufficient to result in an increase in progression-free survival. The term, an “effective amount”, as used herein, refers to an amount of a dendrimer effective to achieve a desired pharmacologic effect or therapeutic improvement without undue adverse side effects. The term “therapeutically effective amount” includes, for example, a prophylactically effective amount. In one embodiment, a prophylactically effective amount is an amount sufficient to prevent metastasis. It is understood that “an effective amount” or “a therapeutically effective amount” can vary from subject to subject, due to variation in metabolism of the compound and any of age, weight, general condition of the subject, the condition being treated, the severity of the condition being treated, and the judgment of the prescribing physician.

The term “diagnosis”, as used herein, may include a process of administering a conjugate of the disclosure to a subject having or suspected of having a condition, disease or disorder, and subsequently using a technique such as single photon emission, positron emission tomography and/or positron emission tomography-magnetic resonance imaging to provide information on the level of radioactivity in various parts of the body, for example imaging a part or parts of the subject's body, in order to enable a decision to be made regarding the existence of a disease, disorder or condition (e.g. a cancer) and/or regarding the status, staging and/or extent of the disease, disorder or condition. In some embodiments, the term “diagnosis” may include the act of identifying and/or classifying the status, staging or extent of a disease, disorder or condition from signs or symptoms. For example, as used herein, the term “diagnosing cancer” may include identifying and/or classifying the status, staging or extent of a cancer in a subject.

Suitable salts of the dendrimers include those formed with organic or inorganic acids or bases. As used herein, the phrase “pharmaceutically acceptable salt” refers to pharmaceutically acceptable organic or inorganic salts. Exemplary acid addition salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. It will also be appreciated that non-pharmaceutically acceptable salts also fall within the scope of the present disclosure since these may be useful as intermediates in the preparation of pharmaceutically acceptable salts or may be useful during storage or transport.

Those skilled in the art of organic and/or medicinal chemistry will appreciate that many organic compounds can form complexes with solvents in which they are reacted or from which they are precipitated or crystallized. These complexes are known as “solvates”. For example, a complex with water is known as a “hydrate”. As used herein, the phrase “pharmaceutically acceptable solvate” or “solvate” refer to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine.

As used herein, the term “5- to 10-membered monocyclic or bicyclic heterocyclic group” refers to a monocyclic or bicyclic aromatic or non-aromatic cyclic group which is analogous to a carbocyclyl group, but in which from one or more of the carbon atoms is/are replaced by one or more heteroatoms independently selected from nitrogen, oxygen, or sulfur. A polycyclic heterocyclyl may for example contain fused rings. In a bicyclic heterocyclyl group there may be one or more heteroatoms in each ring, or heteroatoms only in one of the rings. A heteroatom may be N, O, or S. Heterocyclyl groups containing a suitable nitrogen atom include the corresponding N-oxides. In one example, the heterocycle group is of five to ten atoms (i.e. 5- to 10-membered heterocycle). Examples of monocyclic non-aromatic heterocycle groups include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl. Examples of bicyclic heterocycle groups in which one of the rings is non-aromatic include dihydrobenzofuranyl, indanyl, indolinyl, isoindolinyl, tetrahydroisoquinolinyl, tetrahydroquinolyl, and benzoazepanyl. Examples of monocyclic aromatic heterocycle groups (also referred to as monocyclic heteroaryl groups) include furanyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidinyl. Examples of bicyclic aromatic heterocycle groups (also referred to as bicyclic heteroaryl groups) include quinoxalinyl, quinazolinul, pyridopyrazinyl, benzoxazolyl, benzothiophenyl, benzimidazolyl, naphthyridinyl, quinolinyl, benzofuranyl, indolyl, indazolyl, benzothiazolyl, oxazolyl[4,5-b]pyridyl, pyridopyrimidinyl, isoquinolinyl, and benzohydroxazole.

As used herein, the term “saturated” refers to a group where all available valence bonds of the backbone atoms are attached to other atoms Representative examples of saturated groups include, but are not limited to, butyl, cyclohexyl, piperidine, and the like.

As used herein, the term “unsaturated” refers to a group where at least one valence bond of two adjacent backbone atoms is not attached to other atoms. Representative examples include, but are not limited to, alkenes (e.g., —CH₂—CH₂CH═CH), phenyl, pyrrole, and the like.

As used herein, the term “substituted” refers to a group having one or more hydrogens or other atoms removed from a carbon or suitable heteroatom and replaced with a further group (i.e., substituent).

As used herein, the term “dendrimer” refers to a molecule containing a core and dendrons attached to the core. Each dendron is made up of generations of branched building units resulting in a branched structure with increasing number of branches with each generation of building units. A dendrimer may include pharmaceutically acceptable salts or solvates as defined supra.

As used herein, the term “building unit” refers to a branched molecule comprising functional groups, at least one functional group for attachment to the core or a previous generation of building units and at least two functional groups for attachment to the next generation of building units or forming the surface of the dendrimer molecule.

As used herein, the term “attached” refers to a connection between chemical components by way of covalent bonding. The term “covalent bonding” is used interchangeably with the term “covalent attachment”.

### Conjugates

In a first aspect, there is provided a dendrimer-targeting agent conjugate comprising:
a) a dendrimer comprising
i) a core unit (C); and
ii) building units (BU), wherein the dendrimer has from two to six generations of building units; and wherein the core unit is covalently attached to at least two building units;
b) a targeting agent which is covalently linked to the dendrimer by a spacer group;
c) one or more first terminal groups attached to an outermost building unit of the dendrimer, wherein the first terminal group comprises a complexation group for complexing a radionuclide; and
d) one or more second terminal groups attached to an outermost building unit of the dendrimer, wherein the second terminal group comprises a pharmacokinetic-modifying moiety;
or a salt thereof.

The dendrimer-targeting agent conjugate of the first aspect may have a radionuclide complexed with the complexation group to form a dendrimer-targeting agent therapeutic conjugate. The dendrimer-targeting agent therapeutic conjugate may be for use in therapeutic or imaging/diagnostic applications.

In an aspect, there is provided a dendrimer-targeting agent therapeutic conjugate comprising:
a) a dendrimer comprising
i) a core unit (C); and
ii) building units (BU), each building unit being a lysine residue or an analogue thereof, wherein the dendrimer has from two to six generations of building units; and wherein the core unit is covalently attached to at least two building units;
b) a targeting agent which is covalently linked to the dendrimer by a spacer group;
c) one or more first terminal groups attached to an outermost building unit of the dendrimer, wherein the first terminal group comprises a complexation group complexed with a radionuclide; and
d) one or more second terminal groups attached to an outermost building unit of the dendrimer, wherein the second terminal group comprises a pharmacokinetic-modifying moiety;
or a salt thereof.

The conjugates of the present disclosure, containing a dendrimeric scaffold incorporating a targeting agent and a first terminal group comprising a complexation group for complexing a radionuclide find use as agents with pharmaceutical application, for example as therapeutic agents useful in the treatment of cancer. It is considered that the specific combination of dendrimeric scaffold, with pharmacokinetic-modifying groups such as PEG or PEOX groups, conjugated to a targeting agent, provides for delivery of radionuclide to the site of action in a manner such that it can provide sustained therapeutic effects. The design of the conjugates also permits synthesis in a manner allowing for late introduction of the targeting agent, complexation group for complexing a radionuclide and, particularly, radionuclide.

### Dendrimers

### Core Unit

The core unit (C) of the dendrimer provides an attachment point for dendrons formed of building units. Any suitable core unit which contains functional groups that can form covalent linkages with functional groups present on building units may be utilised.

In some embodiments, the core unit is covalently attached to at least two building units via amide linkages. In some embodiments, each amide linkage is formed between a nitrogen atom present in the core unit and the carbon atom of an acyl group present in a building unit. In other embodiments, each amide linkage is formed between the carbon atom of an acyl group present in the core unit and a nitrogen atom present in a building unit.

In some embodiments, the core unit is covalently attached to 2, 3 or 4 building units. In one particular embodiment, the core unit is covalently attached to 2 building units. The core unit may for example be formed from a core unit precursor comprising amino groups. As another example, the core unit may be formed from a core unit precursor comprising carboxylic acid groups. In the case of a core unit which is attached to 2 building units, the core unit of the dendrimer may for example be formed from a core unit precursor comprising two amino groups.

In some embodiments, the core unit is derivable from a precursor having three reactive nitrogen atoms, two of which may be used for attachment of building units, and one of which may be used for attachment of a spacer group. In some embodiments, the core unit is:

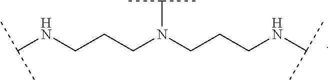

By use of a suitable protecting group strategy, the terminal nitrogens may be functionalised with different groups from the central nitrogen, e.g. building units may be attached to the terminal nitrogens, and the central nitrogen functionalised with a spacer group.

In some embodiments, the core unit is derivable from a precursor having two reactive nitrogen atoms, which may be used for attachments of building units. For example, in some embodiments the core unit may be derivable from ethylenediamine, 1,4-diaminobutane or 1,6-diaminohexane.

In some embodiments, the core unit is:

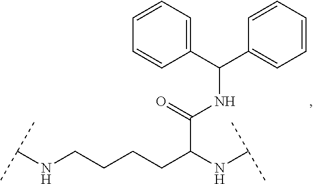

i.e. whereby the core unit comprises a lysine residue in which the acid moity has been capped with a benzyhydrylamine (BHA-Lys) to form the corresponding amide, and may, for example, be formed from a core unit precursor:

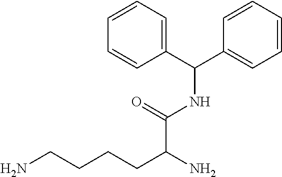 [SMILES: NCCCCC(N)C(=O)NC(c1ccccc1)c1ccccc1]

having two reactive (amino) nitrogens.

Where a core unit precursor with only two reactive nitrogen atoms is used, such as BHA-Lys, the two amino groups are typically functionalised with building units, and the spacer group is typically attached to a surface building unit.

The present dendrimers allow for multiple terminal groups, to be presented on the surface of the dendrimers in a controlled manner. In particular, where lysine building units are used, the placement on alpha or epsilon nitrogen atoms of the building units can be predetermined as described below. In some preferred embodiments, all of the complexation groups (radionuclide-containing moieties), pharmacokinetic groups, targeting agents and, where present, residues of pharmaceutically active agents, are provided on the surface of the dendrimer via attachment through the building units. In other words, in those embodiments, the core unit does not provide an attachment point for a terminal group other than via the building units. It will be understood that, in such embodiments, any functional groups present in the core unit which are not used for covalent attachment to a building unit will either be unreacted (i.e. unreactive in the conditions to which the conjugate has been exposed), or will have been capped with a suitable capping group to prevent further reaction. An example of such a core unit is the BHA-Lys group discussed above.

### Maleimide Core Unit

In some embodiments, the core is:

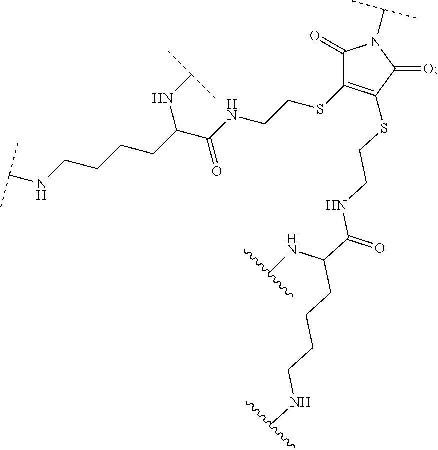

wherein the dotted lines adjacent bonds from the lysine nitrogen atoms indicate attachment of building units and that adjacent the bond from the maleimide nitrogen may indicate an attachment point of, for example, a functional moiety as discussed herein.

An advantage of utilising such a core is that the nature of each arm extending from the thioether bonds attached to the maleimide may be the same or different. This allows for increased flexibility in the synthesis of dendrimers, allowing for the tailoring of, for example, the dendrimer generations, type of building units, and terminal groups.

This may be achieved in a number of ways but, in one embodiment and as shown in Scheme 1, it may be desirable to start from a simple cystamine core on which the dendrimer is initially built via the conjugation of lysine residues, as shown. While the dendrimer may be taken to any desired number of generations with the cystamine core, two three, four or five generations may be most appropriate. As shown in Scheme 2, the cystamine dendrimer can be cleaved into two separate dendrons by reduction of the disulphide bond. This provides the dendrons with a thiol moiety for subsequent coupling to a maleimide unit.

This coupling step may also be achieved in a number of ways but the embodiment shown in Scheme 2 has been found to be useful wherein a dibromomaleimide reagent is provided. The nitrogen of the maleimide ring provides an opportunity for functionalisation with, for example, a functional moiety, spacer group or a precursor or component thereof as discussed below. In Scheme 2, this is shown as a PEG moiety activated with a tetrazine functionality, but it will be appreciated that a wide range of other spacer or linker groups could be used. The thiol groups of the dendrons are allowed to react with the dibromomaleimide unit and so a new dendrimer core is effectively formed. Scheme 2 shows that further iterations can then be added to the dendrimer via additional building units up to the desired generation. The spacer group on the maleimide nitrogen can be further functionalised or otherwise developed at the appropriate time to conjugate a targeting agent, therapeutic or other desired moiety.

It will be appreciated that when dendrons are being exposed to the dibromomaleimide unit it is possible to add a mixture of different dendrons and so have different dendrimer arms on the subsequently formed core. Separate batches of differing dendrons from cystamine cores can be synthesised and removed via reduction and then the dendrons mixed, in the chosen relative amounts, with the maleimide unit to afford the desired dendrimer with a maleimide core.

In one embodiment, the core unit may be or may comprise a methyl maleimide unit and so one carbon of the ring may have a methyl attached and so a double bond is present between ring carbons which may have benefits in stability.

In one embodiment the nitrogen of the maleimide ring may simply present a hydrogen. In one embodiment, the nitrogen of the maleimide ring may be attached to a functional moiety. When the nitrogen is attached to a functional moiety, the maleimide core essentially possesses a further ‘synthetic handle’ by which additional functionality may be introduced to the core. For example, a therapeutic agent, targeting agent, or pharmacokinetic modifying agent may be conjugated to the core as the functional moiety.

The functional moiety may be conjugated directly to the maleimide core. Alternatively, the functional moiety may be conjugated to the maleimide core via a spacer group, as defined herein. An advantage of conjugating the functional moiety to the maleimide core via a spacer group is that the functional moiety is tethered distally to the dendrimer, reducing any steric hindrance from the dendrimer that may otherwise reduce the ability of the functional moiety to exert its function (i.e., the ability of a therapeutic agent or targeting agent to interact with the target receptor or molecule).

In one embodiment, the nitrogen of the maleimide ring is attached to a functional moiety that comprises a targeting agent. The targeting agent may be conjugated to the maleimide core directly, or otherwise through any suitable spacer group, as described herein. In one example, R³ is a HER2 targeting agent conjugated to the maleimide core. In one example, R³ is a HER2 targeting agent conjugated to the maleimide core via a spacer group, as described herein. In one example, R³ is a FAP binding group conjugated to the maleimide core. In one example, R³ is a FAP binding group conjugated to the maleimide core via a spacer group, as described herein.

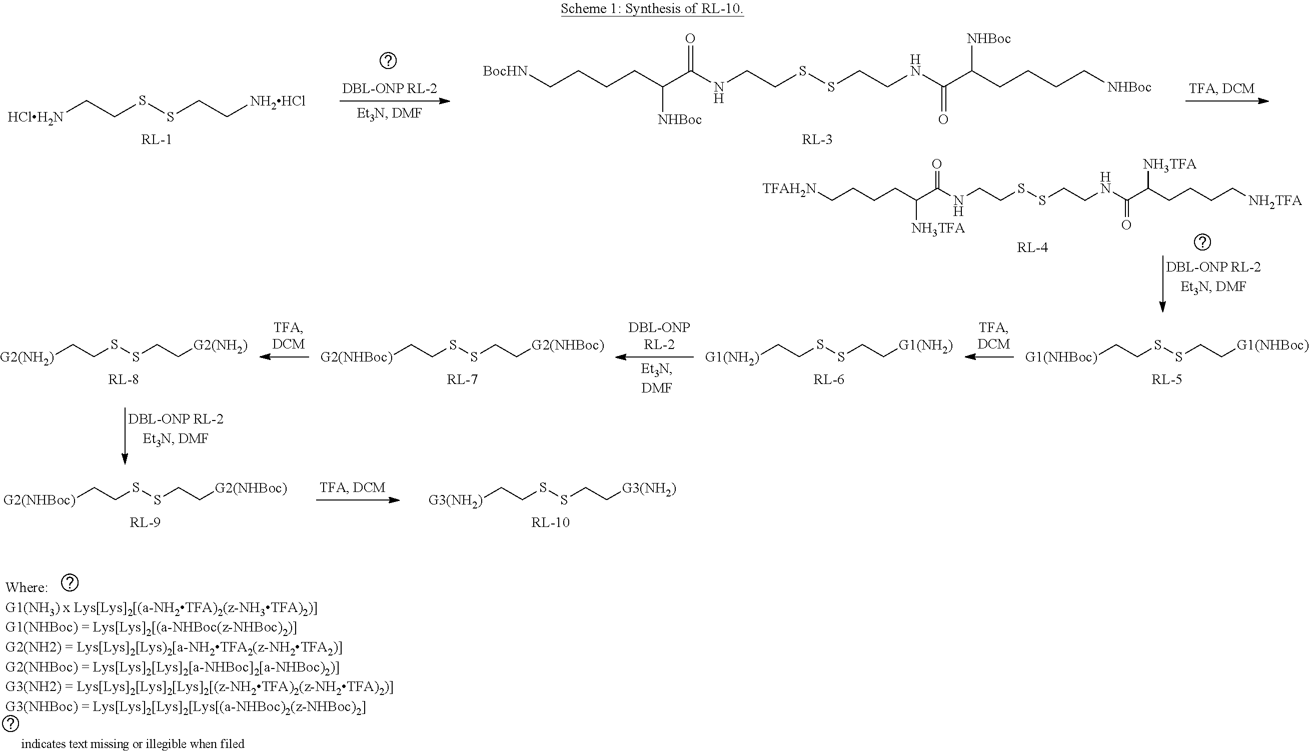

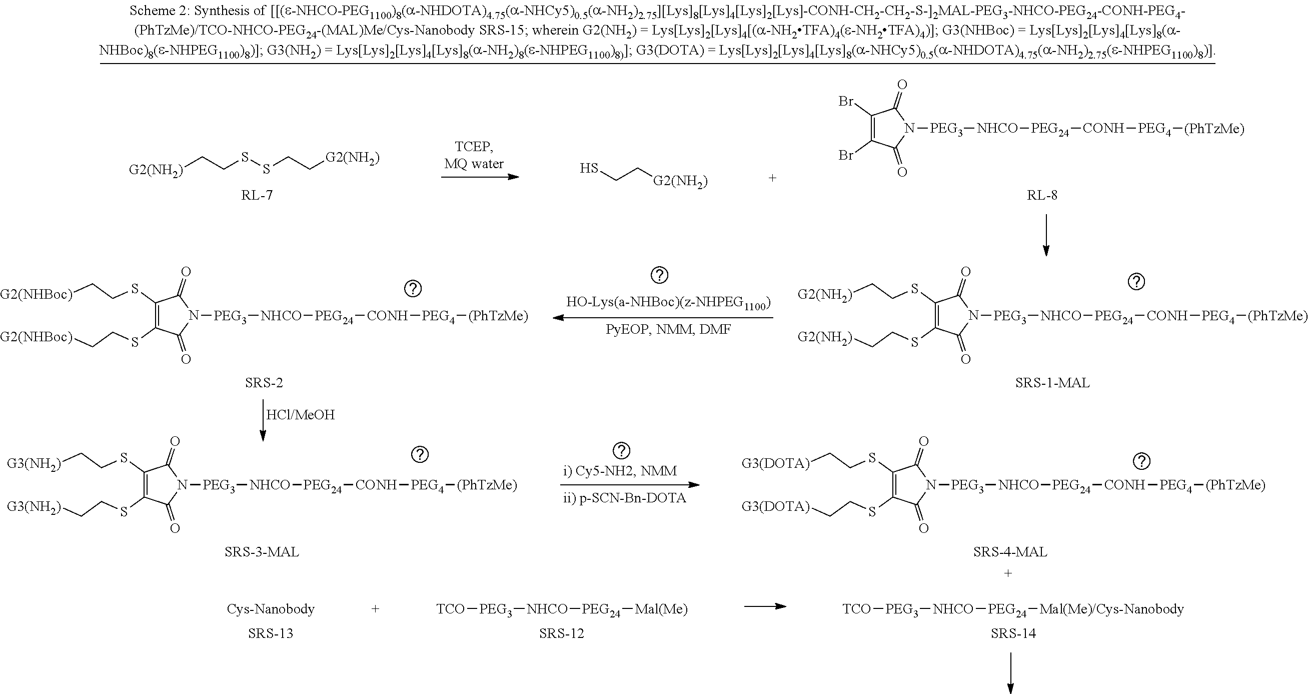

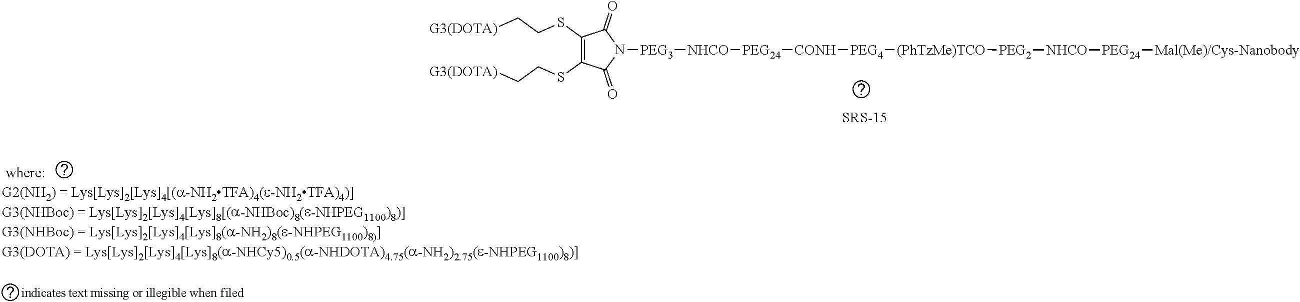

### Building Units

Any suitable building unit (BU) may be used to produce the dendrimers, as long as it contains a first functional group which is capable of forming a linkage with a functional group present on another building unit or a core unit, and contains at least two further functional groups which (e.g. following deprotection) are capable of forming a linkage with a functional group present on another building unit.

In some preferred embodiments, building units of different generations are covalently attached to one another via amide linkages formed between a nitrogen atom present in one building unit and the carbon atom of an acyl group present in another building unit. For example, in some embodiments, the building units are lysine residues or analogues thereof, and may be formed from suitable building unit precursors, e.g. lysine or lysine analogues containing appropriate protecting groups. Lysine analogues have two amino nitrogen atoms for bonding to a subsequent generation of building units and an acyl group for bonding to a previous generation of building units or a core. Examples of suitable building units include:

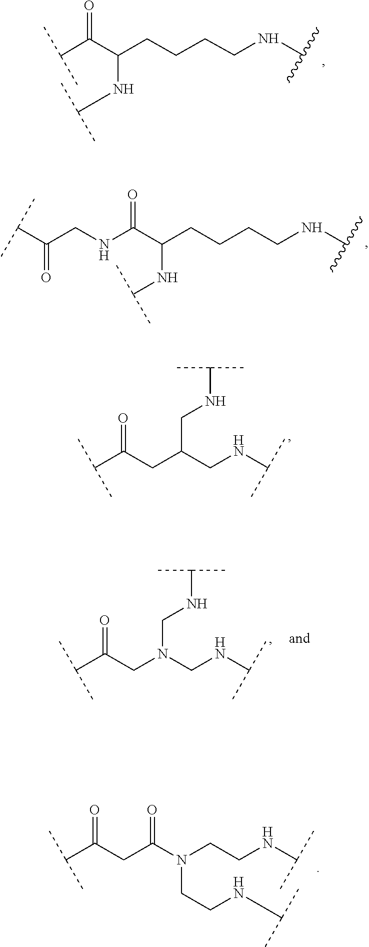

wherein the acyl group of each building unit provides a covalent attachment point for attachment to the core or to a previous generation building unit; and wherein each nitrogen atom provides a covalent attachment point which may be used for covalent attachment to a subsequent generation building unit, or to a terminal group.

In some preferred embodiments, the building units are each:

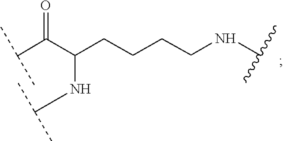

wherein the acyl group of each building unit provides a covalent attachment point for attachment to the core or to a previous generation building unit; and wherein each nitrogen atom provides a covalent attachment point which may be used for covalent attachment to a subsequent generation building unit, or to a terminal group.

In some preferred embodiments, the building units are each:

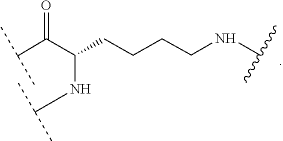

In other embodiments, the building units are aspartic acid residues, glutamic acid residues or analogues thereof, i.e. formed from suitable precursors e.g. aspartic acid, glutamic acid or analogues thereof, containing suitable protecting groups. In such embodiments, the core unit may be formed from a core unit precursor comprising carboxylic acid groups (i.e. which can react with amino groups present in the aspartic acid/glutamic acid/analogues.

The outermost generation of building units (BUₒᵤₜₑᵣ) may be formed by building units as used in the other generations of building units (BU) as described above, for example lysine or lysine analogue building units. The outermost generation of building units (BUₒᵤₜₑᵣ) is the generation of building units that is outermost from the core of the dendrimer, i.e., no further generations of building units are attached to the outermost generation of building units (BUₒᵤₜₑᵣ).

It will be appreciated that the dendrons of the dendrimer may for example be synthesised to the required number of generations through the attachment of building units (BU) accordingly. In some embodiments each generation of building units (BU) may be formed of the same building unit, for example all of the generations of building units may be lysine building units. In some other embodiments, one or more generations of building units may be formed of different building units to other generations of building units.

The dendrimer has from two to six generations of building units, i.e. 2, 3, 4, 5, or 6 generations of building units.

In some embodiments, the dendrimer has three generations of building units. A three generation building unit dendrimer is a dendrimer having a structure which includes three building units that are covalently linked to each other, for example in the case where the building units are lysines, it may comprise the substructure:

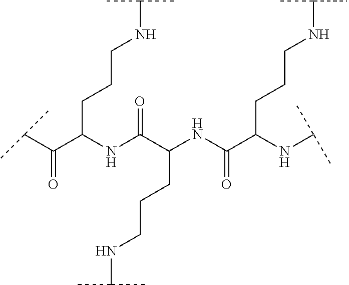

In some embodiments, the dendrimer has five generations of building units. A five generation building unit dendrimer is a dendrimer having a structure which includes five building units that are covalently linked to each another, for example in the case where the building units are lysines, it may comprise the substructure:

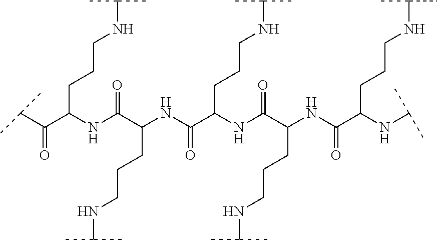

In some embodiments, the dendrimer has four generations of building units. A four generation building unit dendrimer is a dendrimer having a structure which includes four building units that are covalently linked to each another, for example in the case where the building units are lysines, it may comprise the substructure:

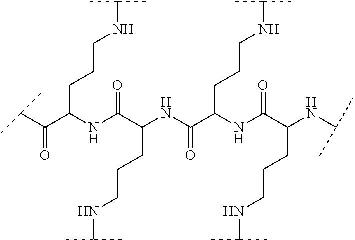

In some embodiments, the generations of building units are complete generations. For example, where the dendrimer has three generations of building units, in some embodiments the dendrimer has three complete generations of building units. With a core having two reactive amine groups, such a dendrimer will comprise 14 building units (i.e. core unit+2 BU+4 BU+8 BU).

Similarly, for example, where the dendrimer has four generations of building units, in some embodiments the dendrimer has four complete generations of building units. With a core having two reactive amine groups, such a dendrimer will comprise 30 building units (i.e. core unit+2 BU+4 BU+8 BU+16 BU).

Similarly, for example, where the dendrimer has five generations of building units, in some embodiments the dendrimer has five complete generations of building units. With a core having two reactive amine groups, such a dendrimer will comprise 62 building units (i.e. core unit+2 BU+4 BU+8 BU+16 BU+32 BU).

However, it will be appreciated that, due to the nature of the synthetic process for producing the dendrimers, one or more reactions carried out to produce the dendrimers may not go fully to completion. Accordingly, in some embodiments, the dendrimer may comprise incomplete generations of building units. For example, a population of dendrimers may be obtained, in which the dendrimers have a distribution of numbers of building units per dendrimer.

In some embodiments, where the dendrimer has three generations of building units, a population of dendrimers is obtained which has a mean number of building units per dendrimer of at least 8, or at least 9, or at least 10, or at least 11, or at least 12, or at least 13. In some embodiments, a population of dendrimers is obtained in which at least 60%, at least 70%, at least 80%, at least 90% or at least 95% of the dendrimers have 10 or more building units. In some embodiments, a population of dendrimers is obtained in which at least 60%, at least 70%, at least 80%, at least 90% or at least 95% of the dendrimers have 12 or more building units.

In some embodiments, where the dendrimer has four generations of building units, a population of dendrimers is obtained which has a mean number of building units per dendrimer of at least 25, or at least 26, or at least 27, or at least 28, or at least 29. In some embodiments, a population of dendrimers is obtained in which at least 60%, at least 70%, at least 80%, at least 90% or at least 95% of the dendrimers have 25 or more building units. In some embodiments, a population of dendrimers is obtained in which at least 60%, at least 70%, at least 80%, at least 90% or at least 95% of the dendrimers have 29 or more building units.

In some embodiments, where the dendrimer has five generations of building units, a population of dendrimers is obtained which has a mean number of building units per dendrimer of at least 55, or at least 56, or at least 57, or at least 58, or at least 59, or at least 60. In some embodiments, a population of dendrimers is obtained in which at least 60%, at least 70%, at least 80%, at least 90%, or at least 95% of the dendrimers have 55 or more building units. In some embodiments, a population of dendrimers is obtained in which at least 60%, at least 70%, at least 80%, at least 90%, or at least 95% of the dendrimers have 60 or more building units.

In some embodiments, each reactive (amino) group of the core unit precursor represents a conjugation site for a dendron comprising building units.

In some embodiments, each generation of building units in each dendron (X) may be represented by the formula [BU]₂^{(b-1)}, wherein b is the generation number. A dendron (X) having three complete generations of building units is represented as [BU]₁-[BU]₂-[BU]₄. A dendron (X) having four complete generations of building units is represented as [BU]₁-[BU]₂-[BU]₄-[BU]s. A dendron (X) having five complete generations of building units is represented as [BU]₁-[BU]₂-[BU]₄-[BU]₈-[BU]₁₆.

In some embodiments, the dendrimer comprises more than one dendron. In some embodiments, the dendrons are the same. In some embodiments, the dendrons are different. In some embodiments the dendrons are the same or different at the level of the building unit, the surface group, the generation size, the first terminal group or the second terminal group.

### First Terminal Group

The first terminal group (T1) comprises a complexation group for complexing a radionuclide. Following exposure to a suitable radionuclide, the complexation group for complexing a radionuclide then comprises a radionuclide and a complexation group and may be referred to as a radionuclide-containing moiety.

In embodiments, the radionuclide-containing moiety may comprise a radionuclide chelated with the complexation group.

In embodiments, the radionuclide-containing moiety may comprise a radionuclide in a coordination complex with the complexation group.

In embodiments, the radionuclide-containing moiety may comprise a radionuclide chelated to at least two different atoms of the complexation group.

In embodiments, the radionuclide-containing moiety may comprise a radionuclide datively bonded with the complexation group.

### Radionuclide

Any suitable radionuclide may be utilised in the present dendrimers. A radionuclide, also known as a radioactive isotope, is an unstable form of a chemical element that radioactively decays, resulting in the emission of nuclear radiation.

Radionuclides have application in treatment of diseases, such as cancers. In such cases, administration of a radionuclide-containing substance to a patient results in delivery of radionuclide to the tumour and, following radioactive decay and emission of radiation, killing of tumour cells.

Preferably, the radionuclide is a metal or a metalloid (for example, astatine is considered a metalloid for the present purposes) radionuclide, e.g. a metal ion or a metalloid ion. In some embodiments, the radionuclide is an alpha emitter (α-emitter). In some embodiments, the radionuclide is a beta emitter (β-emitter). In some embodiments, the radionuclide is a beta and gamma emitter (γ-emitter).

In embodiments, the radionuclide is not an isotope of hydrogen including deuterium and tritium.

In some embodiments, the radionuclide is an actinium (e.g. Ac²²⁵), astatine (e.g. As²¹¹) bismuth (e.g. Bi²¹², Bi²¹³), lead (e.g. Pb²¹²), technetium (e.g. Tc^{99m}), thorium (e.g. Th²²⁷), radium (e.g. Ra²²³), lutetium (e.g. Lu¹⁷⁷), yttrium (e.g. Y⁹⁰), indium (e.g. In¹¹¹, In¹¹⁴), gadolinium (e.g. Gd¹⁵³) gallium (e.g. Ga⁶⁸), zirconium (e.g. Zr⁸⁹), rhenium (e.g. Re¹⁸⁶), iodine (e.g. I¹³¹) or copper (e.g. Cu⁶⁰, Cu⁶¹, Cu⁶², Cu⁶⁴, Cu⁶⁷) radionuclide. In some embodiments, the radionuclide is a lutetium (e.g. Lu¹⁷⁷), gallium (e.g. Ga⁶⁸), zirconium (e.g. Zr⁸⁹), actinium (e.g. Ac²²⁵), bismuth (e.g. Bi²¹², Bi²¹³), astatine (e.g. As²¹¹), technetium (e.g. Tc^{99m}), or copper (e.g. Cu⁶⁰, Cu⁶¹, Cu⁶², Cu⁶⁴, Cu⁶⁷) radionuclide. In some embodiments, the radionuclide is a lutetium (e.g. Lu¹⁷⁷), gallium (e.g. Ga⁶⁸), zirconium (e.g. Zr⁸⁹), or copper (e.g. Cu⁶⁰, Cu⁶¹, Cu⁶², Cu⁶⁴ Cu⁶⁷) radionuclide. In some embodiments, the radionuclide is a gallium (e.g. Ga⁶⁸), zirconium (e.g. Zr⁸⁹), or lutetium (e.g. Lu¹⁷⁷) radionuclide.

In some embodiments, the radionuclide is for treatment of a condition (e.g. a cancer). Examples of such radionuclides include actinium (e.g. Ac²²⁵), astatine (e.g. As²¹¹), bismuth (e.g. Bi²¹², Bi²¹³), thorium (e.g. Th²²⁷), radium (e.g. Ra²²³), lutetium (e.g. Lu¹⁷⁷), yttrium (e.g. Y⁹⁰), gadolinium (e.g. Gd¹⁵³), lead (e.g. Pb²¹²) and copper (e.g. Cu⁶⁰, Cu⁶¹, Cu⁶², Cu⁶⁴)

In some embodiments, the radionuclide is an alpha emitter selected from actinium (e.g. Ac²²⁵), astatine (e.g. As²¹¹), bismuth (e.g. Bi²¹², Bi²¹³) and lead (e.g. Pb²¹²).

In some embodiments, the radionuclide is a beta-emitter selected from lutetium (e.g. Lu¹⁷⁷), yttrium (e.g. Y⁹⁰), iodine (e.g. I¹³¹), copper (e.g., Cu⁶⁷), and Rhenium (e.g. Re¹⁸⁶).

Ideally, the emission characteristics of a therapeutic radionuclide should take into consideration the lesion size to focus energy within the tumour, and have a suitable half-life to align with the extended delivery of the dendrimer. In some embodiments, the radionuclide is an alpha emitter with a half-life of less than 20 days or less than 12 days. In some embodiments, the radionuclide is a beta emitter with a half-life of 2 to 20 days or 5 to 10 days. ¹⁷⁷Lu is a medium-energy β-emitter (490 keV) with a maximum energy of 0.5 MeV and a maximal tissue penetration of <2 mm. ¹⁷⁷Lu also emits low-energy T-rays at 208 and 113 keV, which allows for ex vivo imaging and consequently the collection of information pertaining to tumour localisation and dosimetry. In some embodiments, injected doses of therapeutic radionuclide are from 1 to 50 GBq per single injection. In other embodiments, injected doses are from 2 to 20 GBq per single injection/infusion. In other embodiments, injected doses are from 2 to 10 GBq per single injection. Dose calculations for individual patients may be determined from a combination of disease burden, patient weight and renal function. Image-based dosimetry at each cycle of treatment is recommended, e.g., with SPECT-CT.

Radionuclides also find use in the field of medical diagnosis. Techniques such as single photon emission, positron emission tomography (PET) imaging, and positron emission tomography—magnetic resonance imaging (PET-MRI) can be used to detect a radionuclide within a subject administered a suitable radionuclide-containing substance, and produce images, which inform as to the existence and/or progression of diseases such as tumours.

In some embodiments, the radionuclide is for diagnosis or imaging of a condition (e.g. a cancer). Examples of such radionuclides include gallium (e.g. Ga⁶⁸), indium (e.g. In¹¹¹,), zirconium (e.g. Zr⁸⁹), Iodine (eg ¹²³I, ¹³¹I), technetium (e.g. Tc^{99m}m, yttrium (e.g. Y⁸⁶), fluorine, (e.g. F¹⁸), and copper (e.g. Cu⁶⁰, Cu⁶¹, Cu⁶², Cu⁶⁴). In some embodiments, the radionuclide is an imaging agent selected from gallium (e.g. Ga⁶⁸), technetium (e.g. Tc^{99m}), zirconium (e.g. Zr⁸⁹), and copper (e.g. Cu⁶⁰, Cu⁶¹, Cu⁶², Cu⁶⁴). In some embodiments the radionuclide is selected from gallium (e.g. Ga⁶⁸), zirconium (e.g. Zr⁸⁹), and copper (e.g. Cu⁶⁴)

In some embodiments, the radionuclide is not gadolinium.

In some embodiments, the radionuclide is not a paramagnetic agent.

The type of radionuclide used may be tailored to the dendrimeric structure in order to optimise the level of radioactive exposure received by a subject. For example, it is considered that the body will typically have greater exposure to dendrimers having a greater number of generations of building units. Thus, by pairing such dendrimers with radionuclides having an appropriate half-life, optimal therapeutic and/or diagnostic activity can be achieved whilst avoiding or reducing side-effects associated with exposure of the body to radioactivity.

In some embodiments, the dendrimer is a 4- or 5-generation dendrimer and the radionuclide has a half-life of no more than 10 days, preferably less than 5 days. In some embodiments, the dendrimer is a 4-generation dendrimer and the radionuclide has a half-life of no more than 10 days, preferably less than 5 days. In some embodiments, the dendrimer is a 5-generation dendrimer and the radionuclide has a half-life of less than 10 days, preferably less than 5 days. In some embodiments, the dendrimer is a 4-generation dendrimer and the radionuclide is selected from the group consisting of Y⁹⁰, Tc^{99m}, Th²⁰¹, Rb⁸², Lt¹⁷⁷, Ga⁶⁷, Ga⁶⁸, and In¹¹¹. In some embodiments, the dendrimer is a 3-generation dendrimer and the radionuclide has a half-life of no more than 10 days. In some embodiments, the dendrimer is a 3-generation dendrimer and the radionuclide is selected from the group consisting of Y⁹⁰, Tc^{99m}, Th²⁰¹, Rb⁸², Lt¹⁷⁷, Ga⁶⁷, Ga⁶⁸, Ac²²⁵ and In¹¹¹.

### Radionuclide Complexation Group

Any suitable complexation group may be used so long as it is appropriate to form a complex with the desired radionuclide. The complexation group for complexing a radionuclide provides functional moieties that can complex a radionuclide. Examples of such functional moieties include carboxylic acids, amines, amides, hydroxyl groups, thiol groups, ureas, thioureas, —N—OH groups, phosphate, and phosphinate groups forming a complex with the radionuclide.

In some embodiments, the complexation group is complexed directly to the radionuclide.

In some embodiments, the complexation group is complexed directly to the radionuclide to form a coordination complex. That is, the complexation group is coordinately bonded to the radionuclide to form the complex.

In some embodiments, the complexation group forms only dative covalent bonds to the radionuclide. For example, the complexation group may form at least two separate dative covalent bonds with the radionuclide.

In some embodiments, a complexation group that forms a chelate with the radionuclide is used. As used herein, the phrase “a complexation group that forms a chelate with the radionuclide” means that the complexation group forms at least two separate bonds (i.e. a bond from at least two different atoms of the complexation group) to the radionuclide.

Examples of suitable complexation groups are provided in the table below:

| Ligand | Structure | Chemical Name |
|---|---|---|
| DOTA | 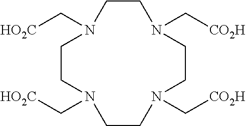 [SMILES: O=C(O)CN1CCN(CC(=O)O)CCN(CC(=O)O)CCN(CC(=O)O)CC1] | 1,4,7,10-Tetraazacyclodecane- N,N′,N″,N′′′-tetraacetic acid |
| NOTA | 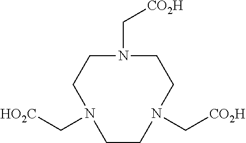 [SMILES: O=C(O)CN1CCN(CC(=O)O)CCN(CC(=O)O)CC1] | 1,4,7-Triazacyclononane- N,N′,N″-triacetic acid |
| DTPA | 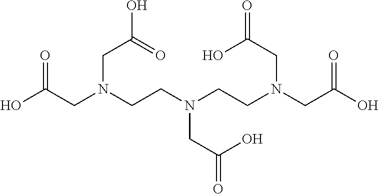 [SMILES: O=C(O)CN(CCN(CC(=O)O)CC(=O)O)CCN(CC(=O)O)CC(=O)O] | Diethylenetriaminepentaacetic anhydride |
| CHX-A DTPA | 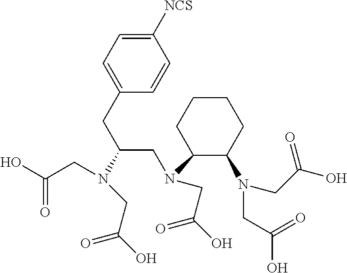 | [(R)-2-Amino-3-(4- isothiocyanatophenyl)propyl]- trans-(S,S)- cyclohexane-1,2-diamine- pentaacetic acid |
| Deferoxamine (DFO) | 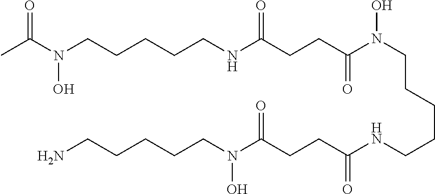 [SMILES: CC(=O)N(O)CCCCCNC(=O)CCC(=O)N(O)CCCCCNC(=O)CCC(=O)N(O)CCCCCN] | N′-[5-[[4-[[5- (Acetylhydroxyamino) pentyl]amino]-1,4-dioxobutyl] hydroxyamino]pentyl]-N-(5- aminopentyl)-N-hydroxy- butanediamide |
| TETA | 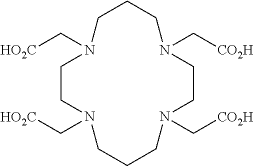 [SMILES: O=C(O)CN1CCCN(CC(=O)O)CCN(CC(=O)O)CCCN(CC(=O)O)CC1] | 1,4,8,11- tetraazacyclotetradecane-1,4,8,11- tetraacetic acid |
| cb-TE2A | 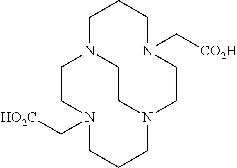 [SMILES: O=C(O)CN1CCCN2CCN(CCCN(CC(=O)O)CC2)CC1] | 1,4,8,11- Tetraazabicyclo[6.6.2] hexadecane-4,11- diacetic acid |
| AAZTA | 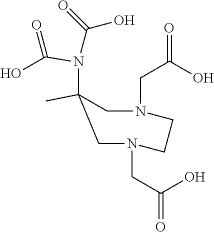 [SMILES: CC1(N(C(=O)O)C(=O)O)CN(CC(=O)O)CCN(CC(=O)O)C1] | 1,4-Bis(carboxymethyl)-6- [bis(carboxymethyl)]amino-6- methylperhydro-1,4-diazepine) |
| TRAP | 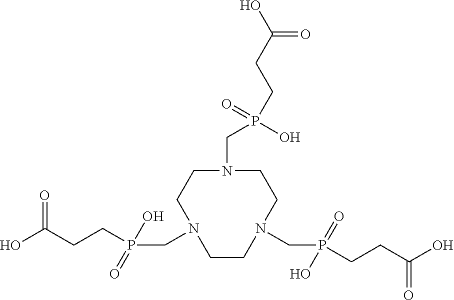 [SMILES: O=C(O)CCP(=O)(O)CN1CCN(CP(=O)(O)CCC(=O)O)CCN(CP(=O)(O)CCC(=O)O)CC1] | 1,4,7-Triazacyclononane phosphinic acid |
| NOPO | 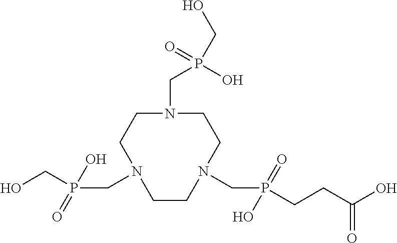 [SMILES: O=C(O)CCP(=O)(O)CN1CCN(CP(=O)(O)CO)CCN(CP(=O)(O)CO)CC1] | 1,4,7-Triazacyclononane-1,4- bis[methylene (hydroxymethyl)phosphonic acid]-7-[methylene(2- carboxyethyl)phosphonic acid] |
| HOPO | 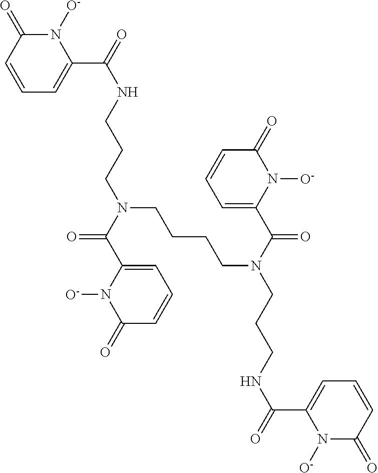 [SMILES: O=C(NCCCN(CCCCN(CCCNC(=O)c1cccc(=O)n1[O-])C(=O)c1cccc(=O)n1[O-])C(=O)c1cccc(=O)n1[O-])c1cccc(=O)n1[O-]] | 6-[(3-{[(1-Oxido-6- oxo-1,6-dihydro-2- pyridinyl)carbonyl] amino}propyl)(4-{[(1- oxido-6-oxo-1,6-dihydro- 2-pyridinyl)carbonyl] (3-{[(1-oxido-6-oxo- 1,6-dihydro-2- pyridinyl)carbonyl]amino} propyl)amino}butyl) carbamoyl]-2-oxo- 1(2H)-pyridinolate |
| NOGADA | 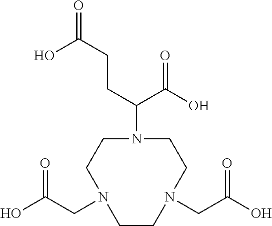 [SMILES: O=C(O)CCC(C(=O)O)N1CCN(CC(=O)O)CCN(CC(=O)O)CC1] | 2-[4,7-Bis(carboxymethyl)- 1,4,7-triazonan- 1-yl]pentanedioic acid |
| HYNIC | 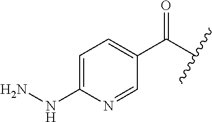 [SMILES: *C(=O)c1ccc(NN)nc1] | 6-Hydrazinonicotinic acid |
| MAG3 | 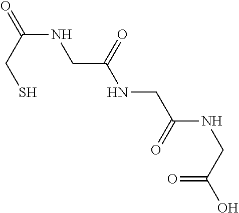 [SMILES: O=C(O)CNC(=O)CNC(=O)CNC(=O)CS] | Mertiatide |
| OPTT | 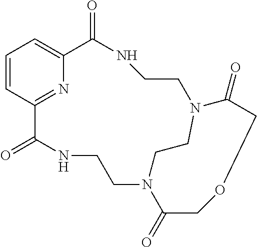 [SMILES: O=C1NCCN2CCN(CCNC(=O)c3cccc1n3)C(=O)COCC2=O] | 9-Oxa-3,6,12,15,21- pentaazatricyclo [15,3,2,1]trieicos- 1(21),17,19-triene- 2,7,11,16-tetradione |
| TBPD | 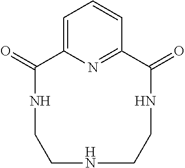 [SMILES: O=C1NCCNCCNC(=O)c2cccc1n2] | 3,6,9,15-Tetraazabicyclo [9.3.1]pentadeca- 1(15),11,13-triene- 2,10-dione |
| TAME-Hex | 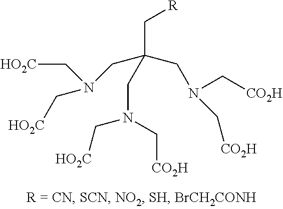 | 1,1,1-Tris(aminomethyl)ethane (basic skeleton) |
| DO3A | 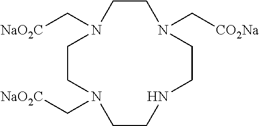 | 1,4,7,10- Tetraazacyclododecane-1,4,7- triacetic acid trisodium salt |
| TRAP | 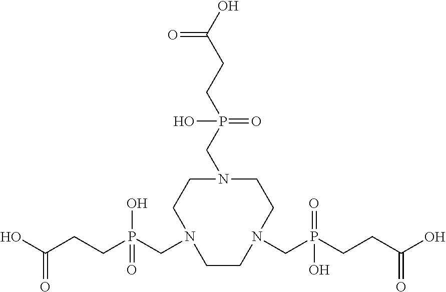 [SMILES: O=C(O)CCP(=O)(O)CN1CCN(CP(=O)(O)CCC(=O)O)CCN(CP(=O)(O)CCC(=O)O)CC1] | 1,4,7-Triazacyclononane- 1,4,7-tris[(2- carboxyethyl) methylenephosphinic acid] |
| DATA | 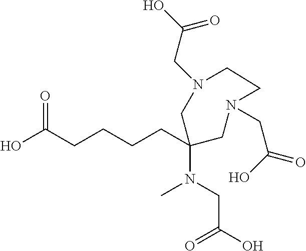 [SMILES: CN(CC(=O)O)C1(CCCCC(=O)O)CN(CC(=O)O)CCN(CC(=O)O)C1] | 2,2′-(6-((Carboxymethyl) amino)-1,4- diazepane-1,4-diyl)diacetic acid) |
| NODAGA | 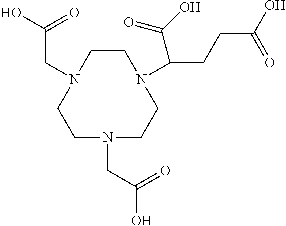 [SMILES: O=C(O)CCC(C(=O)O)N1CCN(CC(=O)O)CCN(CC(=O)O)CC1] | 2-[4,7-Bis(carboxymethyl)- 1,4,7-triazonan- 1-yl]pentanedioic acid |
| DOTAGA | 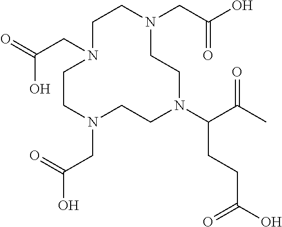 [SMILES: CC(=O)C(CCC(=O)O)N1CCN(CC(=O)O)CCN(CC(=O)O)CCN(CC(=O)O)CC1] | 1,4,7,10- Tetraazacyclododecane, 1- (glutaric acid)- 4,7,10-triacetic acid |
| PhenA | 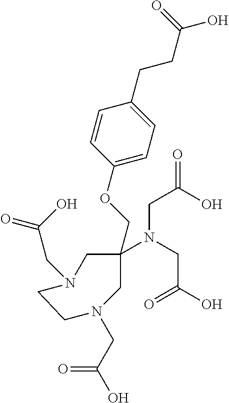 [SMILES: O=C(O)CCc1ccc(OCC2(N(CC(=O)O)CC(=O)O)CN(CC(=O)O)CCN(CC(=O)O)C2)cc1] | 2,2′-(6- (Bis(carboxymethyl)amino)-6-((4- (2-carboxyethyl)phenoxy)methyl)-1,4- diazepane-1,4-diyl)diacetic acid |
| PCB-TE1A1P | 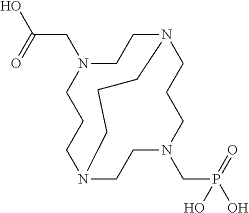 [SMILES: O=C(O)CN1CCCN2CCCN(CCCN(CP(=O)(O)O)CC2)CC1] | 11-Carboxymethyl-1,4,8,11- tetraazabicyclo[6.6.2]hexadecane-4- methanephosphonic acid |
| H3THP | 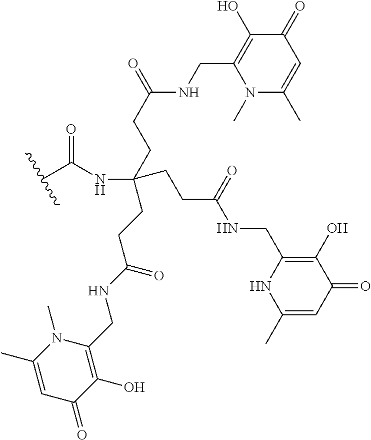 [SMILES: *C(=O)NC(CCC(=O)NCc1[nH]c(C)cc(=O)c1O)(CCC(=O)NCc1c(O)c(=O)cc(C)n1C)CCC(=O)NCc1c(O)c(=O)cc(C)n1C] | Tris(6-hydroxypyridin- 2-ylmethyl)amine |
| DOTA-4AMP | 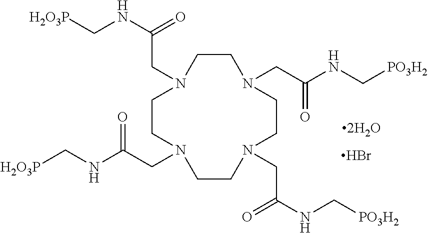 | 1,4,7,10- Tetraazacyclododecane-1,4,7,10- tetrakis(acetamido- methylenephosphonic acid) |
| NOTP | 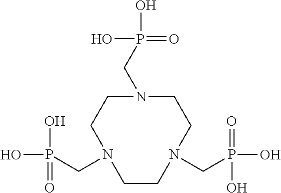 [SMILES: O=P(O)(O)CN1CCN(CP(=O)(O)O)CCN(CP(=O)(O)O)CC1] | 1,4,7-Triazacyclononane-1,4,7- tri(methylene phosphonic acid) |
| CB-Cyclam | 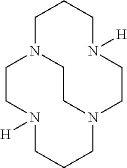 [SMILES: C1CNCCN2CCCNCCN(C1)CC2] | 1,4,8,11- Tetraazabicylco[6.6.2]hexadecan |
| DiAmSar | 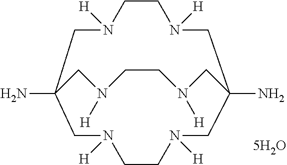 [SMILES: NC12CNCCNCC(N)(CNCCNC1)CNCCNC2] | 1,8-Diamino-3,6,10,13,16,19- hexaazabicyclo[6,6,6]-eicosane |
| DOTMA | 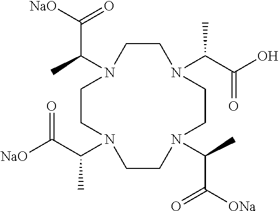 | (1R,4R,7R,10R)- α,α′,α″, α′′′,Tetramethyl- 1,4,7,10- tetraazacyclododecane-1,4,7,10- tetraacetic acid tetrasodium salt |
| DOTP | 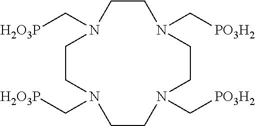 | 1,4,7,10- Tetraazacyclododecane-1,4,7,10- tetra(methylene phosphonic acid) |
| HBED | 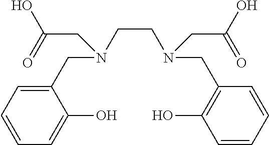 [SMILES: O=C(O)CN(CCN(CC(=O)O)Cc1ccccc1O)Cc1ccccc1O] | 2,2′-{1,2-Ethanediylbis[(2- hydroxybenzyl)imino]}diacetic acid |
| 6SS | 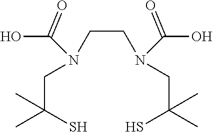 [SMILES: CC(C)(S)CN(CCN(CC(C)(C)S)C(=O)O)C(=O)O] | N,N′-Bis(2,2- dimethyl-2-mercaptoethyl) ethylenediamine-N,N′-diacetic acid |
| SarAr | 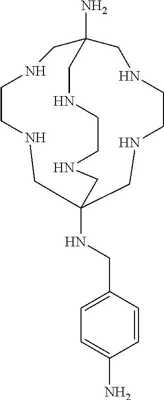 [SMILES: Nc1ccc(CNC23CNCCNCC(N)(CNCCNC2)CNCCNC3)cc1] | 1-N-(4-Aminobenzyl)- 3,6,10,13,16,19- hexaazabicyclo[6.6.6]-eicosane-1,8- diamine |
| MeCOSar | 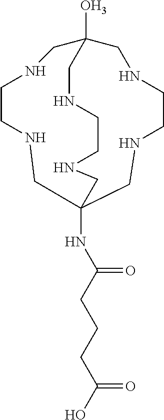 [SMILES: O=C(O)CCCC(=O)NC12CNCCNCC(O)(CNCCNC1)CNCCNC2] | 5-(8-Methyl- 3,6,10,13,16,19-hexaaza- bicyclo[6.6.6]icosan-1-ylamino)-5- oxopentanoic acid |
| Sar (sarcophagine) | 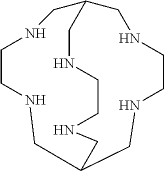 [SMILES: C1CNCC2CNCCNCC(CN1)CNCCNC2] | 3,6,10,13,16,19- Hexaazabicyclo(6,6,6)icosane |
| H₂KTSM | 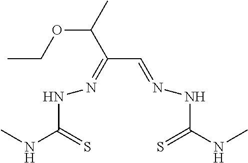 | 3-Ethoxy-2- oxobutyraldehyde-bis(N⁴- methylthiosemicarbazone) |
| H₂ATSM | 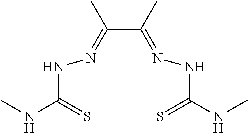 | Diacetyl-2-(N⁴-methyl-3- thiosemicarbazone)-3-(N⁴- amino-3-thiosemicarbazone) |
| TCMC | 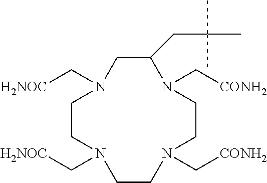 | 1,4,7,10-Tetraaza-1,4,7,10-tetra(2- carbamoylmethyl)cyclododecane |
| MACROPA | 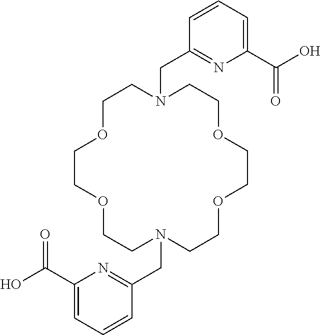 [SMILES: O=C(O)c1cccc(CN2CCOCCOCCN(Cc3cccc(C(=O)O)n3)CCOCCOCC2)n1] | 6-[[16-[(6- Carboxypyridin-2-yl)methyl]- 1,4,10,13-tetraoxa-7,16- diazacyclooctadec-7-yl]methyl]-4- isothiocyanatopyridine- 2-carboxylic acid |
| EDTA | 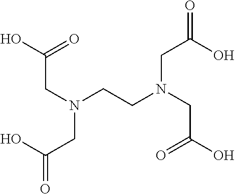 [SMILES: O=C(O)CN(CCN(CC(=O)O)CC(=O)O)CC(=O)O] | Ethylenediaminetetraacetic acid |
| PEPA | 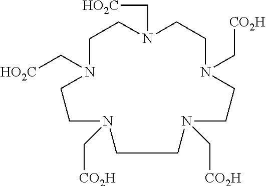 [SMILES: O=C(O)CN1CCN(CC(=O)O)CCN(CC(=O)O)CCN(CC(=O)O)CCN(CC(=O)O)CC1] | 1,4,7,10,13- Pentaazacyclopentadecane-N, N′, N″, N′′′, N″-pentaacetic acid |

In some embodiments, the complexation group is DOTA, NOTA, DTPA, sarcophagine, or DFO.

In some embodiments, the complexation group is a macropa group. Macropa groups are particularly suitable for use with Ac²²⁵ radionuclides, for example. In some embodiments, the complexation group is a macropa group and the radionuclide is Ac²²⁵.

In some embodiments, the complexation group is an EDTA or PEPA group.

The first terminal group is attached to an outermost building unit, e.g. via a nitrogen atom of an outermost building unit where the building units are lysine residues or analogues thereof. In some embodiments, where a complexation group comprises a group that is suitable for direct reaction with an outermost building unit, the complexation group may be reacted directly with the building unit. In other embodiments, a loading group may be utilised to load the complexation group on to the dendrimer, i.e. a group which at a first end is covalently attached to the complexation group, and which at a second end has a functional group suitable for reaction with a functional group present on an outermost building unit (e.g. where the first terminal group is attached via a nitrogen atom of an outermost building unit. For example, the loading group may have a functional group that is suitable for reaction with an amino group.

To form the attachment between the outermost building unit and the first terminal group, a reaction may be carried out between a suitable complexation precursor groups and a dendrimeric intermediate having functional groups (e.g. amine groups) available for reaction. In some embodiments, the complexation precursor is a DOTA-containing, NOTA-containing, DTPA-containing, sarcophagine-containing or DFO-containing group.

In some embodiments, the complexation group is a DOTA-containing group having the structure

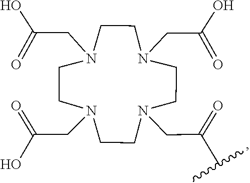

and wherein the DOTA-containing group is attached to the conjugate.

In some embodiments, the complexation group is a NOTA-containing group having the structure

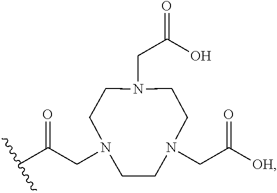 [SMILES: *C(=O)CN1CCN(CC(=O)O)CCN(CC(=O)O)CC1]

and wherein the NOTA-containing group is attached to the conjugate.

In some embodiments, the complexation group is a DTPA-containing group having the structure

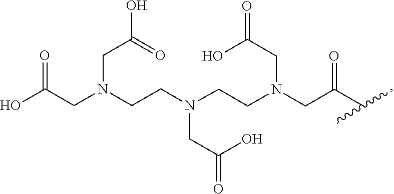

wherein the DTPA-containing group is attached to the conjugate.

In some embodiments the complexation group is a DFO-containing group having the structure

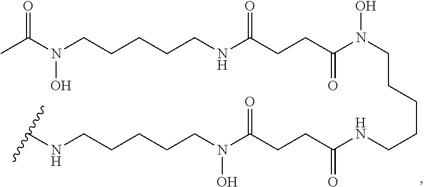 [SMILES: *NCCCCCN(O)C(=O)CCC(=O)NCCCCCN(O)C(=O)CCC(=O)NCCCCCN(O)C(C)=O]

wherein the DFO-containing group is attached to the conjugate.

In some embodiments, the complexation group is a sarcophagine-containing group having the structure

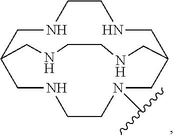 [SMILES: *N1CCNCC2CNCCNCC(CNCCNC2)C1]

wherein the sarcophagine-containing group is attached to the conjugate.

In some embodiments, the complexation group is a sarcophagine-containing group having the structure

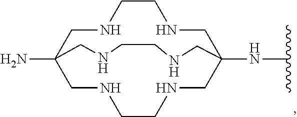 [SMILES: *NC12CNCCNCC(N)(CNCCNC1)CNCCNC2]

wherein the sarcophagine-containing group is attached to the conjugate.

In some embodiments, the complexation group is a macropa-containing group having the structure

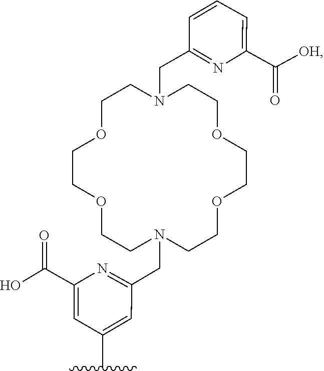 [SMILES: *c1cc(CN2CCOCCOCCN(Cc3cccc(C(=O)O)n3)CCOCCOCC2)nc(C(=O)O)c1]

wherein the macropa-containing group is attached to the conjugate.

Specific examples of suitable complexation precursor groups include the following:

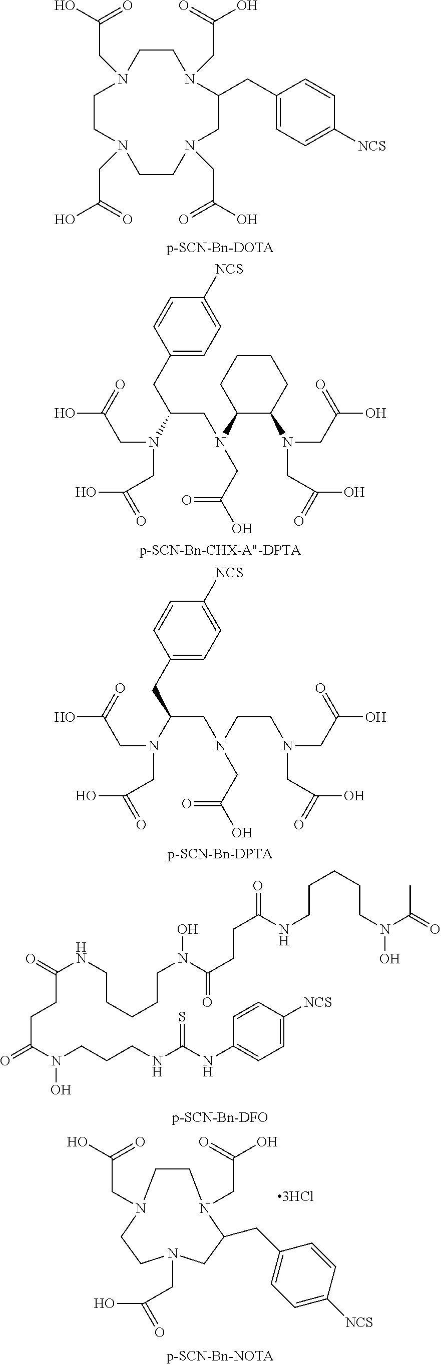

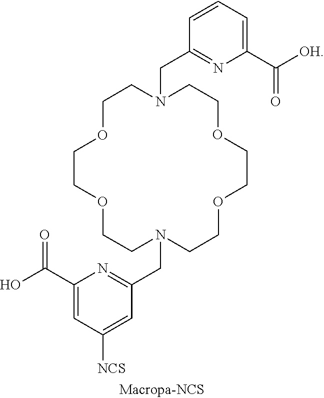

The above such groups can react with an amine group present on an outermost building unit to form a thiourea-linked first terminal group.

### Second Terminal Group

The conjugate comprises a plurality of second terminal groups (T2) each comprising a pharmacokinetic-modifying moiety, i.e. a moiety that can modify or modulate the pharmacokinetic profile of the conjugate. The pharmacokinetic modifying moiety may modulate the absorption, distribution, metabolism, excretion and/or toxicity of the dendrimer. The pharmacokinetic modifying moiety (T2) may change the solubility profile of the dendrimer, either increasing or decreasing the solubility of the dendrimer in a pharmaceutically acceptable carrier. The pharmacokinetic modifying moiety (T2) may for example reduce clearance of the dendrimer.

Where the dendrimer comprises a third terminal group comprising a pharmaceutically active agent, the pharmacokinetic modifying moiety (T2) may influence the rate of release of the pharmaceutically active agent, either by slowing or increasing the rate in which the active agent is released from the dendrimer by either chemical (e.g., hydrolysis) or enzymatic degradation pathways. The pharmacokinetic modifying moiety (T2) may assist the dendrimer in delivering the pharmaceutically active agent to specific tissues (e.g. tumours).

The pharmacokinetic-modifying moiety may for example be an oligomeric or polymeric group, e.g. which is biocompatible, water-soluble. In some embodiments, the pharmacokinetic-modifying moiety is a water-soluble oligomer or polymer having a molecular weight in the range of from 300 to 5000 Daltons.

In some preferred embodiments, the pharmacokinetic-modifying moiety is a polyethylene glycol (PEG) group, or a polyethyloxazoline (PEOX) group, or a poly-(2) methyl-(2)-oxazolamine (POZ), or a polysarcosine (poly (n-methylated glycine)), or a poly(2-hydroxypropyl)methacrylamide (pHPMA) group.

In some embodiments, the second terminal group comprises a PEG group. A PEG group is a polyethylene glycol group, i.e. a group comprising repeat units of the formula —CH₂CH₂O—. PEG materials used to produce the dendrimer of the present disclosure typically contain a mixture of PEGs having some variance in molecular weight (i.e., ±10%), and therefore, where a molecular weight is specified, it is typically an approximation of the average molecular weight of the PEG composition. For example, the term “PEG_{˜2100}” refers to polyethylene glycol having an average molecular weight of approximately 2100 Daltons, i.e. ±approximately 10% (PEG₁₈₉₀ to PEG₂₃₁₀). The term “PEG_{˜2300}” refers to polyethylene glycol having an average molecular weight of approximately 2300 Daltons, i.e. ±approximately 10% (PEG₂₀₇₀ to PEG₂₅₃₀). Three methods are commonly used to calculate MW averages: number average, weight average, and z-average molecular weights. As used herein, the phrase “molecular weight” is intended to refer to the weight-average molecular weight which can be measured using techniques well-known in the art including, but not limited to, NMR, mass spectrometry, matrix-assisted laser desorption ionization time of flight (MALDI-TOF), gel permeation chromatography or other liquid chromatography techniques, light scattering techniques, ultracentrifugation and viscometry.

In some embodiments, the second terminal groups comprise PEG groups having an average molecular weight of between about 200 and 5000 Daltons, or from 200 to 4000 Daltons, or from 300 to 3000 Daltons, or from 300 to 2000 Daltons, or from 400 to 1500 Daltons, or from 400 to 1200 Daltons, or from 400 to 1000 Daltons, or from 400 to 800 Daltons, or from 400 to 600 Daltons. In some embodiments, the second terminal groups comprise PEG groups having an average molecular weight of about 400, about 450, about 500, about 550, about 600, about 700, about 800, about 900, about 1000, about 1100, about 1200, about 1300, about 1400 or about 1500 Daltons. In some embodiments, the second terminal groups comprise PEG groups having an average molecular weight of about 470 Daltons. In some embodiments, the second terminal groups comprise PEG groups having an average molecular weight in the range of from 500 to 3000 Daltons, or from 1500 to 2500 Daltons. In some embodiments, the second terminal groups comprise PEG groups having an average molecular weight in the range of from 220 to 2500 Daltons, or from 570 to 2500 Daltons, or from 220 to 1100 Daltons, or from 570 to 1100 Daltons, or from 1000 to 5500 Daltons, or from 1000 to 2500 Daltons, or from 1000 to 2300 Daltons. In some embodiments, the second terminal groups comprise PEG groups having an average molecular weight in the range of from 1900 to 2300 Daltons. In some embodiments, the second terminal groups comprise PEG groups having an average molecular weight in the range of from 2100 to 2500 Daltons. In some embodiments, the second terminal groups comprise PEG groups having an average molecular weight in the range of from 2400 to 2800 Daltons. In some embodiments, the second terminal groups comprise PEG groups having an average molecular weight of about 1900, about 2000, about 2100, about 2200, about 2300, about 2400, about 2500, about 2600, about 2700 or about 2800 Daltons.

In some embodiments, the PEG group has a polydispersity index (PDI) of between about 1.00 and about 1.50, between about 1.00 and about 1.25, or between about 1.00 and about 1.10. In some embodiments, the PEG group has a polydispersity index (PDI) of about 1.05. The term “polydispersity index” refers to a measure of the distribution of molecular mass in a given polymer sample. The polydispersity index (PDI) is equal to the weight average molecular weight (Mw) divided by the number average molecular weight (M) and indicates the distribution of individual molecular masses in a batch of polymers. The polydispersity index (PDI) has a value equal to or greater than one, but as the polymer approaches uniform change length and average molecular weight, the polydispersity index (PDI) will be closer to one.

Where the second terminal groups comprise a PEG group, the PEG groups may be linear or branched. If desired, an end-capped PEG group may be used. In some embodiments, the PEG group is a methoxy-terminated PEG. In some embodiments, the second terminal group comprises a polysarcosine group, i.e. a group comprising repeat units of the formula

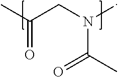

In some embodiments, the second terminal groups comprise polysarcosine groups having an average molecular weight of at least 750 Daltons, at least 1000 Daltons, or at least 1500 Daltons. In some embodiments, the second terminal groups comprise polysarcosine groups having an average molecular weight in the range of from 750 Daltons to 2500 Daltons, or from 1000 Daltons to 2500 Daltons.

In some embodiments, the second terminal group comprises a PEOX group. A PEOX group is a polyethyloxazoline group, i.e. a group comprising repeat units of the formula

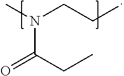

PEOX groups are so named since they can be produced by polymerisation of ethyloxazoline. PEOX materials used to produce the dendrimer of the present disclosure typically contain a mixture of PEOXs having some variance in molecular weight (i.e., +10%), and therefore, where a molecular weight is specified, it is typically an approximation of the average molecular weight of the PEOX composition. In some embodiments, the second terminal groups comprise PEOX groups having an average molecular weight of at least 750 Daltons, at least 1000 Daltons, or at least 1500 Daltons. In some embodiments, the second terminal groups comprise PEOX groups having an average molecular weight in the range of from 750 Daltons to 2500 Daltons, or from 1000 Daltons to 2000 Daltons. If desired, an end-capped PEOX group may be used. In some embodiments, the PEOX group is a methoxy-terminated PEOX.

In some embodiments, the second terminal group comprises a poly-(2) methyl-(2)-oxazolamine (POZ) group.

In some embodiments, the second terminal group comprises a poly(2-hydroxypropyl)methacrylamide (pHPMA) group.

The second terminal group may be attached to the outermost building unit via any suitable means. In some embodiments, where the second terminal group comprises a PEG group, PEOX group, POZ group, or pHPMA group, a linking group is used to attach the PEG group, PEOX group, POZ group, or pHPMA group to the outer building unit.

The second terminal groups are typically attached via use of a second terminal group precursor which contains a reactive group that is reactive with an amine group, such as a reactive acyl group (which can form an amide bond), or an aldehyde (which can form an amine group under reductive amination conditions).

In some embodiments, the second terminal groups each comprise a PEG group covalently attached to a PEG linking group (L1) via an ether linkage formed between a carbon atom present in the PEG group and an oxygen atom present in the PEG linking group, and each second terminal group is covalently attached to a building unit via an amide linkage formed between a nitrogen atom present in a building unit and the carbon atom of an acyl group present in the PEG linking group. In some embodiments, the second terminal groups are each

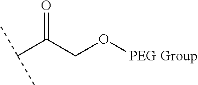

and wherein the PEG group is a methoxy-terminated PEG having an average molecular weight in the range of from about 500 to 3000 Daltons, or from 2000 to 2700 Daltons.

In some embodiments, the second terminal groups each comprise a PEOX group covalently attached to a PEOX linking group (L1′) via a linkage formed between a nitrogen atom present in the PEOX group and a carbon atom present in the PEOX linking group, and each second terminal group is covalently attached to a building unit via an amide linkage formed between a nitrogen atom present in a building unit and the carbon atom of an acyl group present in the PEOX linking group. In some embodiments, the second terminal groups are each

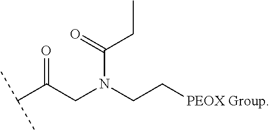

In some embodiments, the second terminal groups are each polysarcosine groups, e.g. of the formula:

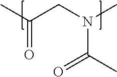

and are attached to a building unit via an amide linkage formed between a nitrogen atom present in a building unit and the carbon atom of an acyl group present in the polysarcosine group.

### Targeting Agent

The dendrimer-targeting agent conjugate as described herein comprises at least one targeting agent, for localisation and concentration of the conjugate at the site or target of interest in the body. Targeting agents include antibodies, antibody fragments, peptide sequences, and other motifs capable of selective binding to the target of interest.

The interaction may occur through any type of bonding or association including, for example, covalent, ionic bonding, hydrogen bonding, and Van der Waals forces.

As used herein, “peptidic” refers to a molecule comprising two or more amino acids linked by peptide bonds.

The targeting agents as described herein are useful for targeting the disclosed dendrimer-targeting agent conjugate to targets such as tumours, cancer cells, and/or the tumour microenvironment.

The targeting agent may, for example, comprise an antigen-binding site or antigen binding domain that specifically binds and/or has an affinity for a target molecule (also referred to herein as a “target” or “antigen”).

In an embodiment, the target is selected from one or more of the following: human epidermal growth factor receptor 2 (HER2), Epidermal growth factor receptor (EGFR), a vascular epithelial growth factor (VEGF) receptor, a G-protein-coupled receptor 161 (GPR161), fibroblast growth factor receptor (e.g. FGFR2), hepatocyte growth factor (HGF), hepatocyte growth factor receptor (HGFR), tyrosine-protein kinase met (C-met), atypical chemokine receptor 3 (CXCR7), C—X—C Motif Chemokine Receptor 4 (CXCR4), carcinoembryonic antigen, mucin 1 (MUC-1), mucin-16 (MUC16), epithelial cell adhesion molecule (EpCAM), trophoblast glycoprotein (5T4), interleukin-2 (IL-2), glycoprotein (gpNMB), Syndecan1 (CD138), prostate-specific membrane antigen (PSMA), Carcinoembryonic Antigen Related Cell Adhesion Molecule 5 (CEACAM5), solute carrier family 44 member 4 (CSLC44A4), granulocyte-colony stimulating factor receptor (G-CSFR), ectonucleotide pyrophosphatase/phosphodiesterase 3 (ENPP3), mesothelin, Nectin Cell Adhesion Molecule 4 (Nectin-4), Fibroblast activation protein (FAP), Folic acid receptor, Hyaluronic acid receptors, integrin receptor (αvβ3), lectin binding glycoproteins, TfR, VEGFR-1 and VEGFR-2, a cytokine, CD56, CD19, CD16, CD74, CD37, CD70, CD52,CD19, CD22, CD20, CD30, CD3, and CD79b.

In an embodiment, the target is HER2, also known as ERBB2; Gene ID no. 2064 (NCBI).

In an embodiment, the target is epidermal growth factor receptor (EGFR), also known as ERBB1 or HER1; Gene ID no. 1956 (NCBI).

In an embodiment, the target is prostate specific membrane antigen (PSMA); Gene ID no. 2346 (NCBI).

In an embodiment, the target is fibroblast activation protein (FAP). Overexpression of serine protease fibroblast activation protein in cancers facilitates selective targeting of tumours (Loktev et al, J Nucl Med, 2019, 60(10), p1421-1429).

In an embodiment, the targeting agent comprises a molecular weight of up to about 200 kDa, or up to about 150 kDa, or up to about 110 KDa, or up to about 80 KDa, or up to about 55 KDa, or up to about 16 kDa. In an embodiment, the targeting agent comprises a molecular weight of up to about 200 kDa. In an embodiment, the targeting agent comprises a molecular weight of up to about 150 kDa. In an embodiment, the targeting agent comprises a molecular weight of up to about 110 kDa. In an embodiment, the targeting agent comprises a molecular weight of up to about 80 kDa. In an embodiment, the targeting agent comprises a molecular weight of up to about 55 kDa. In an embodiment, the targeting agent comprises a molecular weight of up to about 16 kDa. In an embodiment, the targeting agent has a molecular weight of about 3 kDa to about 80 kDa. In an embodiment, the targeting agent has a molecular weight of about 3 kDa to about 60 kDa. In an embodiment, the targeting agent has a molecular weight of about 3 kDa to about 50 kDa. In an embodiment, the targeting agent has a molecular weight of about 3 kDa to about 40 kDa. In an embodiment, the targeting agent has a molecular weight of about 3 kDa to about 30 kDa. In an embodiment, the targeting agent has a molecular weight of about 3 kDa to about 20 kDa. In an embodiment, the targeting agent has a molecular weight of about 3 kDa to about 15 kDa. In an embodiment, the targeting agent has a molecular weight of about 3 kDa to about 13 kDa. In an embodiment, the targeting agent has a molecular weight of about 5 kDa to about 15 kDa. In an embodiment, the targeting agent has a molecular weight of about 5 kDa to about 12 kDa. In an embodiment, the targeting agent has a molecular weight of about 5 kDa to about 10 kDa.

As described herein, “kDA” or “kilodalton” refers to a unit of molecular mass consisting of 1000 Daltons.

In an embodiment, the targeting agent is selected from: an antibody, a heavy chain antibody, ScFV-Fc, Fab, Fab2, Fv, scFv or a single domain antibody. In an embodiment, the targeting agent is selected from: an antibody or an antibody fragment.

In an embodiment, the targeting agent is an antibody. For the purposes for the present disclosure, the term “antibody” includes four chain protein comprising e.g., two light chains and two heavy chains including recombinant or modified antibodies (e.g., chimeric antibodies, humanized antibodies, primatized antibodies, de-immunized antibodies and half antibodies, bispecific antibodies) capable of specifically binding to one or a few closely related antigens by virtue of a Fv. An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (˜50-70 kDa) covalently linked and two light chains (˜23 kDa each). A light chain generally comprises a variable region and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ç, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (V_{H} or V_{L} wherein each are ˜110 amino acids in length) and one or more constant domains at the C-terminus. The constant domain of the light chain (CL which is ˜110 amino acids in length) is aligned with and disulfide bonded to the first constant domain of the heavy chain (CH which is −330-440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a human antibody or a deimmunized or germlined version thereof, or an affinity matured version thereof. The terms “full-length antibody,” or “whole antibody” are used interchangeably to refer to an antibody in its substantially intact form, as opposed to an antigen binding fragment of an antibody. Specifically, whole antibodies include those with heavy and light chains including a constant region. The constant region may be wild-type sequence constant regions (e.g., human wild-type sequence constant regions) or amino acid sequence variants thereof.

In an embodiment, the targeting agent is a fusion protein. As used herein, a “fusion protein” is a protein created by the joining of two or more nucleic acid sequences that originally coded for separate proteins or part thereof (e.g. fusion of a portion of a protein receptor with a portion of an antibody (Etanercept)).

In an embodiment, the targeting agent is an antibody fragment. As used herein, the term “antibody fragment” shall be taken to mean a portion of or a fragment of an antibody capable of specifically binding to an antigen, including for example, a F_{V}, V_{H}, V_{L} or a variable region as defined herein. This term shall be understood to encompass fragments directly derived from an antibody as well as proteins produced using recombinant means. In an embodiment, the antibody fragment is selected from a Fab, Fab2, Fv, scFv, heavy chain antibody, domain antibody, heavy chain antibody, diabody, or triabody.

As used herein, the term “Fv” shall be taken to mean any protein, whether comprised of multiple polypeptides or a single polypeptide (scFV), in which a V_{L} and a V_{H} associate and form a complex having an antigen binding domain, i.e., capable of specifically binding to an antigen. The V_{H} and the V_{L} which form the antigen binding domain can be in a single polypeptide chain or in different polypeptide chains. In an embodiment, an Fv of the disclosure (as well as any protein of the disclosure) may have multiple antigen binding sites which may or may not bind the same antigen. This term shall be understood to encompass fragments directly derived from an antibody as well as proteins produced using recombinant means. In some examples, the V_{H} is not linked to a heavy chain constant domain CH1 and/or the V_{L} is not linked to a light chain constant domain (CL), e.g., a domain antibody. Exemplary Fv containing polypeptides or proteins include a Fab fragment, a Fab′ fragment, a F(ab′) fragment, a scFv, a diabody, a triabody, A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A Fab fragment generally comprises or consists of a V_{H} and C_{H}1 and a V_{L} and C_{L}. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

In some embodiments, the antibody fragment is selected from: a heavy chain antibody, Fab, Fab2, Fv, scFv or a single domain antibody.

As used herein, the “single-domain antibodies (sdAbs)”, also referred to as a “domain antibodies (dAb)” or “nanobodies” comprises a single variable region of a heavy chain V_{H} or light chain V_{L}. In an embodiment, the variable region is camelid-derived. In an embodiment, the variable region is derived from sharks. In an embodiment, the V_{H} is a camelid-derived V_{H}.

In some embodiments, the targeting agent is a single domain antibody. In some embodiments, the targeting agent is a V_{H} single domain antibody. In some embodiments, the targeting agent is a V_{L} single domain antibody.

In an embodiment, the single domain antibody comprises a single domain amino acid sequence as described in for example, EP2215125A1, US20110028695, Hussack et al. (2018), Arezumand et al. (2017), Chanier et al (2019). In an embodiment, the single domain antibody comprises a single domain amino acid sequence as described US20110028695.

In an embodiment, the single domain antibody comprises a single domain amino acid sequence as disclosed in Example 7. In an embodiment, the single domain antibody is 2D3 comprising the amino acid sequence as shown in SEQ ID NO: 1986 of US20110028695.

In an embodiment, the single domain antibody comprises the amino acid sequence EVQLVESGGSLVQPGGSLRLSCAASGFTFDDYAMSWVRQVPGKGLEWVSSINWSGT HTDYADSVKGRFTISRNNANNTLYLQMNSLKSEDTAVYYCAKNWRDAGTTWFEKS GSAGQGTQVTVSS (SEQ ID NO: 1). In one embodiment, the sequence includes an additional cysteine residue for conjugation.

In an embodiment, the single domain antibody comprises the amino acid sequence EVQLVESGGSLVQPGGSLRLSCAASGFTFDDYAMSWVRQVPGKGLEWVSSINWSGT HTDYADSVKGRFTISRNNANNTLYLQMNSLKSEDTAVYYCAKNWRDAGTTWFEKS GSAGQGTQVTVSS #ENLYFQGHHHHHH, wherein #denotes an unnatural amino acid preferably a 4-azidophenylalanine residue (SEQ ID NO: 2).

In an embodiment, the single domain antibody comprises the amino acid sequence EVQLVESGGSLVQPGGSLRLSCAASGFTFDDYAMSWVRQVPGKGLEWVSSINWSGT HTDYADSVKGRFTISRNNANNTLYLQMNSLKSEDTAVYYCAKNWRDAGTTWFEKS GSAGQGTQVTVSS #, wherein #denotes an unnatural amino acid, preferably a 4-azidophenylalanine residue (SEQ ID NO: 3).

In an embodiment, the single domain antibody comprises the amino acid sequence GGSHHHHHHGMASMTGGQQMGRDLYENLYFQGEVQLVESGGSLVQPGGSLRLSC AASGFTFDDYAMSWVRQVPGKGLEWVSSINWSGTHTDYADSVKGRFTISRNNANN TLYLQMNSLKSEDTAVYYCAKNWRDAGTTWFEKSGSAGQGTQVTVSS (SEQ ID NO: 4).

In an embodiment, the single domain antibody comprises the amino acid sequence GGSHHHHHHGMASMTGGQQMGRDLYENLYFQGEVQLVESGGSLVQPGGSLRLSC AASGFTFDDYAMSWVRQVPGKGLEWVSSINWSGTHTDYADSVKGRFTISRNNANN TLYLQMNSLKSEDTAVYYCAKNWRDAGTTWFEKSGSAGQGTQVTVSS #, wherein #denotes an unnatural amino acid preferably a 4-azidophenylalanine residue (SEQ ID NO:5).

In an embodiment, the single domain antibody comprises the amino acid sequence EVQLVESGGSLVQPGGSLRLSCAASGFTFDDYAMSWVRQVPGKGLEWVSSINWSGT HTDYADSVKGRFTISRNNANNTLYLQMNSLKSEDTAVYYCAKNWRDAGTTWFEKS GSAGQGTQVTVSS[X]ₙC, wherein X is any amino acid and n=0 to 20. In one embodiment n=0, 1, 2, 3, 4 or 5. In one embodiment n=4 (SEQ ID NO:6).

In an embodiment, the single domain antibody comprises the amino acid sequence C[X]ₙEVQLVESGGSLVQPGGSLRLSCAASGFTFDDYAMSWVRQVPGKGLEWVSSIN WSGTHTDYADSVKGRFTISRNNANNTLYLQMNSLKSEDTAVYYCAKNWRDAGTT WFEKSGSAGQGTQVTVSS, wherein X is any amino acid and n=0 to 20. In one embodiment n=0, 1, 2, 3, 4 or 5 (SEQ ID NO:7).

In an embodiment, the single domain antibody comprises the amino acid sequence EVQLVESGGSLVQPGGSLRLSCAASGFTFDDYAMSWVRQVPGKGLEWVSSINWSGT HTDYADSVKGRFTISRNNANNTLYLQMNSLKSEDTAVYYCAKNWRDAGTTWFEKS GSAGQGTQVTVSS[X]ₙC[X]ₘ, wherein X is any amino acid, m=0 to 20 and n=0 to 20. In one embodiment n=0, 1, 2, 3, 4 or 5 (SEQ ID NO:8).

In an embodiment, the single domain antibody comprises the amino acid sequence [X]ₙC[X]ₘEVQLVESGGSLVQPGGSLRLSCAASGFTFDDYAMSWVRQVPGKGLEWVS SINWSGTHTDYADSVKGRFTISRNNANNTLYLQMNSLKSEDTAVYYCAKNWRDAG TTWFEKSGSAGQGTQVTVSS, wherein X is any amino acid, m=0 to 20 and n=0 to 20. In one embodiment n=0, 1, 2, 3, 4 or 5 (SEQ ID NO:9). In an embodiment, the single domain antibody comprises the amino acid sequence EVQLVESGGSLVQPGGSLRLSCAASGFTFDDYAMSWVRQVPGKGLEWVSSINWSGT HTDYADSVKGRFTISRNNANNTLYLQMNSLKSEDTAVYYCAKNWRDAGTTWFEKS GSAGQGTQVTVSSLGTLCTPSRENLYFQGHHHHHH (SEQ ID NO:10).

In some embodiments, the targeting agent is a single domain antibody and has a molecular weight of about 4 kDa to about 80 kDa, or about 5 kDa to about 80 kDa, or about 5 kDa to about 60 kDa, or about 5 kDa to about 50 kDa, or about 5 kDa to about 40 kDa, or about 5 kDa to about 30 kDa, or about 5 kDa to about 20 kDa, or about 5 kDa to about 16 kDa, or about 5 kDa to about 15 kDa, or about 5 kDa to about 12 kDa, or about 10 kDa to about 16 kDa or about 15 kDa to 20 kDa.

In some embodiments, the targeting agent comprises less than about 500, less than about 400, less than about 300, less than about 200, less than about 150, less than about 140, less than about 130, less than about 120, less than about 110, less than about 100 amino acid residues. In some embodiments, the targeting agent comprises more than about 50, more than about 75, more than about 100, or more than about 120 amino acid residues. In some embodiments, the targeting agent comprises fewer than 120 amino acid residues. In some embodiments, the targeting agent comprises from about 100 to about 120 amino acid residues.

In some embodiments, the targeting agent is a mimetic of an antibody. As used herein, the term “mimetic” or “mimetics” refers to compounds that like antibodies or antibody fragments, can bind antigens, but are not structurally related to antibodies. This term shall be understood to not encompass antibodies or antibody fragments as described herein. This term shall be understood to encompass synthetic mimetics (produced in vitro) and mimetics produced using recombinant means. This term shall be understood to encompass protein mimetics.

In an embodiment, the mimetic is selected from an: affibody, aptamer, affilins, affimer, affitins, anticalins, avimers, alpha bodies, monobodies, DARPins, aptamer, Fyomers, fibronectin type III-derived protein scaffold, phytocystatin-derived protein scaffold and a paratope mimetic peptide. Like antibodies, mimetics can be used as targeting moieties.

In an embodiment, the mimetic is derived from one of the following protein scaffolds: z domain of protein A, gamma-B crystallin, ubiquitin, cystatin, sac7d, triple helix, coiled coil, lipocalin, cyclotides, A domains of a membrane receptor, ankyrin repeat motif, sh3 domain of Fym, Kunits domians of a protease inhibitor, type III domain of fibronectin and IgG-like, thermostable carbohydrate binding module family 32 (CBM32) from a Clostridium perfringens.

In an embodiment, the mimetic is about 3 kDa to about 20 kDa, or about 4 kDa to about 18 kDa, or about 6 kDa to about 16 kDa, or about 6 kDa to about 14 kDa, or about 6 kDa to about 12 kDa, or about 6 kDa to about 10 kDa, or about 6 kDa to about 8 kDa. In an embodiment, the mimetic is about 3 kDa to about 20 kDa. In an embodiment, the mimetic is about 3 kDa to about 20 kDa. In an embodiment, the mimetic is about 4 kDa to about 18 kDa. In an embodiment, the mimetic is about 6 kDa to about 16 kDa. In an embodiment, the mimetic is about 6 kDa to about 14 kDa. In an embodiment, the mimetic is about 6 kDa to about 12 kDa. In an embodiment, the mimetic is about 6 kDa to about 10 kDa. In an embodiment, the mimetic is about 6 kDa to about 8 kDa.

In some embodiments, the targeting agent is an affibody. As used herein, the term “affibody” refers to any of a class of very small (approximately 6 kDa) polypeptide antibody mimetics based on a three alpha helix bundle domain of about 58 amino acids in length known as a “Z domain”. Typically, the scaffold for affibodies is based on a modified version of the B-domain of Protein A. Affibodies are characterized by very high stability (withstanding temperatures as high as 90° C.) and target affinities ranging from nanomolar to picomolar. See, e.g., Nord et al. (1995), Protein Eng., 8:601-608. Examples of known affibodies include, for example, affibodies against HER2 (e.g., the Anti-HER2 Affibody®, AFFTBODY AB, Bromma, Sweden; U.S. Pat. No. 7,993,650).

In some embodiments, the targeting agent is an affibody and has a molecular weight in the range of about 3 kDa to about 10 kDa, or about 3 kDa to about 8 kDa, or about 4 kDa to about 8 kDa, or about 4 kDa to about 7 kDa, or about 5 kDa to about 7 kDa, or about 6 kDa.

In some embodiments, the targeting agent is an affibody and has fewer than 80 amino acid residues, or fewer than 70 amino acid residues, or fewer than 65 amino acid residues, or fewer than 60 amino acid residues. In some embodiments, the targeting agent is made up of from 40 to 80 amino acid residues, or from 50 to 70 amino acid residues, or from 55 to 65 amino acid residues, or from 56 to 60 amino acid residues, or about 58 amino acid residues.

For the purposes of the present disclosure, the term “antibody” comprises four chain protein comprising e.g., two light chains and two heavy chains including recombinant or modified antibodies (e.g., chimeric antibodies, humanized antibodies, primatized antibodies, de-immunized antibodies and half antibodies, bispecific antibodies) capable of specifically binding to one or a few closely related antigens by virtue of a Fv. An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (˜50-70 kDa) covalently linked and two light chains (˜23 kDa each). A light chain generally comprises a variable region and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ˜110 amino acids in length) and one or more constant domains at the C-terminus. The constant domain of the light chain (CL which is ˜110 amino acids in length) is aligned with and disulfide bonded to the first constant domain of the heavy chain (CH which is −330-440 amino acids in length). The light chain variable region is aligned with the variable region of the heavy chain. The antibody heavy chain can comprise two or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a human antibody or a deimmunized or germlined version thereof, or an affinity matured version thereof.

The terms “full-length antibody”, or “whole antibody” are used interchangeably to refer to an antibody in its substantially intact form, as opposed to an antigen binding fragment of an antibody. Specifically, whole antibodies include those with heavy and light chains including a constant region. The constant region may be wild-type sequence constant regions (e.g., human wild-type sequence constant regions) or amino acid sequence variants thereof.

As used herein, the term “variable region” refers to the portions of the light and/or heavy chains of an antibody as defined herein or of a heavy chain only antibody (e.g., camelid antibodies or cartilaginous fish immunoglobulin new antigen receptors (IgNARs)) that is capable of specifically binding to an antigen and includes amino acid sequences of complementary determining regions “CDRs”; i.e., CDR1, CDR2, and CDR3, and framework regions “FRs”. FR are those variable region residues other than the CDR residues. For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region typically has three CDR regions identified as CDR1, CDR2 and CDR3. Each complementarity determining region may comprise amino acid residues from a “complementarity determining region” as defined by Kabat et al., (1987 and/or 1991). For example, in a heavy chain variable region CDRH1 is between residues 31-35, CDRH2 is between residues 50-65, and CDRH3 is between residues 95-102. In a light chain, CDRL1 is between residues 24-34, CDRL2 is between residues 50-56, and CDRL3 is between residues 89-97. These CDRs can also comprise numerous insertions, e.g., as described in Kabat (1987 and/or 1991). The present disclosure is not limited to FRs and CDRs as defined by the Kabat numbering system, but includes all numbering systems, including the canonical numbering system or of Chothia and Lesk (1987); Chothia et al. (1989); and/or Al-Lazikani et al., (1997); the numbering system of Honnegher and Plukthun (2001); the IMGT system discussed in Giudicelli et al., (1997); or the Enhanced Chothia Numbering Scheme (http://www.bioinfo.org.uk/mdex.html). In one example, the CDRs and/or FRs are defined according to the Kabat numbering system, e.g., as depicted in FIGS. 9A-9D in bold text. Optionally, heavy chain CDR2 according to the Kabat numbering system does not comprise the five C-terminal amino acids listed herein or any one or more of those amino acids are substituted with another naturally-occurring amino acid. In an additional, or alternative, option, light chain CDR1 does not comprise the four N-terminal amino acids listed herein or any one or more of those amino acids are substituted with another naturally-occurring amino acid. In this regard, Padlan et al., 1995 established that the five C-terminal amino acids of heavy chain CDR2 and/or the four N-terminal amino acids of light chain CDR1 are not generally involved in antigen binding. In one example, the CDRs and/or FRs are defined according to the Chothia numbering system, e.g., as depicted in FIGS. 9A-9D in underlined text.

As used herein, the term “Kabat numbering system” refers to the scheme for numbering antibody variable regions and identifying CDRs (hypervariable regions) as set out in Kabat et al. (1987 and/or 1991).

As used herein, the term “Chothia numbering system” refers to the scheme for numbering antibody variable regions and identifying CDRs (structural loops) as set out in Chothia and Lesk (1987) or Al-Lazikani et al. (1997).

As used herein, the term “antigen binding domain” shall be taken to mean the region of a targeting agent that is capable of specifically binding to an antigen (e.g. HER2).

As used herein, the term “binds” or “binding” in reference to the interaction of a protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the antibody, will reduce the amount of labeled “A” bound to the antibody.

As used herein, the term “specifically binds”, “binds specifically”, or similar phrases shall be taken to mean a protein of the disclosure reacts or associates more frequently, more rapidly, with greater duration and/or with greater affinity with a particular antigen (such as HER2) or cell expressing same than it does with alternative antigens or cells. For example, a protein that specifically binds to an antigen binds that antigen with greater affinity (e.g., 20 fold, or 40 fold, or 60 fold, or 80 fold, or 100 fold, or 150 fold, or 200 fold greater affinity), avidity, more readily, and/or with greater duration than it binds to other antigens. It is also understood by reading this definition that, for example, a protein that specifically binds to a first antigen may or may not specifically bind to a second antigen. As such “specific binding” does not necessarily require exclusive binding or non-detectable binding of another antigen, this is meant by the term “selective binding”.

In some embodiments, the targeting agents comprises or consists of an amino acid sequence corresponding to a targeting agent amino acid sequences as defined herein.

In some embodiments, the targeting agent is or comprises an oligomeric peptide sequence, for example of up to 20 amino acids in length. In some embodiments, the targeting agent is a peptide sequence of from 5 to 20 amino acids, from 7 to 18 amino acids, or from 9 to 15 amino acids. In some embodiments, the targeting agent is a peptide sequence of 7, 8, 9, 10, 11, 12, 13, 14, 15, 16, 17, or 18 amino acids.

In some embodiments, the targeting agent has a molecular weight of less than 2000 Da, less than 1000 Da, or less than 500 Da. In some embodiments, the targeting agent is a small molecule which may be considered to be one having a molecular weight of less than about 1,000 Da or less than about 750 Da, or less than about 500 Da.

In one example, the targeting agent is a small molecule that binds to PSMA. The small molecule that binds to PSMA may, in one embodiment, be a peptide. Such binding peptides are known in the art. In one example, the targeting agent is a small molecule that binds to fibroblast activation protein (FAP).

In some embodiments, the targeting agent is a targeting agent that binds specifically to prostate specific membrane antigen (PSMA). For example, the targeting agent may be or may contain a DUPA group, or an analogue thereof. DUPA has the structure:

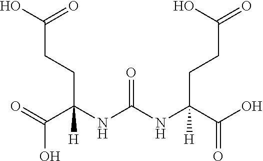 [SMILES: O=C(O)CC[C@H](NC(=O)N[C@@H](CCC(=O)O)C(=O)O)C(=O)O]

In some embodiments, the targeting agent is or contains a DUPA group conjugated via a carboxyl group, e.g.:

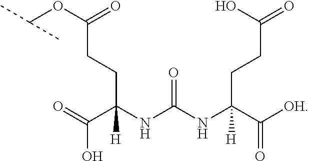

In some embodiments, the targeting agent is a targeting agent that binds specifically to fibroblast activation protein (FAP). In some embodiments, the targeting agent may be or may contain a group that inhibits fibroblast activation protein (FAP). For example, the targeting gent may be or may contain a FAP binding group, or an analogue thereof, having the structure:

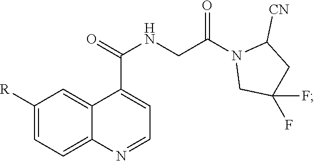

wherein R is a substituent of Formula I

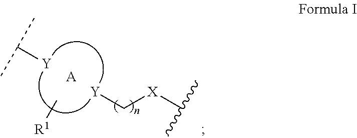

wherein
X is selected from O, NH, N(CH₃), S, and CH₂;
Y is selected from N, and C;
n is an integer selected from the group consisting of 0, 1, 2, 3, 4, 5, and 6;
A is a 5- to 10-membered monocyclic or bicyclic heterocyclic group;
R¹ is selected from the group consisting of C₁-C₆alkyl group optionally substituted with one or more 5- to 10-membered cyclic groups; and
[symbol] represents the point of conjugation to the dendrimer.

In some embodiments, R is a substituent of Formula I wherein X is O. In some embodiments, R is a substituent of Formula I wherein X is NH. In some embodiments, R is a substituent of Formula I wherein X is N(CH₃). In some embodiments, R is a substituent of Formula I wherein X is S. In some embodiments, R is a substituent of Formula I wherein X is CH₂.

In some embodiments, R is a substituent of Formula I wherein Y is N. In some embodiments, R is a substituent of Formula I wherein Y is C. In this embodiment, where Y is C, it will be appreciated that A may be an aromatic or saturated 5- to 10-membered monocyclic or bicyclic heterocyclic group. In some embodiments, R is a substituent of Formula I wherein Y is CH. In this embodiment, where Y is C, it will be appreciated that A is a partially or fully saturated 5- to 10-membered monocyclic or bicyclic heterocyclic group.

In some embodiments, R is a substituent of Formula I wherein n is 0. In this embodiment, when n is 0, it will be understood that X is directly bonded to Y. In some embodiments, R is a substituent of Formula I wherein n is 1. In some embodiments, R is a substituent of Formula I wherein n is 2. In some embodiments, R is a substituent of Formula I wherein n is 3. In some embodiments, R is a substituent of Formula I wherein n is 4. In some embodiments, R is a substituent of Formula I wherein n is 5. In some embodiments, R is a substituent of Formula I wherein n is 6.

In some embodiments, R is a substituent of Formula I wherein A is a 5- to 10-membered monocyclic or bicyclic heterocyclic group. In one example, A is a 5-membered monocyclic heterocyclic group. In one example, A is a 6-membered monocyclic heterocyclic group. In one example, A is a 7-membered monocyclic heterocyclic group. In one example, A is a 7-memebred bicyclic heterocyclic group. In one example, A is an 8-membered bicyclic heterocyclic group. In one example, A is a 9-membered bicyclic heterocyclic group. In one example, A is a 10-membered bicyclic heterocyclic group.

Examples of A groups include, but are not limited to:

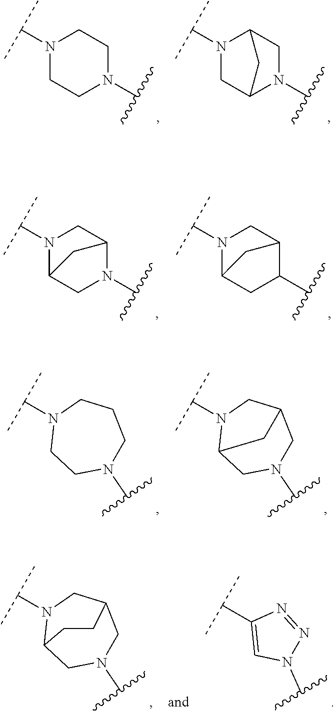

In some embodiments, R is a substituent of Formula I wherein R¹ is a C₁-C₆alkyl group. In one example, R¹ is a C₁-alkyl group (i.e., CH₃). In some embodiments, R is a substituent of Formula I wherein R¹ is a C₁-C₆alkyl group optionally substituted with one or more 5- to 10-membered cyclic groups. In some embodiments, R¹ is a C₁-alkyl group substituted with a 6-membered cyclic group. In one example, R¹ is CH₂-phenyl.

In one example, R is a substituent of Formula I, being:

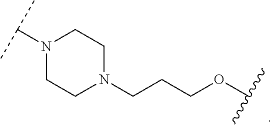

In one example, R is a substituent of Formula I, being:

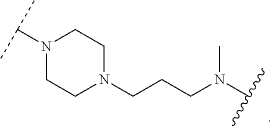

Further examples of R substituents include those published by Loktev et al. (Loktev, A. et al., The Journal of Nuclear Medicine, 60(10), 2019, p1421-1429).

Accordingly, in one embodiment, the targeting agent is a FAP binding group having the structure:

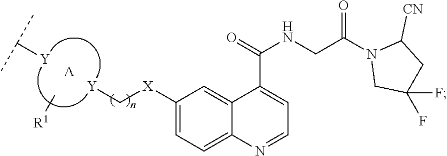

wherein X, Y, n, A, and R¹ are as described herein.

In one example, the targeting agent is a FAP binding group having the structure:

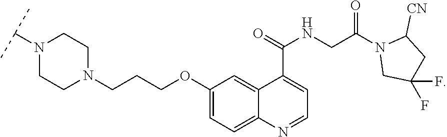

In one example, the targeting agent is a FAP binding group having the structure:

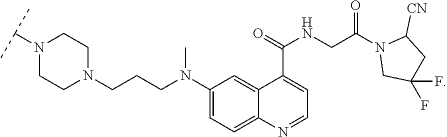

In some embodiments, the targeting agent is a targeting agent which is selective for one or more of HER2, EGFR, PSMA, or FAP. In an embodiment, the targeting agent is a targeting agent which is selective for HER2. In an embodiment, the targeting agent is a targeting agent which is selective for EGFR. In an embodiment, the targeting agent is a targeting agent which is selective for PSMA. In an embodiment, the targeting agent is a targeting agent which is selective for FAP.

In some embodiments, the targeting agent is competitive for binding with other targeting agents, such as those commercially available. In one example, the targeting agent is competitive for binding with a commercially available antibody therapy. In one example, the targeting agent is selective for HER2, and is competitive for binding with a HER2 antibody, for example, trastuzumab, pertuzumab, and margetuximab. In one example, the targeting agent is selective for EGFR, and is competitive for binding with an EGFR antibody, for example, cetuximab, panitumumab, nimotuzumab, and necitumumab. In one example, the targeting agent is selective for FAP, and is competitive with binding with a FAP antibody, for example, sibrotuzumab.

In some embodiments, the conjugate comprises a single targeting agent. In other embodiments, the conjugate comprises multiple targeting agents, e.g. 2, 3, 4, or 5 targeting agents. In some embodiments, the conjugate comprises 1 to 32 targeting agents. In some embodiments, the conjugate comprises at least 5 targeting agents. In some embodiments, the conjugate comprises between 5 and 30 targeting agents.

The targeting agent is attached to the remainder of the conjugate via the spacer. In some embodiments, the covalent attachment, or linkage between the targeting agent and the spacer group has been formed by a reaction between complementary reactive functional groups present on an intermediate comprising the targeting agent and an intermediate comprising the dendrimer.

In some embodiments, the targeting agent is covalently linked to the spacer group at the C-terminus of the targeting agent.

In one embodiment, the FAP binding group is conjugated to the dendrimer via a spacer group, as described herein. Accordingly, in one example, the targeting agent is a FAP binding group conjugated to the dendrimer via a spacer group comprising polyethylene glycol (PEG). In one embodiment, the targeting agent is a FAP binding group conjugated to the dendrimer via a spacer group comprising polyethylene glycol (PEG) having the structure:

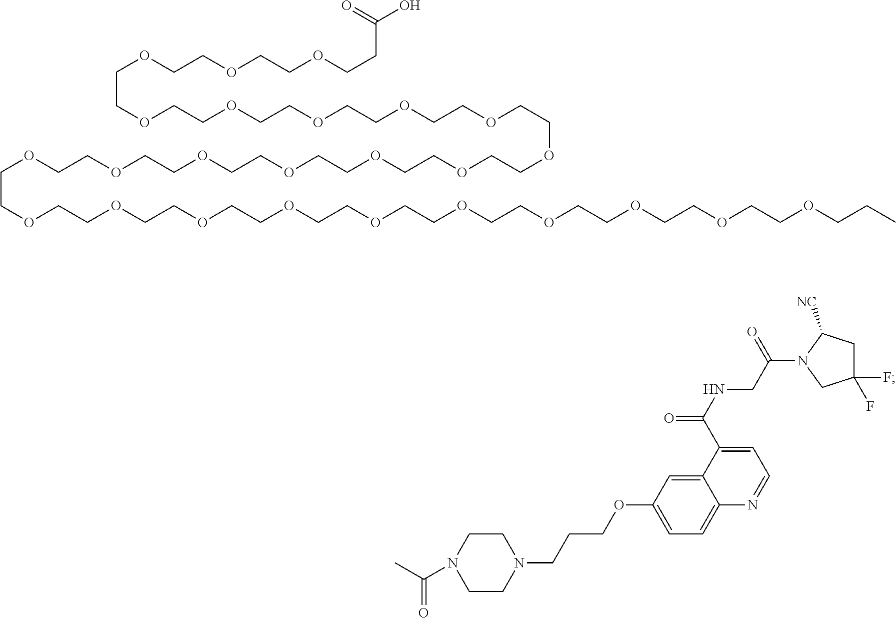

wherein the spacer group is conjugated to the dendrimer via the terminal carboxylic acid group of the spacer.

The covalent attachment site for attaching the targeting agent to the spacer, and thus to the dendrimer, can for example be cysteine, lysine, N-terminal amines, tyrosine, carbohydrates, non-natural amino acids or transaminase or recognition sequences. Binding sites for covalent attachment to proteins are known in the art, (for example, Milla P., et al, Current Drug Metabolism (2012) V13, 1:105-119.) In some embodiments, an intermediate comprising the targeting agent comprises an unnatural amino acid residue for attachment to the spacer. The unnatural amino acid residue may have a side chain that has a reactive functional group that is complementary to a reactive functional group which may be present on a spacer group, or present on an intermediate comprising the dendrimer with a spacer group attached. In some embodiments, the unnatural amino acid residue is one containing an azide group, e.g. it may be a 4-azidophenylalanine residue, e.g.

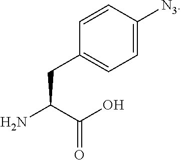 [SMILES: [N-]=[N+]=Nc1ccc(C[C@H](N)C(=O)O)cc1]

Azide groups are capable of undergoing cycloaddition reactions with alkyne groups which may be present in a spacer precursor group. In some embodiments, the unnatural amino acid is a diene-containing amino acid, e.g. a spirocyclopentadiene-containing amino acid such as:

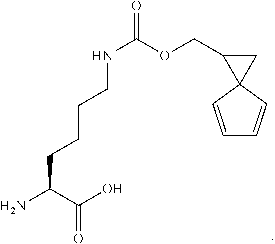 [SMILES: N[C@@H](CCCCNC(=O)OCC1CC12C=CC=C2)C(=O)O]

Diene groups are capable of undergoing cycloaddition (e.g. Diels-Alder) reactions with alkene groups, such as those present on maleimide. Further examples of unnatural amino acids which may be used for attachment to the spacer include those containing a carbonyl group, such as a ketone, and those containing a methylcyclopropylene group. Additional examples of unnatural amino acids include:

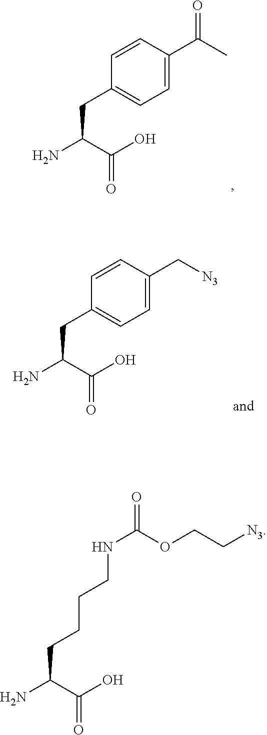 [SMILES: CC(=O)c1ccc(C[C@H](N)C(=O)O)cc1 ; [N-]=[N+]=NCc1ccc(C[C@H](N)C(=O)O)cc1 ; [N-]=[N+]=NCCOC(=O)NCCCC[C@H](N)C(=O)O]

In some embodiments, the targeting agent comprises or consists of any of the amino acid sequences as defined herein.

### Spacer Group

As used herein, the term “spacer group” refers to a chemical entity that serves to attach the targeting agent to the dendrimer. That is, a spacer group joins the targeting agent to the dendrimer. In some embodiments the spacer may simply be an atom or small chemical linking group by which the targeting agent bonds to the dendrimer. In other embodiments the spacer group may be more extensive. In such embodiments, the spacer group is intended to position the targeting agent such that it is capable of binding to, for example, the HER2 receptor without undue deleterious interference from other constituents of the dendrimer.

In some embodiments, the targeting agent is attached to the dendrimer through the core of the dendrimer. That is, the targeting agent, such as an antibody fragment, is covalently attached to the core of the dendrimer by the spacer group. Attachment of a targeting agent to a dendrimer via a spacer attached to the core of the dendrimer may be beneficial if the dendrimer is sterically crowded, wherein the spacer group may be of sufficient length to protrude beyond the surface of the dendrimer, to allow for in vivo binding of the targeting agent to its receptor or similar. For example, if the core is a maleimide-containing core the targeting agent may be attached to the maleimide ring nitrogen and in such a situation a spacer group would be beneficial.

In some other embodiments, the targeting agent is attached to the dendrimer via a surface building unit of the dendrimer. For example, the targeting agent may be linked to a surface nitrogen of a lysine residue via a spacer group, for example by means of an amide bond formed between an amine group of a lysine residue, and a carboxylic acid group present on an intermediate comprising the targeting agent and spacer group.

Any suitable chemical groups which serve to distance the targeting agent at an appropriate distance from the dendrimer such that it is able to bind to its target may be utilised. Exemplary spacer groups include those comprising polyethylene glycol (PEG), polypropylene glycol, polyaryls, amide linkages, peptides, amino acids, alkyloxy, alkylamino, alkyl and alkenyl chains, and saccharides (mono, oligo, and poly), or residues thereof. In some embodiments, the spacer group comprises one or more PEG groups, such as from 2 to 60 ethyleneoxy repeat units, for example, from 2 to 20 or 20 to 48 repeat units. In one embodiment, the PEG is from 8 to 36 repeat units. In a further embodiment, the PEG is 12, 16, 20, 24, or 36 repeat units.

In some embodiments, the spacer group comprises multiple PEG groups interspersed with other functional groups. For example, the spacer group may comprise PEG groups linked via, e.g. amide groups or other functional groups useful for connecting parts of the spacer group.

Any suitable means of attaching an intermediate comprising a spacer group to an intermediate comprising the targeting agent may be utilised. For example, sites for covalent attachment include, but are not limited to, cysteine residues, lysine residues, C-terminal amino acid residues, N-terminal amines, tyrosine residues, carbohydrates, suitable non-natural amino acid residues, or transaminase or recognition sequences. Binding sites for covalent attachment to proteins, such as targeting agents, are known in the art (for example, Milla P., et al., 2012). For example, an intermediate comprising a spacer group may be reacted with an intermediate comprising a targeting agent at the C-terminus of the targeting agent, such that the spacer group is attached to the targeting agent via the C-terminus. In some embodiments, the targeting agent is attached to the spacer group via the C-terminus of the targeting agent. In one embodiment, the targeting agent is covalently attached, or linked, to the spacer group via the C-terminus of the targeting agent.

Accordingly, for attachment of the spacer group to the targeting agent and/or the dendrimer, an intermediate comprising the spacer group may include one or more reactive functional groups.

In particular embodiments, the reactive functional groups may be complementary reactive groups selected from the group consisting of hydroxy, carboxy, active esters such as NHS or pentafluorophenol esters, amino, azide, maleimides (including sulfo-maleimide), dienes (such as cyclopentadienes, e.g. a spiro [2.4]hepta-4,6-diene group), tetrazine, citraconimide, alkyne-containing groups including BCN (bicycle[6.1.0]non-4-yn-9-yl), DBCO (dibenzocyclooctyne-amine), thiol, carbonyl groups such as aldehydes and ketones, alkoxyamines, haloacetate, biotin, tetrazines, alkene-containing groups including TCO (trans-cyclooctene), methyl-cyclopropylene groups, and PTAD or other tyrosine reactive groups.

For example, in some embodiments, a spacer group intermediate may comprise two reactive groups, e.g. one at each end, which are orthogonal, i.e. at least one of the reactive groups is capable of reacting with a complementary group present on either an intermediate comprising the targeting agent, or an intermediate comprising the dendrimer, to attach the spacer group to that constituent of the conjugate, under conditions in which the other reactive group is stable and does not substantially react. This allows for the spacer to be attached to either the dendrimer or the targeting agent and then subsequently, through reaction of the other reactive group with a complementary group present on the remaining constituent, in order to link the targeting agent to the dendrimer.

In some embodiments, the spacer group is attached, directly or indirectly, to the targeting agent by means of reaction of precursors containing alkyne and azide groups respectively (e.g. an intermediate containing the targeting group may contain an azide group, and an intermediate containing the spacer group may contain an alkyne group). Such reaction leads to formation of a triazole-containing group, e.g.:

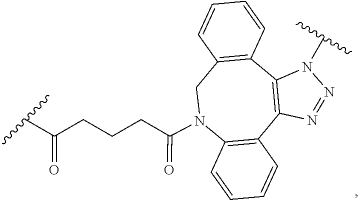 [SMILES: *C(=O)CCCC(=O)N1Cc2ccccc2-c2c(nnn2*)-c2ccccc21]

and may be formed by reaction of precursors having the following structures:

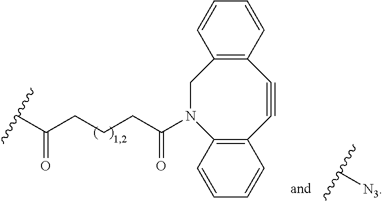

As another example, the spacer group may be attached via formation of a triazole-containing group, e.g.:

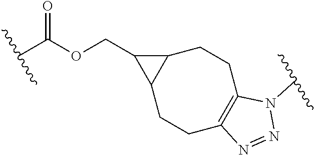 [SMILES: *C(=O)OCC1C2CCc3nnn(*)c3CCC21]

and may be formed by reaction of precursors having the following structures:

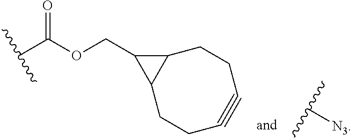

In some embodiments, the spacer group is attached to the targeting agent by means of reaction of precursors containing alkene (e.g. strained alkenes such as trans-cyclooctene) and tetrazine groups respectively. Such reaction leads to formation of a pyridazine-containing group, with extrusion of nitrogen, e.g.

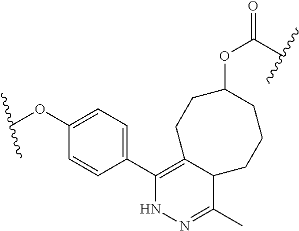 [SMILES: *Oc1ccc(C2=C3CCC(OC(*)=O)CCCC3C(C)=NN2)cc1]

and may be formed by reaction of precursors having the following structures:

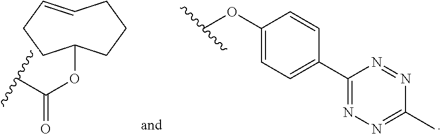 [SMILES: *C(=O)OC1CC/C=C/CCC1 ; *Oc1ccc(-c2nnc(C)nn2)cc1]

In some embodiments, the spacer group is attached to the dendrimer by means of reaction of precursors comprising carboxylic acid and amine groups, e.g. a spacer group intermediate may contain a carboxylic acid group which can react with an amine group present as part of or extending from the core unit, e.g. forming an amide linkage.

In some embodiments, the spacer group is attached to the dendrimer by means of reaction of precursors comprising carboxylic acid and amine groups, e.g. a spacer group intermediate may contain a carboxylic acid group which can react with an amine group present as part of or extending from the core unit, e.g. forming an amide linkage, and is attached to the targeting agent by reaction of precursors containing alkyne and azide groups respectively (e.g. an intermediate containing the targeting group may contain an azide group, and an intermediate containing the spacer group may contain an alkyne group).

As discussed above, a precursor comprising the targeting agent may comprise an unnatural amino acid residue. This unnatural amino acid residue may be, for example, any unnatural amino acid capable of presenting a reactive side-chain, wherein the reactive side-chain bears functional groups that are complementary to a functional group present on a spacer group intermediate. In such a way, it is possible for the complementary functional groups to react, and therefore for attachment of the targeting moiety to the spacer group to occur. In some embodiments, the unnatural amino acid residue is a 4-azidophenylalanine residue. In some embodiments, an intermediate comprising the spacer group contains an alkyne group for conjugating to an intermediate comprising the targeting moiety that contains an unnatural amino acid residue which contains an azide group. In some embodiments, the spacer group-containing intermediate contains an alkyne group for conjugating to an intermediate containing the targeting agent containing a 4-azidophenylalanine residue. In some embodiments, the spacer group-containing intermediate contains an alkyne group that is a dibenzylcyclooctyne-amine (DBCO) group for conjugating to an intermediate containing a targeting moiety containing a 4-azidophenylalanine residue.

In some embodiments, one end of the spacer group is attached to the targeting moiety by cycloaddition reaction of a DBCO group with an azido moiety on a 4-phenylalanine residue that forms part of the targeting moiety, e.g. forming a triazole-containing group such as:

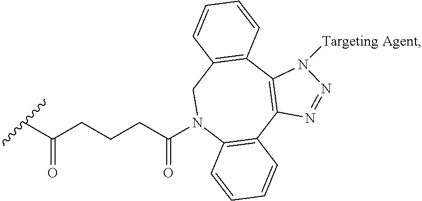

or by reaction of a BCN ((bicycle[6.1.0]non-4-yn-9-yl)) group with an azido moiety on a 4-phenylalanine residue which forms part of the targeting moiety, e.g. forming a triazole-containing group such as:

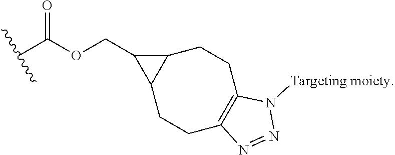

In some embodiments, one end of the spacer group is attached to the dendrimer by an amidation reaction between an amino group present on the core or present on a surface building unit, and between a carboxyl group present on the spacer group (e.g. by reaction of an activated ester).In some embodiments, the intermediate containing a spacer group contains a tetrazine group. In some embodiments, the intermediate containing spacer group comprises a maleimide group, e.g. for conjugating to a diene (such as a cyclopentadiene, e.g. a spiro [2.4]hepta-4,6-diene group).

In some embodiments, an intermediate comprising the spacer group comprises a PEG group, a carboxyl group for reacting with an amine forming part of or extending from the core of the dendrimer, and comprises an alkyne group for reacting with an azide group present in an intermediate containing the targeting moiety. In some embodiments, an intermediate comprising the spacer group contains a PEG chain with a reactive carboxyl group for joining to an amine at the core of the dendrimer and an azide group for conjugating to a targeting agent intermediate containing a reactive alkyne moiety. In some embodiments, an intermediate comprising a spacer group has a PEG chain, a reactive amine group for joining to a carboxyl group at the core of the dendrimer, and an azide group for conjugating to a targeting agent intermediate containing a reactive alkyne moiety. In some embodiments, a spacer group intermediate has a PEG chain with a reactive carboxyl group for joining to an amine at the core of the dendrimer and a maleimide group for conjugating to a targeting agent intermediate containing a reactive thiol moiety. In some embodiments, a spacer group intermediate has a PEG chain with a reactive amine group for joining to a carboxyl group at the core of the dendrimer and a thiol or masked thiol group for conjugating to a targeting agent intermediate containing a reactive maleimide moiety. In some embodiments, a spacer group intermediate contains a PEG chain with a reactive carboxyl group for joining to an amine at the core of the dendrimer and a tetrazine group for conjugating to a targeting agent intermediate containing a reactive alkene moiety. In some embodiments, a spacer group intermediate contains a PEG chain with a reactive carboxyl group for joining to an amine at the core of the dendrimer and a maleimide group for conjugating to a targeting agent intermediate containing a reactive diene (such as a cyclopentadiene, e.g. a spiro [2.4]hepta-4,6-diene group).

In some embodiments, the linkage of the targeting agent to the dendrimer may be accomplished through attaching a first spacer group to a targeting agent intermediate, a second spacer group to the dendrimer (e.g. to the core of the dendrimer), and then reacting together complementary functional groups present on the first and second spacer groups in order to link the targeting agent and the dendrimer. Such an approach may provide for facile connection of dendrimer to targeting agent. For example, a first spacer group intermediate may comprise a first reactive group at one end which is complementary to a reactive group on a targeting agent (e.g. an alkyne group, which is complementary with an azide group, and can react together to form a triazole group), and a second reactive group which is complementary to a reactive group on a second spacer group (e.g. a tetrazine-containing group which is complementary to reaction with a trans-cyclooctene-containing group). A second spacer group intermediate may for example comprise a third reactive group at one end which complementary to reaction with a reactive group on a dendrimer (e.g. a carboxylic acid group, which is complementary to reaction with an amine group), and a fourth reactive group at the other end which is complementary to reaction with a reactive group on the first spacer group intermediate (e.g. a trans-cyclooctene-containing group which is complementary to reaction with a tetrazine group). For example, first and spacer groups may be attached via a group produced by reaction of a trans-cyclooctene group and a tetrazine group, and comprising the structure:

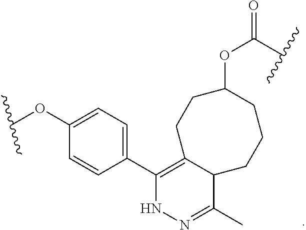 [SMILES: *Oc1ccc(C2=C3CCC(OC(*)=O)CCCC3C(C)=NN2)cc1]

In some embodiments, the targeting moiety may be linked to the dendrimer via a spacer group which is formed by reaction of an azide-moiety present on the targeting agent with an alkyne containing group at one end of a spacer group (e.g. DBCO, BCN), and by reaction of a tetrazine moiety attached to the dendrimer with a strained alkene group at the other end of the spacer group (e.g. trans cyclooctene).

### Third Terminal Group

In some embodiments, the dendrimer comprises one or more third terminal groups (T3) attached to an outermost building unit, the third terminal group comprising a residue of a pharmaceutically active agent that is not a radionuclide-containing moiety. Where the building units are lysine residues or analogues thereof, the third terminal group may for example be attached to the nitrogen atom of an outermost building unit. Incorporation of a pharmaceutically active agent into the dendrimer can provide improved therapeutic properties, and can lead to the same dendrimeric agent being capable of utilisation for both diagnostic/theranostic imaging, and for therapy of disease. For example, in the case of a subject who is suspected of having or who has been diagnosed as having a cancer, the dendrimer of the present disclosure may initially be administered and imaging of the relevant part(s) of the subject's body carried out, in order to diagnose the patient's condition by imaging and/or, where cancer is present, to determine the likely susceptibility of the cancer to a course of therapy with the dendrimer. In the case where the tumour is likely susceptible to treatment with dendrimer, a further course of the same dendrimer, or another dendrimer of the present disclosure, e.g. containing a different radionuclide, may for example then be administered to the subject.

### Pharmaceutically Active Agent

Any suitable pharmaceutically active agent may be conjugated to the dendrimer as the third terminal group, for example via a linking group. In some embodiments, the pharmaceutically active agent is an anti-cancer agent. Examples of anti-cancer agents include, but are not limited to, ultracytotoxic agents, taxanes, and topoisomerase inhibitors. In some embodiments, the anti-cancer agent is an ultracytotoxic agent. In some embodiments, the anti-cancer agent is an auristatin. In some embodiments, the anti-cancer agent is a maytansinoid. In some embodiments, the anti-cancer agent is a taxane. In some embodiments, the anti-cancer agent is a topoisomerase inhibitor.

As used herein, the term “ultracytotoxic agent” refers to agents that exhibit highly potent chemotherapeutic properties, yet themselves are too toxic to administer alone as an anti-cancer agent. That is, an ultracytotoxic agent, although demonstrating chemotherapeutic properties, generally cannot be safely administered to a subject as the detrimental, toxic side-effects outweigh the chemotherapeutic benefit. In some embodiments, the ultracytotoxic has an in vitro IC₅₀ against a cancer cell line (e.g. SKBR3 and/or HEK293 cells and/or MCF7 cells) which is less than 100 nM, or less than 10 nM, or less than 5 nM, or less than 3 nM, or less than 2 nM, or less than 1 nM, or less than 0.5 nM. Ultracytotoxic agents include, for example, the dolastatins (e.g., dolastatin-10, dolastatin-15), auristatins (e.g., monomethyl auristatin-E, monomethyl auristatin-F), maytansinoids (e.g., maytansine, mertansine/emtansine (DM1, ravtansine (DM4)), calicheamicins (e.g., calicheamicin γ1), esperamicins (e.g., esperamicin A1), and pyrrolobenzodiazepines (PDB) amongst others.

In some embodiments, the pharmaceutically active agent is an auristatin. In some embodiments, the pharmaceutically active agent is a monomethyl auristatin. In one embodiment, the pharmaceutically active agent is monomethyl auristatin E (MMAE). In one embodiment, the pharmaceutically active agent is monomethyl auristatin F (MMAF). Both MMAE and MMAF are understood to inhibit cell division by blocking the polymerisation of tubulin.

In some embodiments, the ultracytotoxic agent is a maytansinoid. In one embodiment, the ultracytotoxic agent is maytansine. In one embodiment, the ultracytotoxic agent is ansamitocin. In one embodiment, the ultracytotoxic agent is emtansine/mertansine (DM1). In one embodiment, the ultracytotoxic agent is ravtansine (DM4). The maytansinoids are understood to inhibit the assembly of microtubules by binding to tubulin.

Taxanes include, for example, paclitaxel, cabazitaxel and docetaxel. In some embodiments, the pharmaceutically active agent is paclitaxel. In some embodiments, the pharmaceutically active agent is cabazitaxel. In some embodiments, the pharmaceutically active agent is docetaxel.

Topoisomerase inhibitors include, but are not limited to, the camptothecin actives. In some embodiments, the pharmaceutically active agent is a camptothecin active. Examples of camptothecin actives include, but are not limited to, SN-38, irinotecan (CPT-11), topotecan, silatecan, cositecan, exatecan, lurtotecan, gimatecan, belotecan and rubitecan. In some embodiments, the pharmaceutically active agent is SN-38. In some embodiments, the pharmaceutically active agent is irinotecan.

In some embodiments, the pharmaceutically active agent is an anticancer agent is selected from the group consisting of cabazitaxel, docetaxel, SN-38, monomethyl auristatin A and monomethyl auristatin F.

### Linkers

In some embodiments, where the dendrimer comprises a third terminal group (T3) comprising a residue of a pharmaceutically active agent, the residue of a pharmaceutically active agent is attached to an outermost building unit via a linker. In some embodiments, the linker is a cleavable linker. In some embodiments, the linker is a non-cleavable linker. Linker groups can be used for example to provide suitable groups for attaching a pharmaceutically active agent to the dendrimer, for example where available functionality in the pharmaceutically active agent is not suitable for direct attachment to a building unit. Linker groups can also or instead by used to facilitate controlled release of the pharmaceutically active agent from the dendrimeric scaffold, providing a therapeutically effective concentration and desirable pharmacokinetic profile of the pharmaceutically active agent for a suitable (e.g. prolonged) period of time.

A person skilled in the art will appreciate that any one of a variety of suitable linkers may be used. The linker should provide sufficient stability during systemic circulation, though allow for the rapid and efficient release of the cytotoxic drug in an active form at its site of action, e.g. once internalised into a cancer cell.

In some embodiments, the linker is a cleavable linker which, either itself or in conjunction with its linkage to the pharmaceutically active agent, comprises one or more of the following cleavable moieties: an ester group, a hydrazone group, an oxime group, an imine group or a disulphide group. In some embodiments, the linker is tumour environment cleavable, acid labile, reductive environment labile, hydrolytically labile or protease sensitive.

Chemically labile linkers include, but are not limited to, acid-labile linkers (i.e., hydrazones) and disulphide linkers. Enzymatically cleavable linkers include, but are not limited to, peptide linkers (e.g. dipeptide linkers such as those containing Val-Cit, or Phe-Lys groups), and β-glucuronide linkers. Peptide linkers, and their peptide bonds, are advantageously expected to have good serum stability, as lysosomal proteolytic enzymes have very low activities in blood. Both Val-Cit and Phe-Lys linkers are rapidly hydrolysed by Cathepsin B. In some embodiments, the linker is an enzymatically-cleavable linker. For example, in some embodiments, the linker comprises amino acid residues which are capable of recognition and cleavage by an enzyme.

In some embodiments, the linker comprises a peptide group. In some embodiments, the linker comprises a dipeptide group. In some embodiments, the linker comprises a valine-citrulline-paraaminobenzyl alcohol-containing group (Val-Cit-PAB), e.g. having the structure:

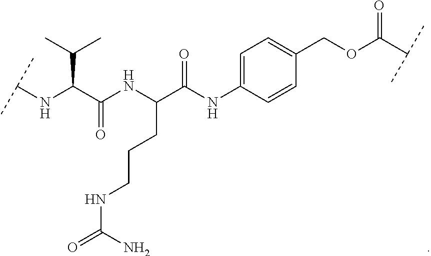

For example, the PAB group may be covalently attached to an amine group present on a therapeutic agent moiety via the carbonyl group, forming a carbamate linkage, and may be attached to an amine group present on an outer building unit via a diacyl linker which forms amide bonds with the valine amino group and the amine group present on the outer building unit.

In some embodiments, the linker comprises or consists of a glutaric acid-valine-citrulline-paraaminobenzyl alcohol group, .e.g. having the structure:

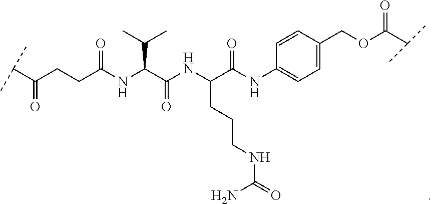

In some embodiments, the pharmaceutically active agent comprises a hydroxyl group, and the residue of the pharmaceutically active agent is attached to a linker via the oxygen atom of the hydroxyl group. This approach allows attachment to the linker via an ester group, and such ester groups have been found to be cleavable in vivo to release pharmaceutically active agent at a desirable rate.

In some embodiments, the core unit is formed from a core unit precursor comprising amino groups, the building units are lysine residues or analogues thereof, the pharmaceutically active agent comprises a hydroxyl group, the residue of the pharmaceutically active agent is attached via the oxygen atom of the hydroxyl group, and the cleavable linker is a diacyl linker, such that there is an ester linkage between the residue of the pharmaceutically active agent and the linker, and an amide linkage between the linker and a nitrogen atom present on an outermost building unit. In some embodiments, the pharmaceutically active agent comprises a hydroxyl group, the residue of the pharmaceutically active agent is attached via the oxygen atom of the hydroxyl group, and the cleavable linker is a diacyl linker group of formula

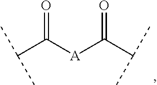

wherein A is a C₂-C₁₀ alkylene group which is optionally interrupted by O, S, S—S, NH, or N(Me), or in which A is a heterocycle selected from the group consisting of tetrahydrofuran, tetrahydrothiophene, pyrrolidine and N-methylpyrrolidine.

As used herein, the term “alkyl” refers to a monovalent straight-chain (i.e. linear) or branched saturated hydrocarbon group. In one example, an alkyl group contains from 1 to 10 carbon atoms ((i.e. C₁₋₁₀alkyl). In one example, an alkyl group contains from 1 to 6 carbon atoms (i.e. C₁₋₆ alkyl). Examples of alkyl groups include methyl, ethyl, propyl (e.g., n-propyl, iso-propyl), butyl (e.g. n-butyl, sec-butyl, tert-butyl), pentyl and hexyl groups.

As used herein, the term “alkylene” refers to a divalent straight-chain (i.e. linear) or branched saturated hydrocarbon group. In one example, an alkylene group contains from 2 to 10 carbon atoms ((i.e. C₂₋₁₀ alkylene). In one example, an alkylene group contains from 2 to 6 carbon atoms (i.e. C₂₋₆ alkylene). Examples of alkylene groups include, for example, —CH₂CH₂—, —CH₂CH₂CH₂—, —CH₂CH(CH₃)—, —CH₂CH₂CH₂CH₂—, —CH₂CH(CH₃)CH₂—, and the like.

In some embodiments, the pharmaceutically active agent comprises a hydroxyl group, the residue of the pharmaceutically active agent is attached via the oxygen atom of the hydroxyl group, and the cleavable linker is a diacyl linker group of formula

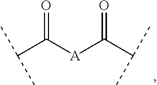

wherein A is a C₂-C₁₀ alkylene group which is interrupted by O, S, NH, or N(Me).

In some embodiments, the pharmaceutically active agent comprises a hydroxyl group, the residue of the pharmaceutically active agent is attached via the oxygen atom of the hydroxyl group, and the diacyl linker is

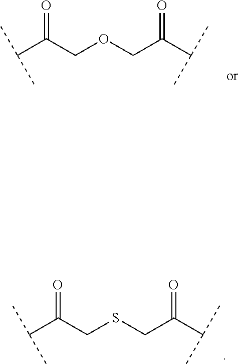

A specific type of cleavable linker is one which contains a disulphide moiety. Such linkers are susceptible to cleavage by glutathione. For example, a linker of this type may comprise two acyl groups linked via an alkyl chain interrupted by a disulphide moiety.

In some embodiments, the linker comprises an alkyl chain interrupted by a disulphide moiety, in which one or both of the carbon atoms which are next to the disulphide group are substituted by one or more methyl groups. For example, one of the carbon atoms next to the disulphide moiety may be substituted by a gem-dimethyl group, e.g. the linker may comprise the group:

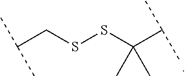

Noncleavable linkers are linking groups which are inert or substantially inert to cleavage on exposure to in vivo conditions over the required time period. Noncleavable linkers are not cleaved under biological conditions.

Examples include diacyl linkers bridged by an alkylene or a cycloalkylene group, e.g. a C₁₋₁₀ alkylene group or C₃₋₁₀ cycloalkylene group. Further examples of noncleavable linkers include thioether linkers. A specific example of a noncleavable linker is one formed by use of SMCC (succinimidyl 4-(N-maleimidomethyl)cyclohexane-1-carboxylate. SMCC can be used to react the maleimide functionality with a thiol group present on the therapeutic agent moiety, or which is attached to the therapeutic agent moiety, forming a thioether linkage. The carboxylic acid functionality can be used to react with an amino group present on an outer building unit.

### Compositions

In some embodiments, the conjugate is presented as a composition, preferably a pharmaceutical composition. Accordingly, there is also provided a composition comprising a plurality of conjugates as described herein.

The composition or pharmaceutical composition may comprise a plurality of dendrimer-targeting agent conjugates and/or dendrimer-targeting agent therapeutic conjugates. If the composition or pharmaceutical composition comprises only dendrimer-targeting agent conjugates then the composition may be exposed to a suitable radionuclide to form dendrimer-targeting agent therapeutic conjugates prior to administration for radiotherapy or imaging or like purposes.

It will be appreciated that there may be some variation in the molecular composition between the conjugates present in a given composition, as a result of the nature of the synthetic process for producing the conjugates. For example, as discussed above one or more synthetic steps used to produce the conjugates may not proceed fully to completion, which may result in the presence of conjugates which do not all comprise the same number of targeting agents, first terminal groups, second terminal groups, or third terminal groups, or which contain incomplete generations of building units in the dendrimeric component of the conjugate.

In some embodiments, where the composition comprises conjugates comprising three generations of building units, the mean number of targeting agents per conjugate is about 1. In some embodiments, where the composition comprises conjugates comprising three generations of building units, the mean number of first terminal groups per conjugate in the composition is in the range of from 1 to 4. In some embodiments, where the composition comprises conjugates comprising three generations of building units, the mean number of second terminal groups per conjugate in the composition is in the range of from 4 to 7. In some embodiments, where the composition comprises conjugates comprising three generations of building units as defined herein, the mean number of targeting agents per conjugate is about 1, the mean number of first terminal groups per conjugate in the composition is in the range of from 1 to 4, and the mean number of second terminal groups per conjugate in the composition is in the range of from 4 to 7.

In some embodiments, where the composition comprises conjugates comprising four generations of building units, the mean number of targeting agents per conjugate is about 1. In some embodiments, where the composition comprises conjugates comprising four generations of building units, the mean number of first terminal groups per conjugate in the composition is in the range of from 1 to 4. In some embodiments, where the composition comprises conjugates comprising four generations of building units, the mean number of second terminal groups per conjugate in the composition is in the range of from 4 to 7. In some embodiments, where the composition comprises conjugates comprising four generations of building units as defined herein, the mean number of targeting agents per conjugate is about 1, the mean number of first terminal groups per conjugate in the composition is in the range of from 1 to 4, and the mean number of second terminal groups per conjugate in the composition is in the range of from 4 to 7.

In some embodiments, at least 50%, at least 60%, at least 70%, at least 80%, at least 90%, or at least 95% of the conjugates in the composition contain a targeting agent.

In some embodiments, at least 50%, at least 60%, at least 70%, at least 80%, at least 90%, or at least 95% of the conjugates in the composition contain a first terminal group.

In some embodiments, at least 50%, at least 60%, at least 70%, at least 80%, at least 90%, or at least 95% of the conjugates in the composition contain a second terminal group.

In some embodiments, at least 50%, at least 60%, at least 70%, at least 80%, at least 90%, or at least 95% of the conjugates in the composition contain a third terminal group.

The present disclosure also provides pharmaceutical formulations or compositions, both for veterinary and for human medical use, which comprise the conjugates of the present disclosure or a pharmaceutically acceptable salt thereof, with one or more pharmaceutically acceptable carriers, and optionally any other therapeutic ingredients, stabilisers, or the like. Accordingly, in some embodiments, the composition is a pharmaceutical composition, and the composition comprises a conjugate as defined herein, and a pharmaceutically acceptable excipient.

The excipient(s) and/or carrier(s) must be pharmaceutically acceptable in the sense of being compatible with the other ingredients of the formulation and not unduly deleterious to the recipient thereof. The compositions of the present disclosure may also include polymeric excipients/additives or carriers, e.g., polyvinylpyrrolidones, derivatised celluloses such as hydroxymethylcellulose, hydroxyethylcellulose, and hydroxypropylmethylcellulose, ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin. The compositions may further include diluents, buffers, citrate, trehalose, binders, disintegrants, thickeners, lubricants, preservatives (including antioxidants), inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the present disclosure are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

The conjugates of the present disclosure may be formulated in compositions including those suitable for administration by any suitable route, including for example by parenteral (including intraperitoneal, intravenous, subcutaneous, or intramuscular injection) administration.

The compositions may conveniently be presented in unit dosage form and may be prepared by any of the methods well known in the art of pharmacy. All methods include the step of bringing the dendrimer into association with a carrier that constitutes one or more accessory ingredients. In general, the compositions are prepared by bringing the dendrimer into association with a liquid carrier to form a solution or a suspension, or alternatively, bring the dendrimer into association with formulation components suitable for forming a solid, optionally a particulate product, and then, if warranted, shaping the product into a desired delivery form. Solid formulations of the present disclosure, when particulate, will typically comprise particles with sizes ranging from about 1 nanometer to about 500 microns. In general, for solid formulations intended for intravenous administration, particles will typically range from about 1 nm to about 10 microns in diameter. The composition may contain dendrimer of the present disclosure that are nanoparticulate having a particulate diameter of below 1000 nm, for example, between 5 and 1000 nm, especially 5 and 500 nm, more especially 5 to 400 nm, such as 5 to 50 nm and especially between 5 and 20 nm. In one example, the composition contains dendrimers with a mean size of between 5 and 20 nm. In some embodiments, the dendrimer is polydispersed in the composition, with PDI of between 1.01 and 1.8, especially between 1.01 and 1.5, and more especially between 1.01 and 1.2. In one example, the dendrimer is monodispersed in the composition.

In some preferred embodiments, the composition is formulated for parenteral delivery. For example, in one embodiment, the formulation may be a sterile, lyophilized composition that is suitable for reconstitution in an aqueous vehicle prior to injection.

In one embodiment, a formulation suitable for parenteral administration conveniently comprises a sterile aqueous preparation of the dendrimer, which may for example be formulated to be isotonic with the blood of the recipient.

In some embodiments, the composition is formulated for intertumoural delivery. Other suitable means of delivery may also be used. For example, in some embodiments delivery may be by lavage or aerosol. In one embodiment the composition is formulated for intraperitoneal delivery, and is for treatment of cancers in the peritoneal cavity, which include malignant epithelial tumours (e.g., ovarian cancer), and peritoneal carcinomatosis (e.g. gastrointestinal especially colorectal, gastric, gynaecologic cancers, and primary peritoneal neoplasms).

Pharmaceutical formulations are also provided which are suitable for administration as an aerosol, by inhalation. These formulations comprise a solution or suspension of the desired conjugate or a salt thereof. The desired formulation may be placed in a small chamber and nebulized. Nebulization may be accomplished by compressed air or by ultrasonic energy to form a plurality of liquid droplets or solid particles comprising the dendrimers or salts thereof.

As discussed below, the conjugates of the present disclosure may for example be administered in combination with one or more additional pharmaceutically active agents. In some embodiments, the conjugate is provided in combination with a further active. In some embodiments, a composition is provided which comprises a conjugate as defined herein or a pharmaceutically acceptable salt thereof, one or more pharmaceutically acceptable carriers, and one or more additional pharmaceutically active agents, e.g. an additional anti-cancer/oncology agent, such as a small molecule cytotoxic, a checkpoint inhibitor, or an antibody therapy. Not only can the conjugates of the present disclosure be administered with other chemotherapy drugs but may also be administered in combination with other medications such as corticosteroids, anti-histamines, analgesics and drugs that aid in recovery or protect from hematotoxicity, for example, cytokines.

In some embodiments, the composition is formulated for parenteral infusion as part of a chemotherapy regimen.

### Therapeutic Uses of Conjugates

The conjugates and compositions as described herein can be used in various applications in the field of medicine. For example, the conjugates find use in the treatment of various conditions, such as cancers.

Accordingly, there is provided a conjugate or pharmaceutical composition as described herein for use in therapy, and more specifically for use in therapy of cancer. In some embodiments, the conjugate is used in a method of treating or preventing cancer, for example for suppressing the growth of a tumour. In some embodiments the conjugate is for use in the treatment of cancer. There is also provided a method of treating cancer comprising administering to a subject in need thereof a therapeutically effective amount of a conjugate or pharmaceutical composition as defined herein. There is also provided use of a conjugate as defined herein, or of a composition as defined herein, in the manufacture of a medicament for the treatment of cancer.

In some embodiments, the cancer is a solid tumour. The cancer may be a primary or metastatic tumour. In some embodiments, the cancer is a primary tumour. In some embodiments, the cancer is a metastatic tumour.

In some embodiments, the cancer is characterised by an abnormal, or overexpression, of HER2 (also referred to as ERBB2). Such abnormal or overexpression of HER2 is known to occur in, for example, breast cancers, testicular cancers, ovarian cancers, stomach cancers, adenocarcinomas of the lung, gastric cancers, pancreatic cancers, salivary duct carcinomas, oesophageal cancers, and uterine cancers (e.g., uterine serious endometrial carcinoma).

In some embodiments, the cancer is characterised by an abnormal, or overexpression, of EGFR. In some embodiments, the cancer is characterised by an abnormal, or overexpression, of PSMA. In some embodiments, the cancer is characterised by an abnormal, or overexpression, of FAP.

In some embodiments, the cancer is selected from the group consisting of prostate cancers, brain cancers, breast cancers, testicular cancers, ovarian cancers, stomach cancers, adenocarcinomas of the lung, gastric cancers, pancreatic cancers, salivary duct carcinomas, oesophageal cancers, and uterine cancers (e.g., uterine serious endometrial carcinoma).

In some embodiments, the cancer is selected from the group consisting of colorectal cancer, stomach cancer, pancreas cancer, prostate cancer and breast cancer.

In some embodiments, the cancer is prostate cancer. In some embodiments, the cancer is breast cancer. In some embodiments, the cancer is testicular cancer. In some embodiments, the cancer is ovarian cancer. In some embodiments, the cancer is stomach cancer. In some embodiments, the cancer is adenocarcinoma of the lung. In some embodiments, the cancer is gastric cancer. In some embodiments, the cancer is pancreatic cancer. In some embodiments, the cancer is salivary duct carcinoma. In some embodiments, the cancer is oesophageal cancer. In some embodiments, the cancer is uterine cancer.

In some embodiments, the cancer is brain cancer. Brain cancers include, but are not limited to, glioblastoma, meningioma, pituitary, nerve sheath, astrocytoma, oligodendroglioma, ependymoma, medulloblastoma, or craniopharyngioma. In some embodiments, the cancer is a brain cancer selected from the group consisting of glioblastoma, meningioma, pituitary, nerve sheath, astrocytoma, oligodendroglioma, ependymoma, medulloblastoma, and craniopharyngioma. In some embodiments, the brain cancer is glioblastoma. In some embodiments, the brain cancer is meningioma. In some embodiments, the brain cancer is pituitary. In some embodiments, the brain cancer is nerve sheath. In some embodiments, the brain cancer is astrocytoma. In some embodiments, the brain cancer is oligodendroglioma. In some embodiments, the brain cancer is ependymoma. In some embodiments, the brain cancer is medulloblastoma. In some embodiments, the brain cancer is craniopharyngioma.

A therapeutically effective amount of the conjugate or composition is used in the therapeutic methods and uses. It will be appreciated that the term “therapeutically effective amount” refers to a conjugate, or composition comprising the conjugate, being administered in an amount sufficient to alleviate or prevent to some extent one or more of the symptoms of the disorder or condition being treated.

The dendrimer-targeting agent therapeutic conjugate may be administered by any suitable route, including for example, intravenously. In some embodiments, the dendrimer-targeting agent therapeutic conjugate is delivered as an IV bolus. In some embodiments the dendrimer-targeting agent therapeutic conjugate is administered IV over a time a period in the range of from 0.5 to 60 minutes, or in the range of from 0.5 to 30 minutes, or in the range of from 0.5 to 15 minutes, or in the range of from 0.5 to 5 minutes. In another example, the dendrimer-targeting agent therapeutic conjugate may be administered intraperitoneally. The route of administration may for example be targeted to the disease or disorder which the subject has. For example, in some embodiments, the disease or disorder may be an intra-abdominal malignancy such as a gynecological or gastrointestinal cancer, and the conjugate may be administered intraperitoneally. In some embodiments the conjugate may be for treatment of a cancer of the peritoneal cavity, such as a malignant epithelial tumors (e.g., ovarian cancer) or peritoneal carcinomatosis (e.g. gastrointestinal especially colorectal, gastric, gynecologic cancers, and primary peritoneal neoplasms), and the conjugate is administered intraperitoneally.

When used for treatment purposes, the conjugate will be administered in an amount sufficient to deliver a therapeutically effective dose of radioactivity to the target (e.g. tumour), whilst at the same time avoiding unacceptable exposure of other parts of the body (e.g. other organs) to radiation. The precise dosage may be dependent on the nature of the radionuclide (e.g. alpha emitter, beta emitter), and the condition to be treated.

In some embodiments, the, or each, dosage of the conjugate contains an amount of the radionuclide having a radioactivity up to about 10 GBq, or up to about 7.5 GBq, or up to 5 GBq or up to 2.5 GBq, or up to 1 GBq, or up to about 500 MBq, or up to about 250 MBq, or up to about 100 MBq, or up to about 50 MBq, or up to about 25 MBq, or up to about 10 MBq, or up to about 5 MBq. In some embodiments, the, or each, dosage of the conjugate contains an amount of the radionuclide having a radioactivity in the range of from 0.1 MBq to 10 GBq, from 0.1 MBq to 7.5 GBq, from 0.1 MBq to 5 GBq, from 0.1 MBq to 2.5 GBq, from 0.1 MBq to 1 GBq, from 0.1 MBq to 500 MBq, from 0.1 MBq to 250 MBq, from 0.1 MBq to 100 MBq, from 0.1 MBq to 50 MBq, from 0.1 MBq to 25 MBq, from 0.1 MBq to 10 MBq, from 0.1 MBq to 5 MBq, from 0.1 MBq to 2 MBq, from 0.1 MBq to 1 MBq, from 0.5 MBq to 10 MBq, from 1 to 10 MBq, from 1 to 5 MBq, from 5 to 10 MBq, or about 1, about 2, about 3, about 4, about 5, about 6, about 7, about 8, about 9, or about 10 MBq. In some embodiments, the, or each, dosage of the conjugate contains an amount of the radionuclide having a radioactivity in the range of from 0.1 MBq to 10 GBq, from 1 MBq to 10 GBq, from 10 MBq to 10 GBq, from 100 MBq to 10 GBq, from 500 MBq to 10 GBq from 1 GBq to 10 GBq, or from 5 GBq to 10 GBq.

For example, in some embodiments where the radionuclide is Lu¹⁷⁷, the, or each, dosage of the conjugate contains an amount of the radionuclide having a radioactivity in the range of from 1 GBq to 25 GBq, more preferably in the range of from 4 to 25 GBq, or in the range of from 4 to 10 GBq. In some embodiments, conjugates containing Lu¹⁷⁷ are dosed providing an amount of radioactivity in the range of from 5 to 20 MBq per kg bodyweight of the subject.

As another example, in some embodiments where the radionuclide is Ga⁶⁸, the, or each, dosage of the conjugate contains an amount of the radionuclide having a radioactivity in the range of from 50 MBq to 1 GBq. In some embodiments, conjugates containing Ga⁶⁸ are dosed at an amount in the range of from 1 to 5 MBq per kg bodyweight of the subject, more preferably in the range of from 1 to 3 MBq per kg, or about 2 MBq per kg.

As a further example, in some embodiments where the radionuclide is Y⁹⁰, the, or each, dosage of the conjugate contains an amount of the radionuclide having a radioactivity in the range of from 500 MBq to 20 GBq or from 10 to 20 GBq. In some embodiments, conjugates containing Y⁹⁰ are dosed at an amount in the range of from 5 to 50 MBq per kg bodyweight of the subject, more preferably in the range of from 10 to 15 MBq per kg.

Where the radionuclide is Ac²²⁵, the, or each, dosage of the conjugate may for example contain an amount of the radionuclude having a radioactivity in the range of from 1 MBq to 20 MBq. In some embodiments, conjugates containing Ac²²⁵ are dosed at an amount in the range of from 15 to 200 KBq per kg bodyweight.

Where the radionuclide is At²¹¹, the, or each, dosage of the conjugate may for example contain an amount of the radionuclide having a radioactivity in the range of from 50 MBq to 400 MBq.

As discussed above, the dose of conjugate administered is sufficient to deliver a therapeutically effective dose of radioactivity to the target (e.g. tumour), whilst at the same time avoiding unacceptable exposure of other parts of the body (e.g other organs).

In some embodiments, the amount of conjugate dosed in a single dose is such that the mean radiation absorbed per organ for the group of organs consisting of lung, spleen, bladder, kidney, heart, bone marrow, liver, and gastrointestinal tract (other than where the cancer or tumour is located in such organ) is less than 5 mGy, or less than 5 mBq, or less than 2 mGy, or less than 2 mBq, or less than 1 mGy, or less than 1 mBq or less than 0.5mGy.

Where the conjugate comprises a third terminal group which is a further pharmaceutically active agent, in some embodiments, the amount of conjugate administered is sufficient to deliver between 2 and 100 mg of active agent/m², between 2 and 50 mg of active agent/m², between 2 and 40 mg of active agent/m², between 2 and 30 mg of active agent/m², between 2 and 25 mg of active agent/m², between 2 and 20 mg of active agent/m², between 5 and 50 mg of active agent/m², between 10 to 40 mg of active agent/m² between 15 and 35 mg of active agent/m², between 10 and 20 mg/m², between 20 and 30 mg/m², or between 25 and 35 mg of active agent/m². A dose of active agent of 10 mg/kg in a mouse should be approximately equivalent to a human dose of 30 mg/m² (FDA guidance 2005). (To convert human mg/kg dose to mg/m², the figure may be multiplied by 37, FDA Guidance 2005).

In some embodiments, a therapeutically effective amount of the conjugate is administered to a subject in need thereof at a predetermined frequency. In some embodiments, the conjugate is administered to a subject in need thereof according to a dosage regimen in which the conjugate is administered once per one to four weeks. In some embodiments, the conjugate is administered to a subject in need thereof according to a dosage regimen in which the conjugate is administered once per three to four weeks. In some embodiments, a dosing regimen involving administration once per three to four weeks for a total of 2, 3, 4, 5, 6, 7, 8, 8, 9, or 10 doses is used.

### Combinations

Drugs are often administered in combination with other drugs, especially during chemotherapy. Accordingly, in some embodiments the conjugate is administered in combination with one or more further pharmaceutically active agents, for example one or more further anti-cancer agents/drugs. The dendrimer-targeting agent conjugate and the one or more further pharmaceutically active agents may be administered simultaneously, subsequently or separately. For example, they may be administered as part of the same composition, or by administration of separate compositions.

The one or more further pharmaceutically active agents may for example be anti-cancer agents for therapy of prostate cancers, brain cancers, breast cancers, testicular cancers, ovarian cancers, stomach cancers, adenocarcinomas of the lung, gastric cancers, pancreatic cancers, salivary duct carcinomas, oesophageal cancers, or uterine cancers (e.g., uterine serious endometrial carcinoma).

The one or more further pharmaceutically active agents may for example be anti-cancer agents for therapy of colorectal cancer, stomach cancer, pancreas cancer, prostate cancer or breast cancer.

Examples of further pharmaceutically active agents include chemotherapeutic and cytotoxic agents, small molecule cytotoxics, tyrosine kinase inhibitors, checkpoint inhibitors, EGFR inhibitors, antibody therapies, taxanes (e.g. paclitaxel, docetaxel, cabazitaxel, nab-paclitaxel), topoisomerase inhibitors (e.g. SN-38, irinotecan (CPT-11), topotecan, silatecan, cositecan, exatecan, lurtotecan, gimatecan, belotecan, or rubitecan), and aromatase inhibitors.

### Diagnosis/Imaging

The conjugates and compositions as described herein also find use as diagnostic agents, for example such as imaging agents. Examples of diagnostic applications include imaging, theranostics, companion diagnostic-therapeutic, monitoring disease progression, evaluating efficacy of therapy, determining patient group outcomes, and developing treatment regimes for specific patients or patient groups.

Accordingly, there is provided a method of determining whether a subject has a cancer, comprising:
administering to a subject a conjugate as defined herein or a pharmaceutical composition comprising the conjugate;
carrying out imaging on the subject's body or a part thereof, and
determining whether the subject has a cancer based on the imaging results.

There is also provided a method of imaging a cancer in a subject, comprising:
administering to a subject a conjugate as defined herein or a pharmaceutical composition comprising the conjugate; and
carrying out imaging on the subject's body or a part thereof.

There is also provided a method of determining the progression of a cancer in a subject, comprising:
administering to a subject a first amount of a conjugate as defined herein or of a pharmaceutical composition comprising the conjugate;
carrying out a first imaging step on the subject's body or a part thereof,
subsequently administering to the subject a second amount of a conjugate as defined herein or of a pharmaceutical composition comprising the conjugate;
carrying out a second imaging step on the subject's body or a part thereof, and
determining whether the cancer has progressed based on the first and second imaging results.

There is also provided a method of determining an appropriate therapy for a subject having a cancer, comprising:
administering to a subject a conjugate as defined herein or a pharmaceutical composition comprising the conjugate;
carrying out imaging on the subject's body or a part thereof, and
if the imaging results indicate susceptibility of the cancer to treatment with a therapy, administering the therapy to the subject.

There is also provided a method of determining the effectiveness of a cancer therapy administered to a subject having a cancer, comprising:
administering to the subject a first amount of a conjugate as defined herein, or a pharmaceutical composition as defined herein;
carrying out a first imaging step on the subject's body or a part thereof,
administering to the subject a cancer therapy;
subsequently administering to the subject a second amount of a conjugate as defined herein or a pharmaceutical composition as defined herein;
carrying out a second imaging step on the subject's body or a part thereof, and
determining the effectiveness of the cancer therapy based on the first and second imaging results.

There is also provided a conjugate as defined herein, or a pharmaceutical composition containing the conjugate, for use in the diagnosis of cancer in a subject, for use in determining an appropriate therapy for a subject having a cancer, for use in determining the effectiveness of a cancer therapy administered to a subject, for use in determining the progression of a cancer in a subject or for use in treating cancer.

There is also provided use of a conjugate as defined herein, or of a pharmaceutical composition containing the conjugate, in the manufacture of a medicament for the diagnosis of cancer, for determining an appropriate therapy for a subject having a cancer, for determining the effectiveness of a cancer therapy administered to a subject, for determining the progression of a cancer in a subject, or for the treatment of cancer.

The cancer may for example be any of the cancers discussed above in relation to therapeutic applications of the conjugates. For example in some embodiments, the cancer is a solid tumour. The cancer may be a primary or metastatic tumour. In some embodiments, the cancer is a primary tumour. In some embodiments, the cancer is a metastatic tumour.

In some embodiments, the cancer is characterised by an abnormal, or overexpression, of HER2 (also referred to as ERBB2). Such abnormal or overexpression of HER2 is known to occur in, for example, breast cancers, testicular cancers, ovarian cancers, stomach cancers, adenocarcinomas of the lung, gastric cancers, pancreatic cancers, salivary duct carcinomas, oesophageal cancers, and uterine cancers (e.g., uterine serious endometrial carcinoma).

In some embodiments, the cancer is selected from the group consisting of prostate cancers, brain cancers, breast cancers, testicular cancers, ovarian cancers, stomach cancers, adenocarcinomas of the lung, gastric cancers, pancreatic cancers, salivary duct carcinomas, oesophageal cancers, and uterine cancers (e.g., uterine serious endometrial carcinoma).

In some embodiments, the cancer is selected from the group consisting of colorectal cancer, stomach cancer, pancreas cancer, prostate cancer and breast cancer.

In the above methods and uses, any suitable means for administering an amount of conjugate or composition sufficient for the diagnostic use may be utilised. For example, the conjugate or composition may administered intravenously to the subject.

Suitable techniques for imaging radionuclide-containing samples, or subjects to whom a radionuclide has administered, and for analysing the results, are known to the person skilled in the art, and may be used in the above methods and uses.

Radionuclide-based imaging methods, especially PET (positron emission tomography), continue to be an active area for both diagnostic and therapeutic applications due to their high sensitivity (picomolar level) and limitless tissue penetration. In some embodiments, PET imaging is used. In some embodiments, PET-MRI, SPECT, SPECT-CT, CT, scintography or PET-CT imaging is used.

Typically, when used for imaging and diagnostic purposes, the conjugate is administered and then the subject, or relevant part of the subject, is imaged after a suitable period of time. The period of time in-between administration and imaging steps may be dependent on aspects including the nature of the targeting agent. For example, in some cases where a small molecule targeting agent is used, it may be beneficial to image the subject within 2 hours, within 1 hour, or within 30 minutes following administration. As a further example, in some cases where an antibody targeting agent is used or the dendimer is large, eg G4 or G5, it may be preferable to allow additional time to pass following administration before carrying out imaging, e.g. a period of 1, 2, 3, 4, 5, 6, or 7 days. In some embodiments, the conjugate is administered, and imaging is carried out approximately 24 hours, or approximately 48 hours, afterwards.

In some embodiments, the conjugate used for diagnostic and imaging is a conjugate having 2-generations or 3-generations of building units.

The present conjugates, and compositions comprising them, have good selectivity for the target of interest (e.g. tumour tissue). To further improve selectivity, and reduce levels of conjugate present in other tissues or organs, such as the kidney or the liver, the diagnostic and therapeutic methods may include additional steps as part of the administration regime.

For example, pre-administration or co-administration of an agent that reduces the potential for nephrotoxicity associated with exposure of the kidneys to radioactive agents may be carried out. This, in some embodiments of the therapeutic and diagnostic methods provided herein, the conjugate or pharmaceutical composition providing the conjugate, is administered in combination with an agent which reduces the potential for nephrotoxicity.

Examples of such agents include amino acids, e.g. basic amino acids such as lysine and/or arginine. In one example, an aqueous solution containing 18-24 g L-lysine and 18-24 g L-arginine per 1.5-2.2 L of solution is used. Such a solution may for example have an osmolarity of less than 1200 mOsmol, or less than 1100 mOsmol, or less than 1060 mOsmol. Further examples of suitable agents include succinylated gelatine (a 4% w/v solution is sold under the trade name Gelofusine by Hausmann Laboratories Ltd). Further examples of such agents include furosemide (sold under the brand name Lasix), and spironolactone (sold under the brand name Aldactone).

In some embodiments, pre-administration or co-administration of an amino acid such as lysine or arginine may be utilised. Thus, in some embodiments of the therapeutic and diagnostic methods provided herein, the conjugate or pharmaceutical composition providing the conjugate, is administered in combination with an amino acid, e.g. lysine, or arginine. In some embodiments, the amino acid (e.g. lysine, arginine) is administered prior to administration of the conjugate or composition containing the conjugate. In some embodiments, the amino acid (e.g. lysine, arginine) is administered simultaneously with the conjugate or composition containing the conjugate.

In some embodiments, succinylated gelatin is administered in combination with the conjugate or pharmaceutical composition providing the conjugate. In some embodiments, succinylated gelatin is administered prior to administration of the conjugate or composition containing the conjugate.

In some embodiments a combination of succinylated gelatin and an amino acid (e.g. lysine, arginine) is administered either prior to or simultaneously with administration of the conjugate.

In some embodiments, furosemide is administered either prior to or simultaneously with administration of the conjugate.

In some embodiments, spironolactone is administered either prior to or simultaneously with administration of the conjugate.

The agent (e.g. an amino acid such as lysine, arginine) is typically administered in the form of a pharmaceutical composition, e.g. an aqueous composition. The agent may for example be administered intravenously, e.g. by injection or infusion.

It will be appreciated that the conjugates suitable for diagnosis, treatment, imaging and other purposes requiring the presence of a radionuclide will be the therapeutic conjugates as described herein even though the end use may not be therapeutic in nature but rather diagnostic or otherwise use in imaging.

### Preparation of Therapeutic Conjugates

Radioactive materials are hazardous substances, and handling steps using such materials are ideally minimised. It is desirable to introduce the radionuclide component into the conjugates only at a late stage, ideally at a time just prior to use of the conjugates.

Accordingly, there is provided a process for producing a therapeutic conjugate as defined herein, comprising:
contacting a suitable dendrimer-targeting agent conjugate, as defined above, with a radionuclide, thereby producing the therapeutic conjugate; wherein the dendrimer-targeting agent conjugate comprises:
a) a dendrimer comprising
i) a core unit (C); and
ii) building units (BU),
wherein the dendrimer has from two to six generations of building units; and wherein the core unit is covalently attached to at least two building units;
b) a targeting agent which is covalently linked to the dendrimer by a spacer group;
c) one or more first terminal groups attached to an outermost building unit of the dendrimer, wherein the first terminal group comprises a complexation group for complexing a radionuclide; and
d) one or more second terminal groups attached to an outermost building unit of the dendrimer, wherein the second terminal group comprises a pharmacokinetic-modifying moiety;
   or a salt thereof.

There is also provided a kit for producing a therapeutic conjugate as defined herein, comprising a) the dendrimer-targeting agent conjugate as defined above; and b) a radionuclide.

It will be appreciated that any one or more various embodiments or examples as described herein for the conjugates, e.g. for the core unit (C), building unit (BU), terminal groups, targeting agent, or dendrimer, may also be provided for the intermediate. Similarly, any of the radionuclides discussed above in relation to the conjugate, may be used in the process for producing the conjugate.

Any suitable means of producing the therapeutic conjugate from the dendrimer-targeting agent conjugate and the radionuclide may be utilised. For example, the dendrimer-targeting agent conjugate and radionuclide (e.g. in the form of a metal salt) may be admixed in a suitable solvent, preferably a solvent which is suitable for administration to a patient. For example, in some embodiments, an aqueous solvent may be used.

In some embodiments, a suitable salt form of a radionuclide (e.g. Zr⁸⁹ oxalate) in aqueous solution may be admixed with a solution of the dendrimer-targeting agent conjugate or intermediate in a suitable buffer (e.g HEPES). Any suitable molar ratio of intermediate to radionuclide salt may be used, e.g at least 25:1, at least 50:1, or about 100:1. Purification to separate from unbound radionuclide may be carried out if needed. If desired, the solution may be exchanged prior to administration, e.g. buffer exchange into phosphate-buffered saline may be carried out.

If other metal ion species are present (e.g. if the intermediate contains significant levels of chelated metals) these may be removed if desired prior to labelling with radionuclide. For example, iron contaminants may be removed by treatment with EDTA (ethylenediamine tetraacetic acid), prior to labelling with radionuclide.

Labelling of dendrimer-targeting agent conjugates or intermediates may for example be carried out in accordance with procedures described in Verel et al, J. Nucl. Med., 2003, 44(8), p1271-1281.

The above described kits, intermediates, and processes can be used to provide an effective preparation of pharmaceutical compositions in the clinic, by allowing for radiolabelling of the intermediates and production of the conjugates in the clinic immediately prior to administration.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

The following nomenclature is used herein in reference to dendrimer conjugate synthesis:

| Abbreviation | Structure |
|---|---|
| Azido- PEG₂₄CO— | 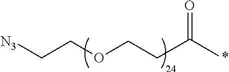 |
| —COPEG₂₄ NH—COPEG₄ (PhTzMe) | 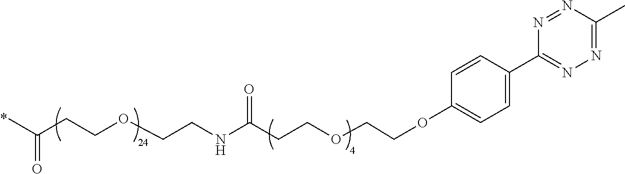 |
| HO-Lys[(α- NHCy5)(ε- NHDFO)] Compound 60 | 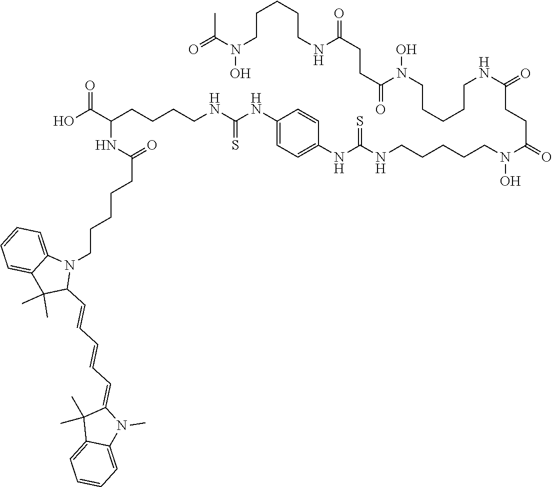 [SMILES: CC(=O)N(O)CCCCCNC(=O)CCC(=O)N(O)CCCCCNC(=O)CCC(=O)N(O)CCCCCNC(=S)Nc1ccc(NC(=S)NCCCCC(NC(=O)CCCCCN2c3ccccc3C(C)(C)C2/C=C/C=C/C=C2/N(C)c3ccccc3C2(C)C)C(=O)O)cc1] |
| —COPEG₂₄ NH—COPEG₄ (PhMeTz)/ TCO— PEG₃NH— COPEG₂₄ NH—Glu- DBCO/N₃— Nanobody) | 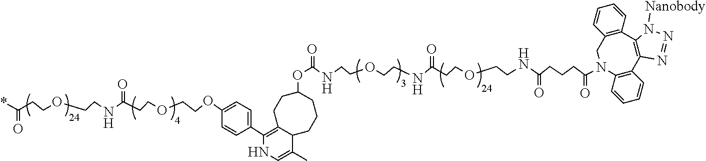 |
| —COPEG₅₇₀N₃ | 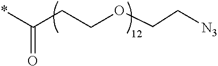 |
| —COPEG₅₇₀ N₃/BCN-DUPA | 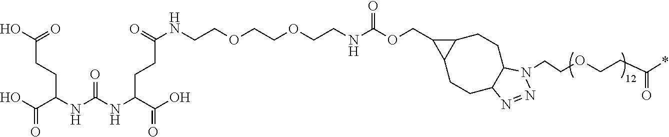 |
| —COPEG₂₄ NH—DUPA (OtBu)₃) | 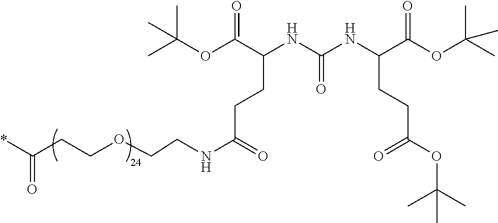 |
| —COPEG₂₄ NH—DUPA (OH)₃) | 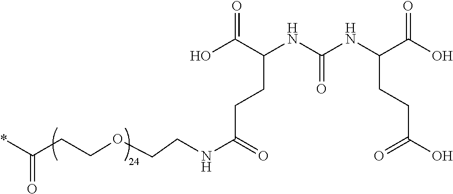 |
| HO-Lys[(α- NH—COPEG₂₄ NH—DUPA (OtBu)₃)(ε- GluNH-p-Bn- DOTA (OtBu)₄)] Compound 45 | 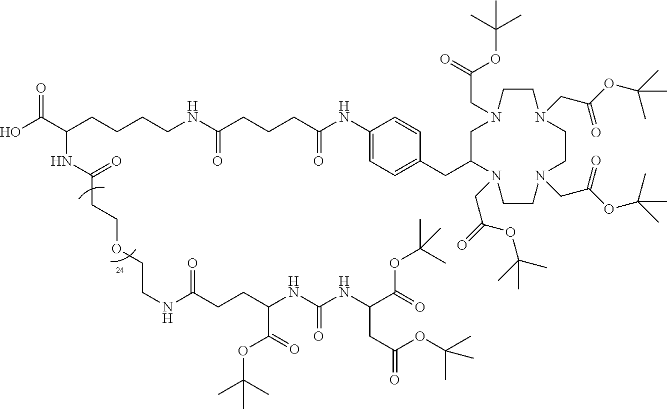 |
| (α-(NH DOTA) | 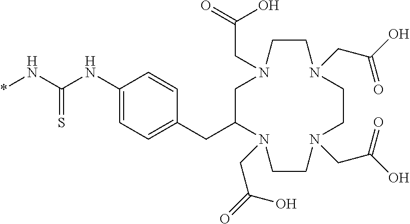 [SMILES: *NC(=S)Nc1ccc(CC2CN(CC(=O)O)CCN(CC(=O)O)CCN(CC(=O)O)CCN2CC(=O)O)cc1] |
| BCN-PEG₂- Glu—CO— NHPEG₂₄CO— NHPEG₃-TCO Compound 50 | 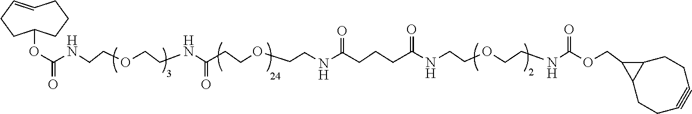 |
| DBCO—Glu— NHPEG₂₄ COOTFP | 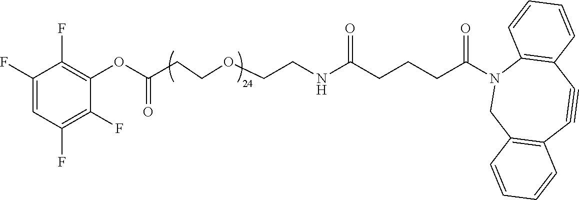 |
| DBCO—Glu- NHPEG₂₄CO— NHPEG₃— TCO Compound 51 | 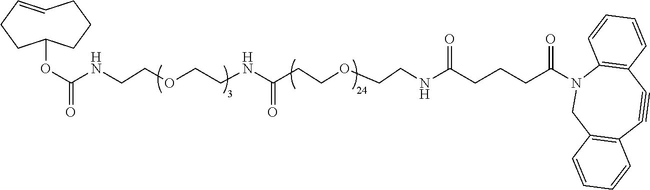 |
| TCO—PEG₃— NH₂ or TCO— PEG₃—Amine | 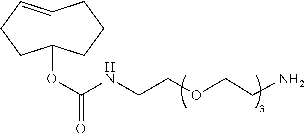 |
| DFO—PEG₄- Sulfo-DBCO Compound 55 | 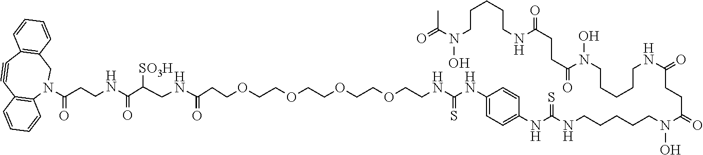 [SMILES: CC(=O)N(O)CCCCCNC(=O)CCC(=O)N(O)CCCCCNC(=O)CCC(=O)N(O)CCCCCNC(=S)Nc1ccc(NC(=S)NCCOCCOCCOCCOCCC(=O)NCC(C(=O)NCCC(=O)N2Cc3ccccc3C#Cc3ccccc32)S(=O)(=O)O)cc1] |
| DBCO-NHS Ester | 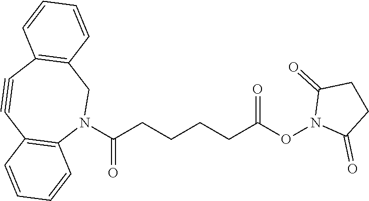 [SMILES: O=C(CCCCC(=O)N1Cc2ccccc2C#Cc2ccccc21)ON1C(=O)CCC1=O] |
| DFO-DBCO Compound 56 | 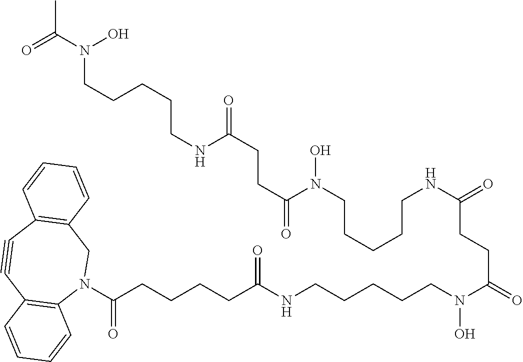 [SMILES: CC(=O)N(O)CCCCCNC(=O)CCC(=O)N(O)CCCCCNC(=O)CCC(=O)N(O)CCCCCNC(=O)CCCCC(=O)N1Cc2ccccc2C#Cc2ccccc21] |
| DUPA-BCN Compound 61 | 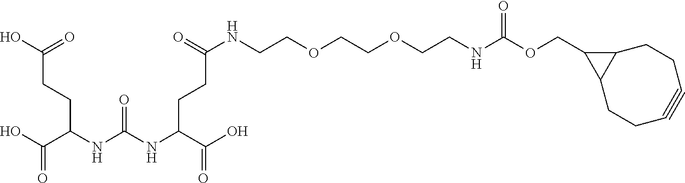 [SMILES: O=C(O)CCC(NC(=O)NC(CCC(=O)NCCOCCOCCNC(=O)OCC1C2CCC#CCCC21)C(=O)O)C(=O)O] |
| Affibody- BCN/N₃— PEG₂₄CO— | 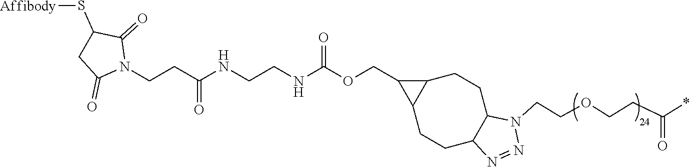 |
| Mal-BCN | 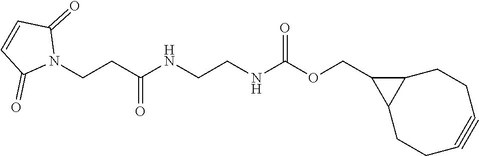 [SMILES: O=C(CCN1C(=O)C=CC1=O)NCCNC(=O)OCC1C2CCC#CCCC21] |
| Glu-vc-PAB- MMAE | 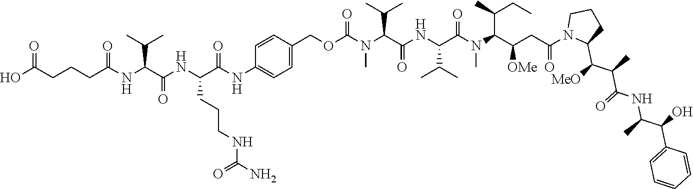 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)O)C(C)C)cc1)C(C)C] |
| DGA- MMAF(OMe) | 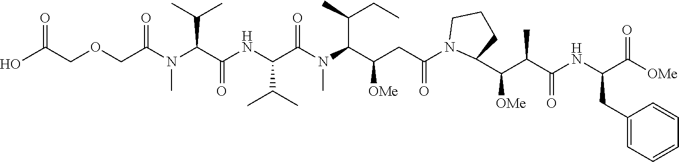 [SMILES: CC[C@H](C)[C@@H]([C@@H](CC(=O)N1CCC[C@H]1[C@H](OC)[C@@H](C)C(=O)N[C@H](Cc1ccccc1)C(=O)OC)OC)N(C)C(=O)[C@@H](NC(=O)[C@H](C(C)C)N(C)C(=O)COCC(=O)O)C(C)C] |
| TCO—PEG₈- Dibromo- maleimide | 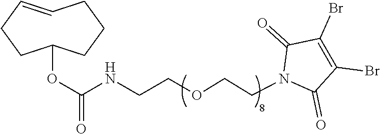 |
| —COPEG₂₄ NH—COPEG₄ (PhTzMe)/ TCO—PEG₈— Nanobody | 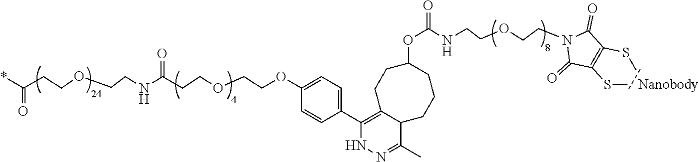 |
| CONH— PEG₄—Sulfo- DBCO | 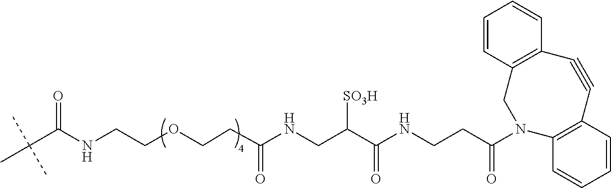 |
| AFDye 647 Azide | 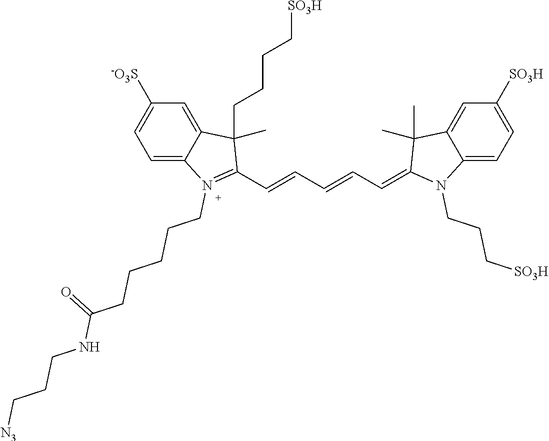 |
| mPEGₙ | 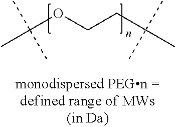 |
| dPEGₙ | 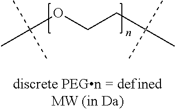 |

| | |
|---|---|
| MeCN | Acetonitrile |
| LCMS | Liquid chromatography mass spectrometry |
| ESI MS | Electrospray mass spectrometry |
| Lys | Lysine |
| Boc | Benzyloxycarbonyl |
| PBS | Phosphate buffered saline |
| DIPEA | diisopropylethylamine |
| PEG | Polyethylene glycol |
| NHPEG | NH-Polyethylene glycol |
| PyBop | Benzotriazol-1-yl-oxytri-pyrrolidinophosphonium |
| | hexafluorophosphate |
| TFA | Trifluoroacetic acid |
| NMM | N-methylmorpholine |
| DMF | Dimethylformamide |
| DBL-ONp | Di-Boc-(L)-lysine para-nitrophenyl ester |
| NHFmoc | NH-Fluorenylmethyloxycarbonyl |
| NHBoc | NH-benzyloxycarbonyl |
| MMAE | Mono-methyl Auristatin E |
| MMAF(OMe) | Mono-methyl Auristatin F (methyl ester) |
| N(PNBoc)2 | diBoc dipropylenetriamine |
| TEA | Triethylamine |
| NMR | Nuclear Magnetic Resonance |
| D₂O | Deuterium oxide |
| CD₃OD | Deuterated methanol |
| Glu | Glutamic acid derivative |
| Val | Valine |
| Cit | Citrulline |
| PAB | Para-aminobenzyl |
| DGA | Diglycolic acid |
| Rₜ | Retention time |
| DMAP | 4-(Dimethylamino)pyridine |
| NHS | N-hydroxysuccinimide |
| DCM | Dichloromethane |
| EtOAc | Ethyl acetate |
| THF | Tetrahydrofuran |
| N₃ | Azide |
| MAL | Maleimide |
| BCN | Bicyclo[6.1.0.]nonyne |
| MAL-EDA- | Maleimido-ethylenediamine-bicyclononyne |
| BCN | |
| DBCO | Dibenzylcyclooctyne |
| UPA | Unique Point of Attachment; A dendrimer with a |
| | selected point of linkage at which a linker or targeting |
| | agent is or can be attached |
| BCNTriazolo | Bicyclo[6.1.0]nonyne-[1,2,3]triazolo |
| DBCOTriazolo | Dibenzocyclooctyne-[1,2,3]triazolo |
| MPED | N-(3-maleimidopropionic acid)-ethylene diamine |
| PSSP | dipropanoic acid disulfide |
| LyP | Heptynoyl¹ Lys(fd)² LyP-1: sequence = Hept- |
| | CGNKRTRGC-NH₂ (cyclic) |
| DTX | Docetaxel |
| t-BuOH | tertiary Butanol |
| TBTA | Tris[(1-benzyl-1H-1,2,3-triazol-4-yl)methyl]amine |
| PEOX | Polyethyloxazoline |
| POZ | Poly-(2) methyl-(2)-oxazolamine |
| pHPMA | Poly(2-hydroxypropyl)methacrylamide |
| nmPEG | number of mPEG (monodispersed PEG) units |
| nFAPI | number of FAPI (discrete PEG-FAPI-04) units |
| DCC | Diccylohexylcarbodiimide |

### Preparative HPLC

Preparative HPLC was performed on Gilson HPLC system using Waters XBridge™ BEH300 Prep C18 5 μm OBD™ 30×150 mm column using a binary solvent system consisting of solvent A (water, water with formic acid or water with TFA) and solvent B (acetonitrile or acetonitrile with formic acid or TFA). The peaks were detected using UV detector at wavelengths 214 nm, 243 nm or 254 nm.

### Prep-HPLC Methods

Prep-HPLC Method 5-60% TFA: Solvent A, 0.05% TFA (v/v) in water; Solvent B, 0.05% TFA (v/v) in MeCN; flow rate: 8.0 mL/min; gradient: 0-5 min, 5% B; 5-35 min, 5-60%, 35-47 min, 60% B; 47-50 min, 60-5% B; 50-60 min, 5%. The peaks were detected using UV detector at wavelengths, 214 and 254 nm.

Prep-HPLC Method 30-50% TFA: Solvent A, 0.05% TFA (v/v) in water; Solvent B, 0.05% TFA (v/v) in MeCN; flow rate: 8.0 mL/min; gradient: 0-5 min, 30% B; 5-35 min, 30-50%, 35-47 min, 50% B; 47-50 min, 50-30% B; 50-60 min, 30%. The peaks were detected using UV detector at wavelengths, 214 and 254 nm.

Prep-HPLC Method 40-70% TFA: Solvent A, 0.05% TFA (v/v) in water; Solvent B, 0.05% TFA (v/v) in MeCN; flow rate: 8.0 mL/min; gradient: 0-5 min, 40% B; 5-35 min, 40-70%, 35-47 min, 70% B; 47-50 min, 70-40% B; 50-60 min, 40%. The peaks were detected using UV detector at wavelengths, 214 and 254 nm.

Prep-HPLC Method 20-60, TFA: Solvent A, 0.05% TFA (v/v) in water; Solvent B, 0.05% TFA (v/v) in MeCN; flow rate: 8.0 mL/min; gradient: 0-5 min, 20% B; 5-35 min, 20-60%, 35-40 min, 60-100% B; 40-47 min, 100% B; 47-50 min, 100-10% B; 50-60 min, 10%. The peaks were detected using UV detector at wavelengths, 214 and 254 nm.

Prep-HPLC Method 20-60: Solvent A, water; Solvent B, MeCN; flow rate: 8.0 mL/min; gradient: 0-7 min, 20% B; 7-37 min, 20-60%, 37-47 min, 60% B; 47-54 min, 60-20% B; 54-60 min, 20%. λ=214 nm and 254 nm.

Prep-HPLC Method 20-60(2): Solvent A, water; Solvent B, MeCN; flow rate: 8.0 mL/min; gradient: 0-5 min, 20% B; 5-55 min, 20-60%, 55-60 min, 60-20% B. Detection at λ=214 nm and 254 nm.

Prep-HPLC Method 20-90, TFA: Solvent A, 0.05% TFA (v/v) in water; Solvent B, 0.05% TFA (v/v) in MeCN; flow rate: 8.0 mL/min; gradient: 0-6 min, 20% B; 6-40 min, 20-90%, 40-47 min, 90% B; 47-51 min, 90-20% B; 51-60 min, 20% B. The peaks were detected using UV detector at wavelengths, 214 and 254 nm.

Prep-HPLC Method 30-90, TFA: Solvent A, 0.05% TFA (v/v) in water; Solvent B, 0.05% TFA (v/v) in MeCN; flow rate: 8.0 mL/min; gradient: 0-5 min, 30% B; 5-40 min, 30-90%, 40-45 min, 90% B; 45-50 min, 90-30% B; 50-60 min, 30% B. The peaks were detected using UV detector at wavelengths, 214 and 254 nm.

Prep-HPLC Method 5-60: Solvent A, water; Solvent B, MeCN; flow rate: 8.0 mL/min; gradient: 0-5 min, 5% B; 5-42 min, 5-60%, 42-49.5 min, 60-80% B; 49.5-55 min, 80%, 55-57 min, 80-5% B; 57-60 min, 5%. The peaks were detected using UV detector at wavelengths, 214 and 254 nm.

Prep-HPLC Method 5-60(2): Solvent A, water; Solvent B, MeCN; flow rate: 8.0 mL/min; gradient: 0-9 min, 5% B; 9-38 min, 5-60%, 38-48 min, 60% B; 48-54 min, 60-5% B; 54-60 min, 5% B. The peaks were detected using UV detector at wavelengths, 214 and 254 nm.

Prep HPLC Method 40-60: Solvent A, water; Solvent B, MeCN; flow rate: 8.0 mL/min; gradient: 0-9 min, 40% B; 9-37 min, 40-60%, 37-47.5 min, 60% B; 47.5-54 min, 60-40% B; 54-60 min, 40% B. The peaks were detected using UV detector at wavelengths, 214 and 254 nm.

Prep-HPLC Method 20-40: Solvent A, water; Solvent B, MeCN; flow rate: 8.0 mL/min; gradient: 0-10 min, 20% B; 10-38 min, 20-40%, 38-47.5 min, 40% B; 47.5-54 min, 40-20% B; 54-60 min, 20%. Detection at X=214 nm and 254 nm

### Automated Flash Chromatography:

Automated flash purification was performed on a Biotage® Selekt 5 automated flash chromatography system using normal phase or reverse phase silica cartridges.

### Automated Flash Chromatography Methods

Autoflash-Method 1: Cartridge=Biotage Sfar C18 D (Duo 100 Å 30 μm) 12 g cartridge; Solvent A, water; Solvent B, methanol; flow rate: 12 mL/min; gradient: 0-3 CV, 10% B; 3-5 CV, 10-60% B; 5-9.8 CV, 60-74% B; 9.8-11.4 CV, 74% B; 11.4-13.6 CV, 74-80% B; 13.6-15.6 CV, 80-100% B; 15.6-20.6 CV, 100% B; 20.6-21.6 CV, 100-10% B and 21.6-24.6 CV, 10% B. Detection at λ=214 nm and 254 nm and λₐₗₗ 198-810 nm.

Autoflash-Method 2: Cartridge=Biotage Sfar C18 D (Duo 100 Å 30 μm) 12 g cartridge; Solvent A, water; Solvent B, methanol; flow rate: 12 mL/min; gradient: 0-3 CV, 5% B; 3-4 CV, 5-30% B; 4-14 CV, 30-100% B and 14-19 CV, 100% B. Detection at λ=214 nm and 254 nm and λₐₗₗ 198-810 nm.

### Analytical LCMS

LCMS was recorded on a Waters 2795 HPLC with Waters 2996 Diode Array detector using a Waters XBridge™ 3.5 μm 3×100 mm C8 column or a Phenomenex Kinetex® 2.6 μm 2.1×75 mm C18 column using a ternary solvent system consisting of solvent A (water), solvent B (acetonitrile) and solvent C [10% of 1% v/v aqueous formic acid (formic buffer) or 10% of 1% v/v TFA (TFA buffer)]. The flow rate was typically 0.4 mL/min and injection volumes were typically 5-10 μL. The peaks were detected using UV detector at λ=214 nm, 243 nm or 254 nm (unless otherwise specified).

MS—Waters ZQ4000 with ESI probe, inlet flow split to give around 50 μL/min to the MS. Mass Spectra data was acquired in positive or negative electrospray ionisation mode as indicated. The raw data was deconvoluted using a Maximum Entropy algorithm (MaxEnt) as implemented in MassLynx software v4.0, supplied by Waters Corporation. The data reported in the experimental details corresponds to the observed value after deconvolution to a theoretical zero charge state.

### LCMS Methods

LCMS Method (philic, TFA/formic buffer): The gradient was: 0-1 min, 5% B; 1-10 min, 5-60% B; 10-11 min, 60% B; 11-13 min, 60-5% B; 13-15 min, 5% B.

LCMS Method (phobic, TFA/formic buffer): The gradient was: 0-1 min, 40% B; 1-7 min, 40-90% B; 7-9 min, 90% B; 9-11 min, 90-40% B; 11-15 min, 40% B.

LCMS Method 40-65, TFA Buffer: The gradient was: 0-1 min, 40% B; 1-10 min, 40-65% B; 10-11 min, 65% B; 11-12 min, 60-45% B; 12-15 min, 45% B

LCMS Method (20-90, 15 min): The gradient was: 0-1 min, 20% B; 1-9 min, 20-90% B; 9-11 min, 90% B; 11-13 min, 90-20% B; 13-15 min, 20% B (with 0.1% HCOOH acid).

LCMS Method 5-60, 8 min, TFA: Gradient was 0-1 min, 5% B; 1-5 min, 5-60% B; 5-6 min, 60% B; 6-6.1 min, 60-5% B, 6.1-8 min 5% B.

LCMS Method 20-90, 8 min, TFA: Gradient was 0-0.5 min, 5% B; 0.5-1 min, 5-20% B; 1-5 min, 20-90% B; 5-6 min, 90% B; 6-6.1 min, 90-5% B, 6.1-8 min 5% B.

LCMS Method 40-90, 8 min, TFA: Gradient was 0-0.5 min, 5% B; 0.5-1 min, 5-40% B; 1-5 min, 40-90% B; 5-6 min, 90% B; 6-6.1 min, 90-5% B, 6.1-8 min 5% B.

LCMS Method 20-60, 8 min, TFA: Gradient was 0-0.5 min, 5% B; 0.5-1 min, 5-20% B; 1-5 min, 20-60% B; 5-6 min, 60% B; 6-6.1 min, 60-5% B, 6.1-8 min 5% B.

LCMS Method 60-90, 8 min, TFA: Gradient was 0-0.5 min, 5% B; 0.5-1 min, 5-60% B; 1-5 min, 60-90% B; 5-6 min, 90% B; 6-6.1 min, 90-5% B, 6.1-8 min 5% B.

LCMS Method 40-60, 8 min, TFA: Gradient was 0-0.5 min, 5% B; 0.5-1 min, 5-40% B; 1-5 min, 40-60% B; 5-6 min, 60% B; 6-6.1 min, 60-5% B, 6.1-8 min 5% B.

LCMS Method 5-80, 8 min, TFA: Gradient was 0-1 min, 5% B; 1-5 min, 5-80% B; 5-6 min, 80% B; 6-6.1 min, 80-5% B, 6.1-8 min 5% B.

LCMS Method 5-80, 15 min, TFA: Gradient was 0-1 min, 5% B; 1-10 min, 5-80% B; 10-11 min, 80% B; 11-13 min. 80-5% B, 13-15 min 5% B, 0.1% TFA.

### Analytical HPLC

HPLC data was recorded on a Waters 2695 separation module with 2996 PDA detector using a Waters XBridge™ C8 3.5 μm 3×100 mm column or a Phenomenex Kinetex® 2.6 μm 2.1×75 mm C18 column. The instrument control software was Waters Empower 3. The three mobile phases used were a) 1% v/v TFA buffer or 1% v/v formic acid buffer or 100 mM ammonium formate, b) water and c) acetonitrile. The flow rate was typically 0.4 mL/min and injection volumes were typically 5-10 μL. The peaks were detected using UV detector at λ=214 nm, 243 nm or 254 nm (unless otherwise specified).

### HPLC Methods

HPLC-Method 5-80, 15 min, formate/TFA: The gradient was 0-1 min, 5% B, 1-7 min, 5-80% B, 7-12 min, 80% B, 12-13 min 80-5% B, 13-15 min, 5% B at a flow rate of 0.40 mL/min. The peaks were detected using UV detector at wavelength, 214, 243 and 254 nm

HPLC—Method 5-80, 8 min, TFA: The gradient was: 0-0.5 min, 5% B; 0.5-3.5 min, 5-80% B; 3.5-6 min, 80% B; 6-6.5 min, 80-5% B; 6.5-8 min, 5% B; at a flow rate of 0.40 mL/min. The peaks were detected using UV detector at wavelength, 214, 243 and 254 nm.

HPLC-Philic method, formate/TFA buffer, 15 min: The gradient was: 0-1 min, 5% B; 1-10 min, 5-60% B; 10-11 min, 60% B; 11-13 min, 60-5% B; 13-15 min, 5% B.

### Analytical UPLC-ToF (Ultra-High Pressure Liquid Chromatography—Time of Flight)

UPLC-ToF data was recorded with a Waters Aquity UPLC binary separation module with Waters Aquity PDA detector and Waters LCT Premiere (ToF) Mass Spectrometer. The column used was Phenomenex Kinetex EVO C18 2.6 μm 2.1×100 mm column. The instrument control software was Waters Masslynx Version 4.1. The two mobile phases used were a) 0.01% v/v TFA in water and b) 0.01% v/v TFA in acetonitrile. The flow rate was typically 0.2 mL/min or 0.4 mL/min and injection volumes were typically 2-5 μL. The peaks were detected within 200 nm-400 nm (unless otherwise specified).

### UPLC-ToF Methods

Method 1: The gradient was 15-35% MeCN/H₂O (1-9 min), 35% MeCN/H₂O (9-11 min), 35-15% MeCN/H₂O (11-12 min), 15% MeCN/H₂O (12-15 min), 0.01% TFA buffer) and UV detection at 254 nm.
Method 2: The gradient was 20-80% MeCN/H₂O (1-10 min), 80% MeCN/H₂O (10-11 min), 80-20% MeCN/H₂O (11-13 min), 20% MeCN/H₂O (13-15 min), 0.01% TFA buffer) and UV detection at 254 nm.

### Size Exclusion Chromatography (SEC)

Size exclusion chromatography was performed on Sephadex™ LH-20 column under gravity using methanol or acetonitrile as the eluent at a flow rate of ˜50-60 drops/min. Each fraction size comprised of 400-600 drops. Fractions containing PEGylated compounds were detected by TLC [TLC plates were developed in aq 5% (w/v) BaCl₂ followed by a solution of I₂ in ethanol] or were analysed by HPLC.

### Tangential Flow Filtration

Tangential flow filtration was carried out either on 50 cm² Pellicon® XL cassette Ultracel® regenerated cellulose membrane using water as the eluting medium or on a 0.11 m² Pellicon® 3 Cassette with Ultracel© regenerated cellulose membrane using water or acetonitrile as the eluting medium.

### Centrifugal Ultrafiltration

Centrifugal ultrafiltration was carried out either on Eppendorf centrifuge 5810R at 4000 rpm or 5415R at 14000 rpm using Amicon® Ultra centrifugal filters with specified molecular weight cut-off (MWCO) Ultracel® regenerated cellulose membrane.

### NMR

NMR spectra were recorded in CD₃OD, CDCl₃, D₂O, CD₃CN or otherwise stated on a Bruker (Bruker Daltonics Inc, NSW, Australia) 300 UltraShield™ 300 MHz NMR instrument.

### IR

IR spectra were recorded on Cary 630 FTIR Agilent Technologies diamond ATR accessory using 16 scans.

### General Procedures:

Preparation of carboxy reactive dendrimer scaffolds have been previously described, in particular refer to WO2008/017125. One skilled in the art can adapt these methods to prepare the various dendrimers outlined herein. In the following examples [Lys] in a formula refers to the lysine building units on the surface layer of the dendrimer.

### General Procedure A. Boc Deprotection

To an ice-cooled, stirred suspension of Boc compound (1.0 equivalent) in water was added TFA (40-200 equivalents/Boc group). After 5 minutes, the ice-bath was removed, and the reaction mixture left to stir at room temperature overnight. The volatiles were removed in vacuo and the remaining aqueous solution diluted further with water and lyophilised to give the deprotected product in quantitative yield.

### General Procedure B. Addition of a Lysine Layer on Dendrimer Surface.

To a stirred solution of the TFA dendrimer (1.0 equivalent) in DMF under an atmosphere of nitrogen was added TEA (6.0 equivalents/NH₂), followed by DBL-ONp (2.0 equivalents/NH₂). The ensuing reaction mixture was left to stir overnight at room temperature. The volatiles were removed in vacuo and the resulting crude material purified using standard methods.

### General Procedure C. Pegylation of the Dendrimer Surface Using HO-Lys[(α-NHBoc)(ε-NH-COPEG₁₁₀₀)] Wedge.

To a stirred solution of TFA dendrimer (1.0 equivalent) in DMF under an atmosphere of nitrogen was added PyBOP (2.0 equivalents/NH₂) and DIPEA (8.0 equivalents/NH₂). After 10 minutes a solution of HO-Lys[(α-NHBoc)(ε-NH-COPEG₁₁₀₀)] (1.35 equivalents/NH₂) in DMF was added and the ensuing reaction mixture stirred overnight at room temperature. The volatiles were removed in vacuo and the resulting crude material purified using standard methods.

General Procedure D. Capping of the Dendrimer Surface with HO-Lys[(α-NHBoc)(ε-NHFmoc)] Wedge Followed by Fmoc Deprotection
Step 1: To a stirred solution of HO-Lys[(α-NHBoc)(ε-NHFmoc)] (1.5 eq/NH₂) and NMM (2.5 eq/NH₂) in DMF was added PyBOP (1.4 eq/NH₂). The ensuing reaction mixture was stirred at room temperature for 15 min then a solution of TFA-dendrimer (1.0 equivalent) and NMM (2.5 eq/NH₂) in DMF was added. The ensuing reaction mixture was left to stir at room temperature for 1 hour then slowly added to ice-cold MeCN and stirred for 15 min The resulting solid was collected by filtration and washed with MeCN (3×) then lyophilised.
Step 2: To a solution of Fmoc/Boc dendrimer (1.0 equivalent) in DMF was added piperidine (21 eq/Fmoc). The solution was allowed to stir at room temperature for 90 minutes then slowly added to ice-cold Et₂O. After 15 min, the precipitated solid was collected by filtration, washed with Et₂O, dissolved in H₂O and lyophilised.
   General Procedure E. Capping Dendrimer Surface with Glu-Vc-PAB-MMAE or DGA-MMAF(OMe)

Azido-PEG₂₄CO—[N(PN)₂[Lys]₂[Lys]₄[Lys]₈[(α-NH₂.TFA)(ε-NH-COPEG_{570/1100/2000})]₈ Compound 10, Compound 14 or Compound 16 (1.0 equivalent) was dissolved in a mixture of DMF and NMM (5.0 equivalents/NH₂) at room temperature. This solution was added to HO-Glu-vc-PAB-MMAE (Levena Biopharma) or DGA-MMAF(OMe) (Concortis Biosystems) (1.2 equivalents/NH₂) and PyBOP (2.0 equivalents/NH₂) and left at room temperature. The resulting crude material purified by SEC.

### General Procedure F. Conjugation of Affibody to MMAE/MMAF Dendrimers

Step 1: A solution of Affibody protein (HER2, Affibody AB, 1.0 mg/mL PBS) was treated with TCEP (50 mM, 39.0 equivalents) and the reaction mixture shaken at 650 rpm for 2 h at room temperature. The resulting solution was purified by SEC.
Step 2: The collected permeate was treated with a solution of ((1R,8S,9s)-bicyclo[6.1.0]non-4-yn-9-yl)methyl (2-(3-(2,5-dioxo-2,5-dihydro-1H-pyrrol-1-yl)propanamido)ethyl)carbamate(Mal-BCN)(Compound 117) (20.0 equivalents) in DMSO. The ensuing reaction mixture was shaken at 650 rpm for 2 h at room temperature. The resulting solution was purified by SEC.
Step 3: The affibody-BCN solution was treated with a solution of azido-PEG₂₄CO—[N(PN)₂][Lys]₂[Lys]₄[Lys][(α-NHGlu-vc-PAB-MMAE)(ε-NH-COPEG_{570/1100/2000})]₈ Compound 64, Compound 65, Compound 66 (240 μM in PBS) or azido-PEG₂₄CO—[N(PN)₂][Lys]₂[Lys]₄[Lys]₈[(α-NHDGA-MMAF(OMe))(ε-NH-COPEG₁₁₀₀)]₈ Compound 67 (365 μM in PBS) (1.3 equivalents affibody-BCN/dendrimer). The ensuing reaction mixture was shaken at 650 rpm overnight at room temperature and then treated with a 9.38 mM (30% EtOH/water) solution of DBCO agarose (5.0 equivalents/dendrimer). The ensuing suspension was shaken at 1200 rpm at room temperature overnight. The suspension was purified using SEC.

### General Procedure G. Conjugation of Nanobody to MMAE Dendrimers

Step 1: A solution of linker (BCN-PEG₂NH-Glu-NHPEG₂₄CO-NHPEG₃TCO Compound 50 or DBCO-Glu-NHPEG₂₄CO-NHPEG₃TCO), Compound 51 was prepared by dissolving linker (1 mg) in 20:80 (DMSO/10 mM PBS, 1 mL).
Step 2: The TCO-linker solution (1 eq.) was added to a solution of the tetrazine-functionalised dendrimer (1.0 eq., 8 mg/mL) in PBS (1). Reaction mixture allowed to stand at room temperature for 30 min. Completion of the reaction was indicated by disappearance of the pink tetrazine colour. The reaction was monitored by HPLC.
Step 3: Once the reaction was complete, the contents were diluted with PBS (to a final volume of 0.5 mL) with PBS. A portion of the BCN/DBCO-MMAE-dendrimer (1.0 eq.) was added to a solution of Nanobody-N₃ (1.0 eq., 9.2 mg/ml) in Tris buffer (20 mM, 1 mL). The resulting solution was left to stand at RT for 7 h, then at 4° C. overnight. Purification of the Nanobody-dendrimer construct was carried out by anion exchange chromatography followed by SEC.
   General Procedure H. Capping of the Dendrimer Surface with N₃-PEG_{570/1100}-NHS Ester Followed by Removal of Boc Group
Step 1: To a stirred solution of BHALys[Lys]₂[Lys]₄[Lys]₈[Lys]₁₆[Lys]₃₂[(α-NHBoc)₃₂(ε-NH₂)₃₂] (Ref 1, WO2007/082331A1, J. Controlled Release 2011, 152, 241-248) and DIPEA (2.0 eq/NH₂) in DMF was added N₃-PEG_{570/1100}-NHS ester (1.5 eq/NH₂) or a solution of N₃-PEG_{570/1100}-acid (1.3 eq/NH₂) and PyBOP (1.3 eq/NH₂) in DMF. The ensuing reaction mixture was stirred at room temperature for 15 h. Deionised water was added to the reaction mixture and the resulting solution was filtered (0.45 μm acrodisc syringe filter). The filtrate was ultrafiltered through 10 kDa regenerated cellulose Pellicone membrane using water as the circulating medium till 20 diafiltration volume (DV) was collected as permeate. The retentate was collected and pooled with line washings and lyophilised.
Step 2: To a solution of azido dendrimer (1.0 equivalent) in DCM was added trifluoroacetic acid (321 eq/NHBoc) (TFA/DCM 1:1 v/v). The solution was stirred at RT for 15 h and the volatiles were removed in vacuo.

### General Procedure I. Conjugation of Cyanine5 to the Dendrimer Followed by Acetylation

Step 1: To a stirred solution of BHALys[Lys]₂[Lys]₄[Lys]₈[Lys]₁₆[Lys]₃₂[(α-NH₂.TFA)₃₂(ε-NH-COPEG_{570/1100}N₃)₃₂], Compound 32 and Compound 33and DIPEA (4.0 eq./NH₂) in DMF was added Cyanine5-NHS ester (2.0 eq.). The reaction mixture was stirred at ambient temperature for 3 h. Volatiles were removed in vacuo and the residue was used without further purification in Step 2.
Step 2: To a stirred solution of dried residue obtained in step1 in pyridine (1 mL) was added acetic anhydride (2 mL). The reaction mixture was stirred at ambient temperature for 15 h. The volatiles were removed in vacuo and the residue was purified by SEC (Sephadex LH-20) using methanol as the eluent.
   General Procedure J. Click Reaction of DUPA-BCN with Azido Dendrimer

To a stirred solution of BHALys[Lys]₂[Lys]₄[Lys]₈[Lys]₁₆[Lys]₃₂[(α-NHCy5)₁(α-NHAc)₃₁(ε-NH-COPEG_{570/1100}N₃)₃₂], Compound 34 and Compound 35 in acetonitrile and water (1:1) was added DUPA-BCN (19). The reaction mixture was stirred at ambient temperature for 15 h. The reaction mixture was lyophilised, and the residue obtained after lyophilisation was purified by SEC (LH-20) using methanol as the eluent or ultrafiltered using water on a 10 KDa MWCO Pellicone regenerated cellulose TFF membrane.

General Procedure K. Conjugation of DOTA Dendrimers with Nanobody-Cys SRS-13.
Step 1: Reduction of nanobody dimer: To a solution of the Nanobody-Cys dimer (3.33 mg/mL in 10 mM PBS, pH 7.4; 1 eq) was added aq 0.5 M TCEP (10 eq). The reaction mixture incubated at 37° C. for 2 h. Excess TCEP was removed from the reaction mixture by Amicon® Ultra centrifugal filters with 10 kDa MWCO Ultracel® regenerated cellulose membrane and concentrated by centrifugation at 4000 rpm for 10 min. The retentate was washed with pH 7.4 PBS, buffer (×5) by centrifugation at 4000 rpm and the retentate obtained was then buffer exchanged with pH 7.2 PBS buffer (PBS 10 mM; EDTA 5 mM; Degassed with nitrogen).
Step 2: Synthesis of Nanobody-Cys/Me(MAL)-PEG₂₄-CONH-PEG₃-TCO SRS-14. To a solution of the reduced nanobody (Step 1, 3.16 mg/mL) in aq PBS pH 7.2 buffer (10 mM PBS, 5 mM EDTA) was added a solution of Me(MAL)-PEG₂₄-CONH-PEG₃-TCO SRS-12 (2 eq; 10 mg/mL) in deionised water. The resulting solution was left to stand at 4° C. and the reaction was monitored by UPLC analysis. (UPLC-Method 2). After 18 h., the excess linker SRS-12 was removed from the reaction mixture using Amicon® Ultra centrifugal filters with 10 kDa MWCO Ultracel® regenerated cellulose membrane and concentrated by centrifugation at 4000 rpm for 10 min. The retentate was washed with aq PBS buffer pH 7.2 (10 mM PBS, 5 mM EDTA) by centrifugation at 4000 rpm (×5).
Step 3: Conjugation reaction: A solution of the tetrazine-substituted dendrimer (10 mg/mL; 1 eq) in deionised water was added to a solution of Nanobody-Cys/Me(MAL)-PEG₂₄-CONH-PEG₃-TCO SRS-14 (1.2 eq) at rt. After 18 h, the Nanobody-dendrimer construct was purified by Nickel affinity column chromatography followed by SEC.

### General Procedure L. Quantification of Cy5 Labelled Dendrimer-Nanobody Conjugates.

Quantitation of purified conjugates was performed by interpolating the measured 650 nm absorbance of samples against a standard curve prepared using the corresponding unconjugated dendrimer. Absorbance measurements were performed on a Nanodrop ND-1000 spectrophotometer (Thermo Fisher). For each dendrimer-nanobody conjugate, an amount of dry unconjugated dendrimer weighed out on a digital microbalance (Mettler-Toledo) was dissolved in an appropriate volume of 10 mM HEPES pH8 buffer. Standard solutions of known concentration between 10 mg/ml and 0.05 mg/ml were prepared by dilution in 10 mM HEPES pH8 buffer and their absorbance at 650 nm measured. Standard curves generated by linear regression and interpolations were performed using Prism 9 software (Graphpad).

TABLE of
Compounds 1

| | Com- |
|---|---|
| | pound |
| | Num- |
| Description | ber |
| Azido-PEG₂₄CO-[N(PNBoc)₂] | 1 |
| Azido-PEG₂₄CO-[N(PNH₂•TFA)₂] | 2 |
| Azido-PEG₂₄CO-[N(PN)₂][Lys]₂[NHBoc]₄, G1 | 3 |
| Azido-PEG₂₄CO-[N(PN)₂][Lys]₂[NH₂•TFA]₄, G1 | 4 |
| Azido-PEG₂₄CO-[N(PN)₂][Lys]₂[Lys]₄[(α- | 5 |
| NHBoc)(ε-NH-COPEG₁₁₀₀)]₄, G2 | |
| Azido-PEG₂₄CO-[N(PN)₂][Lys]₂[Lys]₄ | 6 |
| [(α-NH₂•TFA)(ε-NH-COPEG₁₁₀₀)]₄, G2 | |
| Azido-PEG₂₄CO-[N(PN)₂][Lys]₂[Lys]₄[NHBoc]₈, G2 | 7 |
| Azido-PEG₂₄CO-[N(PN)₂][Lys]₂[Lys]₄[NH₂•TFA]₈, G2 | 8 |
| Azido-PEG₂₄CO-[N(PN)₂][Lys]₂[Lys]₄[Lys]₈[(α- | 9 |
| NHBoc)(ε-NHCOPEG1100)]₈, G3 | |
| Azido-PEG₂₄CO-[N(PN)₂][Lys]₂[Lys]₄[Lys]₈[(α- | 10 |
| NH₂•TFA)(α-NHCOPEG₁₁₀₀)]₈, G3 | |
| Azido-PEG₂₄CO-[N(PN)₂][Lys]₂[Lys]₄[Lys][(α- | 11 |
| NHBoc)(ε-NHFmoc)]₈, G3 | |
| Azido-PEG₂₄CO-[N(PN)₂][Lys]₂[Lys]₄[Lys]₈[(α- | 12 |
| NHBoc)(ε-NH₂)]₈, G3, | |
| Azido-PEG₂₄CO-[N(PN)₂][Lys]₂[Lys]₄[Lys]₈[(α- | 13 |
| NHBoc)(ε-NH-COPEG₅₇₀)]₈, G3 | |
| Azido-PEG₂₄CO-[N(PN)₂][Lys]₄[Lys]₈ | 14 |
| [(α-NH₂•TFA)(ε-NHCOPEG₅₇₀)]₈, G3 | |
| Azido-PEG₂₄CO-[N(PN)₂][Lys]₂[Lys]₄[Lys]₈[(α- | 15 |
| NHBoc)(ε-NH-COPEG₂₀₀₀)]₈, G3 | |
| Azido-PEG₂₄CO-[N(PN)₂][Lys]₂[Lys]₄[Lys]₈[(α- | 16 |
| NH₂•TFA)(ε-NH-COPEG₂₀₀₀)]₈, G3 | |
| BHALys[Lys]₂[Lys]₄[((α-NH-COPEG₂₄NH- | 17 |
| COPEG₄(PhTzMe))₁(α-NH₂)₃)(ε-NH-COPEG₁₀₀₀)₄], G2 | |
| BHALys[Lys]₂[Lys]₄[Lys]₈[((α-NH-COPEG₂₄NH- | 18 |
| COPEG₄(PhTzMe))₁(α-NH₂)₇)(ε-NH-COPEG₄₁₂)₈], G3 | |
| BHALys[Lys]₂[Lys]₄[Lys]₈[((α-NH-COPEG₂₄NH- | 19 |
| COPEG₄(PhTzMe))₁(α-NH₂)₇)(ε-NH-COPEG₁₀₀₀)₈], G3 | |
| BHALys[Lys]₂[Lys]₄[Lys]₈[Lys]₁₆[((α-NH- | 20 |
| COPEG₂₄NH-COPEG₄(PhTzMe))₁(α-NH₂)₁₅)(ε-NH- | |
| COPEG₁₀₀₀)₁₆], G4 | |
| BHALys[Lys]₂[Lys]₄[Lys]₈[Lys]₁₆[Lys]₃₂[((α-NH- | 21 |
| COPEG₂₄NH-COPEG₄(PhTzMe))₁(α-NH₂)₃₁)(ε-NH- | |
| COPEG₁₀₀₀)₃₂], G5 | |
| BHALys[Lys]₂[Lys]₄[(α-NHBoc)₄(ε-NH-COPEG₂₄NH- | 22 |
| COPEG₄(PhTzMe)₄], G2 | |
| BHALys[Lys]₂[Lys]₄[(α-NH₂•HCl)₄(ε-NH- | 23 |
| COPEG₂₄NH-COPEG₄(PhTzMe)₄], G2 | |
| BHALys[Lys]₂[Lys]₄[((α-Lys(α-NHCy5)(α- | 24 |
| NHDFO))₁(α-NH₂)₃)(ε-NH-COPEG₂₄NH- | |
| COPEG₄(PhTzMe)₄], G2 | |
| BHALys[Lys]₂[Lys]4[((α-NH-COPEG₂₄NH- | 25 |
| COPEG₄(PhTzMe))₁(α-Lys(α-NHCy₅)(ε-NHDFO))₁(α- | |
| NH₂)₂)(ε-NH-COPEG₁₀₀₀)₄], G2 | |
| BHALys[Lys]₂[Lys]₄[Lys]₈[((α-NH-COPEG₂₄NH- | 26 |
| COPEG₄(PhTzMe))₁(α-Lys(α-NHCy5)(ε-NHDFO))₁(α- | |
| NH₂)₆)(ε-NH-COPEG₄₁₂)₈], G3 | |
| BHALys[Lys]₂[Lys]₄[Lys]₈[((α-NH-COPEG₂₄NH- | 27 |
| COPEG₄(PhTzMe))₁(α-Lys(α-NHCy5)(ε-NHDFO))₁(α- | |
| NH₂)₆)(ε-NH-COPEG₁₀₀₀)₈], G3 | |
| BHALys[Lys]₂[Lys]₄[Lys]₈[Lys]₁₆[((α-NH- | 28 |
| COPEG₂₄NH-COPEG₄(PhTzMe))₁(α-Lys(α-NHCy5)(ε- | |
| NHDFO))₁(α-NH₂)₁₄)(ε-NH-COPEG₁₀₀₀)₁₆], G4 | |
| BHALys[Lys]₂[Lys]₄[Lys]₈[Lys]₁₆[Lys]₃₂[((α-NH- | 29 |
| COPEG₂₄NH-COPEG₄(PhTzMe))₁(α-Lys(α-NHCy5)(ε- | |
| NHDFO))₁(α-NH₂)₃₀)(ε-NH-COPEG₁₀₀₀)₃₂], G5 | |
| BHALys[Lys]₂[Lys]₄[Lys]₈[Lys]₁₆[Lys]₃₂[(α- | 30 |
| NHBoc)₃₂(ε-NH-COPEG₅₇₀N₃)₃₂] | |
| BHALys[Lys]₂[Lys]₄[Lys]₈[Lys]₁₆[Lys]₃₂[(α- | 31 |
| NHBoc)₃₂(ε-NH-COPEG₁₁₀₀N₃)₃₂] | |
| BHALys[Lys]₂[Lys]₄[Lys]₈[Lys]₁₆[Lys]₃₂[(α- | 32 |
| NH₂•TFA)₃₂(ε-NH-COPEG₅₇₀N₃)₃₂] | |
| BHALys[Lys]₂[Lys]₄[Lys]₈[Lys]₁₆[Lys]₃₂[(α- | 33 |
| NH₂•TFA)₃₂(ε-NH-COPEG₁₁₀₀N₃)₃₂] | |
| BHALys[Lys]₂[Lys]₄[Lys]₈[Lys]₁₆[Lys]₃₂[(α- | 34 |
| NHCy5)₁(α-NHAc)₃₁(ε-NH-COPEG₅₇₀N₃)₃₂] | |
| BHALys[Lys]₂[Lys]₄[Lys]₈[Lys]₁₆[Lys]₃₂[(α- | 35 |
| NHCy5)₁(α-NHAc)₃₁(ε-NH-COPEG₁₁₀₀N₃)₃₂] | |
| BHALys[Lys]₂[Lys]₄[Lys]₈[(α-NHBoc)₈(ε-NH- | 36 |
| COPEG₂₄NH-DUPA(OtBu)₃)₈], G3 | |
| BHALys[Lys]₂[Lys]₄[Lys]₈[(α-NH₂•TFA)₈(ε-NH- | 37 |
| COPEG₂₄NH-DUPA(OH)₃)₈], G3 | |
| BHALys[Lys]₂[Lys]₄[Lys]₈[((a-(NHDOTA))₁) | 38 |
| (α-NHAc)₇)(ε-NH-COPEG₂₄NH-DUPA)₈], G3 | |
| DUPA(OtBu)₃-NHS ester | 39 |
| DUPA(OtBu)₃-NHPEG₂₄CO₂H | 40 |
| DUPA(OtBu)₃-NHPEG₂₄-NHS ester | 41 |
| HO-Lys[(α-NHCOPEG₂₄NH-DUPA(OtBu)₃) | 42 |
| (ε-NHFmoc)] | |
| DOTA(OtBu)₄-p-BnNH-Glu-OH | 43 |
| DOTA(OtBu)₄-BnNH-Glu-NHS ester | 44 |
| HO-Lys[(α-NH-COPEG₂₄NH-DUPA(OtBu)₃) | 45 |
| (ε-GluNH-p-Bn-DOTA(OtBu)₄)] | |
| Di-tert-butyl ((1-(tert-butoxy)-5-((2,5-dioxopyrrolidin-1- | 46 |
| yl)oxy)-1,5-dioxopentan-2-yl)carbamoyl)glutamate | |
| Tri-tert-butyl (18S,22S)-2,2-dimethyl-4,15,20-trioxo- | 47 |
| 3,8,11-trioxa-5,14,19,21-tetraazatetracosane-18,22,24- | |
| tricarboxylate | |
| (13S,17S)-1-Amino-10,15-dioxo-3,6-dioxa-9,14,16- | 48 |
| triazanonadecane-13,17,19-tricarboxylic acid | |
| BCN-PEG₂-Glu-CO-NHPEG₂₄CO₂H | 49 |
| BCN-PEG₂-Glu-CO-NHPEG₂₄CO-NHPEG₃-TCO | 50 |
| DBCO-Glu-NHPEG₂₄CO-NHPEG₃-TCO | 51 |
| (MeTzPh)PEG₄CO-NHPEG₂₄CO- | 52 |
| [N(PN)₂][Lys]₄[Lys]₈[((α-NHCy5)1(α-NHAc)₇) | |
| (ε-NH-COPEG₁₁₀₀)₈], G3 | |
| (MeTzPh)PEG₄CO-NHPEG₂₄CO- | 53 |
| [N(PN)₂][Lys]₂[Lys]₄[Lys]₈[Lys]₁₆[((α-NHCy5)₁(a- | |
| NHAc)₁₅)(ε-NH-COPEG₁₁₀₀)₁₆], G4 | |
| (MeTzPh)PEG₄CO-NHPEG₂₄CO- | 54 |
| [N(PN)₂][Lys]₂[Lys]₄[Lys]₈[Lys]₁₆[Lys]₃₂[((α- | |
| NHCy5)₁(α-NHAc)₃₁)(ε-NH-COPEG₁₁₀₀)₃₂], G5 | |
| DFO-PEG₄-Sulfo-DBCO | 55 |
| Synthesis of DFO-DBCO | 56 |
| Synthesis of TCO-PEG₈-Dibromomaleimide | 57 |
| Synthesis of HO-Lys[(α-NHCy5)(ε-NHDFO)] wedge | 58 |
| HO-Lys[(α-NHCy5)(ε-NH₂)] | 59 |
| HO-Lys[(α-NHCy5)(ε-NHDFO)] | 60 |
| (17S,21S)-1-((1R,8S,9S)-Bicyclo[6.1.0]non-4-yn-9-yl)- | 61 |
| 3,14,19-trioxo-2,7,10-trioxa-4,13,18,20- | |
| tetraazatricosane-17,21,23-tricarboxylic acid | |
| Azido-PEG₂₄CO-[N(PN)₂][Lys]₂[Lys]₄[Lys]₈[(α- | 64 |
| NHGlu-vc-PAB-MMAE)₈(ε-NH-COPEG₅₇₀)₈], G3 | |
| Azido-PEG₂₄CO-[N(PN)₂][Lys]₂[Lys]₄[Lys]₈[(α- | 65 |
| NHGlu-vc-PAB-MMAE)₈(ε-NH-COPEG₁₁₀₀)₈], G3 | |
| Azido-PEG₂₄CO-[N(PN)₂][Lys]₂[Lys]₄[Lys]₈[(α- | 66 |
| NHGlu-vc-PAB-MMAE)₈(ε-NH-COPEG₂₀₀₀)₈], G3 | |
| Azido-PEG₂₄CO-[N(PN)₂][Lys]₂[Lys]₄[Lys]₈[(α- | 67 |
| NHDGA-MMAF(OMe))₈(ε-NH-COPEG₁₁₀₀)₈], G3 | |
| BHA[Lys]₂[Lys]₄[Lys]₈[((α-NH-COPEG₂₄NH- | 69 |
| COPEG₄(PhMeTz))₁(α-NH₂)₇)(ε-NH-COPEG₁₁₀₀)₈], G3 | |
| BHA[Lys]₂[Lys]₄[Lys]₈[((α-NH-COPEG₂₄NH- | 70 |
| COPEG₂-BCN)₁(α-NH₂)₇)(ε-NH-COPEG₁₁₀₀)₈], G3 | |
| (MeTzPh)PEG₄CO-NHPEG₂₄CO- | 71 |
| [N(PN)₂][Lys]₂[Lys]₄[Lys]₈[((α-NHCy5)₁(α-NHGlu- | |
| VC-PAB-MMAE)₇)(ε-NH-COPEG₁₁₀₀)₈], G3 | |
| (MeTzPh)PEG₄CO-NHPEG₂₄CO-[N(PN)₂][Lys]₂[³H- | 72 |
| Lys]₄[Lys]₈[(α-NHGlu-VC-PAB-MMAE)₈(ε-NH- | |
| COPEG₁₁₀₀)₈] | |
| (MeTzPh)PEG₄CO-NHPEG₂₄CO- | 73 |
| [N(PN)₂][Lys]₂[Lys]₄[Lys]₈[((α-NHDFO)₂(α-NHGlu- | |
| VC-PAB-MMAE)₆)(ε-NH-COPEG₁₁₀₀)₈] | |
| BHA[Lys]₂[Lys]₄[Lys]₈[((α-NH-COPEG₂₄NH- | 74 |
| COPEG₄(PhMeTz))₁(α-NHDFO)₂(α-NHGlu-VC-PAB- | |
| MMAE)₅)(ε-NH-COPEG₁₁₀₀)₈] | |
| Affibody-BCN/N₃PEG₂₄CO- | 77 |
| [N(PN)₂][Lys]₂[Lys]₄[Lys]₈[(α-NHGlu-vc-PAB- | |
| MMAE)₈(ε-NH-COPEG₁₁₀₀)₈], G3 | |
| Affibody-BCN/N₃PEG₂₄CO- | 78 |
| [N(PN)₂][Lys]₂[Lys]₄[Lys][(α-NHGlu-vc-PAB- | |
| MMAE):(ε-NH-COPEG₂₀₀₀)₈], G3 | |
| Affibody-BCN/N₃PEG₂₄CO- | 81 |
| [N(PN)₂][Lys]₂[Lys]₄[Lys]₈[(α-NHDGA- | |
| MMAF(OMe)₈(ε-NH-COPEG₁₁₀₀)₈], G3 | |
| Nanobody#-N₃-DBCO-Sulfo-PEG₃-DFO | 82 |
| Nanobody#-N₃/DBCO-Glu-NHPEG₂₄CONHPEG₃- | 83 |
| TCO/(MeTzPh)PEG₄CO-NHPEG₂₄CO- | |
| [N(PN)₂][Lys]₂[Lys]₄[Lys]₈[((α-NHDFO)₂(α-NHGlu- | |
| VC-PAB-MMAE)₆)(ε-NH-COPEG₁₁₀₀)₈] | |
| BHA[Lys]₂[Lys]₄[Lys]₈[((α-NH-COPEG₂₄NH- | 84 |
| COPEG₄(PhMeTz)/TCO-PEG₃NH-COPEG₂₄NH-Glu- | |
| DBCO/N₃-Nanobody#)₁(α-NHDFO)₂(α-NHGlu-VC- | |
| PAB-MMAE)₅)(ε-NH-COPEG₁₀₀₀)₈], G3 | |
| BHALys[Lys]₂[Lys]₄[((α-NH-COPEG₂₄NH- | 85 |
| COPEG₄(PhMeTz)/TCO-PEG₃NH-COPEG₂₄NH-Glu- | |
| DBCO/N₃-Nanobody#)₁(α-Lys(α-NHCy5)(ε- | |
| NHDFO))₁(α-NH₂)₂)(ε-NH-COPEG₁₀₀₀)₄], G2 | |
| BHALys[Lys]₂[Lys]₄[((α-NH-COPEG₂₄NH- | 86 |
| COPEG₄(PhMeTz)/TCO-PEG₃NH-COPEG₂₄NH-Glu- | |
| DBCO/N₃-Nanobody#)₂(α-Lys(α-NHCy5)(ε- | |
| NHDFO))₁(α-NH₂))₁(ε-NHCOPEG₁₀₀₀)₄], G2 | |
| BHALys[Lys]₂[Lys]₄[Lys]₈[((α-NH-COPEG₂₄NH- | 87 |
| COPEG₄(PhMeTz)/TCO-PEG₃NH-COPEG₂₄NH-Glu- | |
| DBCO/N₃-Nanobody#)₁(α-Lys(α-NHCy5)(ε- | |
| NHDFO))₁(α-NH₂)₆)(ε-NH-COPEG₄₁₂)₈], G3 | |
| BHALys[Lys]₂[Lys]₄[Lys][((α-NH-COPEG₂₄NH- | 88 |
| COPEG₄(PhMeTz)/TCO-PEG₃NH-COPEG₂₄NH-Glu- | |
| DBCO/N₃-Nanobody#)₂(α-Lys(α-NHCy5)(ε- | |
| NHDFO))₁(α-NH₂)₅)(ε-NH-COPEG₄₁₂)₈], G3 | |
| BHALys[Lys]₂[Lys]₄[Lys]₈[((α-NH-COPEG₂₄NH- | 89 |
| COPEG4(PhMeTz)/TCO-PEG₃NH-COPEG₂₄NH-Glu- | |
| DBCO/N₃-Nanobody#)₁(α-Lys(α-NHCy5)(ε- | |
| NHDFO))₁(α-NH₂)₆)(ε-NH-COPEG₁₀₀₀)₈], G3 | |
| BHALys[Lys]₂[Lys]₄[Lys]₈[((α-NH-COPEG₂₄NH- | 90 |
| COPEG₄(PhMeTz)/TCO-PEG₃NH-COPEG₂₄NH-Glu- | |
| DBCO/N₃-Nanobody#)₂(α-Lys(α-NHCy5)(ε- | |
| NHDFO))₁(α-NH₂)₅)(ε-NH-COPEG₁₀₀₀)₈], G3 | |
| BHA[Lys]₂[Lys]₄[Lys]₈[Lys]₁₆[((α-NH-COPEG₂₄NH- | 91 |
| COPEG₄(PhMeTz)/TCO-PEG₃NH-COPEG₂₄NH-Glu- | |
| DBCO/N₃-Nanobody#)₁(α-Lys(α-NHCy5)(ε- | |
| NHDFO))₁(α-NH₂)₁₄(ε-NH-COPEG₁₀₀₀)₁₆], G4 | |
| BHA[Lys]₂[Lys]₄[Lys]₈[Lys]₁₆[((α-NH-COPEG₂₄NH- | 92 |
| COPEG₄(PhMeTz)/TCO-PEG₃NH-COPEG₂₄NH-Glu- | |
| DBCO/N₃-Nanobody#)₂(α-Lys(α-NHCy5)(ε- | |
| NHDFO))₁(α-NH₂)₁₃(ε-NHCOPEG₁₀₀₀)₁₆], G4 | |
| BHA[Lys]₂[Lys]₄[Lys]₈[Lys]₁₆[Lys]₃₂[((α-NH- | 93 |
| COPEG₂₄NH-COPEG₄(PhMeTz)/TCO-PEG₃NH- | |
| COPEG₂₄NH-Glu-DBCO/N₃-Nanobody#)₁(α-Lys(α- | |
| NHCy5)(ε-NHDFO))₁(α-NH2)₃₀(ε-NH-COPEG₁₀₀₀)₃₂], | |
| G5 | |
| BHA[Lys]₂[Lys]₄[Lys]₈[Lys]₁₆[Lys]₃₂[((α-NH- | 94 |
| COPEG₂₄NH-COPEG₄(PhMeTz)/TCO-PEG₃NH- | |
| COPEG₂₄NH-Glu-DBCO/N₃-Nanobody#)₂[α-Lys(α- | |
| NHCy5)(ε-NHDFO)]₁(α-NH₂)₂₉(ε-NH-COPEG₁₀₀₀)₃₂], | |
| G5 | |
| BHALys[Lys]₂[Lys]₄[(α-Lys(α-NHCy5)(ε-NHDFO))₁(α- | 95 |
| NH₂)₃)(ε-NH-COPEG₂₄NH-COPEG₄(PhMeTz))₂)(ε-NH- | |
| COPEG₂₄NH-COPEG₄(PhMeTz))/TCO-PEG₃NH- | |
| COPEG₂₄NH-Glu-DBCO/N₃-Nanobody#)₂], G2 | |
| BHALys[Lys]₂[Lys]₄[(α-Lys(α-NHCy5)(ε-NHDFO))₁(α- | 96 |
| NH₂)₃)(ε-NH-COPEG₂₄NH-COPEG₄(PhMeTz))₁(ε-NH- | |
| COPEG₂₄NH-COPEG₄(PhMeTz)/TCO-PEG₃NH- | |
| COPEG₂₄NH-Glu-DBCO/N₃-Nanobody#)₃], G2 | |
| BHALys[Lys]₂[Lys]₄[(α-Lys(α-NHCy5)(ε-NHDFO))₁ | 97 |
| (α-NH₂)₃)(ε-NH-COPEG₂₄NH-COPEG₄ | |
| (PhMeTz)/TCO-PEG₃NH-COPEG₂₄NH-Glu-DBCO/ | |
| N₃-Nanobody#)₄], G2 | |
| BHALys[Lys]₂[Lys]₄[((α-NH-COPEG₂₄NH- | 98 |
| COPEG₄(PhTzMe)/TCO-PEG₈-Nanobody#)₁ | |
| (α-Lys(α-NHCy5)₁(ε-NHDFO)₁)₁(α-NH₂)₂) | |
| (ε-NH-COPEG₁₀₀₀)₄], G2 | |
| BHALys[Lys]₂[Lys]₄[((α-NH-COPEG₂₄-NH- | 99 |
| COPEG₄(PhTzMe)/TCO-PEG₃-Nanobody#)₁ | |
| (α-Lys(α-NHCy5)₁(ε-NHDFO)₁)₁(α-NH₂)₂) | |
| (ε-NH-COPEG₁₀₀₀)₄], G2 | |
| BHALys[Lys]₂[Lys]₄[Lys]₈[Lys]₁₆[Lys]₃₂[((α- | 100 |
| NHCy5)₁(α-NHAc)₃₁)(ε-NH-COPEG₅₇₀N₃)₁₂(ε-NH- | |
| COPEG₅₇₀N₃/BCN-DUPA)₂₀] | |
| BHALys[Lys]₂[Lys]₄[Lys]₈[Lys]₁₆[Lys]₃₂[((α- | 101 |
| NHCy5)₁(α-NHAc)₃₁)((ε-NH-COPEG₅₇₀N₃)₂₀(ε-NH- | |
| COPEG₅₇₀N₃/BCN-DUPA)₁₂)] | |
| BHALys[Lys]₂[Lys]₄[Lys]₈[Lys]₁₆[Lys]₃₂[((α- | 102 |
| NHCy5)₁(α-NHAc)₃₁)(ε-NH-COPEG₁₁₀₀N₃)₂₅(ε-NH- | |
| COPEG₁₁₀₀N₃/BCN-DUPA)₇] | |
| BHALys[Lys]₂[Lys]₄[Lys]₈[Lys]₁₆[Lys]₃₂[((α- | 103 |
| NHCy5)₁(α-NHAc)₃₁)(ε-NH-COPEG₁₁₀₀N₃)₂₂(ε-NH- | |
| COPEG₁₁₀₀N₃/BCN-DUPA)₁₀] | |
| BHALys[Lys]₂[Lys]₄[Lys]₈[Lys]₁₆[Lys]₃₂[((α-Cy5)₁ | 104 |
| (α-NHAC)₃₁(ε-NH-COPEG₁₁₀₀N₃)₁₉(ε-NH- | |
| COPEG₁₁₀₀N3/BCN-DUPA)₁₃] | |
| BHALys[Lys]₂[Lys]₄[Lys]₈[((α-(NHDOTA))₁(α- | 105 |
| NH₂)₇)(ε-NH-COPEG₂₄-N₃/BCN-DUPA)₈], G3 | |
| (MeTzPh)PEG₂₄CO-[N(PNBoc)₂] | 106 |
| (MeTzPh)PEG₂₄CO-[N(PNH₂•HCl)₂] | 107 |
| (MeTzPh)PEG₂₄CO-[N(PNH₂•HCl)₂] | 108 |
| (MeTzPh)PEG₂₄CO-[N(PN)₂[Lys]₂[NHBoc]₄, G1 | 109 |
| (MeTzPh)PEG₂₄CO-[N(PN)₂[Lys]₂[NH₂•HCl]₄, G1 | 110 |
| (MeTzPh)PEG₂₄CO-[N(PN)₂][Lys]₂[Lys]₄[NHBoc]₈, | 111 |
| G2 | |
| (MeTzPh)PEG₂₄CO-[N(PN)₂][Lys]₂[Lys]₄[NH₂•HCl]₈, | 112 |
| G2 | |
| (MeTzPh)PEG₂₄CO- | 113 |
| [N(PN)₂][Lys]₂[Lys]₄[Lys]₈[(NH₂•HCl)(PEG₁₁₀₀)]₈ G3 | |
| Nanobody#/DBCO-DFO | 114 |
| BHALys[Lys]₂[Lys]₄[Lys]₈[(α-NHBoc)₈(ε-NH- | 115 |
| COPEG₂₄-NHFmoc)₈], G3 | |
| BHALys[Lys]₂[Lys]₄[Lys]₈[(α-NHBoc)₈(ε-NH- | 116 |
| COPEG₂₄-NH₂)₈], G3 | |
| ((1R,8S,9s)-bicyclo[6.1.0]non-4-yn-9-yl)methyl(2-(3- | 117 |
| (2,5-dioxo-2,5-dihydro-1H-pyrrol-1- | |
| yl)propanamido)ethyl)carbamate | |
| Nanobody (plain) | 118 |
| GGSHHHHHHGMASMTGGQQMGRDLYENLYFQG | |
| EVOLVESGGSLVQPGGSLRLSCAASGFTFDDYA | |
| MSWVRQVPGKGLEWVSSINWSGTHTDYADSVK | |
| GRFTISRNNANNTLYLQMNSLKSEDTAVYYCAK | |
| NWRDAGTTWFEKSGSAGQGTQVTVSS | |
| (MeTzPh)-PEG₄-PEG₂₄- | 119 |
| CO[N(PN)₂][Lys]₂[Lys]₄[Lys]₈[NH₂•HCl]₁₆, G3 | |
| (MeTzPh)-PEG₄-PEG₂₄- | 120 |
| CO[N(PN)₂][Lys]₂[Lys]₄[Lys]₈[Lys]₁₆[(α-NH₂•HCl)(ε- | |
| NH-COPEG₁₁₀₀)]₁₆, G4 | |
| (MeTzPh)-PEG₄-PEG₂₄- | 121 |
| CO[N(PN)₂][Lys]₂[Lys]₄[Lys]₈[Lys]₁₆[NH₂•TFA]₃₂, G4 | |
| (MeTzPh)-PEG₄-PEG₂₄- | 122 |
| CO[N(PN)₂][Lys]₂[Lys]₄[Lys]₈[Lys]₁₆[Lys]₃₂[(α- | |
| NH₂•TFA)(ε-NH-COPEG₁₁₀₀)]₃₂, G5 | |
| Nanobody*-N₃/DBCO-NHCO- | 123 |
| PEG₁₂TCO/(MeTzPh)PEG₄CO-NHPEG₂₄CO- | |
| [N(PN)₂][Lys]₂[Lys]₄[Lys]₈[((α-NHCy5)₁(α-NHGlu- | |
| VC-PAB-MMAE)₇)(ε-NH-COPEG₁₁₀₀)₈] | |
| Nanobody*-N₃/DBCO-NHCO- | 124 |
| PEG₁₂TCO/(MeTzPh)PEG₄CO-NHPEG₂₄CO- | |
| [N(PN)₂][Lys]₄[Lys]₈[((α-NHCy5)₁(α-NHAc)₇)(ε-NH- | |
| COPEG₁₁₀₀)₈] | |
| Nanobody*-N₃/DBCO-NHCO- | 125 |
| PEG₁₂TCO/((MeTzPh)PEG₄CO-NHPEG₂₄CO- | |
| [N(PN)₂][Lys]₂[Lys]₄[Lys]₈[Lys]₁₆[((α-NHCy5)₁(α- | |
| NHAc)₁₅)(ε-NH-COPEG₁₁₀₀)₁₆ | |
| Nanobody*-N₃/DBCO-NHCO- | 126 |
| PEG₁₂TCO/(MeTzPh)PEG₄CO-NHPEG₂₄CO- | |
| [N(PN)₂][Lys]₂[Lys]₄[Lys]₈[Lys]₁₆[Lys]₃₂[((α- | |
| NHCy5)₁(α-NHAc)₃₁)(ε-NH-COPEG₁₁₀₀)₃₂] | |
| Nanobody*-N₃/DBCO-NHCO- | 127 |
| PEG₁₂TCO/(MeTzPh)PEG₄CO-NHPEG₂₄CO- | |
| [N(PN)₂][Lys]₂[³H-Lys]₄[Lys]₈[(α-NHGlu-VC-PAB- | |
| MMAE)₈(ε-NH-COPEG₁₁₀₀)₈] | |
| BHALys[Lys]₂[Lys]₄[Lys]₈[((α-NH-COPEG₂₄NH- | 128 |
| COPEG₄(PhTzMe)/TCO-PEG₃NH-COPEG₂₄NH-Glu- | |
| DBCO)1(α-Lys(α-NHCy5)(ε-NHDFO))₁(α-NH₂)₆) | |
| (ε-NH-COPEG₄₁₂)₈], G3 | |
| BHALys[Lys]₂[Lys]₄[((α-NH-COPEG₂₄NH- | 129 |
| COPEG₄(PhTzMe/TCO-PEG₃NH-COPEG₂₄NH-Glu- | |
| DBCO))₁(α-Lys(α-NHCy5)(ε-NHDFO))₁(α-NH₂)₂)(ε- | |
| NH-COPEG₁₀₀₀)₄], G2 | |
| BHALys[Lys]₂[Lys]₄[Lys]₈[((α-NH-COPEG₂₄NH- | 130 |
| COPEG₄(PhTzMe)/TCO-PEG₃NH-COPEG₂₄NH-Glu- | |
| DBCO)₁(α-Lys(α-NHCy5)(ε-NHDFO))₁(α-NH₂)₆) | |
| (ε-NH-COPEG₁₀₀₀)₈], G3 | |
| BHALys[Lys]₂[Lys]₄[Lys]₈[Lys]₁₆[((α-NH- | 131 |
| COPEG₂₄NH-COPEG₄(PhTzMe)/TCO-PEG₃NH- | |
| COPEG₂₄NH-Glu-DBCO)₁(α-Lys(α-NHCy5)(ε- | |
| NHDFO))₁(α-NH₂)₁₄)(ε-NH-COPEG₁₀₀₀)₁₆], G4 | |
| BHALys[Lys]₂[Lys]₄[Lys]₈[Lys]₁₆[Lys]₃₂[((α-NH- | 132 |
| COPEG₂₄NH-COPEG₄(PhTzMe)/TCO-PEG₃NH- | |
| COPEG₂₄NH-Glu-DBCO)₁(α-Lys(α-NHCy5)(ε- | |
| NHDFO))₁(α-NH₂)₃₀)(ε-NH-COPEG₁₀₀₀)₃₂], G5 | |
| BHALys[Lys]₂[Lys]₄[Lys]₈[Lys]₁₆[((α-NH- | SRS- |
| COPEG₂₄NH-COPEG₄(PhTzMe)₁(α-NHCy5)₁(α- | 1 |
| NHDOTA))₁₀(α-NH₂)₄)(ε-NH-COPEG₁₀₀₀)₁₆], G4 | |
| TCO-PEG₃NH-COPEG₂₄NH-Glu-DBCO/N₃- | SRS- |
| Nanobody# | 2a |
| BHALys[Lys]₂[Lys]₄[Lys]₈[Lys]₁₆[((α-NH- | SRS- |
| COPEG₂₄NH-COPEG₄(PhTzMe)/TCO-PEG₃NH- | 2-304 |
| COPEG₂₄NH-Glu-DBCO/N₃-Nanobody#)₁(α- | |
| NHCy5)₁(α-NHDOTA))₁₀(α-NH₂)₄)(ε-NH- | |
| COPEG₁₀₀₀)₁₆], G4 | |
| N₃-PEG₇-NHCO-Lys[(α-NHBoc)(ε-NHFmoc)] | SRS-3 |
| N₃-PEG₇-NHCO-Lys[(α-NH₂•HCl)(ε-NHFmoc)] | SRS-3a |
| N₃-PEG₇-NHCO-Lys[(α-NHCy5)(ε-NHFmoc)] | SRS-4 |
| N₃-PEG₇-NHCO-Lys[(α-NHCy5)(ε-NH₂)] | SRS-4a |
| N₃-PEG₄-NHCO-Lys[(α-NHCy5)(ε-NHDOTA)] | SRS-5 |
| Trastuzumab-Deglycosylated | KY-1 |
| Trastuzumab-{CONH-PEG₄-Sulfo-DBCO}₂ | KY-2 |
| Trastuzumab-{CONH-PEG₄-Sulfo-DBCO/N₃-647 | KY-2a |
| (AFDye)}₂ | |
| Trastuzumab-{CONH-PEG₄-Sulfo-DBCO/N₃-PEG₇- | KY- |
| NHCO[Lys(α-NHCy5)(ε-NHDOTA)]}₂ | 3-310 |
| BHALys[Lys]₂[Lys]₄[Lys]₈[Lys]₁₆[Lys]₃₂[(α- | RH-1 |
| NH₂•TFA)₃₂(ε-NHCOPEG₂₀₀₀)₃₂] | |
| BHALys[Lys]₂[Lys]₄[Lys]₈[Lys]₁₆[Lys]₃₂[(α-NH₂)₃₀(α- | RH-2 |
| NHDOTA)₂(ε-NHCOPEG₂₀₀₀)₃₂] | |
| BHALys[Lys]₂[Lys]₄[Lys]₈[Lys]₁₆[Lys]₃₂[(α- | RH-3- |
| NHAc)₃₀(α-NHDOTA)₂(ε-NHCOPEG₂₀₀₀)₃₂] | 160 |

Nanobody# = Nanobody-N3-C-terminal Tag
Nanobody* = N-terminal Tag-Nanobody-N3

Table of Compounds 2

| Com- | | |
|---|---|---|
| pound | Compound | |
| # | Description | Structure or short hand nomenclature |
| RHa-1 | FAPI-04 NBoc | 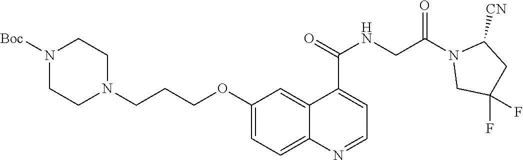 [SMILES: CC(C)(C)OC(=O)N1CCN(CCCOc2ccc3nccc(C(=O)NCC(=O)N4CC(F)(F)C[C@H]4C#N)c3c2)CC1] |
| | | Chemical Formula: C₂₉H₃₆F₂N₆O₅ |
| | | Molecular Weight: 586.63 |
| RHa-2 | FAPI-04 NH | 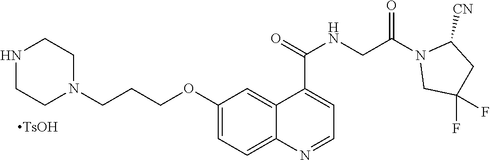 |
| | | Chemical Formula: C2₄H₂₈F₂N₆O₃ |
| | | Molecular Weight: 486.51 |
| | | Chemical Formula: C₃₁H₃₆F₂N₆O₆S |
| | | Molecular Weight: 658.72 |
| RHa-3 | Acid- dPEG₁₁₀₀— NHS Ester | 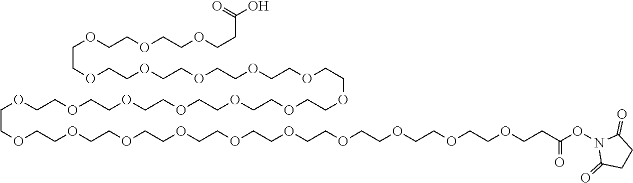 |
| | | Chemical Formula: C₅₈H₁₀₉NO₃₁ |
| | | Molecular Weight: 1316.47 |
| RHa-4 | FAPI-04— NC(O) dPEG₁₁₀₀— CO₂H | 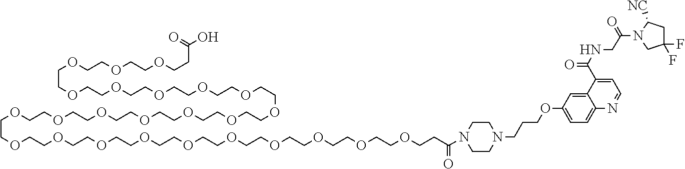 |
| | | Chemical Formula: C₇₈H₁₃₂F₂N₆O₃₁ |
| | | Molecular Weight 1687.90 |
| | | Chemical Formula: C₇₈H₁₃₂F₂N₆O₃₁ |
| | | Molecular Weight 1687.90 |
| RHa-5 | Cy5—NHS Ester | 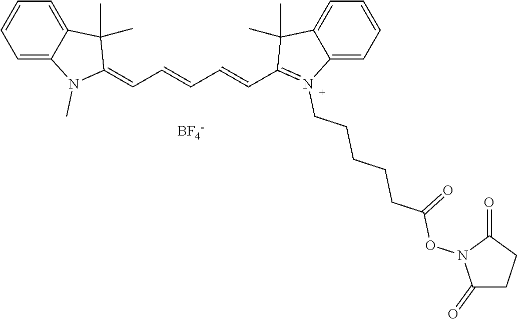 |
| | | Chemical Formula: C₃₆H₄₂BF₄N₃O₄ |
| | | Molecular Weight: 667.54 |
| RHa-6 | HO— Lys[(α- NH₂)(ε- NHBoc)] | NHBoc 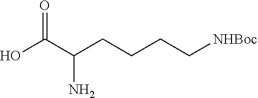 [SMILES: CC(C)(C)OC(=O)NCCCCC(N)C(=O)O] |
| | | Chemical Formula: C₁₁H₂₂N₂O₄ |
| | | Molecular Weight: 246.30 |
| RHa-7 | HO— Lys[(α- NHCy5) (ε-NH₂• TFA)] | 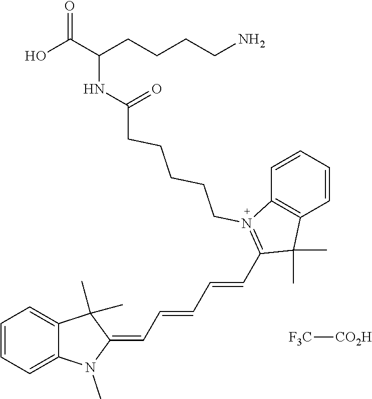 |
| | | Chemical Formula: C₃₈H₅₁N₄O₃⁺ |
| | | Molecular Weight: 611.84 |
| | | Chemical Formula: C₄₀H₅₂F₃N₄O₅⁺ |
| | | Molecular Weight: 725.86 |
| RHa-8 | p-SCN— Ph-DFO | 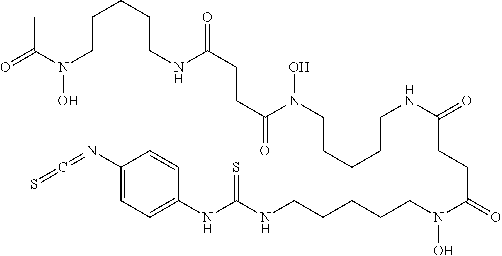 [SMILES: CC(=O)N(O)CCCCCNC(=O)CCC(=O)N(O)CCCCCNC(=O)CCC(=O)N(O)CCCCCNC(=S)Nc1ccc(N=C=S)cc1] |
| | | Chemical Formula: C₃₃H₅₂N₈O₈S₂ |
| | | Molecular Weight: 752.94 |
| RHa-9 | HO— Lys[(α- NHCy5) (ε-NH Ph-DFO)] | 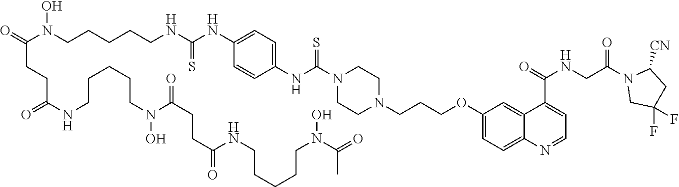 [SMILES: CC(=O)N(O)CCCCCNC(=O)CCC(=O)N(O)CCCCCNC(=O)CCC(=O)N(O)CCCCCNC(=S)Nc1ccc(NC(=S)N2CCN(CCCOc3ccc4nccc(C(=O)NCC(=O)N5CC(F)(F)C[C@H]5C#N)c4c3)CC2)cc1] |
| | | Chemical Formula: C₅₇H₈₀F₂N₁₄O₁₁S₂ |
| | | Molecular Weight: 1239.46 |
| RHa-10 | BHALys | G5 |
| | [Lys]₂[Lys]₄ | ε-(32 NH₂) |
| | [Lys]₈ | α(32 NHBoc) |
| | [Lys]₁₆ | |
| | [Lys]₃₂[(α- | |
| | NHBoc)₃₂ | |
| | (ε-NH₂)₃₂] | |
| RHa-11 | mPEG₁₀₀₀— | Commercially Available |
| | CO₂H | |
| RHa-12 | BHALys | G5 |
| | [Lys]₂[Lys]₄ | ε-(1 PEG₁₁₀₀-FAPI:31 MeO) |
| | [Lys]₈ | α-(32 NHBoc) |
| | [Lys]₁₆ | |
| | [Lys]₃₂[(α- | |
| | NHBoc)₃₂ | |
| | (ε-NHCO | |
| | dPEG₁₁₀₀— | |
| | C(O)N— | |
| | FAPI-04)₁ | |
| | (ε-NHCO— | |
| | mPEG₁₀₀₀)₃₁] | |
| RHa-13 | BHALys | G5 |
| | [Lys]₂[Lys]₄ | ε-(10 PEG₁₁₀₀-FAPI: 19 MeO: 3 NH₂) |
| | [Lys]₈ | α-(32 NHBoc) |
| | [Lys]₁₆ | MW = 49,214 Da |
| | [Lys]₃₂[(α- | |
| | NHBoc)₃₂(ε- | |
| | NHCO | |
| | dPEG₁₁₀₀ | |
| | C(O)N— | |
| | FAPI-04)₁₀ | |
| | (ε-NHCO— | |
| | mPEG₁₀₀₀)₁₉ | |
| | (ε-NH₂)₃] | |
| RHa-14 | BHALys | G5 |
| | [Lys]₂[Lys]₄ | ε-(4 PEG₁₁₀₀-FAPI: 28 MeO) |
| | [Lys]₈[Lys]₁₆ | α-(32 NHBoc) |
| | [Lys]₃₂[(α- | |
| | NHBoc)₃₂ | |
| | (ε-NHCO | |
| | dPEG₁₁₀₀— | |
| | C(O)N— | |
| | FAPI-04)₄ | |
| | (ε-NHCO— | |
| | dPEG₁₁₀₀)₂₈] | |
| RHa-15 | BHALys | G5 |
| | [Lys]₂[Lys]₄ | ε-(8 NH₂) |
| | [Lys]₈[(α- | α-(8 NHBoc) |
| | NHBoc)₈(ε- | |
| | NH₂)₈] | |
| RHa-16 | BHALys | G3 |
| | [Lys]₂[Lys]₄ | ε-(1 PEG₁₁₀₀-FAPI: 5 MeO: 2 NH₂) |
| | [Lys]₈[(α- | α-(8 NHBoc) |
| | NHBoc)₈(ε- | |
| | NHCO— | |
| | dPEG₁₁₀₀— | |
| | C(O)N— | |
| | FAPI-04)₁(ε- | |
| | NHCO- | |
| | mPEG₁₀₀₀)₅ | |
| | (ε-NH₂)₂] | |
| RHa-17 | BHALys | G3 |
| | [Lys]₂[Lys]₄ | ε-(3 PEG₁₁₀₀-FAPI: 5 MeO) |
| | [Lys]₈[(α- | α-(8 NHBoc) |
| | NHBoc)₈ | |
| | (ε-NHCO— | |
| | dPEG₁₁₀₀— | |
| | C(O)N— | |
| | FAPI-04)₃(ε- | |
| | NHCO— | |
| | mPEG₁₀₀₀)₅] | |
| RHa-18 | BHALys | G3 |
| | [Lys]₂[Lys]₄ | ε-(7.4 PEG₁₁₀₀-FAPI: 0.6 MeO) |
| | [Lys]₈[(α- | α-(8 NHBoc) |
| | NHBoc)₈(ε- | |
| | NHCO— | |
| | dPEG₁₁₀₀— | |
| | C(O)N— | |
| | FAPI-04)_{7.4} | |
| | (ε-NH₂)_{0.6}] | |
| RHa-19 | BHALys | G5 |
| | [Lys]₂[Lys]₄ | ε-(10 PEG₁₁₀₀-FAPI: 19 MeO: 3 NH₂) |
| | [Lys]₈[Lys]₁₆ | α-(32 NH₂•TFA) |
| | [Lys]₃₂[(α- | |
| | NH₂•TFA)₃₂ | |
| | (ε-NHCO— | |
| | dPEG₁₁₀₀— | |
| | C(O)N— | |
| | FAPI-04)₁₀ | |
| | (ε-NHCO— | |
| | mPEG₁₀₀₀)₁₉ | |
| | (ε-NH₂)₃] | |
| RHa-20 | BHALys | G5 |
| | [Lys]₂[Lys]₄ | ε-(1 PEG₁₁₀₀-FAPI:31 MeO) |
| | [Lys]₈[Lys]₁₆ | α-(32 NH₂•TFA) |
| | [Lys]₃₂[(α- | |
| | NH₂•TFA)₃₂ | |
| | (ε-NHCO— | |
| | dPEG₁₁₀₀— | |
| | C(O)N— | |
| | FAPI-04)₁ | |
| | (ε-NHCO- | |
| | mPEG₁₀₀₀)₃₁] | |
| RHa-21 | BHALys[ | G5 |
| Inter- | Lys]₂[Lys]₄ | ε-(4 PEG₁₁₀₀-FAPI:28 MeO) |
| mediate | [Lys]₈[Lys]₁₆ | α-(32 NH₂•TFA) |
| | [Lys]₃₂[(α- | |
| | NH₂•TFA)₃₂ | |
| | (ε-NHCO— | |
| | dPEG₁₁₀₀— | |
| | C(O)N— | |
| | FAPI-04)₄(ε- | |
| | NHCO— | |
| | dPEG₁₁₀₀)₂₈] | |
| RHa-22 | BHALys | G3 |
| Inter- | [Lys]₂[Lys]₄ | ε-(1 PEG₁₁₀₀-FAPI: 5 MeO: 2 NH₂) |
| mediate | [Lys]₈[(α- | α-(8 NH₂•TFA) |
| | NH₂•TFA)₈(ε- | |
| | NHCO— | |
| | dPEG₁₁₀₀— | |
| | C(O)N— | |
| | FAPI-04)₁(ε- | |
| | NHCO— | |
| | mPEG₁₀₀₀)₅ | |
| | (ε-NH₂)₂] | |
| RHa-23 | BHALys | G3 |
| Inter- | [Lys]₂[Lys]₄ | ε-(3 PEG₁₁₀₀-FAPI: 5 MeO) |
| mediate | [Lys]₈[(α- | α-(8 NH₂•TFA) |
| | NH₂•TFA)₈ | |
| | (ε-NHCO— | |
| | dPEG₁₁₀₀— | |
| | C(O)N— | |
| | FAPI-04)₃(ε- | |
| | NHCO— | |
| | mPEG₁₀₀₀)₅] | |
| RHa-24 | BHALys | G3 |
| Inter- | [Lys]₂[Lys]₄ | ε-(7.4 PEG₁₁₀₀-FAPI: 0.6 MeO) |
| mediate | [Lys]₈[(α- | α-(8 NH₂•TFA) |
| | NH₂•TFA)₈ | |
| | (ε-NHCO— | |
| | dPEG₁₁₀₀— | |
| | C(O)N— | |
| | FAPI-04)_{7.4} | |
| | (ε-NH₂)_{0.6}] | |
| RHa-25 | BHALys | G5 |
| | [Lys]₂[Lys]₄ | ε-(10 PEG₁₁₀₀-FAPI: 19 MeO: 3 NH₂) |
| | [Lys]₈[Lys]₁₆ | α-(1.5 Cy5/DFO: 30.5 NH₂) |
| | [Lys]₃₂[ | MW = 48,033 Da |
| | (α-NHCO— | |
| | Lys[(α- | |
| | NHCy5) | |
| | (ε-NH—Ph- | |
| | DFO)])_{1.5}(α- | |
| | NH₂)_{30.5}(ε- | |
| | NHCO— | |
| | dPEG₁₁₀₀— | |
| | C(O)N— | |
| | FAPI-04)₁₀ | |
| | (ε-NHCO- | |
| | mPEG₁₀₀₀)₁₉ | |
| | (ε-NH₂)₃] | |
| RHa-26 | BHALys | G5 |
| | [Lys]₂[Lys]₄ | ε-(1 PEG₁₁₀₀-FAPI:31 MeO) |
| | [Lys]₈[Lys]₁₆ | α-(1.5 Cy5/DFO: 30.5 NH₂) |
| | [Lys]₃₂[(α- | MW = 46,467 Da |
| | NHCO— | |
| | Lys[(a- | |
| | NHCy5)(ε- | |
| | NH—Ph- | |
| | DFO)])_{1.5}(α- | |
| | NH₂)_{30.5}(ε- | |
| | NHCO— | |
| | dPEG₁₁₀₀— | |
| | C(O)N— | |
| | FAPI-04)₁ | |
| | (ε-NHCO— | |
| | mPEG₁₀₀₀)₃₁] | |
| RHa-27 | BHALys | G5 |
| | [Lys]₂[Lys]₄ | ε-(4 PEG₁₁₀₀-FAPI:28 MeO) |
| | [Lys]₈[Lys]₁₆ | α-(1.5 Cy5/DFO: 30.5 NH₂) |
| | [Lys]₃₂[(α- | MW = 49,143 Da |
| | NHCO— | |
| | Lys[(α- | |
| | NHCy5) | |
| | (ε-NH—Ph- | |
| | DFO)])_{1.5}(α- | |
| | NH₂)_{30.5}(ε- | |
| | NHCO— | |
| | dPEG₁₁₀₀— | |
| | C(O)N— | |
| | FAPI-04)₄ | |
| | (ε-NHCO— | |
| | dPEG₁₁₀₀)₂₈] | |
| RHa-28 | BHALys | G3 |
| | [Lys]₂[Lys]₄ | ε-(1 PEG₁₁₀₀-FAPI: 5 MeO: 2 NH₂) |
| | [Lys]₈[(α- | α-(1.5 Cy5/DFO: 6.5 NH₂) |
| | NHCO— | MW = 8,768 Da |
| | Lys[(α- | |
| | NHCy5)(ε- | |
| | NH—Ph- | |
| | DFO)])_{1.5} | |
| | (α-NH₂)_{6.5} | |
| | (ε-NHCO— | |
| | dPEG₁₁₀₀— | |
| | C(O)N— | |
| | FAPI-04)₁ | |
| | (ε-NHCO— | |
| | mPEG₁₀₀₀)₅ | |
| | (ε-NH₂)₂] | |
| RHa-29 | BHALys | G3 |
| | [Lys]₂[Lys]₄ | ε-(3 PEG₁₁₀₀-FAPI: 5 MeO) |
| | [Lys]₈[( | α-(1.5 Cy5/DFO: 6.5 NH₂) |
| | α-NHCO— | MW = 12,108 Da |
| | Lys[(α- | |
| | NHCy5)(ε- | |
| | NH—Ph— | |
| | DFO)])_{1.5}(α- | |
| | NH₂)_{6.5}(ε- | |
| | NHCO- | |
| | dPEG₁₁₀₀— | |
| | C(O)N— | |
| | FAPI-04)₃(ε- | |
| | NHCO— | |
| | mPEG₁₀₀₀)₅] | |
| RHa-30 | BHALys | G3 |
| | [Lys]₂[Lys]₄ | ε-(7.4 PEG₁₁₀₀-FAPI: 0.6 MeO) |
| | [Lys]₈[( | α-(1.5 Cy5/DFO: 6.5 NH₂) |
| | α-NHCO— | MW =13,916 Da |
| | Lys[(α- | |
| | NHCy5)(ε- | |
| | NH—Ph— | |
| | DFO)])_{1.5}(α- | |
| | NH₂)_{6.5}(ε- | |
| | NHCO— | |
| | dPEG₁₁₀₀— | |
| | C(O)N— | |
| | FAPI-04)_{7.4}(ε- | |
| | NH₂)_{0.6}] | |
| RHa-31 | FAPI-04-DFO | 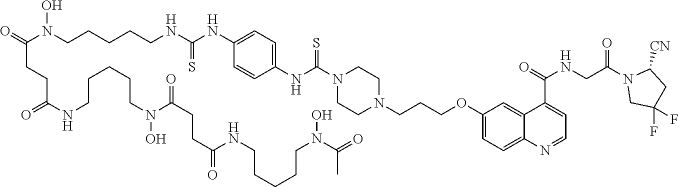 [SMILES: CC(=O)N(O)CCCCCNC(=O)CCC(=O)N(O)CCCCCNC(=O)CCC(=O)N(O)CCCCCNC(=S)Nc1ccc(NC(=S)N2CCN(CCCOc3ccc4nccc(C(=O)NCC(=O)N5CC(F)(F)C[C@H]5C#N)c4c3)CC2)cc1] |
| | | Chemical Formula: C₅₇H₈₀F₂N₁₄O₁₁S₂ |
| | | Molecular Weight: 1239.46 |
| RHa-32 | dPEG₁₀₀₀—CO₂H | 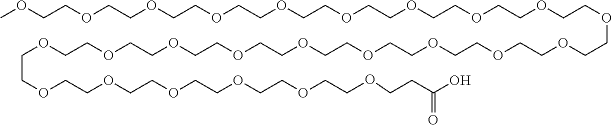 |
| | | Chemical Formula: C₅₂H₁₀₄O₂₇ |
| | | Molecular Weight: 1161.37 |
| RHa-33 | p-SCN-Bn-DOTA | 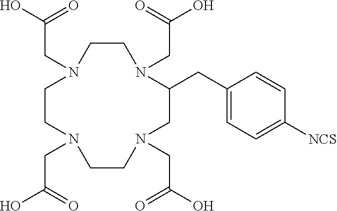 |
| RHa-34 | BHALys | G3 |
| | [Lys]₂[Lys]₄ | ε-(3 PEG₁₁₀₀-FAPI: 5 MeO) |
| | [Lys]₈[( | α-(4 DOTA: 4 NH₂) |
| | α-NH— | MW = 12,127 Da |
| | C(S)—NH— | |
| | Bn-DOTA)₄ | |
| | (α-NH₂)₄ | |
| | (ε-NHCO— | |
| | dPEG₁₁₀₀— | |
| | C(O)N— | |
| | FAPI-04)₃ | |
| | (ε-NHCO— | |
| | mPEG₁₀₀₀)₅] | |
| RHa-35 | BHALys | G5 |
| | [Lys]₂[Lys]₄ | ε-(10 FAPI: 19 MeO: 3 NH₂) |
| | [Lys]₈[Lys]₁₆ | α-(8 DOTA: 24 NH₂) |
| | [Lys]₃₂[(α- | MW = 50,427 Da |
| | NHC(S)— | |
| | NH—Bn- | |
| | DOTA)₈(α- | |
| | NH₂)₂₄(ε- | |
| | NHCO— | |
| | dPEG₁₁₀₀— | |
| | C(O)N— | |
| | FAPI-04)₁₀ | |
| | (ε-NHCO— | |
| | mPEG₁₀₀₀)₁₉ | |
| | (ε-NH₂)₃] | |
| RL-19 | DOTAGA-tetra-(tert-butyl ester) | 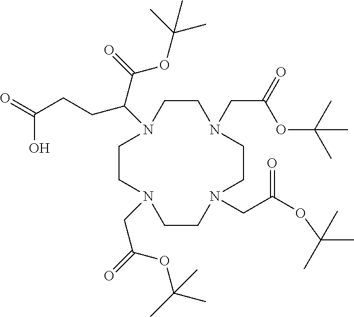 [SMILES: CC(C)(C)OC(=O)CN1CCN(CC(=O)OC(C)(C)C)CCN(C(CCC(=O)O)C(=O)OC(C)(C)C)CCN(CC(=O)OC(C)(C)C)CC1] |
| RL-20 | DOTA (OtBu)₄GA— NHS Ester | 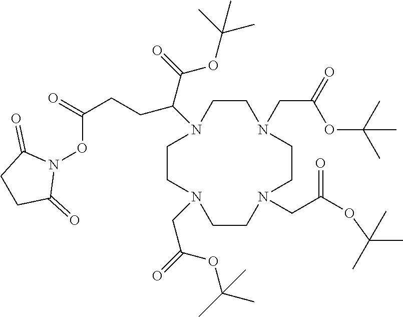 [SMILES: CC(C)(C)OC(=O)CN1CCN(CC(=O)OC(C)(C)C)CCN(C(CCC(=O)ON2C(=O)CCC2=O)C(=O)OC(C)(C)C)CCN(CC(=O)OC(C)(C)C)CC1] |
| RL-21 | DOTA | |
| | (OtBu)₄GA— | |
| | NHCO— | |
| | PEG₂₄— | |
| | COOH | |
| RL-22 | BHALys | G5 |
| | [Lys]₂[Lys]₄ | ε-(32 mPEG₁₀₀₀) |
| | [Lys]₈[Lys]₁₆ | α-(1 Cy5: 15 DUPA: 4 DOTA: 11 |
| | [Lys]₃₂[(α- | NH₂•TFA) |
| | NHCy5)₁(α- | |
| | NHCO— | |
| | PEG₂₄— | |
| | NH— | |
| | DUPA)₁₅ | |
| | (α-NHCO— | |
| | PEG₂₄NH— | |
| | GADOTA)₄ | |
| | (α-NH₂• | |
| | TFA)₁₁(ε- | |
| | NHCO | |
| | PEG₁₀₀₀)₃₂] | |
| RL-23 | BHALys | G5 |
| | [Lys]₂[Lys]₄ | ε-(32 mPEG₁₀₀₀) |
| | [Lys]₈[Lys]₁₆ | α-(1 Cy5: 5 PEG₂₄-DUPA: 16 DGA-SN38: |
| | [Lys]₃₂[(α- | 10 NH₂•TFA) |
| | NHCy5)₁(α- | MW = 62,559 Da |
| | NHCO— | |
| | PEG₂₄NH— | |
| | DUPA)₅(α- | |
| | NHDGA- | |
| | SN38)₁₆(α- | |
| | NH₂•TFA)₁₀ | |
| | (ε-NHCO | |
| | PEG₁₀₀₀)₃₂] | |
| RL-24 | DGA-(C-20) SN38 | 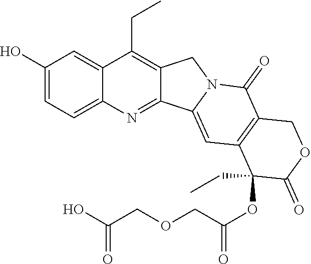 [SMILES: CCc1c2c(nc3ccc(O)cc13)-c1cc3c(c(=O)n1C2)COC(=O)[C@@]3(CC)OC(=O)COCC(=O)O] |
| RL-25 | BHALys | G5 |
| | [Lys]₂[Lys]₄ | ε-(32 mPEG₁₀₀₀) |
| | [Lys]₈[Lys]₁₆ | α-(1 Cy5: 8 PEG₂₄-DUPA: 14 DOTA: 9 |
| | [Lys]₃₂[(α- | NH₂•TFA) |
| | NHCy5)₁(α- | |
| | NHCO— | |
| | PEG₂₄NH— | |
| | DUPA)₈(α- | |
| | NHDOTA)₁₄ | |
| | (α-NH₂•TFA)₉ | |
| | (ε-NHCO | |
| | PEG₁₀₀₀)₃₂] | |
| RL-26 | BHALys | G3 |
| | [Lys]₂[Lys]₄ | a/8-(8 mPEG1000) |
| | [Lys]₈[(α/ε- | α/ε-(1 Cy5: 5 PEG₂₄-DUPA: 14 DOTA: 3 |
| | NHCy5)₁ | NH₂•TFA) |
| | (α/ε-NHCO— | |
| | PEG₂₄NH | |
| | DUPA)₅(α/ε- | |
| | NH— | |
| | DOTAGA)₇ | |
| | (α/ε-NH₂• | |
| | TFA)₃] | |

TABLE of Compounds 3

| Com- | | | |
|---|---|---|---|
| pound | Compound | | |
| # | Description | Structure | |
| RL-1 | Cystamine hydrochlor- ide | 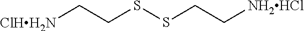 | |
| | | Chemical Formula: C₄H₁₄Cl₂N₂S₂ | |
| | | Molecular Weight: 225.20 | |
| | | C₄H₁₄Cl₂N₂S₂ | |
| | | Molecular Weight: 225.20 | |
| RL-2 | Boc- Lys(Boc)— ONp | 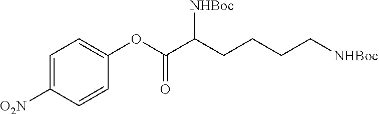 [SMILES: CC(C)(C)OC(=O)NCCCCC(NC(=O)OC(C)(C)C)C(=O)Oc1ccc([N+](=O)[O-])cc1] | |
| | | Chemical Formula: C₂₂H₃₃N₃O₈ | |
| | | Molecular Weight: 467.51 | |
| | | C2₂H₃₃N₃O₈ | |
| | | Molecular Weight: 467.51 | |
| RL-3 | [[ε-NHBoc) (α-NHBoc)] [Lys]— CONH— CH₂— CH₂—S—]₂ | 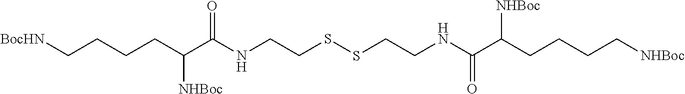 [SMILES: CC(C)(C)OC(=O)NCCCCC(NC(=O)OC(C)(C)C)C(=O)NCCSSCCNC(=O)C(CCCCNC(=O)OC(C)(C)C)NC(=O)OC(C)(C)C] | |
| | | Chemical Formula: C₃₆H₆₈N₆O₁₀S₂ | |
| | | Molecular Weight: 809.09 | |
| | | C₃₆H₆₈N₆O₁₀S₂ | |
| | | Molecular Weight: 809.09 | |
| RL-4 | [[ε-H₂•TFA) (α-NH₂• TFA)] [Lys]— CONH— CH₂— CH₂—S—]₂ | 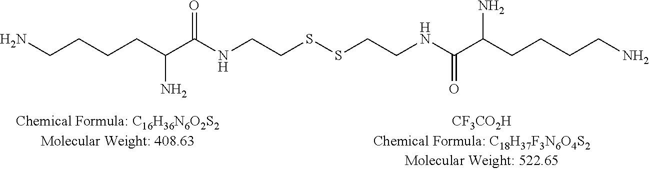 [SMILES: NCCCCC(N)C(=O)NCCSSCCNC(=O)C(N)CCCCN] | |
| | | C₁₆H₃₆N₆O₂S₂ | |
| | | Molecular Weight: 408.63 | |
| | | C₁₈H₃₇F₃N₆O₄S₂ | |
| | | Molecular Weight: 522.65 | |
| RL-5 | [[(ε-HBoc)₂ (α-NHBoc)₂] [Lys]₂ [Lys]— CONH— CH₂— CH₂—S—]₂ | 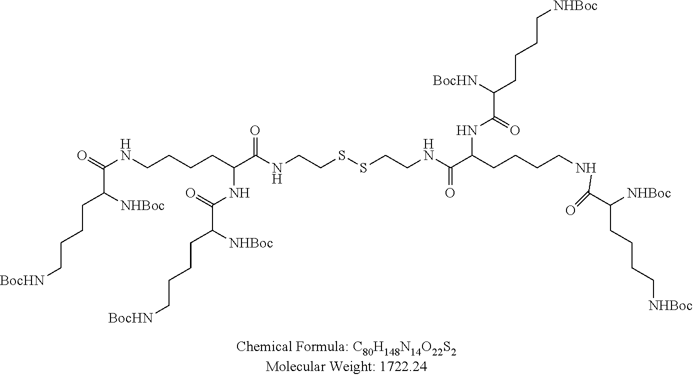 [SMILES: CC(C)(C)OC(=O)NCCCCC(NC(=O)OC(C)(C)C)C(=O)NCCCCC(NC(=O)C(CCCCNC(=O)OC(C)(C)C)NC(=O)OC(C)(C)C)C(=O)NCCSSCCNC(=O)C(CCCCNC(=O)C(CCCCNC(=O)OC(C)(C)C)NC(=O)OC(C)(C)C)NC(=O)C(CCCCNC(=O)OC(C)(C)C)NC(=O)OC(C)(C)C] | |
| | | Chemical Formula: C₈₀H₁₄₈N₁₄O₂₂S₂ | |
| | | Molecular Weight: 1722.24 | |
| RL-6 | [(ε-NH₂• TFA)₂(α- NH₂•TFA)₂] [Lys]₂ [Lys]— CONH— CH₂— CH₂—S—]₂ | 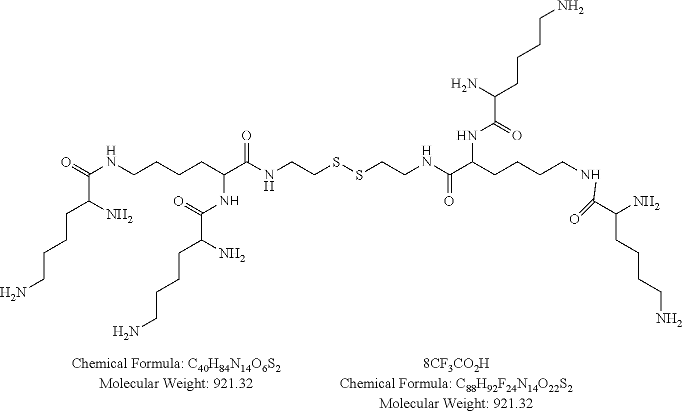 [SMILES: NCCCCC(N)C(=O)NCCCCC(NC(=O)C(N)CCCCN)C(=O)NCCSSCCNC(=O)C(CCCCNC(=O)C(N)CCCCN)NC(=O)C(N)CCCCN] | |
| | | C₄₀H₈₄N₁₄O₆S₂ | |
| | | Molecular Weight: 921.32 | |
| | | C₅₆H₉₂F₂₄N₁₄O₂₂S₂ | |
| | | Molecular Weight: 1833.50 | |
| RL-7 | [[(ε- NHBoc)₄ (α-NHBoc)₄] [Lys]₄[Lys]₂ [Lys]— CONH— CH₂— CH₂—S—]₂ | 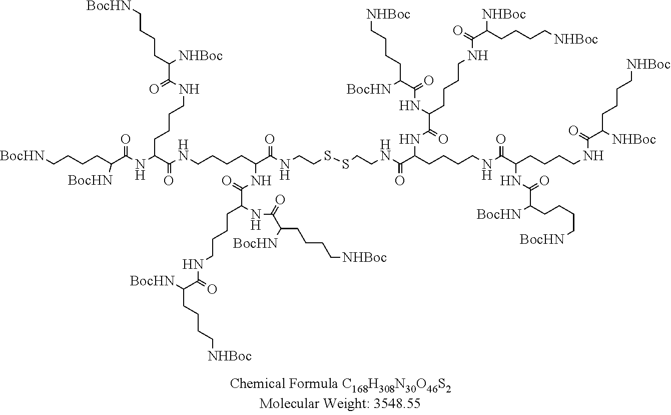 [SMILES: CC(C)(C)OC(=O)NCCCCC(NC(=O)OC(C)(C)C)C(=O)NCCCCC(NC(=O)C(CCCCNC(=O)OC(C)(C)C)NC(=O)OC(C)(C)C)C(=O)NCCCCC(NC(=O)C(CCCCNC(=O)C(CCCCNC(=O)OC(C)(C)C)NC(=O)OC(C)(C)C)NC(=O)C(CCCCNC(=O)OC(C)(C)C)NC(=O)OC(C)(C)C)C(=O)NCCSSCCNC(=O)C(CCCCNC(=O)C(CCCCNC(=O)C(CCCCNC(=O)OC(C)(C)C)NC(=O)OC(C)(C)C)NC(=O)C(CCCCNC(=O)OC(C)(C)C)NC(=O)OC(C)(C)C)NC(=O)C(CCCCNC(=O)C(CCCCNC(=O)OC(C)(C)C)NC(=O)OC(C)(C)C)NC(=O)C(CCCCNC(=O)OC(C)(C)C)NC(=O)OC(C)(C)C] | |
| | | C₁₆₈H₃₀₈N₃₀O₄₆S₂ | |
| | | Molecular Weight: 3548.55 | |
| RL-8 | [[(ε-NH₂• TFA)₄(α- NH₂•TFA)₄] [Lys]₄[Lys]₂ [Lys]— CONH— CH₂— CH₂—S—]₂ | 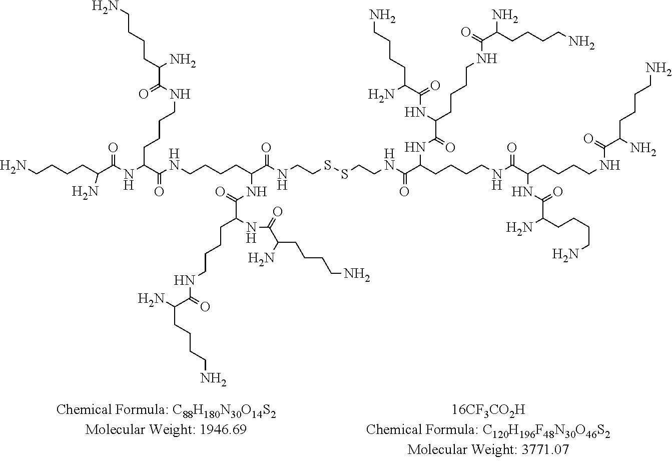 [SMILES: NCCCCC(N)C(=O)NCCCCC(NC(=O)C(N)CCCCN)C(=O)NCCCCC(NC(=O)C(CCCCNC(=O)C(N)CCCCN)NC(=O)C(N)CCCCN)C(=O)NCCSSCCNC(=O)C(CCCCNC(=O)C(CCCCNC(=O)C(N)CCCCN)NC(=O)C(N)CCCCN)NC(=O)C(CCCCNC(=O)C(N)CCCCN)NC(=O)C(N)CCCCN] | |
| | | C₈₈H₁₈₀N₃₀O₁₄S₂ | |
| | | Molecular Weight: 1946.69 | |
| | | C₁₂₀H₁₉₆F₄₈N₃₀O₄₆S₂ | |
| | | Molecular Weight: 3771.07 | |
| RL-9 | [[(ε- NHBoc)₈ (α- NHBoc)₈] [Lys]₈[Lys]₄ [Lys]₂ [Lys]— CONH— CH₂— CH₂—S—]₂ | 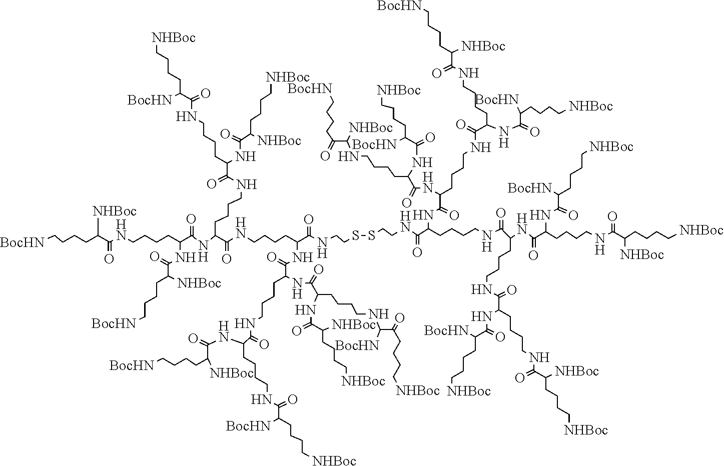 | |
| | | Chemical Formula: C₃₄₄H₆₂₈N₆₂O₉₄S₂ | |
| | | Molecular Weight: 7201.16 | |
| | | C₃₄₄H₆₂₈N₆₂O₉₄S₂ | |
| | | Molecular Weight: 7201.16 | |
| RL-10 | [[(ε-NH₂• TFA)₈(α- NH₂•TFA)₈] [Lys]₈[Lys]₄ [Lys]₂ [Lys]— CONH— CH₂— CH₂—S—]₂ | 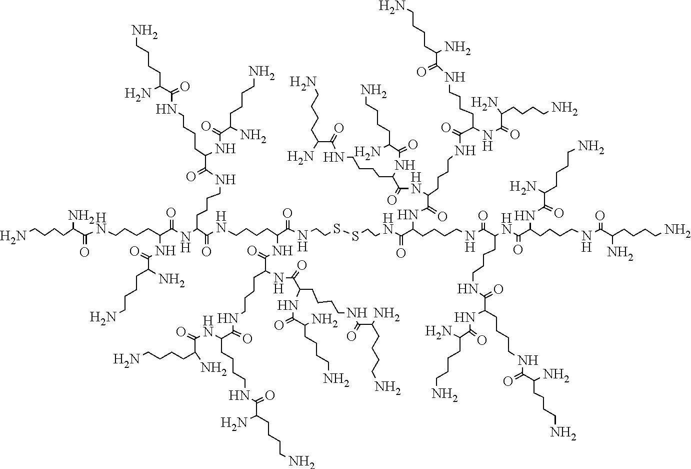 [SMILES: NCCCCC(N)C(=O)NCCCCC(NC(=O)C(N)CCCCN)C(=O)NCCCCC(NC(=O)C(CCCCNC(=O)C(N)CCCCN)NC(=O)C(N)CCCCN)C(=O)NCCCCC(NC(=O)C(CCCCNC(=O)C(CCCCNC(=O)C(N)CCCCN)NC(=O)C(N)CCCCN)NC(=O)C(CCCCNC(=O)C(N)CCCCN)NC(=O)C(N)CCCCN)C(=O)NCCSSCCNC(=O)C(CCCCNC(=O)C(CCCCNC(=O)C(CCCCNC(=O)C(N)CCCCN)NC(=O)C(N)CCCCN)NC(=O)C(CCCCNC(=O)C(N)CCCCN)NC(=O)C(N)CCCCN)NC(=O)C(CCCCNC(=O)C(CCCCNC(=O)C(N)CCCCN)NC(=O)C(N)CCCCN)NC(=O)C(CCCCNC(=O)C(N)CCCCN)NC(=O)C(N)CCCCN] | |
| | | Chemical Formula:C₁₈₄H₃₇₂N₆₂O₃₀S₂ | 32CF₃CO₂H |
| | | Molecular Weight: 3997.45 | C₂₄₈H₄₀₄F₉₆N₆₂O₉₄S₂ |
| | | | Molecular Weight: 7646.20 |
| | | C₁₈₄H₃₇₂N₆₂O₃₀S₂ | |
| | | Molecular Weight: 3997.45 | |
| | | C₂₄₈H₄₀₄F₉₆N₆₂O₉₄S₂ | |
| | | Molecular Weight: 7646.20 | |
| RL-11 | amino- PEG₂₄-acid | 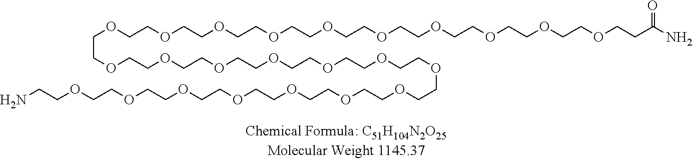 | |
| | | C₅₁H₁₀₄N₂O₂₅ | |
| | | Molecular Weight: 1145.37 | |
| RL-12 | 1H-Pyrrole- 1-carboxylic acid, 3,4-dibromo- 2,5-dihydro- 2,5-dioxo-, ethyl ester | 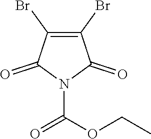 | |
| | | Chemical Formula: C₇H₅Br₂NO₄ | |
| | | Molecular Weight: 326.93 | |
| RL-13 | 4-(6- Methyl- 1,2,4,5- tetrazin-3- yl)benzene- methana- mine | 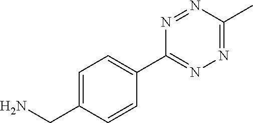 | |
| | | Chemical Formula: C₁₀H₁₁N₅ | |
| | | Molecular Weight: 201.23 | |
| RL-15 | Br₂(MAL)- PEG₃— NHCO— PEG₂₄— CONH— PEG₄- (PhTzMe) | 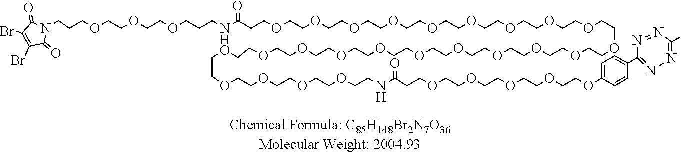 [SMILES: Cc1nnc(-c2ccc(OCCOCCOCCOCCOCCC(=O)NCCOCCOCCOCCOCCOCCOCCOCCOCCOCCOCCOCCOCCOCCOCCOCCOCCOCCOCCOCCOCCOCCOCCOCCOCCC(=O)NCCCOCCOCCOCCCN3C(=O)C(Br)=C(Br)C3=O)cc2)nn1] | |
| | | C₈₅H₁₄₉Br₂N₇O₃₆ | |
| | | Molecular Weight: 2004.93 | |
| RL-16 | Br₂(MAL)- PEG₃— NHBoc | 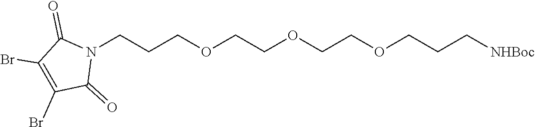 [SMILES: CC(C)(C)OC(=O)NCCCOCCOCCOCCCN1C(=O)C(Br)=C(Br)C1=O] | |
| | | Chemical Formula: C₁₉H₃₀Br₂N₂O₇ | |
| | | Molecular Weight: 558.26 | |
| RL-17 | Br₂(MAL)- PEG₃— NH₂•TFA | 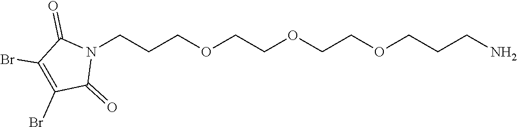 [SMILES: NCCCOCCOCCOCCCN1C(=O)C(Br)=C(Br)C1=O] | |
| | | Chemical Formula: C₁₄H₂₂Br₂N₂O₅ | |
| | | Molecular Weight: 458.14 | |
| | | CF3COOH | |
| | | Chemical Formula: C₁₆H₂₃Br₂F₃N₂O₇ | |
| | | Molecular Weight: 572.17 | |
| RL-18 | Me(MAL)- PEG₂₄— CONH— Bn-Tz(Me) | 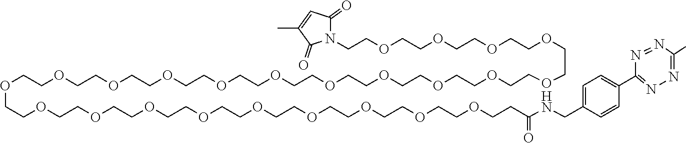 [SMILES: CC1=CC(=O)N(CCOCCOCCOCCOCCOCCOCCOCCOCCOCCOCCOCCOCCOCCOCCOCCOCCOCCOCCOCCOCCOCCOCCOCCOCCC(=O)NCc2ccc(-c3nnc(C)nn3)cc2)C1=O] | |
| | | Chemical Formula: C₆₆H₁₁₄N₆O₂₇ | |
| | | Molecular Weight 1423.64 | |
| SRS-5a | HO₂C— PEG₂₄— CONH— PEG₄- (PhTzMe) | 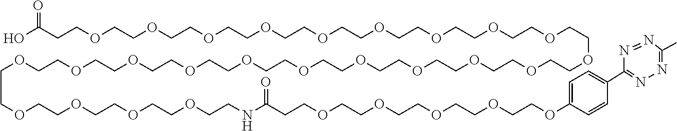 [SMILES: Cc1nnc(-c2ccc(OCCOCCOCCOCCOCCC(=O)NCCOCCOCCOCCOCCOCCOCCOCCOCCOCCOCCOCCOCCOCCOCCOCCOCCOCCOCCOCCOCCOCCOCCOCCOCCC(=O)O)cc2)nn1] | |
| | | Chemical Formula: C₇₁H₁₂₉N₅O₃₂ | |
| | | Molecular Weight: 1584.80 | |
| | | C₇₁H₁₂₉N₅O₃₂ | |
| | | Molecular Weight: 1564.80 | |
| SRS-6 | BocHN— PEG₃—NH₂ | 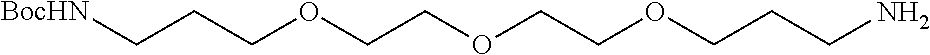 [SMILES: CC(C)(C)OC(=O)NCCCOCCOCCOCCCN] | |
| | | Chemical Formula: C₁₅H₃₂N₂O₅ | |
| | | Molecular Weight: 320.42 | |
| | | C₁₅H₃₂N₂O₅ | |
| | | Molecular Weight: 320.42 | |
| SRS-7 | BocHN— PEG₃— NHCO— PEG₂₄— CONH— PEG₄— (PhTzMe) | 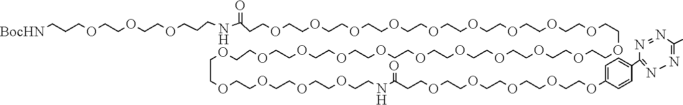 [SMILES: Cc1nnc(-c2ccc(OCCOCCOCCOCCOCCC(=O)NCCOCCOCCOCCOCCOCCOCCOCCOCCOCCOCCOCCOCCOCCOCCOCCOCCOCCOCCOCCOCCOCCOCCOCCOCCC(=O)NCCCOCCOCCOCCCNC(=O)OC(C)(C)C)cc2)nn1] | |
| | | Chemical Formula: C₈₆H₁₅₉N₇O₃₆ | |
| | | Molecular Weight: 1807.21 | |
| | | C₈₆H₁₅₉N₇O₃₆ | |
| | | Molecular Weight: 1867.21 | |
| SRS-8 | H₂N— PEG₃— NHCO— PEG₂₄— CONH— PEG₄— (PhTzMe) | 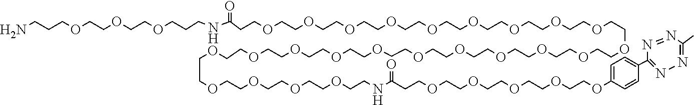 [SMILES: Cc1nnc(-c2ccc(OCCOCCOCCOCCOCCC(=O)NCCOCCOCCOCCOCCOCCOCCOCCOCCOCCOCCOCCOCCOCCOCCOCCOCCOCCOCCOCCOCCOCCOCCOCCOCCC(=O)NCCCOCCOCCOCCCN)cc2)nn1] | |
| | | Chemical Formula: C₈₁H₁₅₁N₇O₃₄ | Chemical Formula: C₈₁H₁₅₂ClN₇O₃₄ |
| | | Molecular Weight: 1787.09 | Molecular Weight: 1809.55 |
| | | C₈₁H₁₅₁N₇O₃₄ | |
| | | Molecular Weight: 1767.09 | |
| | | C₈₁H₁₅₂ClN₇O₃₄ | |
| | | Molecular Weight: 1803.55 | |
| SRS-9 | Citraconic anhydride | 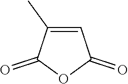 | |
| | | Chemical Formula: C₅H₄O₃ | |
| | | Molecular Weight: 112.08 | |
| SRS-10 | Me(MAL)- PEG₂₄— CO₂H | 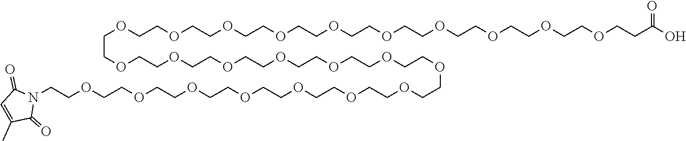 [SMILES: CC1=CC(=O)N(CCOCCOCCOCCOCCOCCOCCOCCOCCOCCOCCOCCOCCOCCOCCOCCOCCOCCOCCOCCOCCOCCOCCOCCOCCC(=O)O)C1=O] | |
| | | Chemical Formula: C₅₆H₁₀₅NO₂₈ | |
| | | Molecular Weight: 1240.42 | |
| | | C₅₆H₁₀₅NO₂₈ | |
| | | Molecular Weight: 1240.42 | |
| SRS-11 | NH₂— PEG₃— TCO | 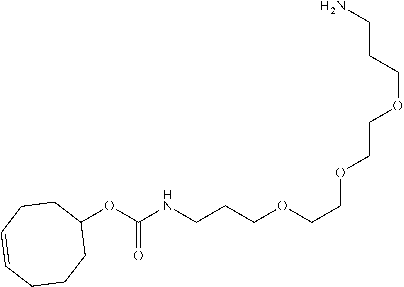 [SMILES: NCCCOCCOCCOCCCNC(=O)OC1CC/C=C\CCC1] | |
| | | Chemical Formula: C₁₉H₃₆N₂O₅ | |
| | | Molecular Weight: 372.50 | |
| SRS-12 | Me(MAL)- PEG₂₄— CONH— PEG₃-TCO | 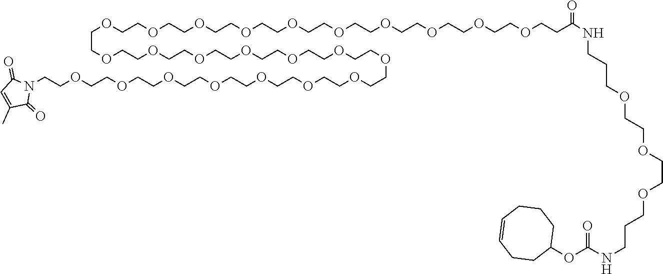 [SMILES: CC1=CC(=O)N(CCOCCOCCOCCOCCOCCOCCOCCOCCOCCOCCOCCOCCOCCOCCOCCOCCOCCOCCOCCOCCOCCOCCOCCOCCC(=O)NCCCOCCOCCOCCCNC(=O)OC2CC/C=C\CCC2)C1=O] | |
| | | Chemical Formula: C₇₅H₁₈₉N₃O₃₂ | |
| | | Molecular Weight: 1594.91 | |
| | | C₇₅H₁₃₉N₃O₃₂ | |
| | | Molecular Weight: 1594.91 | |
| RP-1 | [(ε-NH₂• TFA)₈(α- NH₂•TFA)₈] [Lys]₈[Lys]₄ [Lys]₂ [Lys]— CONH— CH₂— CH₂—S— Me(MAL)- PEG₂₄— CONH— Bn-Tz(Me) | 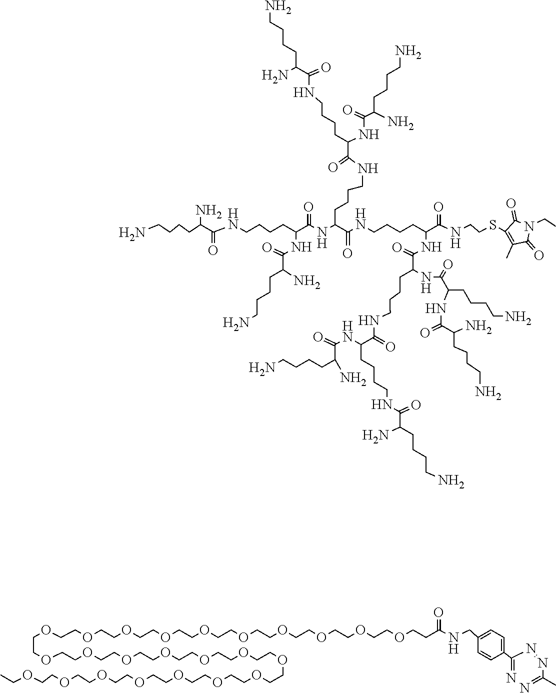 | |
| | | Chemical Formula:C₁₅₈H₃₀₁N₃₇O₄₂S | 18 CF₃CO₂H |
| | | Molecular Weight: 3423.37 | C₁₉₀H₃₁₇F₄₈N₃₇O₇₄S |
| | | | Molecular Weight: 5247.74 |
| | | C₁₅₈H₃₀₁N₃₇O₄₂S | |
| | | Molecular Weight: 3423.37 | |
| | | C₁₉₀H₃₁₇F₄₈N₃₇O₇₄S | |
| | | Molecular Weight: 5247.74 | |
| RP-2 | HO— Lys(Boc) (PEG₁₁₀₀) | 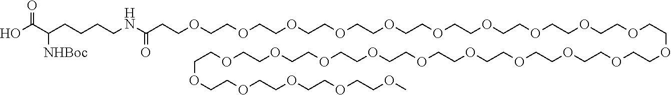 [SMILES: COCCOCCOCCOCCOCCOCCOCCOCCOCCOCCOCCOCCOCCOCCOCCOCCOCCOCCOCCOCCOCCOCCOCCOCCOCCC(=O)NCCCCC(NC(=O)OC(C)(C)C)C(=O)O] | |
| | | Chemical Formula: C₆₃H₁₂₄N₂O₃₀ | |
| | | Molecular Weight 1389.65 | |
| | | C₆₃H₁₂₄N₂O₃₀ | |
| | | Molecular Weight: 1389.65 | |
| RP-3 | [(ε- NHCO— PEG₁₁₀₀)₁₆ (α- NHBoc)₁₆] [Lys]₁₆ [Lys]₈ | [symbol] | |
| | [Lys]₄[Lys]₂ | C₁₁₆₇H₂₂₅₇N₆₉O₅₀₆S | |
| | [Lys] | Molecular Weight: 25385.63 | |
| | CONH | | |
| | CH₂ | | |
| | CH₂—S/ | | |
| | Me(MAL)- | | |
| | PEG₂₄ | | |
| | CONH | | |
| | Bn-Tz(Me) | | |
| RP-4 | [(ε- NHCO— PEG₁₁₀₀)₁₆ (α-NH₂• HCl)₁₆] [Lys]₁₆ [Lys]₈[Lys]₄ [Lys]₂ | [symbol] | |
| | [Lys]₂ | C₁₀₈₇H₂₁₂₉N₆₉0₄₇₄S | |
| | CONH | Molecular Weight: 23783.78 | |
| | CH₂— | C₁₀₈₇H₂₁₄₅Cl₁₆N₆₉O₄₇₄S | |
| | CH₂—S/ | Molecular Weight: 24367.15 | |
| | Me(MAL)- | Molecular Weight: 26665 | |
| | PEG₂₄ | | |
| | CONH | | |
| | Bn-Tz(Me) | | |
| RP-5 | [(ε- | | |
| | NHCO— | | |
| | PEG₁₁₀₀)₁₆ | | |
| | (α-NHCy5)₁ | | |
| | (α- | | |
| | NHDOTA)₈ | | |
| | (α-NH₂)₇] | | |
| | [Lys]₁₆ | | |
| | [Lys]₈[Lys]₄ | | |
| | [Lys]₂ | | |
| | [Lys] | | |
| | CONH— | | |
| | CH₂— | | |
| | CH₂—S/ | | |
| | Me(MAL)- | | |
| | PEG₂₄— | | |
| | CONH— | | |
| | Bn-Tz(Me) | | |
| SRS- 1-Mal | [(ε-NH₂)₄ (α-NH₂)₄] [Lys]₄[Lys]₂ [Lys]— CONH— CH₂— CH₂— S—]₂MAL- PEG₃— NHCO— PEG₂₄— CONH— PEG₄— (PhTzMe) | 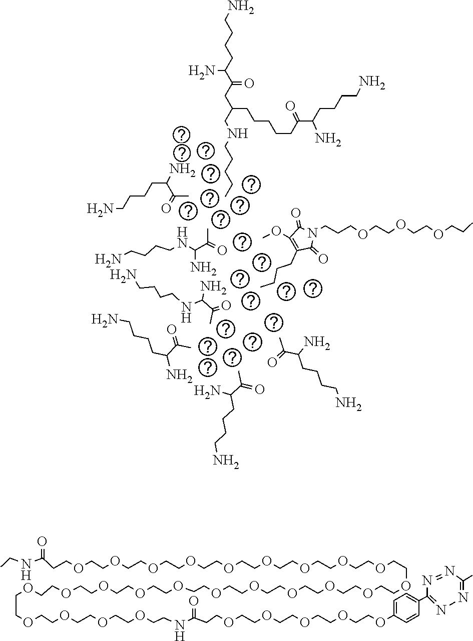 | |
| | | Chemical Formula: C₁₇₃H₃₃₁N₃₉O₅₀S₂ | 32 CF₃CO₂H |
| | | Molecular Weight: 3821.84 | Chemical Formula: C₂₃₇H₃₆₃F₉₆N₃₉O₁₁₄S₂ |
| | | | Molecular Weight: 7470.59 |

| | | |
|---|---|---|
| | | C₁₇₃H₃₃₁N₃₉O₅₀S₂ |
| | | Molecular Weight: 3821.84 |
| | | C₂₃₇H₃₆₃F₉₆N₃₉O₁₁₄S₂ |
| | | Molecular Weight: 7470.59 |
| SRS-2 | [(ε- NHCO— PEG₁₁₀₀)₈ (α- NHBoc)₈] [Lys]₈[Lys]₄ [Lys]₂ [Lys]— CONH | [symbol] |
| | CH₂ | C₁₁₈₁H₂₂₈₃N₇₁O₅₁₄S₂ |
| | CH₂—S—]₂ | Molecular Weight: 25768.06 |
| | MAL- | |
| | PEG₃ | |
| | NHCO | |
| | PEG₂₄ | |
| | CONH | |
| | PEG₄- | |
| | (PhTzMe) | |
| SRS- 3-Mal | [[(ε-NHCO—PEG₁₁₀₀)₈(α- NH₂•HCl)₈][Lys]₈[Lys]₄[Lys]₂ [Lys]—CONH—CH₂— | [symbol] |
| | CH₂—S— | C₁₁₀₂H₂₁₅₉N₇₁O₄₈₂S₂ |
| | MAL-PEG₃- | Molecular Weight: 24182.25 |
| | NHCO- | Chemical Formula: C₁₁₀₂H₂₁₇₅Cl₁₆N₇₁O₄₈₂S₂ |
| | PEG₂₄-]₂ | Molecular Weight: 24765.63 |
| | CONH— | |
| | PEG₄— | |
| | (PhTzMe) | |
| SRS-4- | [[(ε- | |
| Mal | NHCO— | |
| | PEG₁₁₀₀)₈ | |
| | (α-NH | |
| | DOTA)_{4.75} | |
| | (α- | |
| | NHCy5)_{0.5} | |
| | (α-NH₂)_{2.75}] | |
| | [Lys]₈[Lys]₄ | |
| | [Lys]₂ | |
| | [Lys]— | |
| | CONH— | |
| | CH₂— | |
| | CH₂—S—]₂ | |
| | MAL- | |
| | PEG₃— | |
| | NHCO— | |
| | PEG₂₄— | |
| | CONH— | |
| | PEG₄- | |
| | (PhTzMe) | |
| HH-1 | [(ε- NHCO— PEG₁₁₀₀)₁₆ (α-NH₂• HCl)₁₆] [Lys]₁₆ [Lys]₈[Lys]₄ [Lys]₂[Lys]- | [symbol] |
| | (PN)NCO | C₁₀₅₈H₂₀₇₆N₆₈O₄₆₁ |
| | PEG₂₄ | Molecular Weight: 23127.98 |
| | CONH | C₁₀₅₈H₂₀₉₂C1₁₆N₆₈O₄₆₁ |
| | PEG₄- | Molecular Weight: 23711.36 |
| | (PhTzMe) | |
| HH-2 | [(ε- | Molecular Weight: 27420 |
| | NHCO— | |
| | PEG₁₁₀₀)₁₆ | |
| | (α-DOTA)₇ | |
| | (α-NHCy5)₁ | |
| | (α-NH₂)₈] | |
| | [Lys]₁₆ | |
| | [Lys]₈[Lys]₄ | |
| | [Lys]₂[Lys]- | |
| | (PN)NCO— | |
| | PEG₂₄— | |
| | CONH— | |
| | PEG₄- | |
| | (PhTzMe) | |
| SRS- | Nanobody- | Her-2 targeting nanobody (Cys conjugation site) |
| 13 | Cys | MEVQLVESGGSLVQPGGSLRLSCAASGFTFDDYAMSWVRQVPGKGLEWVSSINWS |
| | | GTHTDYADSVKGRFTISRNNANNTLYLQMNSLKSEDTAVYYCAKNWRDAGTTWF |
| | | EKSGSAGQGTQVTVSSLGTLCTPSRENLYFQGHHHHHH |
| | | MW = 16209.87 (calc) |
| SRS- | Nanobody- | Nanobody-Linker Intermediate |
| 14 | Cys/ | MW = 17,803 (calc) |
| | Me(MAL)- | |
| | PEG₂₄— | |
| | CONH— | |
| | PEG₃-TCO | |
| SRS- | [[(ε- | |
| 15 | NHCO— | |
| | PEG₁₁₀₀)₈ | |
| | (α-NH | |
| | DOTA)_{4.75} | |
| | (α- | |
| | NHCy5)_{0.5} | |
| | (α-NH₂)_{2.75}] | |
| | [Lys]₈[Lys]₄ | |
| | [Lys]₂ | |
| | [Lys] | |
| | CONH— | |
| | CH₂— | |
| | CH₂—S—]₂ | |
| | MAL- | |
| | PEG₃— | |
| | NHCO— | |
| | PEG₂₄— | |
| | CONH— | |
| | PEG₄- | |
| | (PhTzMe)/ | |
| | TCO— | |
| | NHCO— | |
| | PEG₂₄- | |
| | (MAL)Me/ | |
| | Cys- | |
| | Nanobody | |
| SRS- | [(ε- | |
| 16 | NHCO— | |
| | PEG₁₁₀₀)₁₆ | |
| | (α-NHCy5)₁ | |
| | (α- | |
| | NHDOTA)₈ | |
| | (α-NH₂)₇] | |
| | [Lys]₁₆ | |
| | [Lys]₈[Lys]₄ | |
| | [Lys]₂ | |
| | [Lys]— | |
| | CONH— | |
| | CH₂— | |
| | CH₂—S— | |
| | (Me)MAL- | |
| | PEG₂₄— | |
| | CONH— | |
| | Bn-Tz(Me)/ | |
| | TCO— | |
| | NHCO— | |
| | PEG₂₄- | |
| | (MAL)Me/ | |
| | Cys- | |
| | Nanobody | |
| SRS- | [(ε- | |
| 17 | NHCO— | |
| | PEG₁₁₀₀)₁₆ | |
| | (α-DOTA)₇ | |
| | (α-NHCy5)₁ | |
| | (α-NH₂)₈] | |
| | [Lys]₁₆ | |
| | [Lys]₈[Lys]₄ | |
| | [Lys]₂[Lys]- | |
| | (PN)NCO— | |
| | PEG₂₄— | |
| | CONH— | |
| | PEG₄- | |
| | (PhTzMe)/ | |
| | TCO— | |
| | NHCO— | |
| | PEG₂₄- | |
| | (MAL)Me/ | |
| | Cys- | |
| | Nanobody | |
| SRS- 18 | MAL-Cy5 | 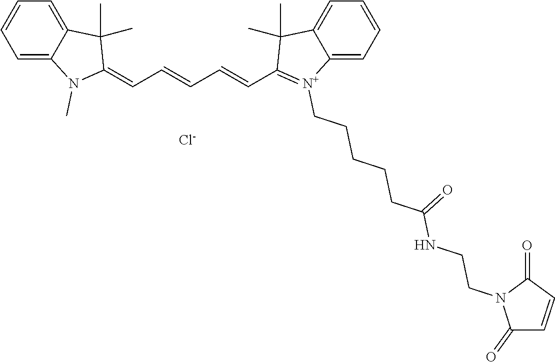 [SMILES: CN1/C(=C/C=C/C=C/C2=[N+](CCCCCC(=O)NCCN3C(=O)C=CC3=O)c3ccccc3C2(C)C)C(C)(C)c2ccccc21 ; [Cl-]] |
| | | Chemical Formula: C₃₈H₄₅ClN₄O₃ |
| | | Molecular Weight: 641.24 |
| SRS- | Nb-Cys/ | Nanobody labelled with Cy5 |
| 19 | MAL-Cy5 | |
| SRS- | [[(ε- | |
| 20 | NHCO— | |
| | PEG₁₁₀₀)₈ | |
| | (α-NH | |
| | DOTA)_{4.75} | |
| | (α- | |
| | NHCy5)_{0.5} | |
| | (α-NH₂)_{2.75}] | |
| | [Lys]₈[Lys]₄ | |
| | [Lys]₂ | |
| | [Lys] | |
| | CONH— | |
| | CH₂—- | |
| | CH₂—S—]₂ | |
| | MAL- | |
| | PEG₃— | |
| | NHCO— | |
| | PEG₂₄— | |
| | CONH— | |
| | PEG₄- | |
| | (PhTzMe)/ | |
| | TCO— | |
| | PEG₃— | |
| | NHCO— | |
| | PEG₂₄-Glu- | |
| | DBCO/N₃- | |
| | Nanobody | |
| SRS- | [[(ε- | |
| 20a | NHCO— | |
| | PEG₁₁₀₀)₈ | |
| | (α-NH | |
| | DOTA)_{4.75} | |
| | (α- | |
| | NHCy5)_{0.5} | |
| | (α-NH₂)_{2.75}] | |
| | [Lys]₈[Lys]₄ | |
| | [Lys]₂ | |
| | [Lys] | |
| | CONH— | |
| | CH₂— | |
| | CH₂—S—]₂ | |
| | MAL- | |
| | PEG₃— | |
| | NHCO— | |
| | PEG₂₄— | |
| | CONH— | |
| | PEG₄- | |
| | (PhTzMe)/ | |
| | TCO— | |
| | PEG₃— | |
| | NHCO— | |
| | PEG₂₄-Glu- | |
| | DBCO | |
| SRS- | BHALys | |
| 21 | [Lys]₂[Lys]₄ | |
| | [Lys]₈ | |
| | [Lys]₁₆[((α- | |
| | NH— | |
| | COPEG₂₄ | |
| | NH— | |
| | COPEG₄ | |
| | (PhTzMe)/ | |
| | TCO- | |
| | PEG₃— | |
| | NHCO— | |
| | PEG₂₄— | |
| | (MAL)Me/ | |
| | Cys- | |
| | Nanobody)₁ | |
| | (α-NHCy5)₁ | |
| | (α-NH | |
| | DOTA))₁₀ | |
| | (α-NH₂)₄) | |
| | (ε-NH—CO | |
| | PEG₁₀₀₀)₁₆ | |
| SRS | BHALys | |
| 21a | [Lys]₂[Lys]₄ | |
| | [Lys]₈ | |
| | [Lys]₁₆[((α- | |
| | NH—CO | |
| | PEG₂₄NH— | |
| | COPEG₄ | |
| | (PhTzMe)/ | |
| | TCO- | |
| | PEG₃— | |
| | NHCO— | |
| | PEG₂₄- | |
| | (MAL)Me)₁ | |
| | (α-NHCy5)₁ | |
| | (α- | |
| | NHDOTA)₁₀ | |
| | (α-NH₂)₄) | |
| | (ε-NH—CO | |
| | PEG₁₀₀₀)₁₆, | |
| SRS- | [(ε- | |
| 22 | NHCO | |
| | PEG₁₁₀₀)₁₆ | |
| | (α- | |
| | DOTA)₇(α- | |
| | NHCy5)₁(α- | |
| | NH₂)₈] | |
| | [Lys]₁₆ | |
| | [Lys]₈[Lys]₄ | |
| | [Lys]₂[Lys]- | |
| | (PN)NCO— | |
| | PEG₂₄— | |
| | CONH— | |
| | PEG₄- | |
| | (PhTzMe)/ | |
| | TCO- | |
| | PEG₃— | |
| | NHCO— | |
| | PEG₂₄-Glu- | |
| | DBCO/ | |
| | N₃— | |
| | Nanobody | |
| SRS- | [(ε- | |
| 22a | NHCO— | |
| | PEG₁₁₀₀)₁₆ | |
| | (α-DOTA)₇ | |
| | (α-NHCy5)₁ | |
| | (α-NH₂)₈] | |
| | [Lys]₁₆ | |
| | [Lys]₈[Lys]₄ | |
| | [Lys]₂[Lys]- | |
| | (PN)NCO— | |
| | PEG₂₄— | |
| | CONH— | |
| | PEG₄- | |
| | (PhTzMe)/ | |
| | TCO— | |
| | PEG₃- | |
| | NHCO— | |
| | PEG₂₄-Glu- | |
| | DBCO | |

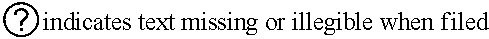

### Example 1: Synthesis of Intermediates and Controls

1a. Synthesis of Bifunctional Linkers and Lysine Wedges
1a.1 BCN-PEG₂-Glu-CO-NHPEG₂₄CO₂H, Compound 49

To a solution of NH₂—PEG₂₄COOH (93.7 mg, 0.082 mmol) in a mixture of water/THF (1:1, 4 mL) was added sodium bicarbonate (15.2 mg, 0.181 mmol). The mixture was stirred for 5 min. at room temperature before a solution of BCN-PEG₂-Glu-NHS ester (50 mg, 0.093 mmol) in THE (2 mL) was added. The resulting reaction mixture was stirred for 15 h at RT. The volatiles were then removed under reduced pressure and MeCN (2.5 mL) was added to the resulting aqueous suspension. This solution was purified by Preparative HPLC (Prep-HPLC Method 5-60% TFA) Rₜ=32.2-34.3 min to give Compound 49 as a white solid (42 mg, 33%) after lyophilisation ¹H NMR (300 MHz, D₂O) δ (ppm): 0.85-0.92 (m, 2H); 1.25-1.35 (m, 1H); 1.43-1.57 (m, 2H); 1.73-1.83 (m, 2H); 2.09-2.25 (m, 9H); 2.39 (t, J=9.0 Hz, 2H); 3.21-3.31 (m, 6H); 3.35-3.85 (m, 106H); 4.10 (d, J=9.0 Hz, 2H). LCMS (philic method, formic buffer) Rₜ=10.28 min.; ESI MS (+ve) m/z 1566.7 [M]⁺.

1a.2 BCN-PEG₂-Glu-CO-NHPEG₂₄CO-NHPEG₃-TCO, Compound 50

To a stirred solution of BCN-PEG₂-Glu-CO-NHPEG₂₄-COOH, Compound 49 (10.0 mg, 0.006 mmol) in DMF (3.0 mL) were added PyBOP (3.64 mg, 0.007 mmol), NMM (1.31 μL, 0.012 mmol) followed by TCO-PEG₃-NH₂ (Click Chemistry Tools, 2.41 mg, 0.007 mmol). The ensuing reaction mixture was stirred at room temperature overnight. The solvent was then removed under reduced pressure and the resulting residue dissolved in MeCN (2 mL) then filtered through 0.45 μm filter. The collected filtrate was purified by preparative HPLC (Prep-HPLC Method 30-50% TFA) Rₜ=40-42 min and the product-containing fractions concentrated under reduced pressure to remove MeCN. The remaining aqueous solution was lyophilized overnight to give Compound 50 as a white solid (4.7 mg, 39%). ¹H NMR (300 MHz, CD₃OD) δ (ppm): 0.89-1.05 (m, 2H), 1.26-1.48 (m, 2H), 1.52-1.79 (m, 5H), 1.83-2.06 (m, 6H), 2.11-2.39 (m, 12H), 2.48 (t, 2H, J=6.0 Hz), 3.35-3.44 (m, 6H), 3.47-3.79 (m, 102H), 3.83-3.92 (m, 1H), 4.16 (d, 2H, J=6.0 Hz), 4.26-4.41 (m, 1H), 4.58 (bs, 10H), 5.39-5.74 (m, 3H). LCMS (philic method, formic buffer) Rₜ=11.0 min. ESI MS (+ve) 1894.0 [M]⁺; calc. m/z for C₉₀H₁₆₅N₅O₃₆ [M]⁺: 1894.2

1a.3 DBCO-Glu-NHPEG₂₄CO-NHPEG₃-TCO, Compound 51

To a stirred solution of DBCO-Glu-NHPEG₂₄COOTFP (50.0 mg, 0.031 mmol) in DMF (3 mL) were added TCO-PEG₃-NH₂ (Click Chemistry Tools, 10.7 mg, 0.031 mmol) followed by NMM (4.08 μL, 0.037 mmol). The ensuing reaction mixture was stirred at room temperature for 3 h, then concentrated under reduced pressure. The resulting residue was dissolved in MeCN (2 mL), filtered through 0.45 μm filter and the filtrate purified by preparative HPLC (Prep-HPLC Method 40-70% TFA) Rt=27-29 min. The product-containing fractions were concentrated under reduced pressure to remove MeCN and the remaining aqueous solution lyophilized overnight to give Compound 51 as a viscous colourless liquid (25.0 mg, 42%). ¹H NMR (300 MHz, CD₃OD) δ (ppm): 1.31-1.62 (m, 4H), 1.60-1.83 (m, 6H), 1.92-2.16 (m, 4H), 2.25 (t, 2H, J=6.0 Hz), 2.91-3.03 (m, 4H), 3.12-3.58 (m, 78H), 3.59-3.69 (m, 1H), 4.01-4.18 (m, 1H), 4.92 (d, 2H, J=15H), 5.15-5.49 (m, 3H), 6.95-7.59 (m, 8H). LCMS (philic method, formic buffer) Rₜ=7.0 min. ESI MS (+ve) 1775.0.0 [M]⁺; calc. m/z for C₈₈H₁₄₈N₄O₃₂ [M]⁺: 1775.12

1a.4 N₃-PEG₇-NHCO-Lys[(α-NHBoc)(ε-NHFmoc)], Compound SRS-3

To a stirred solution of N₃-PEG₇-NH₂ (QuantaBiodesign, 514 mg, 1.30 mmol), HO-Lys[(α-NHBoc)(ε-NHFmoc)] (488 mg, 1.04 mmol) and NMM (286 μL, 2.60 mmol) in DMF (5 mL) was added PyBOP (811 mg, 1.56 mmol) and the reaction mixture stirred at rt for 18 h. The volatiles were removed under reduced pressure and the residue was purified by column chromatography on silica gel eluting with dichloromethane:MeOH (gradient elution from 100:0 to 90:10 v/v) to give SRS-3 as a viscous colourless oil (410 mg, 47%). LCMS (LCMS Method 20-90, 15 min): Rₜ=9.35 min, ESI MS (+ve) 845 [M+H]⁺; calc. m/z for C₄₂H₆₄N₆O₁₂ [M+H]⁺=845; ¹H NMR (300 MHz, MeOD) δ (ppm): 1.17-1.94 (m, 16H), 3.12 (t, 2H, J=6.0 Hz), 3.33-3.44 (m, 4H), 3.45-3.74 (m, 28H), 3.89-4.65 (m, 1H), 4.12 (t, 1H, J=9.0 Hz), 4.38 (d, 2H, J=6.0 Hz), 7.33 (t, 2H, J=9.0 Hz), 7.41 (t, 2H, J=9.0 Hz), 7.66 (d, 2H, J=6.0 Hz), 7.81 (d, 2H, J=9.0 Hz).

1a.5 N₃-PEG₇-NHCO-Lys[(α-NH₂·HCl)(ε-NHFmoc)] Compound SRS-3a

N₃-PEG₇-NHCO-Lys[(α-NHBoc)(ε-NHFmoc)] (200 mg, 0.236 mmol) Compound SRS-3 was dissolved in 1.5 M HCl in methanol (4 mL) and the reaction mixture stirred at rt for 3 h. The volatiles were removed under reduced pressure to give N₃-PEG₇-NHCO-Lys[(α-NH₂·HCl)(ε-NHFmoc)] SRS-3a as a white solid (180 mg, 98%). LCMS (LCMS Method 20-90, 15 min): Rₜ=6.13 min, ESI MS (+ve) 745 [M+H]⁺; calc. m/z for C₃₇H₅₆N₆O₁₀ [M+H]⁺=745.

1a.6 N₃-PEG₇-NHCO-Lys[(α-NHCy5)(ε-NHFmoc)] Compound SRS-4

To a stirred solution of N₃-PEG₇-NHCO-Lys[(α-NH₂·HCl)(ε-NHFmoc)] SRS-3a (58 mg, 0.074 mmol) and NMM (16 μL, 0.148 mmol) in DMF (5 mL) was added Cy5 NHS ester (50 mg, 0.074 mmol). The reaction mixture stirred at rt for 20 h whereupon the volatiles were removed under reduced pressure. The residue was dissolved in MQ water:MeCN (2 mL, 1:1 v/v), filtered (0.45 μm acrodisc filter) and purified using preparative HPLC to give compound SRS-4 as a blue solid (30 mg, 33%). HPLC: C18 column with a gradient 30% MeCN/H₂O (1-10 min), 30-90% MeCN/H₂O (10-35 min), 90% MeCN/H₂O (35-48 min), 90-30% MeCN/H₂O (48-55 min), 0.05% Formic acid buffer) and UV detection at 254 nm. Rₜ=35-40 min. LCMS (LCMS Method 20-90, 15 min) Rₜ=8.20 min, ESI MS (+ve) 1210 [M+H]⁺; calc. m/z for C₆₉H₉₃N₈O₁₁[M+H]⁺=1210.

1a.7 N₃-PEG₇-NHCO-Lys[(α-NHCy5)(ε-NH₂)] Compound SRS-4a

N₃-PEG₇-NHCO-Lys[(α-NHCy5)(ε-NHFmoc)] Compound SRS-4 (30 mg, 0.024 mmol) was dissolved in a DMF:Piperdine (3.0 mL, 4:1 v/v) and the reaction mixture stirred at rt. After 18 h, the volatiles were removed under reduced pressure and the residue purified by preparative HPLC to give N₃-PEG₇-NHCO-Lys[(α-NHCy5)(ε-NH₂)] SRS-4a as a blue solid (18.0 mg, 75%). HPLC: C18 column with a gradient 20% MeCN/H₂O (1-10 min). 20-60% MeCN/H₂O (10-35 min), 60% MeCN/H₂O (35-48 min), 60-20% MeCN/H₂O (48-55 min), 0.05% Formic acid buffer) and UV detection at 214 and 254 nm. Rₜ=24-28 min. LCMS (LCMS Method 20-90, 15 min) Rₜ=4.91 min, ESI MS (+ve) 988 [M+H]⁺; calc. m/z for C₅₄H₈₃N₈O₉ [M+H]⁺=988.

1a.8 N₃-PEG₇-NHCO-Lys[(α-NHCy5)(ε-NHDOTA)] Compound SRS-5

To a stirred solution of amine SRS-4a (18.0 mg, 0.018 mmol) in DMF (4 mL) were added NMM (4 μL, 0.036 mmol) and a solution of p-SCN-Bn-DOTA (13 mg, 0.018 mmol) in DMSO (500 μL). After stirring for 18 h, the volatiles were removed under reduced pressure and the residue was purified by preparative HPLC to give SRS-5 as a blue solid (9.0 mg, 32%). HPLC: C18 column with a gradient 40% MeCN/H₂O (1-10 min), 40-80% MeCN/H₂O (10-35 min), 80% MeCN/H₂O (35-48 min), 80-40% MeCN/H₂O (48-55 min), 0.05% Formic acid buffer) and UV detection at 214 and 254 nm. Rₜ=26-28 min. LCMS (LCMS Method 20-90, 15 min) Rₜ=3.95 min, ESI MS (+ve) 1538 [M+H]⁺; calc. m/z for C₇₈H₁₁₆N₁₃O₁₇S [M+H]⁺=1538.

### 1a.9 DFO-PEG₄-Sulfo-DBCO, Compound 55

To a suspension of Deferoxamine-SCN (22.3 mg, 29.7 mmol) and sulfo-PEG₃-DBCO (20.0 mg, 29.7 mmol) in DMF (2 mL) was added NMM (6.5 μL, 59.1 μmol). Sonication did not provide a homogeneous solution. Additional NMM (10 μL, 91.0 μmol) and DIPEA (10 μL, 57.4 μmol) also did not further solubilize the reaction mixture. DMSO (2 mL) was added and the contents sonicated for 1 min which ultimately provided a transparent solution. The ensuing reaction mixture was stirred at RT overnight. The contents were concentrated under reduced pressure and MeCN (12 mL) added to the residual solution then the contents filtered (0.45 μm acrodisc). The filtrate was purified by preparative HPLC; 1000 μL injection volume, 5-80% MeCN over 60 min, 0.1% formic acid, Rₜ=33.5-34.5 min. The product fractions were combined, the MeCN removed under reduced pressure and the remaining aqueous solution lyophilized to give the title product as a white solid, 12.7 mg (30%). LCMS (philic method, formic buffer) Rₜ=4.70 min; ESI MS (+ve) 1428 [M]⁺; calc. m/z for C₆₅H₉₄N₁₂O₁₈S₃ [M]⁺=1427.7. ¹H NMR (300 MHz, D₂O) δ (ppm): 7.71-6.98 (m, 12H), 5.11-4.95 (m, 1H), 3.90-3.01 (m, 36H), 2.87-2.30 (m, 11H), 2.21-1.90 (m, 4H), 1.80-1.16 (m, 17H).

1a.10 Synthesis of DFO-DBCO compound 56

To a stirred solution of deferoxamine mesylate (Macrocyclics, 200.0 mg, 0.304 mmol) in DMSO (1 mL) was added NMM (100.0 μL, 0.912 mmol) and DBCO-NHS ester (130.7 mg, 304 mmol). The reaction mixture stirred at room temperature overnight and then concentrated by blowing a stream of nitrogen gas over the solution for 2 h. The residue was dissolved in a mixture of MQ water: Acetonitrile (3 mL, 1:1 v/v) and then filtered (0.45 μm acrodisc syringe filter). The filtrate was purified by preparative HPLC: 30-40% MeCN in MQ water+0.1% formic acid (60 min, Rₜ 42-44 min) to give the product as a colourless, viscous liquid (195.0 mg, 73%). LCMS (philic method, TFA buffer) Rₜ=5.64 min; ESI MS (+ve) 876 [M]⁺; calc. m/z for C₄₆H₆₅N₇O₁₀ [M]⁺=876, [M+Fe]⁺=929; ¹H NMR (300 MHz, DMSO-d6) δ (ppm): 9.96-9.46 (bs, 2H), 7.92-7.24 (m, 9H), 5.09 (d, 1H, J=15.0 Hz), 3.66 (d, 1H, J=12.0 Hz), 3.56-3.43 (m, 10H), 3.13-2.87 (m, 4H), 2.69-2.58 (m, 4H), 2.40-2.27 (m, 3H), 2.13 (s, 6H), 2.02 (s, 2H), 1.94-1.75 (m, 2H), 1.68-1.09 (m, 17H).

### 1a.11 Synthesis of TCO-PEGs-Dibromomaleimide Compound 57

To a stirred solution of ethyl 3,4-dibromo-2,5-dioxo-2H-pyrrole-1(5H)-carboxylate (25.0 mg, 0.077 mmol) in THE (2.0 mL) was added TCO-PEG₈-NH₂ (Broadpharm; 44.0 mg, 0.077 mmol) and the reaction mixture stirred at room temperature for overnight. The solvent was removed under reduced pressure and the residue was purified by column chromatography on silica gel eluting with dichloromethane:MeOH (gradient elution from 100:0 to 97:3 v/v) followed by preparative HPLC eluting with 50 to 90% MeCN in MQ water over 60 min to give Compound 57 as a colourless oil (8.0 mg, 13%). ¹H NMR (300 MHz, MeOD) 5.79-5.42 (m, 2H), 4.77-4.62 (m, 1H), 3.80 (t, 2H, J=6.0 Hz), 3.70-3.57 (m, 29H), 3.53 (t, 2H, J=6.0 Hz), 3.27 (t, 2H, J=6.0 Hz), 2.48-2.30 (m, 1H), 2.26-1.46 (m, 9H).

1a.12 Synthesis of HO-Lys[(α-NHCy5)(ε-NHDFO)] wedge, Compound 58

To a stirred solution of HO-Lys[(α-NH₂.TFA)(ε-NHFmoc)] (57.0 mg, 0.118 mmol) in DMF (5 mL) was added NMM (52 μL, 0.472 mmol) and Cy5-NHS ester (Lumiprobes, 40.0 mg, 0.059 mmol). The reaction mixture was stirred at room temperature overnight whereupon the volatiles were removed in vacuo. The residue was dissolved in MeCN:MQ water (3 mL 1:1 v/v) and the solution filtered (0.45 μm acrodisc syringe filter). The filtrate was purified by preparative HPLC; 20-90% MeCN in MQ water+0.1% formic acid (60 min, Rₜ=39.0-42.0 min) to give Compound 58 as a blue solid 33 mg (67%). LCMS (philic method, TFA buffer) Rₜ=10.54 min; ESI MS (+ve) 833 [M]⁺; calc. m/z for C₅₃H₆₁N₄O₅ [M]⁺=833.46 ¹H NMR (300 MHz, D₂O) δ (ppm): 8.21 (t, 2H, J=15.0 Hz), 7.79 (d, 2H, J=6.0 Hz), 7.63 (d, 2H, J=6.0 Hz), 7.49-7.24 (m, 10H), 6.61 (t, 1H, J=12.0 Hz), 6.29-6.22 (m, 2H), 4.49-4.26 (m, 2H), 0.18 (t, 1H, J=6.0 Hz), 4.07 (t, 2H, J=6.0 Hz), 3.68-3.60 (m, 2H), 3.60 (s, 3H), 3.12 (t, 2H, J=6.0 Hz), 2.28 (t, 2H, J=6.0 Hz), 1.94-1.61 (m, 6H), 1.71 (s, 12H), 1.62-1.19 (m, 6H).

### 1a.13 HO-Lys[(α-NHCy5) (ε-NH₂)], Compound 59

To a stirred solution of Compound 58 (36 mg; 0.043 mmol) in DMF (4 mL) was added piperidine (1.5 mL) and the reaction mixture was stirred at room temperature for 1 h. The solvent was removed under reduced pressure and the residue was purified by preparative HPLC; 20-90% MeCN in MQ water+0.1% formic acid (60 min, Rₜ=32.0-33.0 min) to give the product Compound 59 as a blue solid 12 mg (44%). LCMS (philic method, TFA buffer) Rₜ=7.65 min; ESI MS (+ve) 611 [M]⁺; calc. m/z for C₃₈H₅₁N₄O₃ [M]⁺=611.

### 1a.14 HO-Lys[(α-NHCy5)(ε-NHDFO)], Compound 60

To a stirred solution of Compound 59 (20.0 mg, 0.032 mmol) in DMSO (5 mL) was added DIPEA (32 μL, 0.224 mmol) followed by p-SCN-Deferoxamine (Macrocyclics, 24.0 mg, 0.032 mmol). The reaction mixture stirred at room temperature for 4 h and then concentrated by blowing a stream of nitrogen gas over the solution for several hours. The residue obtained was then purified by preparative HPLC: 30-60% MeCN in MQ water+0.1% TFA (60 min, Rₜ 39-42 min) to give the product Compound 60 as a blue solid 15 mg (34%). LCMS (philic method, TFA buffer) Rₜ=5.93 min; ESI MS (+ve) 1364 [M]⁺; calc. m/z for C₇₁H₁₀₃N₁₂O₁₁S₂ [M]⁺=1364 ¹H NMR (300 MHz, D₂O) δ (ppm): 8.25 (t, 2H, J=12.0 Hz), 7.56-7.18 (m, 9H), 6.65 (t, 1H, J=12.0 Hz), 6.37-6.15 (m, 2H), 4.43-4.32 (m, 1H), 4.11 (t, 2H, J=6.0 Hz), 3.70-3.46 (m, 8H), 3.22-3.10 (m, 3H), 2.86-2.69 (m, 3H), 2.56-2.38 (m, 3H), 2.31 (t, 2H, J=6.0 Hz), 2.11 (s, 2H), 1.96-1.18 (m, 32H).

1a.15 Synthesis of DUPA-BCN Linker (17S,21S)-1-((1R,8S,9S)-Bicyclo[6.1.0]non-4-yn-9-yl)-3,14,19-trioxo-2,7,10-trioxa-4,13,18,20-tetraazatricosane-17,21,23-tricarboxylic acid, Compound 61

To a stirred solution of 3.3 (13S,17S)-1-Amino-10,15-dioxo-3,6-dioxa-9,14,16-triazanonadecane-13,17,19-tricarboxylic acid Compound 48 (149 mg, 0.26 mmol) and triethylamine (184 μL, 1.32 mmol) in a mixture of tetrahydrofuran and water (1:1, 4 mL) was added portion wise ((1R,8S,9s)-bicyclo[6.1.0]non-4-yn-9-yl)methyl (2,5-dioxopyrrolidin-1-yl) carbonate dichloromethane (BCN-NHS ester) (84 mg, 0.29 mmol). The reaction mixture was stirred at ambient temperature for 2.5 and the volatiles were removed in vacuo. The residue was purified by preparative HPLC (Gilson HPLC system; Column: X-Bridge BEH300 Prep C18 5 um OBD 30×150 mm; solvents: A=deionised water with 0.05% formic acid; B=MeCN with 0.05% formic acid; flow rate: 8 mL/min) to give the title product, Compound 61 (120 mg, 73%) as white solid after lyophilisation. ¹H NMR (300 MHz, D₂O) δ (ppm): 0.77-0.90 (m, 2H); 1.13-1.36 (m, 1H); 1.45-1.49 (m, 2H); 1.79-1.93 (m, 2H); 2.01-2.19 (m, 9H); 2.24-2.31 (m, 2H); 2.39-2.44 (m, 2H); 3.20-3.30 (m, 4H); 3.48-3.52 (m, 4H); 3.56-3.62 (m, 4H); 4.06-4.19 (m, 4H). LCMS (philic, formic buffer) Rₜ=8.92 min. ESI MS (+ve) 627 [M+1]⁺; calc. m/z for C₂₈H₄₂N₄O₁₂ [M]⁺: 626.28.

1a.16 BocHN-PEG₃-NHCO-PEG₂₄-CONH-PEG₄-(PhTzMe) SRS-7

To a stirred solution of HO₂C-PEG₂₄-CONH-PEG₄-(PhTzMe) SRS-5a (500 mg, 0.32 mmol), BocNH-PEG₃-NH₂ SRS-6 (1-2.5 mg, 0.32 mmol) and NMM (53 μL, 0.48 mmol) in DMF (12.0 mL) at rt was added PyBOP (166 mg, 0.32 mmol). The reaction mixture was stirred for 16 h and the volatiles were removed in vacuo. The residue was dissolved in MeCN (3.0 mL), filtered (0.45 μm filter disc) and purified by preparative HPLC (Prep-HPLC Method 20-60, Rₜ=34-47 min) to give BocHN-PEG₃-NHCO-PEG₂₄-CONH-PEG₄-(PhTzMe) SRS-7 as a red solid (550 mg, 92%). ¹H NMR (300 MHz, CD₃OD): δ (ppm) 1.78 (q, J=6.7 Hz, 2H), 1.95 (q, J=6.2 Hz, 2H), 2.46 (t, J=6.2 Hz, 4H), 3.02 (s, 3H), 3.13 (t, J=6.8 Hz, 2H), 3.26-3.43 (m, 4H), 3.50-3.58 (m, 4H), 3.58-3.78 (m, 128H), 3.89-3.95 (m, 2H), 4.25-4.32 (m, 2H), 7.20 (d, J=9.0 Hz, 2H) and 8.52 (d, J=9.0 Hz, 2H). LCMS (LCMS Method 20-60, 8 min, TFA) Rₜ=6.12 min. ESI MS (+ve) 1885 [M+H₂O]⁺; calc. m/z for C₈₆H₁₅₉N₇O₃₆ [M+H₂O]⁺=1885.

1a.17 HCl·H₂N-PEG₃-NHCO-PEG₂₄-CONH-PEG₄-(PhTzMe) SRS-8

To BocHN-PEG₃-NHCO-PEG₂₄-CONH-PEG₄-(PhTzMe) SRS-7 (450 mg mg, 0.241 mmol) was added 2.0 M HCl in methanol (4.0 mL) at rt. The resulting solution was stirred for 18 h and the volatiles were removed in vacuo. The residue was dissolved in water (4.0 mL) and lyophilised to give HCl·H₂N-PEG₃-NHCO-PEG₂₄-CONH-PEG₄-(PhTzMe) SRS-8 as a pink solid 420 mg (97%). LCMS (LCMS Method 5-60, 8 min, TFA): Rₜ=5.72 min. ESI MS (+ve) 1769 [MH]⁺; calc. m/z for C₈₁H₁₅₁N₇O₃₄ [MH]⁺=1769.

1a.18 Me(MAL)-PEG₂₄-CO₂HSRS-10

To a stirred solution of amino-PEG₂₄-acid RL-11 (510 mg, 0.44 mmol) in in glacial acetic acid (15 mL) was added citraconic anhydride (50 μL, 0.53 mmol). The reaction mixture was then heated with stirring at 120° C. for 2 h and then cooled to rt whereupon an additional aliquot of citraconic anhydride (50 μL, 0.53 mmol) was added. The reaction mixture was heated at 120° C. for 2 h and then cooled to rt. After 16 h, the reaction mixture was concentrated in vacuo and the residual brown oil was purified by preparative HPLC (Prep-HPLC Method 5-60, Rₜ=35-37.5 min) to give Me(MAL)-PEG₂₄-CO₂H SRS-10 as a pale brown solid (236 mg, 43%). ¹H NMR (300 MHz, CD₃OD): δ (ppm) 2.07 (d, J=1.8 Hz, 3H), 2.56 (t, J=6.3 Hz, 2H), 3.48-3.72 (m, 98H), 3.75 (t, J=6.3 Hz, 2H) and 6.46 (q, J=1.8 Hz, 1H). LCMS (LCMS Method 60-90, 8 min, TFA): Rₜ=5.54 min. ESI MS (+ve) 1258 [M+H₂O]⁺; calc. m/z for C₅₆H₁₀₇NO₂₉ [M+H₂O]=1258.

1a.19 Me(MAL)-PEG₂₄-CONH-PEG₃-TCO SRS-12

To a stirred solution of Me(MAL)-PEG₂₄-CO₂H SRS-10 (130 mg, 0.105 mmol) in DMF (2.0 mL) at rt was added NH₂—PEG₃-TCO SRS-11 (47 mg, 0.126 mmol), PyBOP (65 mg, 0.126 mmol) and NMM (23 μL, 0.126 mmol). After stirring for 3.5 h, the volatiles were removed in vacuo and the residue was dissolved in MeCN:Water (1:2 v/v, 3 mL) and purified by preparative HPLC (Prep-HPLC Method 5-60(2), Rₜ=41-43 min) to give Me(MAL)-PEG₂₄-CONH-PEG₃-TCO SRS-12 as a colourless oil (77 mg, 46%). ¹H NMR (300 MHz, CD₃OD): δ (ppm) 1.53-2.40 (m, 15H), 2.07 (d, J=1.85 Hz, 3H), 2.45 (t, J=6.2 Hz, 2H), 3.13-3.21 (m, 2H), 3.28 (t, J=6.8 Hz, 2H), 3.47-3.81 (m, 118H), 5.42-5.84 (m, 2H) and 6.46 (m, 1H). LCMS (LCMS Method 5-60, 8 min, TFA) Rₜ=6.51 min. ESI MS (+ve) 1612 [M+H₂O]⁺; calc. m/z for C₇₅H₁₄₁N₃O₃₃ [M+H₂O]⁺=1612.

1a.20 Br₂(MAL)-PEG₃-NHCO-PEG₂₄-CONH-PEG₄-(PhTzMe) RL-15

To a solution of HCl·H₂N-PEG₃-NHCO-PEG₂₄-CONH-PEG₄-(PhTzMe) SRS-8 (100 mg, 0.057 mmol) in THE (2.0 mL) at rt was added TEA (15 μL, 0.068 mmol) and 1H-pyrrole-1-carboxylic acid, 3,4-dibromo-2,5-dihydro-2,5-dioxo-, ethyl ester RL-12 (22 mg, 0.068 mmol). The reaction mixture was stirred for 18 h and then concentrated in vacuo. The residue was dissolved in MeCN:Water (2.0 ml, 1:1 v/v) and purified by preparative HPLC (Prep-HPLC Method 40-60, Rₜ=25-27 min) to give Br₂(MAL)-PEG₃-NHCO-PEG₂₄-CONH-PEG₄-(PhTzMe) RL-15 (42 mg, 37%).

Alternatively, to a solution of Br₂(MAL)-PEG₃-NH₂.TFA RL-17 (202 mg, 0.35 mmol) in DMF (3.0 mL) at rt was added HO₂C-PEG₂₄-CONH-PEG₄-(PhTzMe) SRS-5a (461 mg, 0.30 mmol), PyBOP (184 mg, 0.35 mmol) and NMM (85 μL, 0.78 mmol). The reaction mixture was stirred for 18 h and then concentrated in vacuo. The residue was dissolved in MeCN:Water (1:1 v/v, 4.0 mL) and purified by preparative HPLC [Prep-HPLC Method 20-60(2)] to give Br₂(MAL)-PEG₃-NHCO-PEG₂₄-CONH-PEG₄-(PhTzMe) RL-15 as a pink solid (37 mg, 6%). ¹H NMR (300 MHz, CD₃OD): δ (ppm) 1.70-1.82 (m, 2H), 1.82-1.93 (m, 2H), 2.45 (m, 4H), 3.02 (s, 3H), 3.20-3.43 (m, 4H), 3.47-3.89 (m, 132H), 3.91 (m, 2H), 4.28 (m, 2H), 7.20 (d, J=9.0 Hz, 2H) and 8.50 (d, J=9.0 Hz, 2H). LCMS (LCMS Method 40-60, 8 min, TFA) Rₜ=5.12 min. ESI MS (+ve) 2024 [M+H₃O]⁺; calc. m/z for C₈₅H₁₄₉Br₂N₇O₃₆ [M+H₃O]⁺=2024.

1a.21 Br₂(MAL)-PEG₃-NHBoc RL-16

To a solution of 1H-pyrrole-1-carboxylic acid, 3,4-dibromo-2,5-dihydro-2,5-dioxo-, ethyl ester RL-12 (212 mg, 0.66 mmol) in dichloromethane (2.0 mL) at rt was added a solution of BocHN-PEG₃-NH₂ SRS-6. The reaction mixture was stirred for 2 d whereupon the volatiles were removed in vacuo. The residue was purified by column chromatography on silica gel eluting with methanol in dichloromethane [gradient elution; % methanol (v/v)]: 0% to 3.3% to 6.7% to give Br₂(MAL)-PEG₃-NHBoc RL-16 as an off-white solid (220 mg, 59%). ¹H NMR (300 MHz, CD₃CN): δ (ppm) 1.41 (s, 9H), 1.67 (m, 2H), 1.82 (m, 2H), 3.10 (q, J=6.7 Hz, 2H), 3.42-4.58 (m, 12H) and 3.64 (t, J=7.1 Hz, 2H). LCMS (LCMS Method 60-90, 8 min, TFA) Rₜ=3.99 min. ESI MS (+ve) 459 [MH-Boc]⁺; calc. m/z for C₁₄H₂₃Br₂N₂O₅ [MH-Boc]⁺=459.

1a.22 Br₂(MAL)-PEG₃-NH₂.TFA RL-17

To a solution of Br₂(MAL)-PEG₃-NHBoc RL-16 (220 mg, 0.40 mmol) in dichloromethane (6.0 mL) was added TFA (609 μL, 7.79 mmol) at rt. The reaction mixture was stirred for 2 h and concentrated in vacuo. The residue was dissovled in deionised water (˜2 mL) and resulting solution lyophilised to give Br₂(MAL)-PEG₃-NH₂.TFA RL-17 as an off-white solid (202 mg, 88%). ¹H NMR (300 MHz, CD₃OD): δ (ppm) 1.84-1.98 (m, 4H), 3.12 (t, J=6.4 Hz, 2H) and 3.49-3.74 (m, 14H). LCMS (LCMS Method 60-90, 8 min, TFA) Rₜ=3.19 min. ESI MS (+ve) 459 [MH]⁺; calc. m/z for C₁₄H₂₃Br₂N₂O₅ [MH]⁺=459.

### 1a.23 Me(MAL)-PEG₂₄-CONH-Bn-Tz(Me) RL-18

To a solution of Me(MAL)-PEG₂₄-CO₂H SRS-10 (50 mg, 0.04 mmol) and 4-(6-methyl-1,2,4,5-tetrazin-3-yl)benzenemethanamine RL-13 (11 mg, 0.06 mmol) in DMF (2.0 mL) at rt was added PyBOP (24 mg, 0.06 mmol) and NMM (6 μL, 0.06 mmol). The reaction was stirred for 16 h whereupon additional 4-(6-methyl-1,2,4,5-tetrazin-3-yl)benzenemethanamine RL-13 (5 mg, 0.02 mmol) in DMF (2 mL) was added followed by PyBOP (12 mg, 0.02 mmol) and NMM (6 μL, 0.06 mmol). After 2 h, the reaction mixture was concentrated in vacuo and the residue was purified by preparative HPLC (Prep-HPLC Method 20-40, Rₜ=45-48 min) to give Me(MAL)-PEG₂₄-CONH-Bn-Tz(Me) RL-18 as a pink solid (24 mg, 42%). ¹H NMR (300 MHz, CD₃OD): δ (ppm) 2.06 (d, J=1.9 Hz, 3H), 2.56 (t, J=6.0 Hz, 2H), 3.05 (s, 3H), 3.58-3.84 (m, 96H), 4.55 (s, 2H), 6.46 (m, 1H), 7.59 (d, J=8.6 Hz, 2H) and 8.53 (d, J=8.6 Hz, 2H). LCMS (LCMS Method 60-90, 8 min, TFA) Rₜ=6.07 min. ESI MS (+ve) 1424 [M]⁺; calc. m/z for C₆₆H₁₁₄N₆O₂₇ [M]=1424.

1b. Synthesis of Dendrimer Intermediates
1b.1 Azido-PEG₂₄CO—[N(PNBoc)₂], Compound 1

To a stirred solution of azido-PEG₂₄-acid (Quanta Biodesign, 2.00 g, 1.71 mmol) and PyBOP (1.33 g, 2.56 mmol) in DMF (20 mL) under an atmosphere of N₂ was added NMM (563 μL, 5.12 mmol). After 10 min, a solution of N(PNBoc)₂ (622 mg, 1.88 mmol) in DMF (5 mL) was added and the ensuing reaction mixture left to stir overnight at room temperature. The volatiles were removed in vacuo and the resulting oil dissolved in MeCN and purified by preparative HPLC (27-50-70% MeCN, Rₜ 47-50 min) to give a pale yellow oily solid (1.37 g, 54%). ¹H NMR (300 MHz, CD₃OD) δ (ppm): 1.44 (m, 18H); 1.65-1.84 (m, 4H); 2.63 (t, J=6.3 Hz, 2H); 3.05 (dt, J=6.9 and 14.7 Hz, 4H); 3.36-3.41 (m, 6H); 3.60-3.78 (m, 98H). LCMS (philic method, formic buffer) Rₜ=9.32 min. ESI MS (+ve) 1486.3 [M]⁺; calc. m/z for C₆₇H₁₃₂N₆O₂₉ [M]⁺: 1486.8.

1b.2 Azido-PEG₂₄CO—[N(PNH₂.TFA)₂], Compound 2

Prepared according to General Procedure A, using azido-PEG₂₄CO—[N(PNBoc)₂], Compound 1 (1.37 g, 922 μmol). The lyophilised product, Compound 2 was obtained as a pale-yellow oil (1.67 g, 119%). ¹H NMR (300 MHz, D₂O) δ (ppm): 1.88-2.06 (m, 4H); 2.74 (t, J=6.0 Hz, 2H); 2.96 (t, J=7.2 Hz, 2H); 3.04 (apparent t, J=7.5 Hz, 2H); 3.42-3.52 (m, 6H); 3.67-3.95 (m, 93H). LCMS (philic method, TFA buffer) Rₜ=8.47 min, ESI MS (+ve) 1286.0 [M]⁺; calc. m/z for C₅₇H₁₁₆N₆O₂₅ [M]⁺=1286.6.

1b.3 Azido-PEG₂₄CO—[N(PN)₂][Lys]₂[NHBoc]₄, G1, Compound 3

Prepared according to General Procedure B, using azido-PEG₂₄CO—[N(PNH₂.TFA)₂], Compound 2 (186 mg, 145 μmol). The crude material was dissolved in MeCN and purified by preparative HPLC (30-80% MeCN, Rₜ 33.5-36 min) to give Compound 3 as a pale-yellow oil (224 mg, 80%). ¹H NMR (300 MHz, CD₃OD) δ (ppm): 1.22-1.87 (m, 56H); 2.64 (t, J=6.0 Hz, 2H); 3.03 (t, J=6.6 Hz, 4H); 3.13-3.23 (m, 4H), 3.36-3.45 (m, 6H); 3.60-3.69 (m, 100H); 3.77 (t, J=6.0 Hz, 2H); 3.85-3.88 (m, 1H); 3.92-4.02 (m, 2H). LCMS (phobic method, formic buffer) Rₜ=6.74 min; ESI MS (+ve) 1942.4, [M]⁺; calc. m/z for C₈₉H₁₇₂N₁₀O₃₅⁺[M+H]⁺=1942.4.

1b.4 Azido-PEG₂₄CO—[N(PN)₂][Lys]₂[NH₂.TFA]₄, G1, Compound 4

Prepared according to General Procedure A, using azido-PEG₂₄CO—[N(PN)₂[Lys]₂[NHBoc]₄, Compound 3 (220 mg, 113 μmol). The lyophilised product Compound 4 was obtained as a pale-yellow oil (251 mg, 111%). LCMS (philic method, formic buffer) Rₜ=6.49 min, ESI MS (+ve) 1542.1 [M]⁺; calc. m/z for C₆₉H₁₄₀N₁₀O₂₇ [M]⁺=1541.90.

1b.5 Azido-PEG₂₄CO—[N(PN)₂][Lys]₂[Lys]₄[(α-NHBoc)(ε-NH-COPEG₁₁₀₀)]₄, G2, Compound 5

Prepared according to General Procedure C, using azido-PEG₂₄CO—[N(PN)₂[Lys]₂[NH₂.TFA]₄, Compound 4 (120 mg, 60.1 μmol). The crude material was dissolved in MeCN/H₂O (1:1) and purified by preparative HPLC (20-70% MeCN, Rₜ 31-32.5 min) to give the product, Compound 5 as a pale-yellow oil (244 mg, 59%). ¹H NMR (300 MHz, CD₃OD) δ (ppm): 1.24-1.91 (m, 74H); 2.42-2.47 (m, 8H); 2.62-2.66 (m, 2H); 3.13-3.25 (m, 12H); 3.36 (s, 12H); 3.52-3.78 (m, 490H); 3.85-3.88 (m, 4H); 3.94-4.11 (m, 4H); 4.25-4.31 (m, 2H). LCMS (philic method, formic buffer) Rₜ=8.70 min; ESI MS (+ve) 1714.0 [M+4H]⁴⁺/4, 1371.7 [M+5H]⁵⁺/5; 1143.0 [M+6H]⁶⁺/6, 980.0 [M+7H]⁷⁺/7. Transforms to 6852.

1b.6 Azido-PEG₂₄CO—[N(PN)₂] [Lys]₂[Lys]₄[(α-NH₂.TFA)(ε-NH-COPEG¹¹⁰⁰)]₄, G2, Compound 6

Prepared according to General Procedure A, using azido-PEG₂₄CO—[N(PN)₂][Lys]₂ [Lys]₄[(α-NHBoc)(ε-NH-COPEG₁₁₀₀)]₄, Compound 5 (244 mg, 35.6 μmol). The crude lyophilised material was redissolved in water and purified by preparative IPLC (22-70% MeCN, 0.01% TFA, Rₜ 27 min) to give the product, Compound 6 as a pale-yellow sticky solid (173 mg, 67%). ¹H NMR (300 MHz, D₂O) δ (ppm): 1.31-1.98 (m, 40H); 2.51-2.56 (m, 8H); 2.72 (broad t, J=6.0 Hz, 2H); 3.16-3.30 (m, 16H); 3.40 (s, 12H); 3.46-3.53 (m, 4H); 3.62-3.97 (m, 490H); 4.03 (t, J=6.6 Hz, 2H); 4.24-4.29 (m, 2H). LCMS (philic method, TFA buffer) Rₜ=9.85 min; ESI MS (+ve) 1614.1 [M+4H]⁴⁺/4, 1291.6 [M+5H]⁵⁺/5; 1076.5 [M+6H]⁶⁺/6. Transforms to 6452.

1b. 7 Azido-PEG₂₄CO—[N(PN)₂][Lys]₂[Lys]₄[NHBoc]₈, G2, Compound 7

Prepared according to General Procedure B, using azido-PEG₂₄CO—[N(PN)₂][Lys]₂[NH₂.TFA]₄, Compound 6 (117 mg, 58.6 μmol). The crude material, Compound 7 was obtained as a pale-yellow oil (167 mg, 100%). LCMS (phobic method, formic buffer) Rₜ=8.35 min; ESI MS (+ve) 1328.6 [M+2H]²⁺/2-Boc; calc. m/z for C₁₃₃H₂₅₂N₁₈O₄₇ [M]⁺=2855.6.

1b.8 Azido-PEG₂₄CO—[N(PN)₂][Lys]₂[Lys]₄[NH₂.TFA]₈, G2, Compound 8

Prepared according to General Procedure A, using azido-PEG₂₄CO—[N(PN)₂][Lys]₂[Lys]₄[NHBoc]₈, Compound 7 (167 mg, 58.6 μmol). The crude aqueous solution was purified by preparative HPLC (10-60% MeCN, 0.1% TFA buffer; Rₜ 27-29 min) to give the product, Compound 8 as a pale-yellow sticky solid (124 mg, 71% over 2 steps). ¹H NMR (300 MHz, D₂O) δ (ppm): 1.30-1.96 (m, 42H); 2.71 (t, J=6.0 Hz, 2H); 2.98-3.04 (m, 8H); 3.13-3.30 (m, 8H); 3.36-3.53 (m, 7H); 3.68-3.84 (m, 100H); 3.93 (t, J=6.6 Hz, 2H); 4.04 (t, J=6.6 Hz, 2H); 4.25 (t, J=7.2 Hz, 2H). LCMS (philic method, TFA buffer) Rₜ=7.76 min, ESI MS (+ve) 1028.3 [M+2H]²⁺/2, 685.9 [M+3H]³⁺/3; calc. m/z for C₉₃H₁₉₁N₁₈O₃₁²⁺[M+2H]²⁺/2: 1028.3, calc m/z for C₉₃H₁₉₁N₁₈O₃₁³⁺[M+3H]³⁺/3: 685.9.

1b.9 Azido-PEG₂₄CO—[N(PN)₂][Lys]₂[Lys]₄[Lys]₈[(α-NHBoc)(ε-NH-COPEG₁₁₀₀)]₈, G3, Compound 9

Prepared according to General Procedure C, using azido-PEG₂₄CO—[N(PN)₂][Lys]₂[Lys]₄[NH₂.TFA]₈, Compound 8 (123 mg, 41.5 μmol) to give the crude material, Compound 9 as a brown oil. LCMS (philic method, formic buffer) Rₜ=11.52 min, ESIMS (+ve) 2113 [M+6H]⁶⁺/6, 1812 [M+7H]⁷⁺/7, 1585 [M+8H]⁸⁺/8, 1409 [M+9H]⁹⁺/9, 1268 [M+10H]¹⁰⁺/10, 1153 [M+11H]¹¹⁺/11, 1057 [M+12H]¹²⁺/12. Transforms to 12,673.

1b.10 Azido-PEG₂₄CO—[N(PN)₂][Lys]₂[Lys]₄[Lys]₈[(α-NH₂.TFA)(ε-NH-COPEG₁₁₀₀)]₈, G3, Compound 10

Prepared according to General Procedure A, using azido-PEG₂₄CO—[N(PN)₂][Lys]₂[Lys]₄[Lys]₈[(α-NHBoc)(ε-NH-COPEG₁₁₀₀)]₈, Compound 9 (526 mg, 41.5 μmol). The crude aqueous solution was purified by preparative IPLC (3-60% MeCN, 0.1% TFA buffer; Rₜ 38-39 min) to give the product, Compound 10 as a pale-yellow sticky solid (359 mg, 68% over 2 steps). LCMS (philic method, TFA buffer) Rₜ=10.27 min. Transforms to 11,880.

1b.11 Azido-PEG₂₄CO—[N(PN)₂][Lys]₂[Lys]₄[Lys]₈[(α-NHBoc)(ε-NH-Fmoc)]₈, G3, Compound 11

Prepared according to General Procedure D, using azido-PEG₂₄CO—[N(PN)₂][Lys]₂[Lys]₄[NH₂.TFA]s, Compound 8 (105 mg, 35.4 μmol). The product, Compound 11 was obtained as a white solid (166 mg, 83%). ¹H NMR (300 MHz,(CD₃)₂S═O) δ (ppm): 1.23-1.49 (m, 160H); 2.73-2.95 (m, 36H); 3.44-3.60 (m, 94H); 3.83 (m, 8H); 4.05-4.28 (m, 29H); 6.33-6.90 (m, 8H, NH); 7.24-7.87 (m, 84H).

1b.12 Azido-PEG₂₄CO—[N(PN)₂][Lys]₂[Lys]₄[Lys]₈[(α-NHBoc)(ε-NH₂)]₈, G3, Compound 12

Prepared according to General Procedure D, using azido-PEG₂₄CO—[N(PN)₂][Lys]₂[Lys]₄[Lys]₈[(α-NHBoc)(ε-NHFmoc)]₈, Compound 11 (169 mg, 29.9 μmol) to give, Compound 12 as a fluffy solid (95 mg, 82%). ¹H NMR (300 MHz, CD₃OD) δ (ppm): 1.46-1.49 (m, 160H); 2.69 (br s, 14H); 3.09-3.19 (m, 18H); 3.38-3.41 (m, 8H); 3.55-3.78 (m, 96H), 3.89-4.33 (m, 14H).

1b.13 Azido-PEG₂₄CO—[N(PN)₂][Lys]₂[Lys]₄[Lys]₈[(α-NHBoc)(ε-NH-COPEG₅₇₀)]₈, G3, Compound 13 and Azido-PEG₂₄CO—[N(PN)₂][Lys]₂[Lys]₄[Lys]₈[(α-NH₂.TFA)(ε-NH-COPEG₅₇₀)]₈, G3, Compound 14

To a solution of mPEG₅₇₀-CO₂H (205 mg, 348 μmol), NMM (60 μL, 546 μmol) and PyBOP (171 mg, 329 μmol) in DMF (1.5 mL) was added azido-PEG₂₄CO—[N(PN)₂][Lys]₂[Lys]₄[Lys]₈[(α-NHBoc)(ε-NH₂)]₈, Compound 12 in DMF (0.5 mL). The ensuing reaction mixture was allowed to stir at room temperature overnight, then concentrated in vacuo to yield crude Compound 13 which was taken directly dissolved in water, treated with TFA and stirred overnight at room temperature. The mixture was concentrated and taken up in water then purified using a Millipore Centrifugation filtration units (3K MWCO regenerated cellulose) and the freeze dried product, Compound 14 was obtained as an off-white fluffy material (68% over 2 steps). ¹H NMR (300 MHz, D₂O) δ (ppm): 1.33-1.90 (m, 88H); 2.51-2.55 (m, 16H); 2.67-2.75 (m, 4H), 3.16-3.23 (m, 32H); 3.40-3.53 (m, 32H), 3.62-4.03 (m, 470H); 4.21-4.39 (m, 7H). LCMS (philic method, formic buffer) Rₜ=7.50 min.

1b.14 Azido-PEG₂₄CO—[N(PN)₂][Lys]₂[Lys]₄[Lys]₈[(α-NHBoc)(ε-NH-COPEG₂₀₀₀)]₈, G3, Compound 15

To a stirred solution of azido-PEG₂₄CO—[N(PN)₂][Lys]₂[Lys]₄[Lys]₈[(α-NHBoc)(ε-NH₂)]₈, Compound 12 (95.0 mg, 24.5 μmol) in DMF (4 mL) was added DIPEA (85 μL, 488 μmol) followed by mPEG₂₀₀₀-NHS (720 mg, 313 μmol). The ensuing reaction mixture was allowed to stir at room temperature overnight. The crude residue was dissolved in water and purified by ultrafiltration (5K, Pall PES membrane). The retentate was collected and freeze dried to give Compound 15 as an off-white fluffy material (76%). ¹H NMR (300 MHz, D₂O) δ (ppm): 1.33-1.63 (m, 160H); 3.05-3.15 (m, 35H); 3.29 (s, 24H); 3.35-3.96 (m, 1370H); 4.13-4.19 (m, 6H). LCMS (philic method, formic buffer) Rₜ=11.24 min.

1b.15 Azido-PEG₂₄CO—[N(PN)₂][Lys]₂[Lys]₄[Lys]₈[(α-NH₂.TFA)(ε-NH-COPEG₂₀₀₀)]s, G3, Compound 16

Prepared according to General Procedure A, using azido-PEG₂₄CO—[N(PN)₂][Lys]₂[Lys]₄[Lys]₈[(α-NHBoc)(ε-NH-COPEG₂₀₀₀)]₈, Compound 15 (40.0 mg, 1.87 μmol) to give the product as an off-white fluffy material (35 mg, 88%). ¹H NMR (300 MHz, D₂O) δ (ppm): 1.28-1.79 (m, 88H); 2.51-2.58 (m, 4H); 3.04-3.18 (m, 35H); 3.28 (s, 24H); 3.35-3.97 (m, 1348H), 4.14-4.25 (m, 6H). LCMS (philic method, formic buffer) Rₜ=9.12 min.

1b.16 BHALys[Lys]₂[Lys]₄[((α-NH-COPEG₂₄NH-COPEG₄(PhTzMe))₁(α-NH₂)₃)(ε-NH-COPEG₁₀₀₀)₄], G2, Compound 17

Prepared according to the General Procedure C using BHALys[Lys]₂[Lys]₄[(α-NH₂.TFA)₄(ε-NH-COPEG₁₀₀₀)₄] (Ref. 1) (50.0 mg, 0.007 mmol) and HOOCPEG₂₄NH-COPEG₄(PhTzMe) (Click Chemistry Tools, 13.39 mg, 0.010 mmol) except the reaction vessel was wrapped in foil to exclude light and the residue was purified by SEC (Sephadex™ LH-20) using methanol as the eluent to give the product Compound 17 (44.00 mg, 77%); ¹H NMR (300 MHz, D₂O) δ (ppm): 8.42-8.26 (m, 2H), 7.44-7.11 (m, 12H), 6.04 (bs, 1H), 4.44-4.07 (m, 8H), 4.07-3.37 (m, 556H), 3.33 (s, 12H), 3.25-2.90 (m, 17H), 2.62-2.43 (m, 2H), 1.96-0.97 (m, 48H); HPLC (C8 XBridge, 3×100 mm) gradient (formate buffer): 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=8.08-9.01 min.

1b.17 BHALys[Lys]₂[Lys]₄[Lys]₈[((α-NH-COPEG₂₄NH-COPEG₄(PhTzMe))₁(α-NH₂)₇)(s-NH-COPEG₄₁₂)₈], G3, Compound 18

Prepared according to the General Procedure C using BHALys[Lys]₂[Lys]₄[Lys]₈[(α-NH₂.TFA)₈(ε-NH-COPEG₄₁₂)₈] (Ref 1) (50.0 mg, 0.008 mmol) and HOOC-PEG₂₄NH-COPEG₄(PhTzMe) (Click Chemistry Tools, 11.2 mg, 0.008 mmol) except the reaction vessel was wrapped in foil to exclude light and the residue was purified by SEC (Sephadex™ LH-20) using methanol as the eluent to give the product Compound 18 (29 mg, 55%); ¹H NMR (300 MHz, D₂O) δ (ppm): 8.43-8.20 (m, 2H), 7.50-7.09 (m, 10H), 6.03 (bs, 1H), 4.41-4.05 (m, 10H), 4.01-3.41 (m, 258H), 3.31 (s, 16H), 3.24-2.83 (m, 28H), 2.41 (bs, 13H), 2.00-0.98 (m, 75H); HPLC (C8 XBridge, 3×100 mm) gradient (formate buffer): 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=8.16-8.93 min.

1b.18 BHALys[Lys]₂[Lys]₄[Lys]₈[((α-NH-COPEG₂₄NH-COPEG₄(PhTzMe))₁(α-NH₂)₇)(ε-NH-COPEG₁₀₀₀)₈], G3, Compound 19

Prepared according to the General Procedure C using BHALys[Lys]₄[Lys]₈[(α-NH₂.TFA)₈(ε-NH-COPEG₁₀₀₀)₈] (Ref. 1) (100.0 mg, 0.007 mmol) and HOOCPEG₂₄NH-COPEG₄(PhTzMe) (Click Chemistry Tools, 15.97 mg, 0.010 mmol) except the reaction vessel was wrapped in foil to exclude light and the residue was purified by SEC (Sephadex™ LH-20) using methanol as the eluent to give the product Compound 19 (69 mg, 66%);¹H NMR (300 MHz, D₂O) δ (ppm): 8.45-8.25 (m, 2H), 7.47-7.07 (m, 12H), 6.07 (bs, 1H), 4.45-4.05 (m, 12H), 4.04-3.37 (m, 936H), 3.32 (s, 25H), 3.23-2.88 (m, 32H), 2.59-2.32 (m, 6H), 1.90-0.98 (m, 90H); HPLC (C8 XBridge, 3×100 mm) gradient (formate buffer): 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=8.21-9.15 min.

1b.19 BHALys[Lys]₂[Lys]₄[Lys]₈[Lys]₁₆[((α-NH-COPEG₂₄NH-COPEG₄(PhTzMe))₁(α-NH₂)₁₅)(ε-NH-COPEG₁₀₀₀)₁₆], G4, Compound 20

Prepared according to the General Procedure C using BHALys[Lys]₂[Lys]₄[Lys]₈[Lys]₁₆[(α-NH₂.TFA)₁₆(ε-NH-COPEG₁₀₀₀)₁₆] (Ref. 1)(100.0 mg, 0.004 mmol) and HOOCPEG₂₄NH-COPEG₄(PhTzMe) (Click Chemistry Tools, 6.74 mg, 0.005 mmol) except the reaction vessel was wrapped in foil to exclude light and the residue was purified by SEC (Sephadex™ LH-20) using methanol as the eluent to give the product Compound 20 (63 mg, 65%); ¹H NMR (300 MHz, D₂O) δ (ppm): 8.45-8.28 (m, 2H), 7.47-7.07 (m, 12H), 6.03 (bs, 1H), 4.40-4.08 (m, 23H), 4.06-3.38 (m, 1906H), 3.33 (s, 56H), 3.29-2.91 (m, 78H), 2.63-2.45 (m, 3H), 1.98-0.98 (m, 200H); HPLC (C8 XBridge, 3×100 mm) gradient (formate buffer): 5% MeCN/H₂O (0-1 min), 5-80% MeCN (1-7 min), 80% MeCN (7-12 min), 80-5% MeCN (12-13 min), 5% MeCN (13-15 min), 243 nm, 0.4 mL/min, Rₜ=8.51-9.02 min.

1b.20 BHALys[Lys]₂[Lys]₄[Lys]₈[Lys]₁₆[Lys]₃₂[((α-NH-COPEG₂₄NH-COPEG₄(PhTzMe))₁(α-NH₂)₃₁)(ε-NH-COPEG₁₀₀₀)₃₂], G5, Compound 21

Prepared according to the General Procedure C using BHALys[Lys]₂[Lys]₄[Lys]₈[Lys]₁₆[Lys]₃₂[(α-NH₂.TFA)₃₂(ε-NH-COPEG₁₀₀₀)₃₂] (Ref 1) (100.0 mg, 0.002 mmol) and HOOCPEG₂₄NH-COPEG₄(PhTzMe) (Click Chemistry Tools, 3.38 mg, 0.005 mmol) except the reaction vessel was wrapped in foil to exclude light and the residue was purified by SEC (Sephadex™ LH-20) using methanol as the eluent to give the product Compound 21 (68 mg, 72%);¹H NMR (300 MHz, D₂O) δ (ppm): 8.44-8.29 (m, 2H), 7.44-7.09 (m, 12H), 6.02 (bs, 1H), 4.37-4.11 (m, 31H), 4.08-3.37 (m, 2937H), 3.32 (s, 83H), 3.27-2.88 (m,116H), 2.59-2.42 (m, 3H), 2.18-0.92 (m, 313H); HPLC (C8 XBridge, 3×100 mm) gradient (formate buffer): 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=8.77 min.

1b.21 BHALys[Lys]₂[Lys]₄[(α-NHBoc)₄(ε-NH-COPEG₂₄NH-COPEG₄(PhTzMe)₄], G2, Compound 22

To a stirred solution of BHALys[Lys]₂[Lys]₄[(α-NHBoc)₄(ε-NH₂)₄] (Ref. 1) (46.0 mg, 0.031 mmol) in DMF at RT was added NMM (68.0 μL, 0.620 mmol), HOOCPEG₂₄NH-COPEG₄(PhTzMe) (Click Chemistry Tools; 43.0 mg, 0.155 mmol) and PyBOP (81.0 mg, 0.155 mmol). After 16 h, the reaction mixture was dissolved in MeCN:MQ water (3 mL, 1:1 v/v) and the solution filtered (0.45 μm acrodisc syringe filter). The filtrate was purified by preparative HPLC; 30-80% MeCN over 60 min, Mobile phase: MQ water and acetonitrile, Rₜ 33.0-36.0 min. to give Compound 22 as a pink solid 52 mg (22%). LCMS (philic method, TFA buffer) Rₜ=5.66 min; ¹H NMR (300 MHz, D₂O) δ (ppm): 8.52 (d, 8H, J=9.0 Hz), 7.40-7.26 (m, 10H), 7.20 (d, 8H, J=9.0 Hz), 6.23-6.17 (m, 1H), 4.36-4.21 (m, 10H), 3.99-3.83 (m, 13H), 3.82-3.46 (m, 487H), 3.46-3.32 (m, 22H), 3.27-3.02 (m, 15H), 3.02 (s, 12H), 2.54-2.38 (m, 17H), 1.92-1.14 (m, 89H).

1b.22 BHALys[Lys]₂[Lys]₄[(α-NH₂·HCl)₄(ε-NH-COPEG₂₄NH-COPEG₄(PhTzMe)₄], G2, Compound 23

To a stirred solution of BHALys[Lys]₂[Lys]₄[(α-NHBoc)₄(ε-NH-COPEG₂₄NH-COPEG₄(PhTzMe)₄], G2, Compound 22 (48.0 mg) in methanol (2 mL) was added a solution of 3M HCl in methanol (2 mL) and the reaction mixture stirred at room temperature for 20 hrs. The volatiles were removed under reduced pressure to give BHALys[Lys]₂[Lys]₄[(α-NH₂·HCl)₄(ε-NH-COPEG₂₄NH-COPEG₄(PhTzMe)₄], G2, Compound 23 as a pink solid 41.0 mg (91%). LCMS (LCMS Method 20-90, 8 min, TFA) Rₜ=5.27 mins.

1b.23 BHALys[Lys]₂[Lys]₄[((α-Lys(α-NHCy5)(α-NHDFO))₁(α-NH₂)₃)(ε-NH-COPEG₂₄NH-COPEG₄(PhTzMe)₄], G2, Compound 24

To a stirred solution of BHALys[Lys]₂[Lys]₄[(α-NH₂·HCl)₄(ε-NH-COPEG₂₄NH-COPEG₄(PhTzMe)₄], G2, Compound 23 (11.0 mg, 0.0015 mmol) at RT in DMF (3 mL) was added NMM (3.32 μL, 0.039 mmol), HO-Lys[(α-NHCy5)(ε-NHDFO)] Compound 60 (2.06 mg, 0.0015 mmol) and PyBOP (1.18 mg, 0.0022 mmol). After 18 h, the volatiles were removed under reduced pressure and the blue solid residue was dissolved in MQ water and the solution filtered (0.45 μm acrodisc syringe filter). The filtrate was concentrated by spin column (Amicon Ultra, 0.5 mL, 3 kDa MW cut off) and the retentate was washed repeatedly with MQ water (10×450 μL) to give Compound 24 (concentration 10 mg/mL in MQ water; 1.3 mL. LCMS (LCMS Method 20-90, 8 min, TFA) Rₜ=5.64 mins; ¹H NMR (300 MHz, D₂O) δ (ppm): 8.52 (d, 8H, J=9.0 Hz), 8.32 (s, 1H), 8.29-8.17 (m, 2H), 7.78-7.70 (m, 2H), 7.69-7.62 (m, 2H), 7.57-7.47 (m, 2H), 7.48-7.25 (m, 16H), 7.21 (d, 8H, J=9.9 Hz), 7.15-7.08 (m, 1H), 7.06-7.00 (m, 1H), 6.70-6.60 (m, 2H), 6.21-6.12 (m, 2H), 4.37-4.20 (m, 14H), 4.18-4.06 (m, 8H), 3.98-3.50 (m, 480H), 3.48-3.34 (m, 15H), 3.27-3.06 (m, 18H), 3.06-2.97 (m, 19H), 2.88 (s, 8H), 2.54-2.40 (m, 19H), 2.08-1.98 (m, 28H), 1.93-1.12 (m, 99H), 1.00-0.82 (m, 8H).

1b.24 BHALys[Lys]₂[Lys]₄[((α-NH-COPEG₂₄NH-COPEG₄(PhTzMe))₁(α-Lys(α-NHCy5)(ε-NHDFO))₁(α-NH₂)₂)(ε-NH-COPEG₁₀₀₀)₄], G2, Compound 25

Prepared according to General Procedure C using BHALys[Lys]₂[Lys]₄[(α-NH-COPEG₂₄NH-COPEG₄(PhTzMe))(α-NH₂)₃)(ε-NH-COPEG₁₀₀₀)₄], G2, Compound 17 (3.0 mg, 0.414 μmol) and HO-Lys[(α-NHCy5)(ε-NHDFO)] Compound 60 (0.56 mg, 0.414 μmol) and purified by spin column (10 kDa MW cut off washing with 10×450 μL MQ water) to give the desired product Compound 25 (final concentration of 3.56 mg in 300 μL MQ water).

1b.25 BHALys[Lys]₂[Lys]₄[Lys]₈[((α-NH-COPEG₂₄NH-COPEG₄(PhTzMe))₁(α-Lys(α-NHCy5)(ε-NHDFO))₁(α-NH₂)₆)(ε-NH-COPEG₄₁₂)₈], G3, Compound 26

Prepared according to General Procedure C using BHALys[Lys]₂[Lys]₄[Lys]₈[((α-NHCOPEG₂₄NH-COPEG₄(PhTzMe))₁(α-NH₂)₇))(ε-NH-COPEG₄₁₂)₈], G3, Compound 18 (2.97 mg, 0.452 μmol) and HO-Lys[(α-NHCy5)(ε-NHDFO)] Compound 60 (0.61 mg, 0.452 μmol) and purified by spin column (10 kDa MW cut off washing with 10×450 μL MQ water) to give the product Compound 26 at (final concentration of 3.60 mg in 300 μL MQ water).

1b.26 BHALys[Lys]₂[Lys]₄[Lys]₈[((α-NH-COPEG₂₄NH-COPEG₄(PhTzMe))₁(α-Lys(α-NHCy5)(ε-NHDFO))₁(α-NH₂)₆)(ε-NH-COPEG₁₀₀₀)₈], G3, Compound 27

Prepared according to General Procedure C, using BHALys[Lys]₂[Lys]₄[Lys]₈[((α-NHCOPEG₂₄NH-COPEG₄(PhTzMe))(α-NH₂)₇)(ϵ-NHCOPEG₁₀₀₀)₈], G3, Compound 19 (3.14 mg, 0.234 μmol) and HO-Lys(α-NHCy5)(ε-NHDFO) Compound 60 (0.32 mg, 0.234 μmol) and purified by spin column (10 kDa MW cut off washing with 10×450 μL MQ water) and purified by spin column (10 kDa MW cut off washing with 10×450 μL MQ water) to give the product Compound 27 (final concentration of 3.45 mg in 300 μL MQ water).

1b.27 BHALys[Lys]₂[Lys]₄[Lys]₈[Lys]₁₆[((α-NH-COPEG₂₄NH-COPEG₄(PhTzMe))₁(α-Lys(α-NHCy5)(ε-NHDFO))₁(α-NH₂)₁₄)(ε-NH-COPEG₁₀₀₀)₁₆], G4, Compound 28

Prepared according to General Procedure C, using BHALys[Lys]₂[Lys]₄[Lys]₈-[Lys]₁₆[((α-NH-COPEG₂₄NH-COPEG₄(PhTzMe))(α-NH₂)₁₅)(ε-NH-COPEG₁₀₀₀)₁₆], G4, Compound 20 (11.8 mg, 0.454 μmol) and HO-Lys(α-NHCy5)(ε-NHDFO) Compound 60 (0.62 mg, 0.454 μmol) and purified by spin column (10 kDa MW cut off washing with 10×450 μL MQ water) to give 9.3 mg of Compound 28 as a blue solid (after lyophilisation).

1b.28 BHALys[Lys]₂[Lys]₄[Lys]₈[Lys]₁₆[Lys]₃₂[((α-NH-COPEG₂₄NH-COPEG₄(PhTzMe))₁(α-Lys(α-NHCy5)(ε-NHDFO))₁(α-NH₂)₃₀)(ε-NH-COPEG₁₀₀₀)₃₂], G5, Compound 29

Prepared according to General Procedure C, using BHALys[Lys]₂[Lys]₄[Lys]s-[Lys]₁₆[((α-NH-COPEG₂₄NH-COPEG₄(PhTzMe))(α-NH₂)₃₀)(ε-NH-COPEG₁₀₀₀)₃₂], G4, Compound 21 (12.9 mg, 0.271 μmol) and HO-Lys(α-NHCy5)(ε-NHDFO) Compound 60 (0.37 mg, 0.271 μmol) and purified by spin column (10 kDa MW cut off washing with 10×450 μL MQ water) to give 8.4 mg of product Compound 29 as a blue solid after lyophilisation.

1b.29 BHALys[Lys]₂[Lys]₄[Lys]₈[Lys]₁₆[Lys]₃₂[(α-NHBoc)₃₂(ε-NH-COPEG₅₇₀N₃)₃₂], Compound 30

Prepared according to General Procedure H, step 1 using BHALys[Lys]₂[Lys]₄[Lys]₈[Lys]₁₆[Lys]₃₂[(α-NHBoc)₃₂(s-NH₂)₃₂] (Ref 1)), 75 mg, 6.5 μmol). The lyophilised product Compound 30 was obtained as an off-white sticky solid (40 mg, 19%). ¹HNMR (300 MHz, D₂O) δ (ppm): 0.39-2.11 (m, 666H); 2.25-2.53 (m, 58H); 2.53-2.70 (m, 12H); 2.70-4.42 (m, 1540H); 6.89-7.48 (m, 12H). HPLC (HPLC-Method 5-80, 15 min, formate) Rₜ=10.21 min.

1b.30 BHALys[Lys]₂[Lys]₄[Lys]₈[Lys]₁₆[Lys]₃₂[(α-NHBoc)₃₂(ε-NH-COPEG₁₁₀₀N₃)₃₂], Compound 31

Prepared according to General Procedure H, step 1 using BHALys-[Lys]₂[Lys]₄[Lys]₈[Lys]₁₆[Lys]₃₂[(α-NHBoc)₃₂(ε-NH₂)₃₂] (Ref 1) (0, 50 mg, 4.4 μmol) and N₃-PEG₁₁₀₀-COOH. The lyophilised product Compound 31 was obtained as an off-white sticky solid (159 mg, 75%). ¹H NMR (300 MHz, CD₃OD) δ (ppm): 0.91-2.08 (m, 666H); 2.27-2.58 (m, 64H); 3.01-3.27 (m, 113H); 3.33-3.92 (m, 3018H); 3.95-4.18 (m, 33H); 4.20-4.50 (m, 31H); 7.10-7.55 (m, 12H), 7.62-8.15 (m, 24H). HPLC (HPLC-Method 5-80, 15 min, formate) Rₜ=9.62 min.

1b.31 BHALys[Lys]₂[Lys]₄[Lys]₈[Lys]₁₆[Lys]₃₂[(α-NH₂.TFA)₃₂(ε-NH-COPEG₅₇₀N₃)₃₂], Compound 32

Prepared according to General Procedure H, step 2 using BHALys[Lys]₂[Lys]₄[Lys]₈[Lys]₁₆[Lys]₃₂[(α-NHBoc)₃₂(ε-NH-COPEG₅₇₀N₃)₃₂] Compound 30 (40 mg, 1.3 μmol). The product, Compound 32 was obtained as a pale-yellow oil (41 mg, quant.). ¹H NMR (300 MHz, D₂O) δ (ppm): 0.87-1.93 (m, 378H); 2.24-2.51 (m, 67H); 2.52-2.69 (m, 20H); 2.77-3.20 (m, 122H); 3.20-4.05 (m, 1312H); 4.05-4.32 (m, 34H); 5.97 (s, 1H); 6.98-7.34 (m, 10H).

1b.32 BHALys[Lys]₂[Lys]₄[Lys]₈[Lys]₁₆[Lys]₃₂[(α-NH₂.TFA)₃₂(ε-NH-COPEG₁₁₀₀N₃)₃₂], Compound 33

Prepared according to General Procedure H, step 2 using BHALys[Lys]₂[Lys]₄[Lys]₈[Lys]₁₆[Lys]₃₂[(α-NHBoc)₃₂(ε-NH-COPEG₁₁₀₀N₃)₃₂] Compound 31 (145 mg, 3.0 μmol). The product Compound 33 was obtained as a pale-yellow sticky solid (146 mg, quant.). ¹H NMR (300 MHz, CD₃OD) δ (ppm): 1.20-2.03 (m, 378H); 2.37-2.59 (m, 63H); 3.00-3.28 (m, 121H); 3.35-3.96 (m, 3036H); 3.96-4.13 (m, 22H); 4.18-4.56 (m, 39H); 6.19 (s, 1H); 7.20-7.42 (m, 10H). HPLC HPLC-Method 5-80, 15 min, formate) Rₜ=9.41 min.

1b.33 BHALys[Lys]₂[Lys]₄[Lys]₈[Lys]₁₆[Lys]₃₂[(α-NHCy5)₁(α-NHAc)₃₁(ε-NH-COPEG₅₇₀N₃)₃₂], Compound 34

Prepared according to General Procedure I, step 1 and 2 using BHALys[Lys]₂[Lys]₄[Lys]₈[Lys]₁₆[Lys]₃₂[(α-NH₂.TFA)₃₂(s-NH-COPEG₅₇₀N₃)₃₂] (46.1 mg, 1.6 μmol). The product, Compound 34 was obtained as a blue solid (31 mg, 72%). ¹H NMR (300 MHz, D₂O) δ (ppm): 0.93-1.85 (m, 400H); 1.89-2.02 (m, 89H); 2.04-2.09 (m, 23H); 2.30-2.52 (m, 59H); 2.53-2.73 (m, 13H); 2.91-3.21 (m, 121H); 3.24-3.94 (m, 1313H); 3.97-4.37 (m, 67H); 5.87-6.31 (m, 6H); 6.83-7.60 (m, 25H). IR (cm¹): 2100 (—N₃).

1b.34 BHALys[Lys]₂[Lys]₄[Lys]₈[Lys]₁₆[Lys]₃₂[(α-NHCy5)₁(α-NHAc)₃₁(ε-NH-COPEG₁₁₀₀N₃)₃₂], Compound 35

Prepared according to General Procedure I, step 1 and 2 using BHALys[Lys]₂[Lys]₄[Lys]₈[Lys]₁₆[Lys]₃₂[(α-NH₂.TFA)₃₂(ε-NH-COPEG₁₁₀₀N₃)₃₂], Compound 33 (112.5 mg, 2.3 μmol). The product, Compound 35 was obtained as a blue solid (98 mg, 85%). ¹H NMR (300 MHz, CD₃OD) δ (ppm): 1.04-2.18 (m, 507H); 2.38-2.55 (m, 64H); 2.99-3.27 (m, 122H); 3.37-3.96 (m, 3026H); 4.15-4.51 (m, 67H); 6.10-6.48 (m, 5H); 7.15-7.58 (m, 24H). HPLC HPLC-Method 5-80, 15 min, formate) Rₜ=9.52 min.

1b.35 BHALys[Lys]₂[Lys]₄[Lys]₈[Lys]₁₆[((α-NH-COPEG₂₄NH-COPEG₄(PhTzMe))₁(α-NHCy5)₁(α-NHDOTA)₁₀(α-NH₂)₄(ε-NH-COPEG₁₀₀₀)₁₆], G4, Compound SRS-1

To a stirred solution of BHALys[Lys]₂[Lys]₄[Lys]₈[Lys]₁₆[((α-NH-COPEG₂₄NH-COPEG₄(PhTzMe))₁(α-NH₂)₁₅)(ε-NH-COPEG₁₀₀₀)₁₆], G4, Compound 20 (148.0 mg, 0.0054 mmol) and NMM (38.0 μL, 0.474 mmol) in DMF (4 mL) was added Cy5-NHS ester (Lumiprobes, 3.66 mg, 0.0054 mmol) and the reaction mixture stirred at rt and monitored by LCMS for consumption of Cy5 NHS ester. After 18 h, a solution of p-SCN-Bn-DOTA (Macrocyclics, 75.0 mg, 0.109 mmol) in DMSO (1 mL) was added and stirring continued for 24 h. The volatiles were removed in vacuo and the residue was dissolved in MQ water (15.0 mL), the solution then filtered (0.45 μm acrodisc filter). The filtrate was purified by spin column (Amicon Ultra-15, 10 kDa MWCO) and the retentate was washed repeatedly with MQ water (5×15 mL). The retentate was dried by lyophilisation to give SRS-1 as a blue solid (173.0 mg, 96%). HPLC: XBridge C8 column with gradient 5-80% MeCN/H₂O (1-7 min), 80% MeCN/H₂O (7-12 min), 80-5% MeCN/H₂O (12-13 min), 5% MeCN/H₂O (13-15 min), 10 mM HCOONH₄) and UV detection at 214 nm. Rₜ=5.07 min. ¹H NMR (300 MHz, D₂O) δ (ppm): 0.33-2.08 (m, 177H), 2.26-4.46 (m, 1979H), 6.87-7.66 (bs, 59H), 8.34 (bs, 2H).

1b.36 BHALys[Lys]₂[Lys]₄[Lys]₈[Lys]₁₆[Lys]₃₂[(α-NH₂)₃₀(α-NHDOTA)₂(ε-NHCOPEG₂₀₀₀)₃₂] G5 RH-2

To a stirred solution of BHALys[Lys]₂[Lys]₄[Lys]₈[Lys]₁[Lys]₃₂[(α-NH₂.TFA)₃₂(α-(ε-NHCOPEG₂₀₀₀)₃₂] (as described in WO20200014750, Example 1) RH-1 (301 mg, 3.97 μmol) in DMF (6.0 mL) was added p-SCN-Bn-DOTA (8.13 mg, 11.8 μmol, 2.98 eq), followed by addition of NMM (114 μL, 1.03 mmol). The resulting reaction mixture was stirred at ambient temperature for 4.5 h, then a portion of the reaction mixture (2.0 mL) was removed and placed in another reaction flask equipped with a stirrer bar and stirring continued. After 24 h, the smaller aliquot of the reaction mixture was concentrated in vacuo to dryness, dissolved in MeOH (1.0 mL) and purified by SEC (Stationary phase=Sepahdex LH-20™, mobile phase=acetonitrile, elution rate=˜1 drop s⁻¹, fraction size=400 drops). The product-containing fractions were combined and concentrated in vacuo, and the resulting residue dissolved in MQ water, filtered (0.45 um acrodisc filter) and lyophilised to give compound RH-2 as a white solid (82.5 mg). ¹H NMR (300 MHz, CD₃OD-d₄) δ (ppm): 1.17-2.29 (m, 401H), 3.36 (s, 96H), 3.39-3.43 (m, 43H), 3.50-4.08 (m, 5564H), 4.21-4.67 (m, 84H), 6.17 (broad s, 1H), 7.18-7.64 (m, 18H), 8.09 (s, 1H). ¹H NMR analysis suggests approx. 2.1 DOTA/dendrimer; % (w/w) of DOTA=2.0%.

1b.37 [(ε-NHBoc)(α-NHBoc)][Lys]-CONH—CH₂—CH₂—S-]₂ RL-3

To a stirred solution of cystamine hydrochloride RL-1 (1.26 g, 5.6 mmol) and Boc-Lys(Boc)-ONp RL-2 (5.68 g, 12.2 mmol) in DMF (100 mL) was added TEA (5.44 mL, 39.0 mmol). The reaction mixture was stirred for 3 d at rt whereupon a solution of glycine (1.82 g, 24.4 mmol) in deionised water (10 mL) whereupon a white solid precipitated from solution. The suspension was stirred for 2 h and the volatiles were removed in vacuo. The residue suspended in ethyl acetate (50 mL) and the organics were washed sequentially with aq saturated sodium carbonate solution (5×20 mL), aq 0.1 M HCl (20 mL), brine (20 mL), dried (MgSO₄) and the volatiles removed in vacuo. The residue was dissolved in dichloromethane (50 mL), washed with aq 0.2M NaOH (4×50 mL), brine (50 mL), dried (MgSO₄) and the volatiles were removed in vacuo to give [(ε-NHBoc)(α-NHBoc)][Lys]-NHCO—CH₂—CH₂—S-]₂ RL-3 as an off-white solid (3.66 g, 81%). ¹H NMR (300 MHz, CD₃OD): δ (ppm) 1.13-1.82 (m, 48H), 2.83 (t, J=6.6 Hz, 4H), 3.02 (t, J=6.6 Hz, 4H), 3.52 (m, 4H), 3.98 (m, 2H). LCMS (LCMS Method 40-90, 8 min, TFA): Rₜ=5.36 min. ESI MS (+ve) 809 [M]⁺; calc. m/z for C₃₆H₆₈N₆O₁₀S₂ [M]⁺=809.

1b.38 [(ε-NH₂.TFA)(α-NH₂.TFA)][Lys]-CONH—CH₂—CH₂—S-]₂ RL-4

To a solution of [(ε-NHBoc)(α-NHBoc)][Lys]-NHCO—CH₂—CH₂—S-]₂ RL-3 (3.66 g, 4.5 mmol) in dichloromethane (30 mL) at rt was added TFA (27.7 mL, 362.0 mmol) dropwise over 5 min. The reaction mixture was stirred for 18 h at rt and concentrated in vacuo. The residue was dissolved in deionised water (50 mL) and lyophilised to give [(ε-NH₂.TFA)(α-NH₂.TFA)][Lys]-CONH—CH₂—CH₂—S-]₂ RL-4 as pale brown solid (3.91 g, quant). ¹H NMR (300 MHz, CD₃OD): δ (ppm) 1.28-1.42 (m, 4H), 1.54-1.68 (m, 4H), 1.76-1.88 (m, 4H), 2.70-2.86 (m, 4H), 2.90 (t, J=7.7 Hz, 2H), 3.39-3.62 (m, 4H), 3.88 (t, J=6.6 Hz, 2H). LCMS (LCMS Method 5-60, 8 min, TFA) Rₜ=0.80 min. ESI MS (+ve) 409 [M]⁺; calc. m/z for C₁₆H₃₆N₆O₂S₂[M]⁺=409.

1b.39 [[(ε-NHBoc)₂(α-NHBoc)₂][Lys]₂[Lys]-CONH—CH₂—CH₂—S-]₂RL-5

To a solution of [(ε-NH₂.TFA)(α-NH₂.TFA)][Lys]-CONH—CH₂—CH₂—S-]₂ RL-4 (3.91 g, 4.52 mmol) in DMF (18 mL) was added Boc-Lys(Boc)-ONp RL-2 (10.13 g, 21.7 mmol). The mixture was heated at 40° C. until a clear solution was obtained and then cooled to rt and TEA (7.55 mL, 54.2 mmol) added. After stirring at rt for 18 h, a solution of glycine (509 mg, 4.46 mmol) in water (1.28 mL) was added and the reaction mixture was then heated at 40° C. with stirring. After 2 h, the reaction mixture was cooled to rt and then added dropwise to water (with stirring) over 20 min. The resulting precipitate was collected by filtration, washed with deionised water (5×30 mL) and dried in a stream of air for 30 min. The solid was dissolved in DMF (18 mL) and the solution added dropwise to water (180 mL) with stirring. The resultant white solid was collected by filtration, washed with water (3×30 mL) and dried in a vacuum oven for 20 h at 40° C. to give [[(ε-NHBoc)₂(α-NHBoc)₂][Lys]₂[Lys]-CONH—CH₂—CH₂—S-]₂ RL-5 as a white solid (6.49 g, 83.3%). ¹H NMR (300 MHz, CD₃OD): δ (ppm) 1.26-1.93 (m, 108H), 2.84 (m, 4H), 3.04 (m, 8H), 3.20 (m, 4H), 3.51 (m, 4H), 4.00 (m, 4H), 4.33 (m, 2H). LCMS (LCMS Method 40-90, 8 min, TFA) Rₜ=6.34 min. ESI MS (+ve) 1722 [M]⁺; calc. m/z for C₈₀H₁₄₈N₁₄O₂₂S₂[M]⁺=1722.

1b.40 [[(ε-NH₂. TFA)₂(α-NH₂.TFA)₂][Lys]₂[Lys]-CONH—CH₂—CH₂—S-]₂ RL-6

To a stirred suspension of [[(ε-NHBoc)₂(α-NHBoc)₂][Lys]₂[Lys]-CONH—CH₂—CH₂—S-]₂ RL-5 (6.49 g, 3.8 mmol) in dichloromethane (65 mL) at 0° C. was added TFA (34.6 mL, 451.2 mmol) dropwise over 10 min. The resultant solution was allowed to warm to rt and stirred for 4 h whereupon the volatiles were removed in vacuo. The residue was dissolved in the minimum amount of deionised water and lyophilised twice (N.B. during the second lyophilization process, the cloudy aqueous solution was filtered through a 0.45 μm filter disc and the filtrate was lyophilised) to give [[(ε-NH₂.TFA)₂(α-NH₂.TFA)₂][Lys]₂[Lys]-CONH—CH₂—CH₂—S-]₂ RL-6 as a light brown foam (6.20 g, 96.9%). ¹H NMR (300 MHz, CD₃OD): δ (ppm) 1.13-1.87 (m, 36H), 2.72 (m, 4H), 2.87 (m, 8H), 3.09 (t, J=7.0 Hz, 4H), 3.30-3.53 (m, 4H), 3.79 (t, J=6.6 Hz, 2H), 3.90 (t, J=6.5 Hz, 2H), 4.13 (t, J=7.1 Hz, 2H).

1b.41 [[(ε-NHBoc)₄(α-NHBoc)₄][Lys]₄[Lys]₂[Lys]-CONH—CH₂—CH₂—S-]₂RL-7

To a solution of [[(ε-NH₂.TFA)₂(α-NH₂.TFA)₂][Lys]₂[Lys]-CONH-CH2-CH2-S-]₂ RL-6 (4.02 g, 2.20 mmol) in DMF (16 mL) was added Boc-Lys(Boc)-ONp RL-2 (9.02 g, 19.3 mmol). The mixture was heated at 40° C. until a clear solution was obtained and then cooled to rt and TEA (7.35 mL, 52.8 mmol) added. After stirring at rt for 18 h, DMF (25 mL) was added to dissolve any precipitated solids and the resultant solution was heated at 55° C. and a solution of glycine (250 mg, 3.3 mmol) in water (1.20 mL) was added. After stirring for 2 h, the reaction mixture was cooled to rt and then added dropwise to water (200 mL) with stirring over 10 min. The resulting precipitate was collected by filtration and dried in a stream of air for 30 min. The solid was dissolved in DMF (40 mL) and the resultant solution added dropwise to deionised water (200 mL) with stirring. The resultant white solid was collected by filtration, dried in a stream of air for 30 min and then suspended in MeCN and the volatiles were removed in vacuo to give [[(ε-NHBoc)₄-(α-NHBoc)₄][Lys]₄[Lys] 2[Lys]-CONH—CH₂—CH₂—S-]₂ RL-7 as a white solid (4.80 g, 37.0%). ¹H NMR (300 MHz, CD₃OD): δ (ppm) 1.17-1.95 (m, 228H), 2.86 (m, 4H), 3.05 (m, 16H), 3.20 (m, 12H), 3.53 (m, 4H), 4.03 & 4.32 (14H). LCMS (LCMS Method 40-90, 8 min, TFA) Rₜ=7.69 min. ESI MS (+ve) 1773 [M/2]⁺; calc. m/z for C₈₄H₁₅₄N₁₅O₂₃S₁[M/2]⁺=1773.

1b.42 [[(ε-NH₂. TFA)₄(α-NH₂.TFA)₄][Lys]₄[Lys]₂[Lys]-CONH—CH₂—CH₂—S-]₂ RL-8

To a stirred suspension of [[(ε-NHBoc)₄-(α-NHBoc)₄][Lys]₄[Lys]₂[Lys]-CONH—CH₂—CH₂—S-]₂ RL-7 (4.80 g, 1.4 mmol) in dichloromethane (50 mL) at 0° C. was added TFA (50.0 mL, 648.0 mmol) dropwise over 15 min. The resultant solution was allowed to warm to rt and stirred for 4 h, whereupon the volatiles were removed in vacuo. The residue was dissolved in the minimum amount of deionised water and lyophilised (twice) to give a light brown foam (5.38 g), which was subsequently dissolved in methanol (˜15 mL) and the resultant solution added dropwise to diethyl ether (300 mL) with stirring. The white precipitate so formed was collected by filtration, dissolved in methanol and the volatiles removed in vacuo to give [[(ε-NH₂.TFA)₄(α-NH₂. TFA)₄]₄[Lys]₄[Lys]₂[Lys]-CONH—CH₂—CH₂—S-]₂ RL-8 as an off-white hygroscopic foam (3.50 g, 74.1%). ¹H NMR (300 MHz, CD₃OD): δ (ppm) 1.27-2.01 (m, 84H), 2.82 (m, 4H), 2.90-3.04 (m, 16H), 3.06-3.29 (m, 12H), 3.52 (m, 4H), 3.87 (m, 4H), 3.99 (t, J=6.32 Hz, 4H), 4.30 (m, 4H), 4.38 (m, 2H). LCMS (LCMS Method 5-60, 8 min, TFA) Rₜ=0.65 min. ESI MS (+ve) 975 [(M/2)+H]⁺; calc. m/z for C₄₄H₉₁N₁₅O₇S₁[(M/2)+H]⁺=975.

1b.43 [[(ε-NHBoc)₈(α-NHBoc)₈][Lys]₈[Lys]₄[Lys]₂[Lys]-CONH—CH₂—CH₂—S-]₂RL-9 and [[(ε-NH₂. TFA)₈(α-NH₂.TFA)₈][Lys]₈[Lys]₄[Lys]₂[Lys]-CONH—CH₂—CH₂—S-]₂ RL-10

To a solution of [[(ε-NH₂.TFA)₄(α-NH₂.TFA)₄]₄[Lys]₄[Lys]₂[Lys]-CONH—CH₂—CH₂—S-]₂ RL-8 (1.5 g, 0.398 mmol) in DMF (17 mL) was added Boc-Lys(Boc)-ONp RL-2 (3.27 g, 6.99 mmol) and NMM (2.10 mL, 19.1 mmol) at rt. The reaction mixture was stirred for 2 d whereupon a solution of glycine (66 mg) in water (1.0 mL) was added. After 4 h, the reaction mixture was added dropwise to water (200 mL) with stirring. The resulting yellow precipitate was collected by filtration and washed with water (5×50 mL) and dried in a stream of air. The solid was dissolved in DMF (20 mL) and the resulting solution added to water (200 mL) to give an off-white precipitate, which was collected and dried as described above. The solid was dissolved in DMF (4.0 mL) and TEA (2.1 mL) and heated with stirring at 55° C. whereupon a solution of glycine (66 mg) in water (1 mL) was added. After 4 h, the reaction mixture was cooled to rt and stirred for 2 d and then added dropwise to water (250 mL) with stirring. The resulting precipitate was collected by filtration, washed with water (3×50 mL) and dried in vacuo to give an off-white solid, [[(ε-NHBoc)₈(α-NHBoc)₈][Lys]₈[Lys]₄[Lys]₂[Lys]-CONH—CH₂—CH₂—S-]₂ RL-9 (˜0.8 g), which was used without further purification.

To a solution of [[(ε-NHBoc)₈(α-NHBoc)₈][Lys]₈[Lys]₄[Lys]₂[Lys]-CONH—CH₂—CH₂—S-]₂ RL-9 (˜0.8 g) in dichloromethane at 0° C. (16 mL) was added TFA (16 mL). The reaction mixture was warmed to rt, stirred for 18 h and the volatiles were removed in vacuo. The residue was dissolved in water (20 mL) and concentrated by centrifugation at 4000 rpm for 20 min using 15 mL Amicon® Ultra centrifugal filters with 3 kDa MWCO Ultracel® regenerated cellulose membranes. The retentate was diluted water and the centrifugation/concentration process repeated (×10) and the final retentate was lyophilised to give [[(ε-NH₂.TFA)₈(α-NH₂.TFA)₈][Lys]₈[Lys]₄[Lys]₂[Lys]-CONH—CH₂—CH₂—S-]₂ RL-10 as an off-white solid (606 mg, 20%, 2 steps). ¹H NMR (300 MHz, CD₃OD): δ (ppm) 1.24-2.00 (m, 180H), 2.81 (m, 4H), 2.90-3.04 (m, 32H), 3.12-3.33 (m, 28H), 3.52 (m, 4H), 3.83 (m, 8H), 3.94 (m, 8H) and 4.23-4.42 (m, 14H). LCMS (LCMS Method 5-60, 8 min, TFA) Rₜ=3.76 min. ESI MS (+ve) 1000 [M/4+H]⁺; calc. m/z for [C₁₈₄H₃₇₃N₆₂O₃₀S₂]/4 [M/4+H]⁺=1000.

1b.44 [(ε-NH₂.TFA)₈(α-NH₂.TFA)₈][Lys]₈[Lys]₄[Lys]₂[Lys]-CONH—CH₂—CH₂—S-Me(MAL)-PEG₂₄-CONH-Bn-Tz(Me) RP-1

To a stirred solution of [[Lys(ε-NH₂.TFA)(α-NH₂.TFA)]s-[Lys]₄-[Lys]₂-[Lys]-CONH—CH₂—CH₂—S-]₂ RL-10 (128 mg, 0.017 mmol) in water (6.0 mL) at rt was added a solution of 0.5M of TCEP in water (335 μL, 0.170 mmol). The reaction mixture was stirred for 1 h whereupon the pH of the reaction mixture was measured (pH 5.2) and adjusted to pH 6.5 by dropwise addition of aq. 0.1M NaOH. Me(MAL)-PEG₂₄-CONH-Bn-Tz(Me) RL-18 (28.6 mg, 0.020 mmol) was then added in a single portion and stirring continued. After 2 h, the reaction mixture was transferred into Amicon® Ultra centrifugal filters with 3 kDa MWCO Ultracel® regenerated cellulose membrane and concentrated by centrifugation at 4000 rpm for 20 min. The retentate was washed with water (×5) by centrifugation at 4000 rpm and lyophilised to give a yellow solid (109 mg). The solid was dissolved in water (6 mL) and air was bubbled through the solution for 18 h, which resulted in a pink solution. The solution was lyophilised to give a pink solid (111 mg), which was further purified by preparative HPLC (HPLC-Method 5-80, 8 min, TFA) to give [(ε-NH₂.TFA)₈(α-NH₂.TFA)₈][Lys]₈[Lys]₄[Lys]₂[Lys]CONH—CH₂—CH₂—S-(Me)MAL-Me(MAL)-PEG₂₄-CONH-Bn-Tz(Me) RP-1 as a pink solid (32.5 mg, 32.5%). ¹H NMR (300 MHz, CD₃OD): δ (ppm) 1.21-2.05 (m, 87H), 2.90-3.04 (m, 14H), 3.06 (s, 3H), 3.06-3.26 (m, 11H), 3.48-4.07 (m, 89H), 4.17-4.53 (m, 11H), 7.59 (d, J=7.6 Hz, 2H) and 8.53 (d, J=8.0 Hz, 2H). LCMS (LCMS Method 40-90, 8 min, TFA) Rₜ=3.98 min. ESI MS (+ve) 1141 [(M/3)+H]⁺; calc. m/z for C₁₅₈H₃₀₁N₃₇O₄₂S [(M/3)+H]⁺=1141.

1b.45 [(ε-NHBoc)₁₆(α-NHCOPEG₂₅)₁₆][Lys]₁₆[Lys]₈[Lys]₄[Lys]₂[Lys]CONH—CH₂—CH₂—S-Me(MAL)-PEG₂₄-CONH-Bn-Tz(Me) RP-3

To a stirred solution of [(ε-NH₂.TFA)₈(α-NH₂.TFA)₈][Lys]₈[Lys]₄[Lys]₂[Lys]CONH—CH₂—CH₂—S-(Me)MAL-PEG₂₄-CONH-Bn-Tz(Me) RP-1 (31.4 mg, 6 μmol) in anhydrous DMF (3.0 mL) was added NMM (105 μL, 955 μmol) followed by a solution of PyBOP (59.8 mg, 115 μmol) and HO-Lys(Boc)(PEG₁₁₀₀) RP-2 in anhydrous DMF (2.0 mL). The reaction mixture was stirred for 18 h and then purified by TFF (Pellicon 10 kDa MWCO membrane) using water as the circulating medium until 10 diafiltration volumes were collected as permeate. The retentate was collected and pooled with line washings and lyophilised to give [(ε-NHBoc)₁₆(α-NHCOPEG₂₅)₁₆][Lys]₁₆[Lys]₈[Lys]₄[Lys]₂[Lys]CONH—CH₂—CH₂—S-(Me)MAL-PEG₂₄-CONH-Bn-Tz(Me) RP-3 as a pink solid (104 mg, 65%). ¹H NMR (300 MHz, CD₃OD): δ (ppm) 1.19-1.97 (m, 383H), 2.38-2.54 (m, 36H), 3.06 (s, 3H), 3.09-3.29 (m, 64H), 3.38 (s, 56H), 3.39-3.48 (m, 8H), 3.51-3.59 (m, 36H), 3.59-3.70 (m, 1889H), 3.70-3.80 (m, 32H), 3.85-3.92 (m, 6H), 3.94-4.15 (m, 7H), 4.21-4.44 (m, 10H), 7.60 (d, J=8.1 Hz, 2H) and 8.53 (d, J=8.1 Hz, 2H). HPLC (HPLC-Method 5-80, 8 min, TFA) Rₜ=5.10 min.

1b.46 [(ε-NH₂·HCl)₁₆(α-NHCOPEG₂₅)₁₆][Lys]₁₆[Lys]₈[Lys]₄[Lys]₂[Lys]CONH—CH₂—CH₂—S-Me(MAL)-PEG₂₄-CONH-Bn-Tz(Me) RP-4

To [(ε-NHBoc)₁₆(α-NHCOPEG₂₅)₁₆][Lys]₁₆[Lys]₈[Lys]₄[Lys]₂[Lys]CONH—CH₂—CH₂—S-(Me)MAL-PEG₂₄-CONH-Bn-Tz(Me) RP-3 (83 mg, 3.3 μmol) in a chilled vial was added 3.0 M HCl in methanol (1.75 mL). The resulting solution was stirred for 18 h and allowed to warm to rt whereupon the volatiles were removed in vacuo. The residue was dissolved in water and lyophilised to give [(ε-NH₂·HCl)₁₆(α-NHCOPEG₂₅)₁₆][Lys]₁₆[Lys]₈[Lys]₄[Lys]₂[Lys]CONH—CH₂—CH₂—S-Me(MAL)-PEG₂₄-CONH-Bn-Tz(Me) RP-4 as a pink solid (82.6 mg, quantitative). ¹H NMR (300 MHz, CD₃OD): δ (ppm) 1.27-2.05 (m, 198H), 2.39-2.57 (m, 36H), 3.15-3.29 (m, 44H), 3.38 (s, 39H), 3.39-3.59 (m, 9H), 3.59-3.73 (m, 32H), 3.59-3.70 (m, 1217H), 3.73-3.83 (m, 33H) and 3.85-4.15 (m, 31H) and 4.21-4.55 (m, 26H). HPLC (HPLC-Method 5-80, 8 min, TFA) Rₜ=4.78 min.

1b.47 [(ε-NHCO-PEG₁₁₀₀)₁₆(α-NHCy5)₁(α-NHDOTA)₈(α-NH₂)₇]][Lys]₁₆[Lys]₈[Lys]₄[Lys]₂[Lys]-CONH—CH₂—CH₂—S-(Me)MAL-PEG₂₄-CONH-Bn-Tz(Me) RP-5

To a stirred solution of [(ε-NHCO-PEG₁₁₀₀)₁₆(α-NH₂·HCl)₁₆][Lys]₁₆[Lys]₈[Lys]₄[Lys]₂[Lys]-CONH—CH₂—CH₂—S-(Me)MAL-PEG₂₄-CONH-Bn-Tz(Me) RP-4 (82.6 mg, 3.2 μmol) in DMF (2.0 mL) and NMM (24 μL, 0.21 mmol) was added a solution of cyanine5 NHS ester (2.3 mg, 3.2 μmol) in DMF (1.0 mL) at rt. The reaction mixture was monitored by LCMS (LCMS Method 20-90, 8 min, TFA) for the consumption of cyanine5 NHS ester. After 18 h, a solution of p-SCN-Bn-DOTA (44.7 mg, 68 μmol) in DMSO (1.0 mL) was added to the reaction mixture and stirring continued. After 2 d, water (50 mL) was added and the resulting solution was filtered through a 0.45 μm filter disc and purified by TFF (Pellicon 10 kDa MWCO membrane) using water as the circulating medium until 11 diafiltration volumes were collected as permeate. The retentate was collected and pooled with line washings and lyophilised to give [(ε-NHCO-PEG₁₁₀₀)₁₆(α-NHCy5)₁(α-NHDOTA)₈(α-NH₂)₇][Lys]₁₆[Lys]₈[Lys]₄[Lys]₂[Lys]-CONH—CH₂—CH₂—S-(Me)MAL-PEG₂₄-CONH-Bn-Tz(Me) RP-5 as a blue solid (74.5 mg). ¹H NMR (300 MHz, CD₃OD): δ (ppm) 0.74-2.23 (m, 230H), 2.36-2.65 (m, 53H), 2.91-3.28 (m, 102H), 3.38 (s, 63H), 3.40-3.46 (m, 19H), 3.52-3.94 (m, 1959H), 6.17-6.86 (m, 3H), 6.96-7.74 (m, 46H). The loading of DOTA was determined by qNMR using 3,4,5-trichloropyridine as an internal standard by comparing the integrals of the aromatic region (δ 6.96-7.74 ppm) to the aromatic protons of the internal standard (δ 8.62 ppm, 2H_{IS}). In this way, the number of DOTA groups per molecule was found to be 8.6. The number of cyanine 5 groups per dendrimer was set at 1 since complete consumption of cyanine5 NHS ester was observed. The molecular weight of the dendrimer was calculated to be 26,665 Da. HPLC (HPLC-Method 5-80, 8 min, TFA) Rₜ=4.76 min.

1b.48 [[(ε-NH₂)₄(α-NH₂)₄[Lys]₄[Lys]₂[Lys]-CONH—CH₂—CH₂—S-]₂MAL-PEG₃-NHCO-PEG₂₄-CONH-PEG₄-(PhTzMe) SRS-1-Mal

To a solution of [[(ε-NH₂.TFA)₄(α-NH₂.TFA)₄][Lys]₄[Lys]₂[Lys]-CONH—CH₂—CH₂—S-]₂ RL-8 (65 mg, 17.2 μmol) in water (1.0 mL) at rt was added a solution of 0.5M TCEP in water (17.0 μL, 8.5 μmol, pH 7.0). The reaction mixture was monitored by LCMS (LCMS Method 2) and 0.5M TCEP solution was added in increments until complete reduction of the disulfide bond was observed (in total, an additional 17.0 μL, 8.5 μmol of TCEP was added over approximately 1 h). The pH of the reaction mixture was adjusted to 6.2 using 0.1 N NaOH (aq) whereupon a solution of Br₂(MAL)-PEG₃-NHCO-PEG₂₄-CONH-PEG₄-(PhTzMe) RL-15 (32 mg, 16.4 μmol) in water (0.5 mL) was added. The reaction mixture was stirred for 1 h, diluted with water (30 mL), filtered (0.45 μm syringe filter disc) and the filtrate transferred to 15 mL Amicon® Ultra centrifugal filters with 3 kDa MWCO Ultracel® regenerated cellulose membrane and concentrated by centrifugation at 4000 rpm for 20 min. The retentate was washed with water (6×15 mL) by centrifugation at 4000 rpm and lyophilised to give [[(ε-NH₂)₄(α-NH₂)₄][Lys]₄[Lys]₂[Lys]-CONH—CH₂—CH₂—S-]₂MAL-PEG₃-NHCO-PEG₂₄-CONH-PEG₄-Bn-Tz(Me) SRS-1-Mal as an orange solid (65 mg, 99%). ¹H NMR (300 MHz, CD₃OD): δ (ppm) 1.31-1.66 (m, 37H), 1.66-2.02 (m, 45H), 2.05-2.26 (m, 7H), 2.38-2.59 (11H), 2.91-3.08 (m, 18H), 3.12-3.26 (m, 8H), 3.27-3.41 (m, 8H), 3.40-3.62 (m, 20H), 3.68-3.80 (m, 20H), 3.80-4.05 (m, 9H), 4.23-4.47 (m, 7H), 7.21 (d, J=9.0 Hz, 2H) and 8.52 (d, J=9.0 Hz, 2H). LCMS (LCMS Method 5-60, 8 min, TFA) Rₜ=5.04 min. ESI MS (+ve) 1265 [(M/3)+H]⁺; calc. m/z for C₁₇₁H₃₂₇N₃₉O₅₀S₂ [(M/3)+H]⁺=1265.

1b.49 [[(ε-NHCO-PEG₁₁₀₀)₄(α-NH₂)₈][Lys]₈[Lys]₄[Lys]₂[Lys]-CONH—CH₂—CH₂—S-]₂MAL-PEG₃-NHCO-PEG₂₄-CONH-PEG₄-(PhTzMe)SRS-2

To a solution of [[(ε-NH₂)₄(α-NH₂)₄][Lys]₄[Lys]₂[Lys]-CONH—CH₂—CH₂—S-]₂MAL-PEG₃-NHCO-PEG₂₄-CONH-PEG₄-Bn-Tz(Me) SRS-1-Mal (65 mg, 17.1 μmol), HO-Lys(Boc)(PEG₁₁₀₀) RP-2 (289 mg, 208 μmol) and NMM (119 μL, 1.08 mmol) in DMF (4.0 mL) was added PyBOP (108 mg, 208 μmol) at rt. After 18 h, the reaction mixture was diluted with water (50 mL), filtered (0.45 μm syringe filter disc) and purified by TFF (Pellicon 10 kDa MWCO membrane) using water as the circulating medium until 10 diafiltration volumes were collected as permeate. The retentate was collected and pooled with line washings and lyophilised to give [[(ε-NHCO-PEG₁₁₀₀)₈(α-NH₂)₈][Lys]₈[Lys]₄ [Lys]₂[Lys]-CONH—CH₂—CH₂—S-]₂MAL-PEG₃-NHCO-PEG₂₄-CONH-PEG₄-(PhTzMe) SRS-2 as a dark orange solid (158 mg, 36%). ¹H NMR (300 MHz, CD₃OD): δ (ppm): 1.19-2.01 (m, 307H), 2.40-2.55 (m, 34H), 3.03 (s, 3H) 3.10-3.30 (m, 52H), 3.38 (s, 45H), 3.39-3.44 (m, 9H) 3.53-3.59 (m, 35H), 3.59-3.70 (m, 1522H), 3.70-3.80 (m, 37H), 3.84-3.94 (m, 7H), 3.95-4.14 (m, 8H), 4.26-4.44 (m, 11H), 7.22 (d, J=9 Hz, 2H) and 8.53 (d, J=9 Hz, 2H). HPLC (HPLC-Method 5-80, 8 min, TFA) Rₜ=5.09 min.

1b.50 [[(ε-NHCO-PEG₁₁₀₀)₈(α-NH₂·HCl)₈][Lys]₈[Lys]₄[Lys]₂[Lys]-CONH—CH₂—CH₂—S-]₂MAL-PEG₃-NHCO-PEG₂₄-CONH-PEG₄-(PhTzMe) SRS-3-Mal

[[(ε-NHCO-PEG₁₁₀₀)₈(α-NH₂)₈][Lys]₈[Lys]₄[Lys]₂[Lys]-CONH—CH₂—CH₂—S-]₂MAL-PEG₃-NHCO-PEG₂₄-CONH-PEG₄-(PhTzMe) SRS-2 (156 mg, 6.1 μmol) was dissolved in 3.0 M HCl in methanol (3.23 mL) at rt. The resultant solution was stirred for 18 h whereupon the volatiles were removed in vacuo. The residue was dissolved in water (˜10 mL) and the solution lyophilised to give [[(s-NHCO-PEG₁₁₀₀)₈(α-NH₂·HCl)₈][Lys]₈[Lys]₄[Lys]₂[Lys]-CONH—CH₂—CH₂—S-]₂MAL-PEG₃-NHCO-PEG₂₄-CONH-PEG₄-(PhTzMe) SRS-3-Mal as a dark orange solid (142 mg, 95%). HPLC (HPLC-Method 5-80, 8 min, TFA) Rₜ=4.77 min.

1b.51 [[(ε-NHCO-PEG₁₁₀₀)₈(α-NHDOTA)_{4.75}(α-NHCy5)_{0.5}(α-NH₂)_{2.75}][Lys]₈[Lys]₄[Lys]₂[Lys]-CONH—CH₂—CH₂—S-]₂MAL-PEG₃-NHCO-PEG₂₄-CONH-PEG₄-(PhTzMe) SRS-4-Mal

To a solution of [[(ε-NHCO-PEG₁₁₀₀)₈(α-NH₂·HCl)₈][Lys]₈[Lys]₄[Lys]₂[Lys]-CONH—CH₂—CH₂—S-]₂MAL-PEG₃-NHCO-PEG₂₄-CONH-PEG₄-(PhTzMe) SRS-3-Mal (130 mg, 5.3 μmol) and NMM (37 μL, 337 μmol) in DMF (mL) was added Cyanine5 NHS ester (3.5 mg, 5.2 μmol). The reaction mixture was stirred for 18 h at rt whereupon a solution of p-SCN-Bn-DOTA (72 mg, 105 μmol) in DMSO (500 μL) was added and stirring continued for a further 24 h. The volatiles were removed in vacuo and the residue was dissolved in water (50 mL) and the solution then filtered (0.45 μm syringe filter disc) and purified by TFF (Pellicon 10 kDa MWCO membrane) using water as the circulating medium until 10 diafiltration volumes were collected as permeate. The retentate was collected and pooled with line washings and lyophilised to give [[(ε-NHCO-PEG₁₁₀₀)₈(α-NHDOTA)_{4.75}(α-NHCy5)_{0.5}(α-NH₂)_{2.75}][Lys]₈[Lys]₄[Lys]₂[Lys]-CONH—CH₂—CH₂—S-]₂MAL-PEG₃-NHCO-PEG₂₄-CONH-PEG₄-(PhTzMe) SRS-4-Mal as a blue solid (120 mg). ¹H NMR (300 MHz, CD₃OD): δ (ppm): 1.02-2.14 (m, 110H), 2.36-2.61 (m, 26H), 3.03 (s, 3H), 3.07-3.30 (m, 39H), 3.38 (s, 32H), 3.39-3.45 (m, 11H), 3.53-3.59 (m, 33H), 3.69-3.97 (m, 61H), 6.87-7.79 (m, 22H) and 8.52 (d, J=9.0 Hz, 2H). The loading of DOTA was determined by qNMR using 3,4,5-trichloropyridine as an internal standard by comparing the integrals of the aromatic region (δ 6.80-7.92 ppm) to the aromatic protons of the internal standard (δ 8.62 ppm, 2H_{IS}). In this way, the number of DOTA groups per molecule was found to be 9.5. The number of cyanine 5 groups per dendrimer was set at 1 since complete consumption of cyanine5 NHS ester was observed. The molecular weight of the dendrimer was calculated to be 29,817 Da. LCMS (LCMS Method 20-90, 8 min, TFA, no MS): Rₜ=5.03 min.

1b.52 [(ε-NHCO-PEG₁₁₀₀)₁₆(α-DOTA)₇(α-NHCy5)₁(α-NH₂)₈][Lys]₁₆[Lys]₈[Lys]₄[Lys]₂[Lys]-(PN)NCO-PEG₂₄-CONH-PEG₄-(PhTzMe) HH-2

To a stirred solution of [(ε-NHCO-PEG₁₁₀₀)₁₆(α-NH₂·HCl)₁₆][Lys]₁₆[Lys]₈[Lys]₄[Lys]₂[Lys]-(PN)NCO-PEG₂₄-CONH-PEG₄-(PhTzMe) HH-1 (129 mg, 5.6 μmol) in DMF (4.0 mL) was added and NMM (39 μL, 357 μmol) followed by Cyanine5 NHS ester (3.7 mg, 5.5 5.5 μmol). After stirring for 18 h, analysis of the reaction mixture (LCMS method 3) showed complete consumption of Cyanine5 NHS ester. A solution of p-SCN-Bn-DOTA (77 mg, 112 μmol) in DMSO (1.0 mL) was added and stirring continued for a further 24 h. The reaction mixture was then diluted with and the resulting solution was transferred into an Amicon® Ultra centrifugal filters with 10 kDa MWCO Ultracel® regenerated cellulose membrane and concentrated by centrifugation at 4000 rpm for 10 min. The retentate was washed with water (×5) by centrifugation at 4000 rpm and the retentate lyophilised to give a blue gum (117 mg). ¹H NMR (300 MHz, CD₃OD): δ (ppm): 1.05-2.16 (m, 172H), 2.37-2.58 (m, 32H), 2.98-3.29 (m, 61H), 3.38 (s, 48H), 3.40-3.44 (m, 9H), 3.58-3.70 (m, 1351H), 3.70-3.83 (m, 43H), 3.83-3.97 (m, 15H) and 6.99-7.70 (m, 25H). The loading of DOTA was determined by qNMR using 3,4,5-trichloropyridine as an internal standard by comparing the integrals of the aromatic region (δ 6.99-7.70 ppm) to the aromatic protons of the internal standard (δ 8.62 ppm, 2H_{IS}). In this way, the number of DOTA groups per molecule was found to be 7. The number of cyanine 5 groups per dendrimer was set at 1 since complete consumption of cyanine5 NHS ester was observed. The molecular weight of the dendrimer was calculated to be 27,420 Da. LCMS (LCMS Method 20-90, 8 min, TFA, no MS): Rₜ=5.11 min.

1b.53 (MeTzPh)PEG₄CO-NHPEG₂₄CO—[N(PN)₂][Lys]₂[Lys]₄[Lys]₈[((α-NHCy5)₁(α-NHAc)₇)(ε-NH-COPEG₁₁₀₀)₈], G3, Compound 52

A solution of Cy5-NHS ester (1.0 mL of a 1 mg/mL solution in DMF; 1.0 mg, 1.59 μmol) was added to vial containing (MeTzPh)PEG₄CO-NHPEG₂₄CO—[N(PN)₂][Lys]₂[Lys]₄[Lys]₈[((α-NH₂)₈)(ε-NH-COPEG₁₁₀₀)₈], Compound 113 (20 mg, 1.59 μmol) in DMF (0.5 mL). To this solution was added NMM (10 μL, 91.0 μmol) and the ensuing reaction mixture protected from light and stirred at RT. After 3.5 h acetic anhydride (20 μL, 212 μmol) was added and the reaction mixture left to stir overnight. The reaction mixture was concentrated under reduced pressure then taken up in MQ water (5 mL) and divided in two for purification through two (pre-equilibrated) PD10 columns. Once the sample entered the column bed, the sample was eluted with 3.5 mL MQ water and the filtrate collected. The filtrates were combined and freeze dried overnight. Lyophilisation gave the title product as a bright blue powder, 19.3 mg (93%). HPLC (C8 XBridge, 3×100 mm) gradient: 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=8.30 min.

1b.54 (MeTzPh)PEG₄CO-NHPEG₂₄CO—[N(PN)₂][Lys]₂[Lys]₄[Lys]₈[Lys]₁₆[((α-NHCy5)₁(α-NHAc)₁₅)(ε-NH-COPEG₁₁₀₀)₁₆], G4, Compound 53

A solution of Cy5-NHS ester (520 μL of a 1 mg/mL solution in DMF; 0.52 mg, 844 nmol) was added to vial containing (MeTzPh)PEG₄CO-NHPEG₂₄CO—[N(PN)₂][Lys]₂[Lys]₄[Lys]₈[Lys]₁₆[((α-NH₂·HCl)₁₆)(ε-NH-COPEG₁₁₀₀)₁₆] Compound 120 (20 mg, 844 nmol) in DMF (1.0 mL). To this solution was added NMM (10 μL, 94.5 μmol) and the ensuing reaction mixture protected from light and stirred at RT. After 3.5 h acetic anhydride (20 μL, 230 μmol) was added and the reaction mixture left to stir overnight. The reaction mixture was concentrated under reduced pressure then taken up in MQ water (5 mL) and divided in two for purification through two (pre-equilibrated) PD10 columns. Once the sample entered the column bed, the sample was eluted with 3.5 mL MQ water and the filtrate collected. The filtrates were combined and freeze dried overnight. Lyophilisation gave the title product as a bright blue powder, 19.9 mg (98%). HPLC (C8 XBridge, 3×100 mm) gradient: 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=8.40 min.

1b.55 (MeTzPh)PEG₄CO-NHPEG₂₄CO—[N(PN)₂][Lys]₂[Lys]₄[Lys]₈[Lys]₁₆[Lys]₃₂[((α-NHCy5)₁(α-NHAc)₃₁)(ε-NH-COPEG₁₁₀₀)₃₂], G5, Compound 54

A solution of Cy5-NHS ester (300 μL of a 1 mg/mL solution in DMF; 0.30 mg, 487 nmol) was added to vial containing (MeTzPh)PEG₄CO-NHPEG₂₄CO—[N(PN)₂][Lys]₂[Lys]₄[Lys]₈[Lys]₁₆[Lys]₃₂[(α-NH₂.TFA)₃₂(ε-NH-COPEG₁₁₀₀)₃₂] Compound 122 (20 mg, 435 nmol) in DMF (1.2 mL). To this solution was added NMM (11 μL, 97.5 μmol) and the ensuing reaction mixture protected from light and stirred at RT. After 3.5 h acetic anhydride (22 μL, 237 μmol) was added and the reaction mixture left to stir overnight. The reaction mixture was concentrated under reduced pressure then taken up in MQ water (5 mL) and divided in two for purification through two (pre-equilibrated) PD10 columns. Once the sample entered the column bed, the sample was eluted with 3.5 mL MQ water and the filtrate collected. The filtrates were combined and freeze dried overnight. Lyophilisation gave the title product as a bright blue powder, 18.7 mg (91%). HPLC (C8 XBridge, 3×100 mm) gradient: 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=8.50 min.

1b.56 Azido-PEG₂₄CO—[N(PN)₂][Lys]₂[Lys]₄[Lys]₈[(α-NHGlu-vc-PAB-MMAE)₈(ε-NH-COPEG₅₇₀)₈], G3, Compound 64

Prepared according to General Procedure E, using azido-PEG₂₄CO—[N(PN)₂][Lys]₂[Lys]₄[Lys]₈[(α-NH₂.TFA)₈(ε-NH-COPEG₅₇₀)₈] Compound 14 (7.2 mg, 842 nmol) and HO-Glu-vc-PAB-MMAE (10.0 mg, 8.08 μmol) to give the product Compound 64 at a concentration of 14.6 mg/3.5 mL (240 μM).

1b.57 Azido-PEG₂₄CO—[N(PN)₂][Lys]₂[Lys]₄[Lys]₈[(α-NHGlu-vc-PAB-MMAE)₈(ε-NH-COPEG₁₁₀₀)₈], G3, Compound 65

Prepared according to General Procedure E, using azido-PEG₂₄CO—[N(PN)₂][Lys]₂[Lys]₄[Lys]₈[(α-NH₂.TFA)₈(ε-NH-COPEG₁₁₀₀)₈] Compound 10 (10.8 mg, 842 nmol) and HO-Glu-vc-PAB-MMAE (10.0 mg, 8.08 μmol) to give the product Compound 65 at a concentration of 18 mg/3.5 mL (240 μM).

1b.58 Azido-PEG₂₄CO—[N(PN)₂][Lys]₂[Lys]₄[Lys]₈[(α-NHGlu-vc-PAB-MMAE)₈(ε-NH-COPEG₂₀₀₀)₈], G3, Compound 66

Prepared according to General Procedure E, using azido-PEG₂₄CO—[N(PN)₂][Lys]₂[Lys]₄[Lys]₈[(α-NH₂.TFA)₈(ε-NH-COPEG₂₀₀₀)₈] Compound 16 (18.1 mg, 842 nmol) and HO-Glu-vc-PAB-MMAE (10.0 mg, 8.08 μmol) to give the product Compound 66 at a concentration of 25.5 mg/3.5 mL (240 μM).

1b.59 Azido-PEG₂₄CO—[N(PN)₂][Lys]₂[Lys]₄[Lys]₈[(α-NHDGA-MMAF(OMe))₈(ε-NH-COPEG₁₁₀₀)₈], G3, Compound 67

Prepared according to General Procedure E, using azido-PEG₂₄CO—[N(PN)₂][Lys]₂[Lys]₄[Lys]₈[(α-NH₂.TFA)₈(ε-NH-COPEG₁₁₀₀)₈] Compound 10 (16.3 mg, 1.28 μmol) and DGA-MMAF(OMe) (10.6 mg, 12.3 μmol) to give the product Compound 67 at a concentration of 23.8 mg/3.5 mL (365 μM).

1b.60 BHA[Lys]₂[Lys]₄[Lys]₈[((α-NH-COPEG₂₄NH-COPEG₄(PhMeTz))₁(α-NH₂)₇)(ε-NH-COPEG₁₀₀₀)₈], G3, Compound 69

A stirred solution of BHA[Lys]₂[Lys]₄[Lys]₈[(α-NH₂.TFA)₈(ε-NH-COPEG₁₀₀₀)₈] (Ref 1) (100 mg, 0.00786 mmol, 1.0 eq) in DMF (300 μL) was prepared at RT. To this was added (MeTzPh)PEG₄CO-NHPEG₂₄CO₂H (Click Chemistry Tools; 16 mg, 0.01 mmol, 1.3 eq), PyBOP (8 mg, 0.013 mmol, 1.6 eq) and DMF (200 μL). The reaction mixture was stirred for 3 min before addition of NMM (40 mg, 50 μL, 0.38 mmol, 48 eq). The contents were protected from light and stirred overnight at RT. The reaction mixture was diluted with MQ water and lyophilized overnight. The lyophilized material was taken up in MeOH (1 mL) and purified by SEC (400 drops/tube, MeOH sephadex LH20, 35 drops/min). The product-containing fractions were checked by HPLC and collected in 2 different fractions. Each fraction was concentrated under reduced pressure, then the resulting residue taken up in MQ water, filtered (0.45 μm acrodisc filter) and freeze dried to yield Compound 69 as a pink solid (69 mg, 66%). HPLC (C8 XBridge, 3×100 mm) gradient: 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=8.4 min (broad peak). ¹H NMR (300 MHz, D₂O) δ (ppm): 1.00-2.00 (m, 90H), 2.51 (t, 3H), 2.60 (br s, 3H), 3.00-3.12 (m, 6H), 3.12-3.35 (br s, 27H), 3.35-3.45 (m, 26H), 3.45-4.15 (m, 937H), 4.15-4.45 (m, 12H), 6.12 (s, 1H), 7.15-7.50 (m, 12H), 8.40-8.50 (m, 2H).

1b.61 BHA[Lys]₂[Lys]₄[Lys]₈[((α-NH-COPEG₂₄NH-COPEG₂-BCN)₁(α-NH₂)₇) (ε-NH-COPEG₁₀₀₀)s], G3, Compound 70

A stirred solution of BCN-PEG₂CO-NHPEG₂₄-CO₂H Compound 19 (9.6 mg, 0.006 mmol, 1.3 eq) in DMF (200 μL) was prepared at RT. To this was added PyBOP (4 mg, 0.008 mmol, 1.6 eq) and NMM (23 mg, 25 μL, 0.226 mmol, 48 eq) and after 5 min BHA[Lys]₂[Lys]₄[Lys]₈[(α-NH₂.TFA)₈(ε-NH-COPEG₁₀₀₀)₈] (Ref 1) (60 mg, 0.006 mmol, 1.0 eq) was added followed by DMF (200 μL). The contents were protected from light and stirred at overnight at RT. The reaction mixture was diluted with MeCN (10 mL) then purified by SEC (400 drops/tube, MeCN sephadex LH20, 35 drops/min). The product-containing fractions were checked by HPLC, collected, filtered (0.45 μm acrodisc filter), concentrated under reduced pressure and freeze dried overnight to give Compound 70 as a pale-yellow solid (58 mg, yield 92%). HPLC (C8 XBridge, 3×100 mm) gradient: 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=8.43 min (broad peak). ¹H NMR (300 MHz, CD₃OD) δ (ppm): 0.75-1.12 (m, 15H), 1.12-2.15 (m, 95H), 2.15-2.35 (m, 7H), 2.55 (br s, 3H), 3.00-3.35 (m, 90H), 3.35-3.38 (s, 17H), 3.38-4.09 (m, 592H), 4.13 (d, 2H), 4.18-4.63 (br s, 7H), 6.18 (s, 0.9H), 7.12-7.48 (m, 7H).

1b.62 (MeTzPh)PEG₄CO-NHPEG₂₄CO—[N(PN)₂][Lys]₂[Lys]₄[Lys]₈[((α-NHCy5)₁(α-NHGlu-VC-PAB-MMAE)₇)(ε-NH-COPEG₁₁₀₀)₈], G3, Compound 71

A solution of Cy5-NHS (214 μL of a 4.2 mg/mL solution in DMF; 1.43 μmol, 1.0 eq.) was added to neat (MeTzPh)PEG₄CO-NHPEG₂₄CO—[N(PN)₂][Lys]₂[Lys]₄[Lys]₈[(α-NH₂·HCl)₈(ε-NH-COPEG₁₁₀₀)₈] Compound 113 (18 mg, 1.43 μmol). Once fully dissolved, NMM (10 μL, 91.0 μmol) was added and the ensuing reaction mixture stirred and protected from light. After 2 h, NMM (10 μL, 91.0 μmol) and PyBOP (7.3 mg, 14.0 μmol) were added to the dendrimer solution, followed by a solution of Glu-VC-PAB-MMAE in DMF (290 μL of a 60 mg/mL solution, 14.0 μmol, 9.8 eq.). The ensuing reaction mixture was protected from light and stirred at RT overnight. The reaction mixture was diluted with PBS (2.0 mL) to make a final volume of 2.5 mL. The diluted solution was then passed through a PD10 de-salting column (pre-equilibrated with PBS). Once the entire solution had entered the bed of the column, PBS (3.5 mL) was added to elute the product (which appeared as a blue band). Final theoretical concentration of Compound 71=8.7 mg/mL in PBS. Material stored frozen at −80° C. HPLC (C8 XBridge, 3×100 mm) gradient: 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=95-10 min (broad peak); 83% conjugate-related peak with 17% MMAE/MMAE-linker.

1b.63 (MeTzPh)PEG₄CO-NHPEG₂₄CO—[N(PN)₂][Lys]₂[³H-Lys]₄[Lys]₈[(α-NHGlu-VC-PAB-MMAE)₈(ε-NH-COPEG₁₁₀₀)₈], G3, Compound 72

To a solution of (MeTzPh)PEG₄CO-NHPEG₂₄CO—[N(PN)₂][Lys]₂[³H-Lys]₄[Lys][(α-NH₂·HCl)₈(g-NH-COPEG₁₁₀₀)₈] (synthesised according to procedure used for synthesis of Compound 114 but using tritiated DBL-OPNP (Ref 1) for synthesis of G2 layer) (36.8 mg, 2.93 μmol) in NMM/DMF (7.8 μL/0.5 mL) was added solid PyBOP (24.7 mg, 47.5 μmol). Once fully dissolved, the dendrimer solution was added to a solution of HO-Glu-VC-PAB-MMAE (40 mg, 32.3 μmol) in NMM/DMF (7.8 μL/0.5 mL). The ensuing reaction mixture was protected from light and stirred at RT overnight. The reaction mixture was diluted with PBS (4.0 mL) to make a final volume of 5 mL. The diluted solution was then passed through two PD10 de-salting columns (pre-equilibrated with PBS, 2.5 mL through each column). Once all solutions had entered the bed of the columns, PBS (3.5 mL) was added to each column to elute the product. The resulting crude mixtures were further purified using regenerated cellulose Amicon Ultra-0.5 mL centrifugation units (10K MWCO). Final theoretical concentration of Compound 72=29-30 mg/mL in PBS. The material stored frozen at −20° C. HPLC (C₈ XBridge, 3×100 mm) gradient: 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=9.5-12 min (broad peak); 91.4% conjugate-related peak with 8.6% MMAE/MMAE-linker related peaks.

1b.64 (MeTzPh)PEG₄CO-NHPEG₂₄CO—[N(PN)₂][Lys]₂[Lys]₄[Lys]₈[((α-NHDFO)₂(α-NHGlu-VC-PAB-MMAE)₆)(ε-NH-COPEG₁₁₀₀)₈], Compound 73

A stirred solution of p-SCN-Deferoxamine (2.1 mg, 2.79 μmol) in DMSO (100 μL) was prepared at RT. To this was added (MeTzPh)PEG₄CO-NHPEG₂₄CO[N(PN)₂][Lys]₂[Lys]₄[Lys]₈[(α-NH₂·HCl)₈(ε-NH-COPEG₁₁₀₀)₈] Compound 113 (17.0 mg, 1.35 μmol) in DMF (200 μL). The ensuing reaction mixture was stirred for 3 min before addition of NMM (10 μL, 91.0 μmol). The resulting solution was protected from light and stirred for 4 h at RT. PyBOP (7.0 mg, 13.5 μmol) was added and after 5 min the reaction mixture was added to neat HO-Glu-VC-PAB-MMAE (9.76 mg, 7.89 μmol). The ensuing reaction mixture was left to stand overnight. The reaction mixture was diluted with PBS buffer (4. 5 mL) and divided across 4 Amicon Ultra centrifugal filters (10K MWCO) and the filters centrifuged (14K rcf, 15 min). The retentate was diafiltered against PBS (400 μL, 14K rcf, 15 min×10 times). The retentate was combined to give a pink coloured solution, approximate concentration of 16 mg of Compound 73 in 2 mL. HPLC (C8 XBridge, 3×100 mm) gradient: 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=8.7-9.8 min (broad peak).

1b.65 BHA[Lys]₂[Lys]₄[Lys]₈[((α-NH-COPEG₂₄NH-COPEG₄(PhMeTz))₁(α-NHDFO)₂(α-NHGlu-VC-PAB-MMAE)₅)(ε-NH-COPEG₁₁₀₀)₈], Compound 74

A stirred solution of p-SCN-Deferoxamine (2.0 mg, 2.66 μmol) in DMSO (100 μL) was prepared at RT. To this was added BHALys[Lys]₂[Lys]₄[Lys]₈[((α-NH-COPEG₂₄NH-COPEG₄(PhMeTz))₁(α-NH₂)₇)(ε-NHPEG₁₁₀₀)₈] Compound 69 (17.0 mg, 1.27 μmol) in DMF (200 μL). The ensuing reaction mixture was stirred for 3 min before addition of NMM (10 μL, 91.0 μmol). The resulting solution was protected from light and stirred for 4 h at RT. PyBOP (7.0 mg, 13.5 μmol) was added and after 5 min the reaction mixture was added to neat HO-Glu-VC-PAB-MMAE (9.17 mg, 7.41 μmol). The ensuing reaction mixture was left to stand overnight. The reaction mixture was diluted with PBS buffer (4. 5 mL) and divided across 4 Amicon Ultra centrifugal filters (10K MWCO) and the filters centrifuged (14K rcf, 15 min). The retentate was diafiltered against PBS (400 μL, 14K rcf, 15 min×10 times). The retentate was combined to give a pink coloured solution, approximate concentration of 16 mg of Compound 74 in 2 mL. HPLC (C8 XBridge, 3×100 mm) gradient: 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=9.3-9.7 min (broad peak).

1b.66 (MeTzPh)-PEG₂₄-CO[N(PNBoc)₂] Compound 106

To a stirred solution of (MeTzPh)-PEG₂₄-CO₂H (0.402 g, 0.305 mmol), PyBOP (0.205 g, 0.394 mmol) and NMM (130 μL, 1.18 mmol) in DMF (3 mL) was added NH(PNBoc)₂ (0.147 g, 0.444 mmol) under an atmosphere of N₂. The ensuing reaction mixture left to stir overnight at room temperature. The volatiles were removed in vacuo and the resulting oil was purified on silica chromatography (5% to 10% MeOH/DCM) to give the desired product Compound 106 as a red residue (0.474 g, 95%). ¹H NMR (300 MHz, CD₃OD) δ (ppm): 1.43-1.44 (m, 18H); 1.64-1.80 (m, 4H); 2.63 (t, J 6.0 Hz, 2H); 3.00 (s, 3H); 3.02-3.09 (m, 4H); 3.34-3.40 (m, 4H), 3.58-3.77 (m, 99H); 3.88-3.91 (m, 2H); 4.25-4.28 (m, 2H); 7.15-7.20 (m, 2H); 8.46-8.51 (m, 2H). LCMS (phobic method, formic buffer) Rₜ=6.20 min. ESI MS (+ve) 1631.0 [M+H]⁺; calc. m/z for C₇₆H₁₄₀N₇O₃₀ [M+H]⁺: 1631.0.

1b.67 (MeTzPh)-PEG₂₄-CO[N(PNH₂·HCl)₂] Compound 107

To (MeTzPh)-PEG₂₄-CO[N(PNBoc)₂] Compound 106 (0.420 g, 0.258 mmol) in ice/water bath, 1.25 M HCl/MeOH solution (8 mL, 10.0 mmol) was slowly added. After 5 min, the ice-bath was removed and the ensuing reaction mixture left to stir at room temperature overnight. The volatiles were removed in vacuo to give product Compound 107 as a red residue (0.388 g, 100%). ¹H NMR (300 MHz, CD₃OD) δ (ppm): 1.91-2.07 (m, 4H); 2.68 (t, J 6.0 Hz, 2H); 2.94-3.09 (m, 7H); 3.53-3.80 (m, 108H); 3.88-3.91 (m, 2H); 4.25-4.28 (m, 2H); 7.16-7.20 (m, 2H); 8.47-8.51 (m, 2H). LCMS (philic method, formic buffer) Rₜ=8.12 min. ESI MS (+ve) 1430.9 [M+H]⁺; calc. m/z for C₆₆H₁₂₄N₇O₂₆ [M+H]⁺: 1430.8.

1b.68 (MeTzPh)-PEG₄-PEG₂₄-CO[N(PNH₂·HCl)₂] Compound 108

To a stirred solution of H₂N-PEG₂₄-CO[N(PNBoc)₂] (0.418 g, 0.286 mmol) in DMF (2.0 mL) was added (MeTzPh)-PEG₄-CO₂H (0.15 g, 0.344 mmol), PyBOP (0.178 g, 0.342 mmol) and NMM (80 μL, 0.727 mmol). The ensuing reaction mixture left to stir overnight at room temperature. The volatiles were removed in vacuo and the resulting oil was cooled in ice/water bath then 1.25 M HCl/MeOH solution (10.0 mL, 12.5 mmol) was slowly added. After 5 min, the ice-bath was removed and the ensuing reaction mixture left to stir at room temperature overnight. The volatiles were removed in vacuo and the resulting oil was dissolved in MeCN/H2O (8 mL, 1:1) and purified by preparative HPLC (10-50% MeCN, 0.1% formic acid buffer, RT 35 min) to give red solid Compound 108 (1.37 g, 54%). ¹H NMR (300 MHz, CD₃OD) δ (ppm): 1.91-2.04 (m, 4H); 2.43 (t, J 6.0 Hz, 2H); 2.68 (t, J 6.0 Hz, 2H); 2.94-3.07 (m, 4H); 3.00 (s, 3H); 3.35 (t, J 6.0 Hz, 2H); 3.51-3.80 (m, 119H); 3.88-3.91 (m, 2H); 4.25-4.28 (m, 2H); 7.16-7.20 (m, 2H); 8.46-8.51 (m, 2H). LCMS (philic method, formic buffer) Rₜ=8.15 min. ESI MS (+ve) 1677.9 [M+H]⁺; calc. m/z for C₇₇H₁₄₅N₈O₃₁ [M+H]⁺: 1678.0.

1b.69 (MeTzPh)-PEG₄-PEG₂₄-CO[N(PN)₂[Lys]₂[NHBoc]₄ G1 Compound 109

To a stirred solution of (MeTzPh)-PEG₄-PEG₂₄-CO[N(PNH₂·HCl)₂] Compound 108 (0.195 g, 0.111 mmol) and DBL-OPNP (Ref.1) (0.146 g, 0.312 mmol) in DMF (3.0 mL) under an atmosphere of N₂ was added NMM (125 μL, 0.864 mmol). The ensuing reaction mixture was then left to stir overnight at room temperature. The volatiles were removed in vacuo and the resulting oily residue was purified by column chromatography on silica gel (5%-10%-15% MeOH/DCM) to give the desired product Compound 109 as a red oil (0.186 g, 72%). ¹H NMR (300 MHz, CD₃OD) δ (ppm): 1.28-1.87 (m, 56H); 2.43 (t, J 6.0 Hz, 2H); 2.63 (t, J 6.0 Hz, 4H); 3.00 (s, 3H); 3.02-3.06 (m, 4H); 3.10-3.21 (m, 4H);). 3.35-3.41 (m, 6H); 3.51-3.78 (m, 110H); 3.84-3.99 (m, 4H); 4.25-4.28 (m, 4H); 7.15-7.20 (m, 2H); 8.47-8.51 (m, 2H). LCMS (phobic method, formic buffer) Rₜ=6.68 min; ESI MS (+ve) 2335.3, [M+H]⁺; calc. m/z for C₁₀₉H₂₀₁N₁₂O₄1+[M+H]⁺=2335.4.

1b.70 (MeTzPh)-PEG₄-PEG₂₄-CO[N(PN)₂[Lys]₂[NH₂·HCl]₄ GI Compound 110

To an ice-cooled (MeTzPh)-PEG₄-PEG₂₄-CO[N(PN)₂[Lys]₂[NHBoc]₄ Compound 109 (0.215 g, 0.0921 mmol), was slowly added a solution of 1.25 M HCl/MeOH (6.0 mL, 7.50 mmol). After 5 min, the ice-bath was removed and the ensuing reaction mixture left to stir at room temperature overnight. The volatiles were removed in vacuo to give the product Compound 110 as a red oil (0.208 g, 108%). %). ¹H NMR (300 MHz, CD3OD) δ (ppm): 1.51-1.99 (m, 20H); 2.47 (t, J 6.0 Hz, 2H); 2.67 (t, J 6.0 Hz, 4H); 2.90-3.04 (m, 7H); 3.35-3.41 (m, 7H); 3.51-3.78 (m, 106H); 4.25-4.28 (m, 2H); 7.17-7.20 (m, 2H); 8.47-8.51 (m, 2H); LCMS (philic method, formic buffer) Rₜ=7.28 min, ESI MS (+ve) 1934.9 [M+H]⁺; calc. m/z for C₈₉H₁₆₉N₁₂O₃₃ [M]⁺=1935.2.

1b.71 (MeTzPh)-PEG₄-PEG₂₄-CO[N(PN)₂][Lys]₂[Lys]₄[NHBoc]₈ G2 Compound 111

To a stirred solution of (MeTzPh)-PEG₄-PEG₂₄-CO[N(PN)₂[Lys]₂[NH₂·HCl]₄ Compound 110 (0.192 g, 0.0822 mmol) and DBL-OPNP (Ref.1) (0.215 g, 0.460 mmol) in DMF (3.0 mL) under an atmosphere of N₂ was added NMM (215 μL, 0.1.96 mmol). The ensuing reaction mixture was then left to stir overnight at room temperature. The volatiles were removed in vacuo and the resulting oily residue was purified on silica chromatography (5%-10%-15% MeOH/DCM) to give the desired product Compound 111 as a red oil (0.231 g, 87%). ¹H NMR (300 MHz, CD₃OD) δ (ppm): 1.28-1.87 (m, 140H); 2.44 (t, J 6.0 Hz, 2H); 2.63 (t, J 6.0 Hz, 2H); 3.00 (s, 3H); 3.02-3.06 (m, 10H); 3.10-3.21 (m, 16 kH)). 3.33-3.40 (m, 8k H); 3.51-3.77 (m, 120H); 3.84-3.99 (m, 14H); 3.94-4.10 (m, 4H); 4.25-4.28 (m, 4H); 7.15-7.20 (m, 2H); 8.47-8.52 (m, 2H). LCMS (phobic method, formic buffer) Rₜ=8.15 min; ESI MS (+ve) [M+2]⁺=1624.5; [(M-3Boc)+3]=1016.6; calc. m/z for C₁₅₃H₂₈₁N₂₀O₅₃⁺[M+H]⁺=3247.99.

1b.72 (MeTzPh)-PEG₄-PEG₂₄-CO[N(PN)₂][Lys]₂[Lys]₄[NH₂·HCl]₈ G2 Compound 112

To an ice-cooled (MeTzPh)-PEG₄-PEG₂₄-CO[N(PN)₂][Lys]₂[Lys]₄[NHBoc]₈ Compound 111 (0.231 g, 0.0711 mmol), was slowly added a solution of 1.25 M HCl/MeOH (9.0 mL, 11.3 mmol) was slowly added. After 5 min, the ice-bath was removed and the ensuing reaction mixture left to stir at room temperature overnight. The volatiles were removed in vacuo to give the product Compound 112 as a red oil (0.226 g, 100%). ¹H NMR (300 MHz, CD₃OD) δ (ppm): 1.51-1.96 (m, 40H); 2.53 (t, J=6.0 Hz, 2H); 2.96-3.04 (m, 10H); 3.15-3.20 (m, 8H); 3.39-3.45 (m, 7H); 3.54-4.10 (m, 102H); 4.25-4.28 (m, 2H); 7.17-7.20 (m, 2H); 8.48-8.51 (m, 2H); LCMS (philic method, formic buffer) Rₜ=6.38 min, ESI MS (+ve) 2447.6 [M+H]⁺; calc. m/z for C₁₁₃H₂₁₇N₂₀O₃₇ [M+H]⁺=2447.6.

1b.72 (MeTzPh)-PEG₄-PEG₂₄-CO[N(PN)₂][Lys]₂[Lys]₄[Lys]₈[(α-NH₂·HCl)(ε-NH—CO PEG₁₁₀₀)]₈ G3 Compound 113

To a stirred solution of HO-Lys(Boc)(PEG₁₁₀₀) (Ref. 1) (0.541 g, 0.402 mmol) and PyBOP (0.195 g, 0.375 mmol) in DMF (2 mL) under an atmosphere of N₂ was added NMM (225 μL, 2.96 mmol). After 10 min, that solution was added to (MeTzPh)-PEG₄-PEG₂₄-CO[N(PN)₂][Lys]₂[Lys]₄[NH₂·HCl]s Compound 112 (0.109 g, 0.0398 mmol) in DMF (1 mL). The ensuing reaction mixture was left to stir overnight at room temperature. The volatiles were removed in vacuo and the resulting crude was cooled in ice/water bath. To the cooled residue, 1.25 M (8.0 mL, 10 mmol) was slowly added. After 10 min, the cold bath was removed and the reaction was left stirring at room temperature overnight. The volatiles were removed in vacuo then dissolved in H₂O (16 mL). The solution was purified by centrifugation* using Millipore Amicon Ultra-15 Centrifugal Filter Units (4 units×10 kDa MWCO units). The retentate was freeze-dried overnight to give product Compound 113 (0.327 g, 65%) as a red solid. ¹H NMR (300 MHz, CD₃OD) δ (ppm): 1.40-1.87 (m, 88H); 2.49-2.56 (m, 18H); 2.68-2.72 (m, 2H); 3.08 (s, 3H); 3.17-3.30 (m 30H); 3.37-3.51 (m, 10H); 3.41 (s, 24H); 3.59-4.01 (m, 816H); 4.25-4.40 (m, 8H); 7.29-7.33 (m, 2H); 8.44-8.49 (m, 2H); LCMS (philic method, TFA buffer) Rₜ=10.28 min.

*[The units were pre-rinsed with H₂O (5 mL) and spinning at 4000 rpm for 5 min. This process was repeated. The crude solution was filtered through 0.45 μm syringe filter then placed in the centrifugation units. The units were then spun at 4000 rpm for 15 min. The retentate was then diluted with H₂O (4 mL) then spun again at 4000 rpm for 15 min. This process was repeated 8 times.]

### 1b.73 Nanobody-N₃/DBCO-DFO Compound 114

Solution of DBCO-DFO Compound 56 is prepared by dissolving 0.4 mg of compound in 200 μL of DMSO, 124 μL of DBCO-DFO solution (48 μg, 0.054 μmol) added to the Nanobody-N₃ (“Nanobody-N3-C-terminal Tag”) solution (228 μg; 0.015 μmol) in Tris buffer (pH 8) and the solution left at 4° C. overnight. UPLC analysis of the reaction mixture showed unreacted azido nanobody suggesting that the reaction is not completed. Another 24 μg (0.027 μmol) of the DBCO-DFO compound added to the reaction mixture and the reaction mixture left at 4° C. over the weekend. UPLC analysis of the reaction mixture showed that the reaction is completed. UPLC: 30 to 40% MeCN with 0.01 TFA over 15 mins, Rₜ: 9.91 mins for azido Nanobody-N₃ with a m/z 14308 and Rₜ: 10.79 for NB-DFO product with a m/z 15184.

Sample diluted to 1 mL using Tris buffer (pH 8) and then purified using spin column (Amicon 0.5 mL; 10 kDa cut off, Tris as a mobile phase; 10 washes (450 μL/wash). The NB-DFO product 114 obtained with a concentration of ˜250 μg/300 μL in Tris Buffer (pH 8).

1b. 74 BHALys[Lys]₂[Lys]₄[Lys]₈[(α-NHBoc)₈(ε-NH-COPEG₂₄-NHFmoc)₈], G3 Compound 115

To a solution of BHALys[Lys]₂[Lys]₄[Lys]₈[α-NHBoc)₈(ε-NH₂)s] (Ref.1) (100 mg, 0.0344 mmol) in DMF (2 mL) was added Fmoc-NH-PEG₂₄-COOH (451 mg, 0.329 mmol), PyBOP (171 mg, 0.329 mmol) and NMM (90 μL, 0.0826 mmol) and the reaction was stirred overnight at RT. The reaction progress was checked by HPLC, and once deemed complete was concentrated in vacuo and the product Compound 115 was used in the next step without further purification.

1b. 75 BHALys[Lys]₂[Lys]₄[Lys]₈[(α-NHBoc)₈(ε-NH-COPEG₂₄-NH₂)₈], G3 Compound 116

To a solution of BHALys[Lys]₂[Lys]₄[Lys]₈[(α-NHBoc)₈(F—NH-COPEG₂₄-NHFmoc)₈]Compound 115 (470 mg, 0.0343 mmol, treating as 100% yield from prior step) in DMA (4 mL) was added piperidine (1 mL) and the reaction was monitored by HPLC analysis. Upon consumption of the starting material the reaction was concentrated under reduced pressure and purified by SEC (400 drops/tube, MeOH sephadex LH20, 35 drops/min). Fractions were checked by TLC analysis (5% BaCl₂ solution followed by iodine stain; dark brown spot) followed by HPLC, and those containing the product were combined and concentrated under reduced pressure. The residue was taken up in MQ water filtered (0.45 μm acrodisc filter) and freeze dried to yield Compound 116 as a light brown film (190 mg, 46% yield over two steps). ¹H NMR (300 MHz, CD₃OD) δ (ppm): 0.9-1.93 (m, 162H), 2.44 (m, 16H), 2.93-3.24 (m, 46H), 3.40 (m, 7H), 3.48-3.78 (m, 800H), 3.87 (m, 6H), 4.00 (m, 9H), 4.3 (m, 7H), 4.45 (m, 1H), 6.19 (s, 1H), 7.23-7.37 (m, 10H). HPLC (C8 XBridge, 3×100 mm) gradient: 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=7.9-8.1 min (broad peak)

1b.76 ((1R,8S,9s)-bicyclo[6.1.0]non-4-yn-9-yl)methyl (2-(3-(2,5-dioxo-2,5-dihydro-1H-pyrrol-1-yl)propanamido)ethyl)carbamate Compound 117

To a solution of MPS-EDA.TFA (0.204 g, 0.627 mmol, from Quanta BioDesign) and BCN-NHS (0.224 g, 0.769 mmol, from SynAffix) in DMF (3.0 mL) was added NMM (0.210 mL, 1.91 mmol). The reaction was allowed to stir at room temperature for 2 h then concentrated in vacuo. The residue was purified by silica chromatography (100% EtOAc, 1% Et₃N) to obtain product (0.147 g, 61%). ¹H NMR (300 MHz, d₆-DMSO) δ (ppm): 0.85-0.87 (m, 2H); 1.23-1.28 (m, 2H); 1.49-1.51 (m, 2H); 2.15-2.32 (m, 8H); 2.98-3.02 (m, 4H); 3.57-3.61 (m 2H);); 4.01-4.03 (m 2H); 6.98 (brs, 2H); 7.05 (brs, 1H); 7.97 (brs, 1H).

1b. 77 Nanobody (plain with purification TAG) GGSHHHHHHGMASMTGGQQMGRDLYENLYFQGEVQLVESGGSLVQPGGSLRLSC AASGFTFDDYAMSWVRQVPGKGLEWVSSINWSGTHTDYADSVKGRFTISRNNANN TLYLQMNSLKSEDTAVYYCAKNWRDAGTTWFEKSGSAGQGTQVTVSS Compound 118
1b.78 (MeTzPh)-PEG₄-PEG₂₄-CO[N(PN)₂][Lys]₂[Lys]₄[Lys]₈[NH₂·HCl]₁₆ G3 Compound 119

To a stirred solution of (MeTzPh)-PEG₄-PEG₂₄-CO[N(PN)₂][Lys]₂[Lys]₄[NH₂·HCl]s Compound 112 (0.098 g, 0.0358 mmol) and DBL-OPNP (Ref.1) (0.207 g, 0.443 mmol) in DMF (2.0 mL) under an atmosphere of N₂ was added NMM (190 μL, 1.973 mmol). The ensuing reaction mixture was then left to stir overnight at room temperature. The volatiles were removed in vacuo and the resulting oily residue was purified on silica chromatography (5%-10%-15% MeOH:dichloromethane) to give the Boc protected product as a red oil (0.128 g, 70%). The purified material was cooled in ice/water bath then a solution of 1.25 M HCl/MeOH (6.5 mL, 8.5 mmol) was slowly added. After 5 min, the ice-bath was removed and the ensuing reaction mixture left to stir at room temperature overnight. The volatiles were removed in vacuo to give the product Compound 119 as a red oil (0.107 g, 100%). LCMS (philic method, TFA buffer) Rₜ=7.48 min, ESI MS (+ve) 3471 [M+H]⁺; calc. m/z [M+H]⁺=3472.

1b.79 (MeTzPh)-PEG₄-PEG₂₄-CO[N(PN)₂][Lys]₂[Lys]₄[Lys]₈[Lys]₁₆[(α-NH₂·HCl)(ε-NH—CO PEG₁₁₀₀)]₁₆ G4 Compound 120

To a stirred solution of HO-Lys(Boc)(PEG₁₁₀₀) (Ref. 1) (0.107 g, 0.132 mmol) and PyBOP (0.065 g, 0.125 mmol) in DMF (1.5 mL) under an atmosphere of N₂ was added NMM (50 μL, 0.455 mmol). After 10 min, that solution was added to (MeTzPh)-PEG₄-PEG₂₄-CO[N(PN)₂][Lys]₂[Lys]₄[Lys]₈[NH₂·HCl]₁₆ Compound 119 (0.0221 g, 0.00545 mmol). The ensuing reaction mixture was left to stir overnight at room temperature. The volatiles were removed in vacuo and the resulting crude was cooled in ice/water bath. To the cooled residue, 1.25 M HCl/MeOH (1.5 mL, 1.88 mmol) was slowly added. After 10 min, the cold bath was removed and the reaction was left stirring at room temperature overnight. The volatiles were removed in vacuo then dissolved in H₂O (5 mL). The solution was purified by centrifugation using Millipore Amicon Ultra-15 Centrifugal Filter Unit (10 kDa MWCO). The retentate was freeze-dried overnight and further purified by preparative HPLC on a Gilson instrument [Single Gradient 60 min, 10>60% MeCN (0.1% TFA buffer, RT 36 min] to give product Compound 120 (0.021 g, 25%) as red solid. ¹H NMR (300 MHz, CD₃OD) δ (ppm): 1.28-1.87 (m, 184H); 2.42-249 (m, 35H); 3.00 (s, 3H); 3.12-3.23 (m 60H); 3.34-3.40 (m, 63H); 3.52-4.01 (m, 826H); 4.25-4.39 (m, 16H); 7.17-7.20 (m, 2H); 8.48-8.51 (m, 2H); LCMS (philic method, TFA buffer) Rₜ=8.96 min. ESI MS (+ve) 2095 [M+7H]⁺; 1834 [M+8H]⁺; 1630 [M+9H]⁺.

1b.80 (MeTzPh)-PEG₄-PEG₂₄-CO[N(PN)₂][Lys]₂[Lys]₄[Lys]₈[Lys]₁₆[NH₂.TFA]₃₂ G4 Compound 121

Prepared as per compound 119 and deprotected using TFA/AcOH to give Compound 121 (0.078 g, 78%) as a red solid. LCMS (philic method, TFA buffer) RT=7.74 min. ESI MS (+ve) 2095 [M+7H]⁺; 1834 [M+8H]⁺; 1630 [M+9H]⁺.

1b.81 (MeTzPh)-PEG₄-PEG₂₄-CO[N(PN)₂][Lys]₂[Lys]₄[Lys]₈[Lys]₁₆[Lys]₃₂[(α-NH₂.TFA)(ε-NH-COPEG₁₁₀₀)]₃₂ G5 Compound 122

To a stirred solution of HO-Lys(Boc)(PEG₁₁₀₀) (Ref.1) (0.476 g, 0.354 mmol) and PyBOP (0.170 g, 0.327 mmol) in DMF (3.0 mL) under an atmosphere of N₂ was added NMM (180 μL, 0.500 mmol). After 10 min, that solution was added to (MeTzPh)-PEG₄-PEG₂₄—CO[N(PN)₂] [Lys]₂[Lys]₄[Lys]₈[Lys]₁₆[NH₂.TFA]₃₂ Compound 121 (0.078 g, 0.00850 mmol). The ensuing reaction mixture was left to stir overnight at room temperature. The volatiles were removed in vacuo and the resulting crude was dissolved in H₂O (1.0 mL) and TFA (1.0 mL) was slowly added. The reaction was left stirring at room temperature overnight. The reaction was diluted with H₂O (12 mL) and purified by centrifugation using Millipore Amicon Ultra-15 Centrifugal Filter Unit (10 kDa MWCO). The retentate was freeze-dried overnight to give product Compound 122 (0.374 g, 91%) as pink solid. ¹H NMR (300 MHz, D₂O) δ (ppm): 1.41-1.87 (m, 376H); 2.52-2.56 (m, 64H); 3.09 (s, 3H); 3.17-3.24 (m, 126H); 3.41 (s, 93H) 3.48-3.98 (m, 2960H); 4.27-4.39 (m, 32H); 7.30-7.33 (m, 2H); 8.46-8.49 (m, 2H); HPLC (HPLC-Philic method, formate buffer, 15 min) Rₜ=8.60 min.

1b.82 BHALys[Lys]₂[Lys]₄[Lys]₈[Lys]₁₆[Lys]₃₂[(α-NHAc)₃₀(α-NHDOTA)₂(ε-NHCOPEG₂₀₀₀)₃₂] Compound RH-3

To a stirred solution of BHALys[Lys]₂[Lys]₄[Lys][Lys]₁[Lys]₃₂[(α-NH₂)₃₀(α-NHDOTA)₂(ε-NHCOPEG₂₀₀₀)₃₂] Compound RH-2 (63 mg, 860 μmol) in DMF (0.6 mL) was added TEA (25 μL, 228 μmol), followed by acetic anhydride (41 μL, 430 μmol). The ensuing reaction mixture was stirred at ambient temperature overnight. The reaction mixture was concentrated in vacuo, dissolved in MeOH (1.0 mL) and purified by SEC. The product-containing fractions were combined and concentrated in vacuo, and the resulting residue dissolved in MQ water, filtered (0.45 μm acrodisc filter) and lyophilised The resulting solid was dissolved in MQ water (50 mL) and purified by ultrafiltration (minimate) using MQ water. After collecting 11 DV of permeate, the retentate was concentrated, filtered (0.22 μm acrodisc filter) and lyophilised to give Compound RH-3-160 (52.2 mg). HPLC (HPLC-Philic method, formate buffer, 15 min) Rₜ=8.56 min. ¹H NMR (300 MHz, D₂O) δ (ppm): 1.16-1.90 (m, 359H), 2.02 (broad s, 101H), 3.01-3.31 (m, 133H), 3.38 (s, 96H), 3.45-3.48 (m, 43H), 3.52-4.40 (m, 5267H), 6.09 (broad s, 1H), 7.13-7.54 (m, 17H). ¹H NMR analysis suggests approx. 1.8 DOTA/dendrimer; % (w/w) of DOTA=1.7%.

1c. Synthesis of Nanobody Controls
1c.1 Nanobody-N₃/DBCO-Sulfo-PEG₃-DFO, Compound 82

A solution of Nanobody-N₃ (“Nanobody-N3-C-terminal Tag”) (5.78 mg in 2 ml of 20 mM Tris, pH8) was reacted with DBCO-sulfo-PEG₄-DFO Compound 55 (535 μl of a 1 mg/ml solution in water, 1 eq.). The ensuing reaction mixture was left to stand at RT overnight. Excess unreacted DBCO-sulfo-PEG₃-DFO was removed by buffer exchange, performed using 10 k MWCO Amicon Ultra centrifugal filters. The nanobody-dendrimer conjugate was then buffer exchanged into 10 mM HEPES, pH 8 for radioactive labelling. The removal of unreacted DBCO-sulfo-PEG₃-DFO was confirmed by HPLC.

### 1c.2 Nanobody-Cys/MAL-Cy5 SRS-19

Nanobody-Cys SRS-13 was conjugated to sulfo-Cyanine5 maleimide (Lumiprobe catalogue 13380) following reduction with TCEP. 10 Molar equivalents of TCEP was added to Nanobody-Cys SRS-13 in pH 7.2 10 mM PBS. The reaction tube was flushed with nitrogen and the reaction mixture was left for 2 h at rt. The excess TCEP was removed by desalting using a 7 k MWCO Zebaspin desalting column (Thermo Fisher catalogue 89882). 5 Molar equivalents of sulfo-Cyanine5 maleimide was then added. The reaction tube was flushed with nitrogen and the reaction mixture was left for 20 h at rt. Any unreacted sulfo-Cyanine5 maleimide was removed using a 7 k MWCO Zebaspin desalting column.

1d. Synthesis of Trastuzumab Controls

### 1d.1 Trastuzumab-Deglycosylated Compound KY-1

A solution of Herceptin© (Trastuzumab) for SC injection (200 μL of 120 mg/mL herceptin) was buffer exchanged via 7K Zeba™ Spin Desalting Column, 0.5 mL (Thermo Scientific™) into 10 mM PBS, pH 7.4. The concentration was adjusted to 10 mg/mL with 10 mM PBS. PNGase F (New England Biolabs, 3.5 μL) was added to the solution and incubated at 37° C. with shaking (400 rpm) and the reaction was monitored by UPLC. For the reduction of KY-1, β-mercaptoethanol, 10% v/v (Sigma-Aldrich) was used. UPLC-ToF analysis of 0-mercaptoethanol reduced product (Method 1): Heavy chain: 9.06 mins (Rₜ) with m/z 49153 (m/z 50593 before reaction); Light chain: 8.55 (Rₜ) with m/z 23439 (m/z 25377 before reaction). After 22 h, the PNGase F was removed using centrifugation (Amicon Ultra-0.5, 50 kDa MWCO), samples were run at 5,000× g for 5 minutes for a total of 4 washes.

1d.2 Trastuzumab-{CONH-PEG₄-Sulfo-DBCO}₂ Compound KY-2 (and KY-2a)

To a solution of deglycosylated Trastuzumab KY-1 in PBS buffer pH 7.4 (2 mL, 10 mg/mL) was added a solution of sulfo DBCO-PEG₄-amine (Click Chemistry Tools, 7.2 mg, 0.01 mmol) in MQ water (240 μL) at rt. The reaction mixture was heated to 37° C. followed by the addition of transglutaminase (Microbial-Transglutaminase, [MTGase], Zedira GmbH), resuspended in MQ water (300 μL). The final concentration is 6.7 units of MTGase per 1 mg of KY-1. Sample was incubated at 37° C. for 12-18 hours or until completion of the modification (Reaction monitored via UPLC-T of (Method 1). An increase in heavy chain mass of KY-1 can be observed by UPLC analysis with a peak at 9.62 mins (Rₜ) with m/z at 49,885 Da (m/z 49153 before reaction). Upon completion, MTGase and unreacted linker were removed with Superdex® 200 Increase 10/300 GL (Cytiva) using size exclusion column (SEC) buffer 10 mM HEPES, 150 mM NaCl, pH 8. Purification was monitored based at 280 nm absorbance. Peaks of interest were collected and concentrated with 50 kDa MWCO Amicon Ultra-0.5 centrifugal filter unit (Merck) to 1 mL (16.43 mg/mL in 10 mM HEPES buffer). Evidence of trans glutamation was confirmed by treating an aliquot of KY-2 with AFDye 647 azide (Click Chemistry Tools) to give KY-2a and by subsequent analysis using SDS page gel (see FIG. 21, lanes 1 and 3, and reduced product lanes 2 and 4). The corresponding cell binding data is shown in Example 7.

1d.3 Trastuzumab-{PEG₄-DBCO N₃-PEG₇-Lys[(α-Cy5)₁(ε-NHDOTA)₁]}2 Compound KY-3-310

To a solution of Trastuzumab-{CONH-PEG₄-Sulfo-DBCO}₂ KY-2 (16 mg in 1.2 mL Tris Buffer pH 8) was added N₃-PEG₇-NHCO-Lys[(α-NHCy5)(ε-NHDOTA)] Compound SRS-5 (0.65 mg, 0.42 μmol, 10 mg/mL) in DMSO. The reaction mixture was left at rt for 18 h and then diluted with MQ water to a volume of 15 mL. The resulting solution was concentrated by centrifugation (Amicon Ultra-15, 10 kDa MWCO) and washed with Tris Buffer pH 8 (5×15 mL) to give a solution of Trastuzumab-{PEG₄-DBCO/N₃-PEG₇-Lys[(α-Cy5)₁(F-NHDOTA)₁]}₂ (Compound KY-3-310) in Tris buffer pH 8 (16.3 mg/mL). UPLC-Tof analysis of β-mercaptoethanol reduction products (Method 1): Heavy chain: 10.19 mins (Rₜ) with m z 51345 (m/z 49885 before reaction); Light chain: 8.72 (Rₜ) with m/z 23439 (m/z 23439 before reaction).

1e. Synthesis of FAPI Intermediates

### 1e.1 FAPI-04-NH RHa-2

To a solution of FAPI-04-NBoc RHa-1 (425 mg, 0.72 mmol) in MeCN (10 mL) was added p-toluene sulfonic acid (206 mg, 1.09 mmol). The resulting cloudy mixture was stirred at 50-60° C. and the progress of the reaction was monitored by LCMS (LCMS Method 20-90, 8 min, TFA). The reaction mixture was heated for a total of 31.5 h with additional portions of p-toluene sulfonic acid (144 mg, 0.72 mmol) added at the 3.5 h and 30 h time points. The reaction mixture was cooled to rt, concentrated in vacuo to give FAPI-04-NH RHa-2. The material was used without further purification. LCMS (LCMS Method 20-90, 8 min, TFA) Rₜ=0.661 min and 3.36 min. ESI MS (+ve) 487 [M]⁺; calc. m/z for C₂₄H₂₈F₂N₆O₃ [M]⁺=487. ¹The majority of product appears to elute directly from the column upon injection (Rₜ=0.66 min) using this method but is also retained (Rₜ=3.36 min).

1e.2 FAPI-04-NC(O)-dPEG₁₁₀₀-CO₂HRHa-4

To a stirred solution of FAPI-04-NH RHa-2 (0.72 mmol) in DMF (10 mL) was added acid-dPEG₁₁₀₀-NHS ester RHa-3 (795 mg, 0.60 mmol) at rt. Once all the solids had dissolved, NMM (1.3 mL, 11.15 mmol) was added and the reaction mixture and stirring continued. After 18 h, the reaction mixture was concentrated in vacuo. The residue was dissolved in MeCN (8 mL), filtered (0.45 μm acrodisc syringe filter) and the filtrate was purified by preparative HPLC (Prep-HPLC Method 20-60, TFA; Rₜ=26-29 min) to give FAPI-04-NC(O)-dPEG₁₁₀₀-CO₂H RHa-4 as a pale-yellow oil (1.1 g, 99%). ¹H NMR (300 MHz, CD₃OD): δ (ppm) 2.33-2.47 (m, 2H), 2.55 (t, J=9.0 Hz, 2H), 2.62-3.22 (m, 4H), 3.34-3.54 (m, 4H), 3.56-3.68 (m, 100H), 3.68-3.91 (m, 5H), 3.92-4.57 (m, 7H), 5.12-5.16 (m, 1H), 7.72 (dd, J=3.0, 9.0 Hz, 1H), 7.90 (d, J=5.1 Hz, 1H), 8.15-8.26 (m, 2H) and 9.02 (d, J=6 Hz, 1H). LCMS (LCMS-Method 5-60, 8 min TFA) Rₜ=5.22 min. ESI MS (+ve) 1688 [M]⁺; calc. m z for C₇₈H₁₃₂F₂N₆O₃₁ [M]⁺=1688.

### 1e.3 HO-Lys[(α-NHCy5)(ε-NH₂.TFA)] RHa-7

To a solution of Cy5-NHS ester RHa-5 (51 mg, 0.077 mmol) in DMF (2.0 mL) at rt was added HO-Lys[(α-NH₂)(ε-NHBoc)] RHa-6 (38 mg, 0.154 mmol) followed by NMM (70 μL, 0.154 mmol). The reaction mixture was sonicated for 5 min and then stirred for 2 d whereupon the volatiles were removed in vacuo. The residue was dissolved in dichloromethane (2.0 mL) and TFA (0.5 mL) added at rt. The reaction mixture was stirred for 4.5 h, diluted with MeCN (˜5 mL), concentrated in vacuo and purified by preparative HPLC (Prep-HPLC Method 30-90, TFA; Rₜ=24-26 min) to give HO-Lys[(α-NHCy5)(ε-NH₂.TFA)] RHa-7 as a dark blue solid (46 mg, 82%). LCMS (LCMS Method 20-90, 8 min, TFA) Rₜ=5.09 min. ESI MS (+ve) 612 [M]⁺; calc. m/z for C₃₈H₅₁N₄O₃ [M]⁺=612.

### 1e.4 HO-Lys[(α-NHCy5)(ε-NH-Ph-DFO)] RHa-9

To a HO-Lys[(α-NHCy5)(ε-NH₂.TFA)] RHa-7 (93 mg, 0.13 mmol) at rt was added a solution of p-SCN-Ph-DFO RHa-8 (120 mg, 0.16 mmol) in DMSO (2.0 mL) and DIPEA (150 μL, 0.86 mmol). The resulting solution was stirred for 18 h and then concentrated to approximately half the volume by blowing nitrogen over the surface of the reaction mixture (16 h). Water (˜20 mL) was added to the concentrated solution followed by MeCN (˜2 mL) to dissolve any solids. The resultant solution was lyophilised to dryness and then purified by automated flash chromatography (Autoflash-Method 1, R_{CV}=11-13 CV) to give HO-Lys[(α-NHCy5)(F—NH-Ph-DFO)] RHa-9 as a dark blue solid (108 mg, 57%). LCMS (LCMS Method 5-60, 8 min, TFA): Rₜ=6.73 min. ESI MS (+ve) 1365 [M]⁺; calc. m/z for C₇₁H₁₀₃N₁₂O₁₁S₂ [M]⁺=1365.

1e.5 BHALys[Lys]₂[Lys]₄[Lys]₈[Lys]₁₆[Lys]₃₂[(α-NHBoc)₃₂(ε-NHCOPEG₁₁₀₀-C(O)N-FAPI-04)₁(ε-NHCO-mPEG₁₀₀₀)₃₁] RHa-12

To a stirred solution of FAPI-04-NC(O)-PEGn₁₁₀₀-CO₂H RHa-4 (100 mg, 0.06 mmol) and mPEG₁₀₀₀-CO₂H RHa-11 in DMF (5 mL) was added PyBOP (200 mg, 0.38 mmol) at rt. The reaction mixture was stirred for 5 min whereupon a solution of BHALys[Lys]₂[Lys]₄[Lys]₈[Lys]₁₆[Lys]₃₂[(α-NHBoc)₃₂(ε-NH₂)₃₂] RHa-10 (110 mg, 0.01 mmol) and NMM (150 μL, 1.35 mmol) in DMF (3 mL). The resulting solution was stirred for 2 d and concentrated in vacuo. Water (20 mL) was added to the residue and the resulting cloudy solution was filtered. The filtrate was diluted with water to a total volume of ˜ 70 mL and subjected to TFF (Pellicon XL cassette, 50 cm², 10 kDa MWCO Ultracel membrane) eluting with deionised water (10 DV). The retentate was further concentrated by spin column (Amicon Ultra, 10 kDa MWCO, 15 mL) and lyophilised to give BHALys[Lys]₂[Lys]₄[Lys]₈[Lys]₁₆[Lys]₃₂[(α-NHBoc)₃₂(ε-NHCO-dPEG₁₁₀₀-C(O)N-FAPI-04)₁(ε-NHCO-mPEG₁₀₀₀)₃₁] RHa-12 as a pale-yellow gum (317 mg). The number (n_{FAPI}) of ε-NHCO-dPEG₁₁₀₀-C(O)N-FAPI-04 groups and the number of ε-NHCO-mPEG₁₀₀₀ groups (n_{mPEG}) was determined using ¹H NMR spectroscopy.

BHALys[Lys]₂[Lys]₄[Lys]₈[Lys]₁₆[Lys]₃₂[(α-NHBoc)₃₂(ε-NHCO-dPEG₁₁₀₀-C(O)N-FAPI-04)₁(ε-NHCO-mPEG₁₀₀₀)₃₁] RHa-12 (25 mg) was dissolved in CD₃OD (600 μL) and the resulting solution was shaken and allowed to stand at rt for 24 h. The ¹H NMR spectrum was recorded [number of scans=500, dl (pulse delay)=30 s], processed and the integral of the resonances associated with the benzhydryl (BHA) protons at δ=7.16-7.44 ppm was set to 10H. The integral value of the resonances at δ=8.70-8.86 ppm was attributed to the (1H) x n_{FAPI} quinoline proton and the resonance at δ=3.36 ppm was attributed to (3H)×n_{mPEG} protons. Indoing so, n_{FAPI}=1 and n_{mPEG}=31. HPLC (HPLC Method, 5-80, 8 min, TFA) Rₜ=5.06 min. ¹H NMR (300 MHz, CD₃OD): δ (ppm): 0.73-2.15 (m, 649H), 2.90-3.29 (m, 100 H), 3.36 (s, 93H), 3.37-3.92 (m, 2991H), 3.93-4.66 (m, 103H), 6.16-6.28 (m, 1H), 7.16-7.44 (m, 10H), 7.78-8.08 (m, 26H) and 8.70-8.86 (m, 1H).

1e.6 BHALys[Lys]₂[Lys]₄[Lys]₈[Lys]₁₆[Lys]₃₂[(α-NHBoc)₃₂(ε-NHCOPEG₁₁₀₀-C(O)N-FAPI-04)₁₀(ε-NHCO-mPEG₁₀₀₀)₁₉(ε-NH₂)₃] RHa-13

Synthesis as described for BHALys[Lys]₂[Lys]₄[Lys]₈[Lys]₁₆[Lys]₃₂[(α-NHBoc)₃₂(ε-NHCO-dPEG₁₁₀₀-C(O)N-FAPI-04)₁(ε-NHCO-mPEG₁₀₀₀)₃₁] RHa-12 except that FAPI-04-NC(O)-PEG₁₁₀₀-CO₂H RHa-4 (355 mg, 0.21 mmol), mPEG₁₀₀₀-CO₂H RHa-11 (218 mg, 0.42 mmol), PyBOP (218 mg, 0.42 mmol), BHALys[Lys]₂[Lys]₄[Lys]₈[Lys]₁₆[Lys]₃₂[(α-NHBoc)₃₂(ε-NH₂)₃₂] RHa-10 (125 mg, 0.011 mmol) and NMM (192 μL, 1.75 mmol) in DMF (5+3 mL) were used to give BHALys[Lys]₂[Lys]₄[Lys]₈[Lys]₁₆[Lys]₃₂[(α-NHBoc)₃₂(ε-NHCO-dPEG₁₁₀₀-C(O)N-FAPI-04)₁₀(ε-NHCO-mPEG₁₀₀₀)₁₉(ε-NH₂)₃] RHa-13 as a thick yellow gum (460 mg, 85%, MW_{Calc}=49,214 Da). HPLC (HPLC Method, 5-80, 8 min, TFA) Rₜ=4.83 min. ¹H NMR (300 MHz, CD₃OD+D₂O): δ (ppm): 1.01-2.01 (m, 681H), 2.14-2.29 (m, 16H), 2.39-2.52 (m, 19H), 2.59-2.77 (m, 17H), 2.77-3.29 (m, 168H), 3.36 (s, 71H), 3.38-3.92 (m, 3239H), 3.92-4.48 (m, 149H), 5.03-5.21 (m, 10H), 7.12-7.38 (m, 10H), 7.44-7.56 (m, 10H), 7.56-7.64 (m, 9H), 7.79-8.20 (m, 88H) and 8.73-8.86 (m, 10H). n_{FAPI}=10 and n_{mPEG}=19.

1e.7 BHALys[Lys]₂[Lys]₄[Lys]₈[Lys]₁₆[Lys]₃₂[(α-NHBoc)₃₂(ε-NHCO-dPEG₁₁₀₀-C(O)N-FAPI-04)₄(ε-NHCO-dPEG₁₁₀₀)₂s] RHa-14

To a stirred solution of FAPI-04-NC(O)-PEG₁₁₀₀-CO₂H RHa-4 (53 mg, 31.4 μmol) in DMF (3.5 mL) at rt was added PyBOP (16.3 mg, 31.4 μmol). The reaction mixture was stirred for 5 min whereupon BHALys[Lys]₂[Lys]₄[Lys]₈[Lys]₁₆[Lys]₃₂[(α-NHBoc)₃₂(ε-NH₂)₃₂] RHa-10 (75 mg, 6.5 μmol) followed by NMM (20 μL, 18.2 μmol). The resulting solution was stirred for 18 h whereupon a premixed solution of dPEG₁₁₀₀-CO₂H RHa-32 (255 mg, 0.22 mmol), PyBOP (114 mg, 0.22 mmol) and NMM (121 μL, 1.10 mmol) in DMF (1.7 mL) was added and stirring continued. After 18 h, the volatiles were concentrated in vacuo and the residue was dissolved in deionised water (20 mL). The resulting solution was distributed evenly into 4× Amicon® Ultra 15, 10 kDa MWCO spin columns and concentrated by centrifugation (4000 rpm for 20 min). The retentate was washed with water (10×3 mL @ 4000 rpm for 10 min) and the final retentate then lyophilised to give a light-yellow solid (277 mg, 84%, MW_{Calc}=50,156 Da). HPLC (HPLC Method, 5-80, 8 min, TFA) Rₜ=4.98 min. ¹H NMR (300 MHz, CD₃OD): δ (ppm): 1.00-2.03 (m, 698H), 2.25-2.57 (m, 70H), 2.61-2.81 (m, 6H), 2.98-3.27 (m, 115H), 3.36 (s, 90H), 3.37-3.92 (m, 3361H), 3.92-4.16 (m, 25H), 4.16-4.50 (m, 46H), 5.05-5.20 (m, 4H), 7.15-7.41 (m, 10H), 7.45-7.54 (m, 4H), 7.56-7.63 (m, 4H), 7.73-8.19 (m, 33H), 8.73-8.86 (m, 4H); n_{FAPI}=4 and n_{mPEG}=28.

1e. 8 BHALys[Lys]₂[Lys]₄[Lys]₈[(α-NHBoc)₈(ε-NHCO-dPEG₁₁₀₀-C(O)N-FAPI-04)₁(ε-NHCO-mPEG₁₀₀₀)₈(ε-NH₂)₂] RHa-16

Synthesis as described for BHALys[Lys]₂[Lys]₄[Lys]₈[Lys]₁₆[Lys]₃₂[(α-NHBoc)₃₂(ε-NHCO-dPEG₁₁₀₀-C(O)N-FAPI-04)₁(ε-NHCO-mPEG₁₀₀₀)₃₁] RHa-12 except that FAPI-04-NC(O)-PEG₁₁₀₀-CO₂H RHa-4 (627 mg, 0.37 mmol), mPEG₁₀₀₀-CO₂H RHa-11 (450 mg, 0.37 mmol), BHALys[Lys]₂[Lys]₄[Lys]₈[(α-NHBoc)₈(ε-NH₂)₈] RHa-15 (225 mg, 0.077 mmol), PyBOP (387 mg, 0.74 mmol) and NMM (340 μL, 3.10 mmol) in DMF (10 mL) were used to synthesise BHALys[Lys]₂[Lys]₄[Lys]₈[(α-NHBoc)₈(ε-NHCO-dPEG₁₁₀₀-C(O)N-FAPI-04)₁(g-NHCO-mPEG₁₀₀₀)₈(ε-NH₂)₂] RHa-16 as a yellow gum (220 mg, 25%, MW_{Calc}=11,225 Da). HPLC (HPLC Method, 5-80, 8 min, TFA) Rₜ=5.08 min. ¹H NMR (300 MHz, CD₃OD+D₂O): δ (ppm): 1.03-1.99 (m, 165H), 2.20-2.38 (m, 1H), 2.38-2.52 (m, 2H), 2.63-2.76 (m, 1H), 2.92-3.29 (m, 33H), 3.36 (s, 19H), 3.38-3.92 (m, 721H), 3.93-4.11 (m, 18H), 4.11-4.42 (m, 9H), 5.07-5.20 (m, 1H), 6.14-6.25 (m, 1H), 7.20-7.40 (m, 10H), 7.44-7.54 (m, 1H), 7.56-7.64 (m, 1H), 7.77-8.21 (m, 18H) and 8.75-8.82 (m, 11H). n_{FAPI}=1 and n_{mPEG}=5.

1e.9 BHALys[Lys]₂[Lys]₄[Lys]₈[(α-NHBoc)₈(ε-NHCO-dPEG₁₁₀₀-C(O)N-FAPI-04)₃(ε-NHCO-mPEG₁₀₀₀)₅] RHa-17

The permeate from the TFF process used to purify BHALys[Lys]₂[Lys]₄[Lys]₈[(α-NHBoc)₈(ε-NHCO-dPEG₁₁₀₀-C(O)N-FAPI-04)₁(ε-NHCO-mPEG₁₀₀₀)₈(ε-NH₂)₂] RHa-16 was concentrated in vacuo, dissolved in deionised water (24 mL) and filtered using Amicon @Ultra 15 Centrifugation 3 kDa MWCO spin columns (4000 rpm for 30 min). The retentate was washed with deionised water (10×15 mL @ 4000 rpm for 15 min per cycle) and lyophilised to give BHALys[Lys]₂[Lys]₄[Lys]₈[(α-NHBoc)₈(ε-NHCO-dPEG₁₁₀₀-C(O)N-FAPI-04)₃(ε-NHCO-mPEG₁₀₀₀)s] RHa-17 as a yellow gum (670 mg, 64%, MW_{Calc}=13,457 Da). HPLC (HPLC Method, 5-80, 8 min, TFA) Rₜ=5.08 min. ¹H NMR (300 MHz, CD₃OD+D₂O): δ (ppm): 0.82-2.05 (m, 170H), 2.22-2.54 (m, 9H), 2.64-2.77 (m, 4H), 3.01-3.29 (m, 34H), 3.36 (s, 18H), 3.38-3.82 (m, 764H), 3.82-4.10 (m, 18H), 4.10-4.52 (m, 19H), 5.07-5.19 (m, 2H), 7.15-7.42 (m, 10H), 7.42-7.70 (m, 5H), 7.81-8.12 (m, 8H) and 8.72-8.87 (m, 3H). n_{FAPI}=3 and n_{mPEG}=5.

1e.10 BHALys[Lys]₂[Lys]₄[Lys]₈[(α-NHBoc)₈(ε-NHCO-dPEG₁₁₀₀-C(O)N-FAPI-04)_{7.4}(ε-NH₂)_{0.6}] RHa-18

To a solution of FAPI-04-NC(O)-PEG₁₁₀₀-CO₂H RHa-4 (77 mg, 0.05 mmol) in DMF (2.0 mL) was added PyBOP (23 mg, 0.050 mmol) at rt. The reaction mixture was stirred for 5 min whereupon BHALys[Lys]₂[Lys]₄[Lys]₈[(α-NHBoc)₈(ε-NH₂)s] RHa-15 (13 mg, 0.005 mmol) and NMM (21 μL, 0.19 mmol) were added. After 18 h, the volatiles were removed in vacuo and the residue was purified by SEC (stationary phase=Sephadex LH-20, mobile phase=MeCN, h=30 cm, dia=2.5 mm, drop rate ˜1 drop/second, fraction size=400 drops, fractions analysed by HPLC (HPLC-Method 1)). Fractions containing the purified product were pooled and concentrated in vacuo. The residue was dissolved in deionised water, filtered (0.45 μm acrodisc syringe filter) and lyophilised to give BHALys[Lys]₂[Lys]₄[Lys]₈[(α-NHBoc)₈(ε-NHCO-dPEG₁₁₀₀-C(O)N-FAPI-04)_{7.4}(ε-NH₂)_{0.6}] RHa-18 (20 mg). Impure fractions were pooled separately and resubjected to SEC as described above (except the mobile phase=MeOH) to give BHALys[Lys]₂[Lys]₄[Lys]₈[(α-NHBoc)₈(ε-NHCO-dPEG₁₁₀₀-C(O)N-FAPI-04)_{7.4}(ε-NH₂)_{0.6}] RHa-18 as a gummy solid (47 mg, combined yield=67 mg, 98%, MW_{Calc}=15,265 Da). HPLC (HPLC Method, 5-80, 8 min, TFA) Rₜ=4.62 min. ¹H NMR (300 MHz, CD₃OD+D₂O): δ (ppm): 1.15-1.94 (m, 171H), 1.97-2.21 (m, 14H), 2.36-2.74 (m, 72H), 2.74-3.27 (m, 39H), 3.35-3.92 (m, 813H), 3.91-4.72 (m, 58H), 5.05-5.29 (m, 5H), 6.16-6.22 (m, 1H), 7.20-7.38 (m, 10H), 7.41-7.53 (m, 7H), 7.53-7.64 (m, 7H), 7.89-8.06 (m, 15H) and 8.70-8.83 (m, 7.4H). n_{FAPI}=7.4.

1e.11 BHALys[Lys]₂[Lys]₄[Lys]₈[Lys]₁₆[Lys]₃₂[(α-NH₂.TFA)₃₂(ε-NHCO-dPEG₁₁₀₀-C(O)N-FAPI-04)₁₀(ε-NHCO-mPEG₁₀₀₀)₁₉(ε-NH₂)₃] RHa-19

To a solution of BHALys[Lys]₂[Lys]₄[Lys]₈[Lys]₁₆[Lys]₃₂[(α-NHBoc)₃₂(ε-NHCO-dPEG₁₁₀₀-C(O)N-FAPI-04)₁₀(ε-NHCO-dPEG₁₁₀₀)₁₉(ε-NH₂)₃] RHa-13 (83 mg, 1.6 μmol) in dichloromethane (2.5 mL) at rt was added TFA (1.5 mL). The reaction mixture was stirred for 18 h and concentrated in vacuo to give BHALys[Lys]₂[Lys]₄[Lys]₈[Lys]₁₆[Lys]₃₂[(α-NH₂.TFA)₃₂(ε-NHCO-dPEG₁₁₀₀-C(O)N-FAPI-04)₁₉(ε-NHCO-mPEG₁₀₀₀)₉(ε-NH₂)₃] RHa-19, which was used without further purification. HPLC (HPLC Method, 5-80, 8 min, TFA) Rₜ=4.61 min. ¹H NMR (300 MHz, CD₃OD): δ (ppm): 1.21-2.18 (m, 348H), 2.28-2.54 (m, 39H), 3.03-3.38 (m, 85H), 3.36 (s, 64H), 3.40-4.15 (m, 2118H), 4.15-4.51 (m, 79H), 7.13-7.42 (m, 10H), 7.48-7.73 (m, 19H), 7.90-8.11 (m, 21H) and 8.67-8.88 (m, 10H).

1e.12 BHALys[Lys]₂[Lys]₄[Lys]₈[Lys]₁₆[Lys]₃₂[(α-NH₂.TFA)₃₂(ε-NHCO-dPEG₁₁₀₀-C(O)N-FAPI-04)₁(ε-NHCO-mPEG₁₀₀₀)₃₁] RHa-20

Prepared according to the procedure described for BHALys[Lys]₂[Lys]₄[Lys]₈[Lys]₁₆[Lys]₃₂[(α-NH₂.TFA)₃₂(ε-NHCO-dPEG₁₁₀₀-C(O)N-FAPI-04)₁₀(ε-NHCO-mPEG₁₀₀₀)₁₉(ε-NH₂)₃] RHa-19 using BHALys[Lys]₂[Lys]₄[Lys]₈[Lys]₁₆[Lys]₃₂[(α-NHBoc)₃₂(ε-NHCO-dPEG₁₁₀₀-C(O)N-FAPI-04)₁(ε-NHCO-mPEG₁₀₀₀)₃₁] RHa-12 (80 mg, 1.7 μmol) and used without further purification. HPLC (HPLC Method, 5-80, 8 min, TFA) Rₜ=4.74 min.

1e.13 BHALys[Lys]₂[Lys]₄[Lys]₈[(α-NH₂.TFA)₈(ε-NHCO-dPEG₁₁₀₀-C(O)N-FAPI-04)₃(ε-NHCO-mPEG₁₀₀₀)₅] RHa-23

A solution of BHALys[Lys]₂[Lys]₄[Lys]₈[(α-NHBoc)₈(ε-NHCO-dPEG₁₁₀₀-C(O)N-FAPI-04)₃(ε-NHCO-mPEG₁₀₀₀)₅] RHa-17 (60 mg, 4.5 μmol) in TFA:dichloromethane (1.8 mL, 1:2 v/v) was stirred for 20 h at rt whereupon the reaction mixture was concentrated in vacuo. The residue was dissolved in water (˜5 mL) and lyophilised to give BHALys[Lys]₂[Lys]₄[Lys]₈[(α-NH₂.TFA)₈(ε-NHCO-dPEG₁₁₀₀-C(O)N-FAPI-04)₃(ε-NHCO-mPEG₁₀₀₀)₅] RHa-23 as a yellow gum (quant). HPLC (HPLC Method 5-80, 8 min, TFA) Rₜ=4.52 min.

1f. Synthesis of DUPA-Targeted Intermediates
1f.1 DUPA(O-tBu)₃-NHS ester, Compound 39

To a solution of DUPA(O-tBu)₃-OH (248 mg, 0.507 mmol) in MeCN (3 mL) was added N,N′-disuccinimidyl carbonate (200 mg, 0.778 mmol) and pyridine (250 μL, 3.21 mmol) and the reaction was stirred at RT overnight. The reaction was concentrated under reduced pressure and purified by silica gel column chromatography using a gradient of 1:4 to 1:1 EtOAc/hexane as eluent, which yielded the title compound, Compound 39 as a white solid (223 mg, 75%). ¹H NMR (300 MHz, CDCl₃) δ (ppm): 1.43 (two overlapping singlets, 18H), 1.47 (s, 9H), 1.58-2.18 (m, 4H), 2.20-2.44 (m, 3H), 2.52-2.69 (m, 2H), 2.72-3.10 (m, 4H), 4.34 (m, 1H), 4.54 (m, 1H), 5.22 (d, J=8.2 Hz, 1H), 5.53 (d, J=8.2 Hz, 1H).

1f.2 DUPA(O-tBu)₃-NHPEG₂₄CO₂H Compound 40

To a solution of NH₂PEG₂₄CO₂H (193 mg, 0.170 mmol) in DCM was added DUPA(0-tBu)-NHS ester Compound 39 (100 mg, 0.170 mmol) and NMM (20 μL, 0.187 mmol) and the reaction was stirred at room temperature overnight. The reaction was concentrated under reduced pressure and purified using preparative HPLC (20-90% MeCN, Rₜ=33 min) ¹H NMR (300 MHz, CDCl₃) δ (ppm): 1.37-1.45 (m, 27H), 1.75-2.11 (m, 5H), 2.27 (m, 4H), 2.57 (m, 2H), 3.32-3.84 (m, 103H), 4.26 (m, 2H). LCMS (philic method, TFA buffer) Rₜ=5.49 min. ESI MS (+ve) 1617.5 [M]⁺; calc. m/z for C₇₄H₁₄₁N₃O₃₄ [M]⁺=1616.9

1f.3 DUPA(O-tBu)₃-NHPEG₂₄-NHS ester, Compound 41

To a solution of DUPA(O-tBu)₃-NHPEG₂₄CO₂H Compound 40 (110 mg, 0.0538 mmol) in DCM (3 mL) was added DCC (11 mg, 0.0538 mmol) and N-hydroxysuccinimide (6 mg, 0.0538 mmol) and the reaction was monitored by LCMS. Upon consumption of the starting material the reaction was concentrated under reduced pressure. The crude residue was suspended in MeCN (˜1-2 mL) and filtered through a 0.45 μm filter and the clear solution was concentrated under reduced pressure and the crude residue of Compound 41 was used without further purification. LCMS (philic method, TFA buffer) Rₜ=5.66. ESI MS (+ve) 1736 [M]⁺; calc. m/z for C₇₈H₁₄₄N₄NaO₃₆ [M]⁺=1735.95

1f.4 HO-Lys[(α-NH-COPEG₂₄NH-DUPA(O-tBu)₃)(ε-NHFmoc)], Compound 42

To a mixture of DUPA(O-tBu)₃-NHPEG₂₄-NHS ester Compound 41 (116 mg, 0.0676 mmol) and Lys-α-NH₂.TFA-ε-NHFmoc (30 mg, 0.0676 mmol) in DMF (3 mL) was added triethylamine (9 μL, 0.203 mmol). The suspension gradually turned clear and was stirred at room temperature overnight. LCMS analysis indicated formation of the product and the reaction was concentrated under reduced pressure and purified by preparative HPLC (20-90% MeCN, Rₜ=37 min). The title compound, Compound 42 was collected as a colourless oil (23 mg, 17%). LCMS (philic, TFA buffer) Rₜ=6.09. ESI MS (+ve) 1989 [M]⁺; calc. m/z for C₉₅H₁₆₃N₅NaO₃₇ [M]⁺=1989.09.

### 1f.5 DOTA(O-tBu)₄-p-BnNH-Glu-OH, Compound 43

To a solution of DOTA(O-tBu)₄-p-BnNH₂.TFA (50 mg, 0.0589 mmol) and glutaric anhydride (8 mg, 0.0708 mmol) in DMF (2 mL) was added NMM (10 μL, 0.0884 mmol) and the reaction was stirred at room temperature overnight. The reaction was monitored by LCMS and upon complete consumption of the starting material was concentrated under reduced pressure. The crude residue taken up in EtOAc (5 mL) and washed with pH 3 phosphate buffer (3×5 mL), brine (3 mL), dried over MgSO₄ and concentrated under reduced pressure to yield Compound 43. ¹H NMR (300 MHz, CDCl₃) δ (ppm): 1.39-1.52 (m, 36H), 1.79-3.52 (m, 36H), 6.95 (m, 2H), 7.75 (d, J=8.35 Hz, 2H).

1f.6 DOTA(O-tBu)₄-BnNH-Glu-NHS ester, Compound 44

To a solution of DOTA(O-tBu)₄-p-BnNH-Glu-OH, Compound 43 (30 mg, 0.0354 mmol) in DCM (2 mL) was added DCC (7 mg, 0.0354 mmol) and N-hydroxysuccinimide (4 mg, 0.0354) and the reaction was stirred at room temperature overnight. The reaction was monitored by LCMS for consumption of starting material and formation of the product. Once the reaction was deemed complete, the reaction was concentrated under reduced pressure and suspended in a small amount of MeCN (˜1-2 mL) and filtered through a 0.45 μm acrodisc filter and the clear solution was concentrated under reduced pressure. The residue was used directly in the next step without further purification. LCMS (LCMS Method 40-65, TFA Buffer) Rₜ=5.29. ESI MS (+ve) 944 [M]⁺; calc. m/z for C₄₈H₇₇N₆O₁₃ [M]⁺=944.6.

1f.7 HO-Lys[(α-NH-COPEG₂₄NH-DUPA(O-tBu)₃)(ε-GluNH-p-Bn-DOTA(OtBu)₄)J Compound 45

To a solution of Lys[(α-NH-COPEG₂₄NH-DUPA(O-tBu)₃)(ε-NHFmoc)] Compound 42 (30 mg, 17.6 μmol) in DMF (3 mL) was added piperidine (1 mL) and the reaction was stirred at RT. After 2 h the volatiles were concentrated in vacuo and the crude material Lys[(α-NH-COPEG₂₄NH-DUPA(O-tBu)₃)(ε-NH₂)] stored at 4° C. until required.

To a solution of Lys[(α-NH-COPEG₂₄NH-DUPA(O-tBu)₃)(ε-NH₂)] (26 mg, 15.6 μmol) in DMF (1 mL) was added DOTA(O-tBu)₄-p-Bn-NH-Glu-NHS, Compound 44 (14 mg, 15.6 μmol) and NMM (5 μL, 46.8 μmol). After 2 h the reaction was checked by LCMS, which showed consumption of the starting material and the reaction was concentrated under reduced pressure and purified using preparative HPLC (50-65% MeCN, Rₜ=49 mins). The title compound, Compound 45 was isolated as a colourless oil (4 mg, 9%). LCMS (LCMS Method 40-65, TFA Buffer) Rₜ=4.40. ESI MS (+ve) 2575 [M]⁺; calc. m/z for C₁₂₄H₂₂₅N₁₀O₄₅ [M]⁺=2575.6.

1f.8 Di-tert-butyl ((1-(tert-butoxy)-5-((2,5-dioxopyrrolidin-1-yl)oxy)-1,5-dioxopentan-2-yl) carbamoyl) glutamate, Compound 46

To a stirred solution of 5-(tert-butoxy)-4-(3-(1,5-di-tert-butoxy-1,5-dioxopentan-2-yl)ureido)-5-oxopentanoic acid (Amadis Chemicals, 100 mg, 0.20 mmol) in acetonitrile (5 mL) was added a solution of N,N-disuccinimidyl carbonate (79 mg, 0.31 mmol) in pyridine (80 μL, 0.45 mmol). The reaction mixture was stirred at ambient temperature for 15 h. The progress of the reaction was monitored by TLC (EtOAc: Hexane 1:1). The volatiles were removed in vacuo and the crude mixture was purified by column chromatography (SiO₂, gradient 0-60% EtOAc/hexane) to give Compound 46 (85 mg, 71%) as white solid. ¹H NMR (300 MHz, CDCl₃) δ (ppm): 1.30-1.62 (3 x br s, 27H); 1.64-1.94 (m, 4H); 1.97-2.22 (m, 2H); 2.25-2.52 (m, 3H); 2.55-2.74 (m, 2H); 2.77-3.14 (m, 4H); 4.26-4.44 (m, 1H); 4.48-4.67 (m, 1H); 5.08-5.85 (2 x br s, 2H).

1f.9 Tri-tert-butyl (18S, 22S)-2,2-dimethyl-4,15,20-trioxo-3,8,11-trioxa-5,14,19,21-tetraazatetracosane-18,22,24-tricarboxylate, Compound 47

To a stirred solution of Compound 46 (85 mg, 0.15 mmol) in dichloromethane (5 mL) was added tert-Butyl (2-(2-(2-aminoethoxy) ethoxy)ethyl) carbamate (54 mg, 0.22 mmol) and NMM (24 μL, 0.22 mmol) in succession. The reaction mixture was stirred at ambient temperature for 30 min. The volatiles were removed in vacuo and the crude mixture was purified by column chromatography (SiO₂, gradient 0-60% acetone/hexane) to give Compound 47 (98 mg, 67% over two steps) as colourless viscous oil. ¹HNMR (300 MHz, CDCl₃) δ (ppm): 1.14-1.20 (m, 4H); 1.42-1.51 (m, 32H); 1.76-2.38 (m, 8H); 2.55-2.81 (m, 8H); 2.93-3.09 (m, 1H); 3.24-3.35 (m, 2H); 3.63-3.74 (m, 1H); 3.82-3.94 (m, 1H); 4.30-4.50 (m, 2H); 6.14-6.34 (m, 2H); 9.22-9.28 (m, 1H). LCMS (Method 20-90, 15 min, formic) Rₜ=9.52 min. ESI MS (+ve) 719 [M+1]⁺; calc. m/z for C₃₄H₆₂N₄O₁₂ [M]⁺: 718.44.

1f.10 (13S,17S)-1-Amino-10,15-dioxo-3,6-dioxa-9,14,16-triazanonadecane-13,17,19-tricarboxylic acid, Compound 48

To a stirred solution of tri-tert-butyl (18S,22S)-2,2-dimethyl-4,15,20-trioxo-3,8,11-trioxa-5,14,19,21-tetraazatetracosane-18,22,24-tricarboxylate (90 mg, 0.13 mmol) in dichloromethane (3 mL) was added trifluoroacetic acid (3 mL). The reaction mixture was stirred at ambient temperature for 4 h whereupon the volatiles were removed in vacuo. To the residue was added deionised water (5 mL) and the resulting solution was filtered (0.45 μm acrodisc syringe filter) and the filtrate lyophilised to obtain Compound 48 (71 mg, quant.) as white solid. ¹H NMR (300 MHz, D₂O) δ (ppm): 1.78-1.93 (m, 2H); 2.00-2.13 (m, 2H); 2.25-2.30 (m, 2H); 2.37-2.44 (m, 2H); 3.06-3.11 (m, 2H); 3.25-3.29 (m, 2H); 3.49-3.53 (m, 2H); 3.59 (s, 4H); 3.60-3.66 (m, 2H); 4.09 (dd, J=6 Hz and 9 Hz, 1H); 4.16 (dd, J=6 Hz and 9 Hz, 1H).

1f.11 BHALys[Lys]₂[Lys]₄[Lys]₈[(α-NHBoc)₈(ε-NH-COPEG₂₄NH-DUPA(OtBu)₃)₈, G3, Compound 36

Prepared according to general procedure D using BHALys[Lys]₂[Lys]₄[Lys]₈[(α-NHBoc)₈(s-NH-COPEG₂₄-NH₂)₈] Compound 116 (44 mg, 3.68 μmol) and, DUPA(OtBu)₃OH (17 mg, 35.3 μmol). The reaction is concentrated under reduced pressure and purified by SEC (400 drops/tube, MeOH sephadex LH20, 35 drops/min). Fractions were checked by TLC analysis (5% BaCl₂ solution followed by iodine stain; dark brown spot) and HPLC and those containing the product were combined and concentrated under reduced pressure. The title compound was obtained as a very light brown film (47 mg, 81%). ¹H NMR (300 MHz, CD₃OD) δ (ppm): 0.76-1.92 (m, 402H) 1.99-2.16 (m, 14H), 2.23-2.39 (m, 26H), 2.44 (m, 16H), 3.02-3.26 (m, 32H), 3.34-3.41 (m, 21H), 4.34-4.38 (m, 861H), 6.20 (s, 1H), 7.20-7.39 (m, 10H).

### 1f.12 DOTA(OtBu)₄GA-NHS Ester RL-20

To a solution of DOTAGA-tetra-(tert-butyl ester) RL-19 (300 mg, 0.428 mmol) in dichloromethane (5 mL) was added DCC (88 mg, 0.428 mmol) and N-hydroxysuccinimide (49 mg, 0.428 mmol) and the ensuing reaction was stirred overnight at room temperature. The reaction was concentrated under a stream of N₂ and taken up in a small amount of MeCN (˜2 mL) and filtered (0.45 μm acrodisc syringe filter). The filtrate was concentrated in vacuo to give DOTA(OtBu)₄GA-NHS Ester RL-20 (380 mg, quantitative yield) as a light brown foam which was used without further purification. LCMS (LCMS Method 40-90, 8 min TFA) Rₜ=4.70 min. ESI MS (+ve) 799 [M]⁺; calc. m/z for C₃₉H₆₇N₅O₁₂ [M+H]⁺: 799.

1f.13 DOTA(OtBu)₄GA-NHCO-PEG₂₄-COOH RL-21

To a solution of DOTA(OtBu)₄GA-NHS ester RL-20 (100 mg, 0.143 mmol) in DMF (5 mL) was added Amino-PEG₂₄-Acid (163 mg, 0.143 mmol) and NMM (47 μL, 0.429 mmol) and the ensuing reaction was stirred for 18 h at rt. The reaction was concentrated under reduced pressure and purified using preparative HPLC (Prep-HPLC Method 20-90, TFA, Rₜ=28 min) to give the title compound RL-21 as a colourless oil (47 mg). ¹H NMR (300 MHz, CD₃OD): δ (ppm) 1.27-1.90 (m, 37H), 2.56 (t, J 6.01, 2H), 2.72 (m, 4H), 2.94-2.62 (m, 120H). LCMS (LCMS Method 40-90, 8 min, TFA Rₜ=5.20 min. ESI MS (+ve) 1830 [M]⁺; calc. m/z for C₈₆H₁₆₅N₅O₃₅ [M+H⁺]⁺=1830.

### Example 2. Targeted Dendrimer Conjugates

### 2a HER-2 Nanobody Conjugates

2a.1 Nanobody Sequence information PGP183,DNA

Nanobody 2D3 sequence Compound 118 (SEQ ID NO: 1)

EVQLVESGGSLVQPGGSLRLSCAASGFTFDDYAMSWVRQVPGKGLEWVSS
INWSGTHTDYADSVKGRFTISRNNANNTLYLQMNSLKSEDTAVYYCAKNW
RDAGTTWFEKSGSAGQGTQVTVSS

Nanobody 2D3 N-terminal tag, TEV protease cleavage site, C-terminal azide (“N-terminal Tag-Nanobody-N3”) (SEQ ID NO: 5)

GGSHHHHHHGMASMTGGQQMGRDLYENLYFQGEVQLVESGGSLVQPGGSL
RLSCAASGFTFDDYAMSWVRQVPGKGLEWVSSINWSGTHTDYADSVKGRF
TISRNNANNTLYLQMNSLKSEDTAVYYCAKNWRDAGTTWFEKSGSAGQGT
QVTVSS#

Nanobody 2D3, with C-terminal tag, TEV protease cleavage site, azide (“Nanobody-N3-C-terminal Tag”) (SEQ ID NO: 2)

EVQLVESGGSLVQPGGSLRLSCAASGFTFDDYAMSWVRQVPGKGLEWVSS
INWSGTHTDYADSVKGRFTISRNNANNTLYLQMNSLKSEDTAVYYCAKNW
RDAGTTWFEKSGSAGQGTQVTVSS#ENLYFQGHHHHHH Where # =
unnatural amino acid.

Nanobody 2D3, with C-terminal histidine tag, TEV protease cleavage site, additional cysteine (“Nanobody-Cys” SRS-13) (SEQ ID NO:10)

EVQLVESGGSLVQPGGSLRLSCAASGFTFDDYAMSWVRQVPGKGLEWVSS
INWSGTHTDYADSVKGRFTISRNNANNTLYLQMNSLKSEDTAVYYCAKNW
RDAGTTWFEKSGSAGQGTQVTVSSLGTLCTPSRENLYFQGHHHHHH

2a.2 Nanobody-N3 expression plasmid

The coding sequence of anti-HER2 nanobody clone 2D3 (US20110028695A1 SEQ ID NO: 1986) was inserted into the expression plasmid pET-His6-TEV-1B (Addgene plasmid 29653) using standard molecular biology techniques. E. coli K12 codon optimised DNA sequences were synthesised and then cloned into plasmid pET-His6-TEV-1B.

For incorporation of unnatural amino acids into the recombinant protein, the amber stop codon (TAG) was inserted in-frame at the last position of the nanobody coding sequence, followed then by an ochre (TAA) or opal (TGA) stop codon for translational termination. Where a C-terminal his6 tag was used, the amber stop codon (TAG) was inserted in-frame at the last position of the nanobody coding sequence, and the sequence encoding the TEV protease cleavage site and 6his tag followed by an ochre (TAA) or opal (TGA) stop codon for translational termination was then appended to this sequence.

### 2a.3 Expression and Purification of Nanobody-N₃

Anti-HER2 nanobody 2D3 expression was performed in E. coli strain B95(DE3) (Mukai et al., Scientific Reports (2015), 5, 9699) transformed with nanobody expression plasmid and orthogonal pair expression plasmid pEVOL-pAcFRS.2.t1 (Amiram et al., Nat. Biotechnol (2015), 33 (12). Cells were cultured in Terrific broth (25 g/L tryptone, 30 g/L yeast and 5 g/L glycerol, 0.017 M KH₂PO₄, 0.072 M K₂HPO₄ 50 μg/mL kanamycin sulphate, 30 μg/mL chloramphenicol) contained in baffled shake flasks at 37° C. until a cell density of OD₆₀₀ 0.7-1.0 was reached. Recombinant protein expression was induced by addition of 1.5 mM IPTG and 0.05% w/v L-(+)-arabinose with addition of 1.5 mM p-azido-phenylalanine, and allowed to proceed for 20 hours at 25° C. Bacteria were harvested by centrifugation and a cell lysate generated by high pressure homogenisation followed by addition of a protease inhibitor cocktail, lysozyme and DNAse treatment. The insoluble material can be dissolved in refolding buffer (6 M Guanidine HCl, 50 mM NaH2PO4, 300 mM NaCl, 20 mM Imidazole, pH 8). Following clarification by high speed centrifugation and filtration through a 0.45 μM membrane filter, his6-tagged nanobody was purified by immobilised metal affinity chromatography (IMAC) using nickel-charged nitrilotriacetic acid-agarose. On-column refolding was achieved by first washing the bound protein with a column volume of 3 M Guanidine HCl, 50 mM NaH₂PO₄, 300 mM NaCl, 20 mM Imidazole, pH 8, followed by two column volumes of 50 mM NaH₂PO₄, 300 mM NaCl, 20 mM Imidazole, pH 8. Bound proteins were eluted with 2 column volumes of 50 mM NaH₂PO₄, 300 mM NaCl, 250 mM Imidazole, pH 8. Following IMAC, -nanobody-N₃ was further purified by anion exchange chromatography using a HiTrap Q HP column (GE Healthcare, catalogue 17115301). Binding and washing steps were performed using Tris buffer (20 mM, pH 8) and elution performed using a gradient of 0% to 50% of 1 M NaCl buffer (1M NaCl, 20 mM Tris, pH 8). Relevant nanobody fractions obtained were buffer exchanged into 20 mM Tris, pH 8 using Amicon 10 k MWCO filter units (Merck, catalogue UFC901008).

### 2a.4 Conjugation of Nanobody-N₃-C-Terminal Tag to Dendrimers

Nanobody-N₃—C-terminal Tag was conjugated to the dendrimers Compounds 128-132, SRS-4-Mal and HH-2 using bio-orthogonal click chemistry to generate Compounds 85, 86, 87, 89, 91, 92, 93, 94, 96, 97, SRS-20 and SRS-22 according to the procedure described for Compounds 83 and 84 except that the linker DBCO-Glu-NHPEG₂₄CO-NHPEG₃-TCO (Compound 51) was used.

Gels were run for compounds 85 to 97, 123 to 127, SRS-20 and SRS-22 (FIG. 24) which showed bands at the appropriate MW for the nanobody-dendrimer conjugates. The purity of each nanobody-dendrimer conjugate was confirmed using non-reducing sodium dodecyl sulfate-polyacrylamide gel electrophoresis (SDS-PAGE). Following electrophoretic separation on a 4-15% polyacrylamide gel (Bio-rad, catalogue 4561086), the nanobody-dendrimer conjugates appear as fluorescent bands corresponding approximately to the size of the dendrimer plus the additional mass of the nanobodies when imaged using a Typhoon Biomolecular Imager (GE Healthcare). Subsequent staining with Coomassie Brilliant Blue (CBB) revealed the presence of nanobody at the same locations. No other bands were revealed by CBB indicating the absence of unreacted nanobody and the purity of the preparation.

2a.4.1 Nanobody-N₃ DBCO-Glu-NHPEG₂₄CONHPEG₃-TCO (MeTzPh)PEG₄CO-NHPEG₂₄CO—[N(PN)₂][Lys]₄[Lys][((α-NHDFO)₂(α-NHGlu-VC-PAB-MMAE)₆)(ε-NH-COPEG₁₁₀₀)₈], Compound 83

A solution of DBCO-Glu-NHPEG₂₄CO-NHPEG₃-TCO Compound 51 (167 μL of a 1 mg/mL solution in DMSO:PBS buffer (20:80); 1 eq.) was added to a solution of (MeTzPh)PEG₄CO-NHPEG₂₄CO—[N(PN)₂][Lys]₄[Lys]₈[((α-NHDFO)₂(α-NHGlu-VC-PAB-MMAE)₆)(ε-NH-COPEG₁₁₀₀)₈], Compound 73 (250 μL of a 8 mg/mL solution in PBS; 1 eq.) and the mixture allowed to stand at RT for 30 min. The reaction mixture was diluted to 500 μL with PBS buffer and the resulting solution analysed by HPLC which showed no remaining linker in the reaction mixture. A portion of this reaction mixture (177 μL; 1 eq.) was added to a solution of Nanobody-N₃ (“Nanobody-N3-C-terminal Tag”) (62.5 μL of a 9.2 mg/mL solution in Tris buffer; 1 eq.) and the ensuing reaction mixture left to stand at RT for 7 h and then at 4° C. overnight. The resulting nanobody-dendrimer conjugate was purified using anion-exchange chromatography to remove unreacted dendrimer using a HiTrap Q HP column (GE Healthcare, catalogue 17115301). Binding and wash steps were performed using Tris buffer (20 mM, pH 8) and elution performed using a gradient of 0% to 50% of 1M NaCl buffer (1M NaCl, 20 mM Tris, pH 8). Size exclusion chromatography was then used to remove unreacted nanobody. Separation was achieved using a Superdex 75 10/300 column (GE Healthcare, catalogue 17517401) equilibrated with column buffer (50 mM NaH₂PO₄, 150 mM NaCl, pH 8). The nanobody-dendrimer conjugate was then concentrated and buffer exchanged using a 10 k MWCO Amicon Ultra centrifugal filter into 10 mM HEPES pH 8 for radioactive labelling.

2a.4.2 BHA[Lys]₂[Lys]₄[Lys]₈[((α-NH-COPEG₂₄NH-COPEG₄(PhMeTz)/TCO-PEG₃NH-COPEG₂₄NH-Glu-DBCO/N₃-Nanobody)₁(α-NHDFO)₂(α-NHGlu-VC-PAB-MMAE)₅)(ε-NH-COPEG₁₀₀₀)₈], G3, Compound 84

A solution of DBCO-Glu-NHPEG₂₄CO-NHPEG₃-TCO (202 μL of a 1 mg/mL solution in DMSO:PBS buffer (20:80); 1 eq.) was added to a solution of BHA[Lys]₂[Lys]₄[Lys]₈[((α-NH-COPEG₂₄NH-COPEG₄(PhMeTz)₁(α-NHDFO)₂(α-NHGlu-VC-PAB-MMAE)s)(F-NHCOPEG₁₀₀₀)₈] Compound 74 (250 μL of a 8 mg/mL solution in PBS, 1 eq.) and the mixture allowed to stand at RT for 30 min.

The reaction mixture diluted to 500 μL with PBS buffer and the resulting solution analysed by HPLC which showed no remaining linker in the reaction mixture. A portion of this reaction mixture (175 μL; 1 eq.) was added to a solution of Nanobody-N₃ (“Nanobody-N3-C-terminal Tag”) (62.5 μL of a 9.2 mg/mL solution in Tris buffer; 1 eq.) and the ensuing reaction mixture left to stand at RT for 7 h and then at 4° C. overnight. The resulting nanobody-dendrimer conjugate was purified using anion-exchange chromatography to remove unreacted dendrimer, using a HiTrap Q HP column (GE Healthcare, catalogue 17115301). Binding and wash steps were performed using Tris buffer (20 mM, pH 8) and elution performed using a gradient of 0% to 50% of 1M NaCl buffer (1M NaCl, 20 mM Tris, pH 8). Size exclusion chromatography was then used to remove unreacted nanobody. Separation was achieved using a Superdex 75 10/300 column (GE Healthcare, catalogue 17517401) equilibrated with column buffer (50 mM NaH₂PO₄, 150 mM NaCl, pH 8). The nanobody-dendrimer conjugate was then concentrated and buffer exchanged using a 10 k MWCO Amicon Ultra centrifugal filter into 10 mM HEPES, pH 8 for radioactive labelling.

2a.4.3 BHALys[Lys]₂[Lys]₄[((α-NH-COPEG₂₄NH-COPEG₄(PhMeTz)/TCO-PEG₃NH-COPEG₂₄NH-Glu-DBCO/N₃-Nanobody)₁(α-Lys(α-NHCy5)(ε-NHDFO))₁(α-NH₂)₂)(ε-NH-COPEG₁₀₀₀)₄], G2, Compound 85 and BHALys[Lys]₂[Lys]₄[((α-NH-COPEG₂₄NH-COPEG₄(PhMeTz)/TCO-PEG₃NH-COPEG₂₄NH-Glu-DBCO/N₃-Nanobody)₂(α-Lys(α-NHCy5)(ε-NHDFO))₁(α-NH₂))₁(ε-NHCOPEG₁₀₀₀)₄], G2, Compound 86

Prepared according to General Procedure G using BHALys[Lys]₂[Lys]₄[((α-NH-COPEG₂₄NH-COPEG₄(PhMeTz))₁(α-Lys(α-NHCy5)(ε-NHDFO))₁(α-NH₂)i)(ε-NH-COPEG₁₀₀₀)₄], G2, Compound 25 except:
Step 1: The TCO linker solution 51 was prepared in neat DMSO; and
Step 2: The dendrimer was dissolved in Tris buffer at pH 8 and, upon complete reaction of the TCO linker with the dendrimer (UPLC or LCMS), the reaction mixture was diluted with Tris buffer such that the final concentration of DMSO was ≤5% (v/v) and purified by centrifugal ultrafiltration (Amicon, 0.5 mL regenerated cellulose membrane, 10 kDa MWCO).

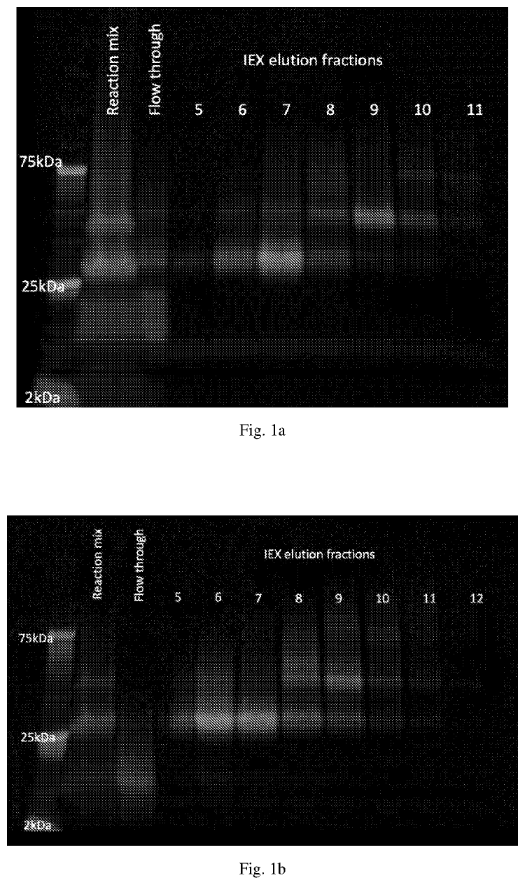

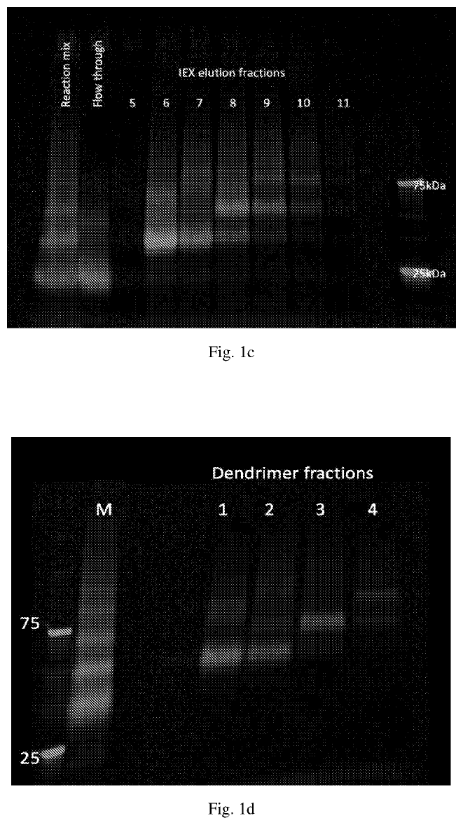

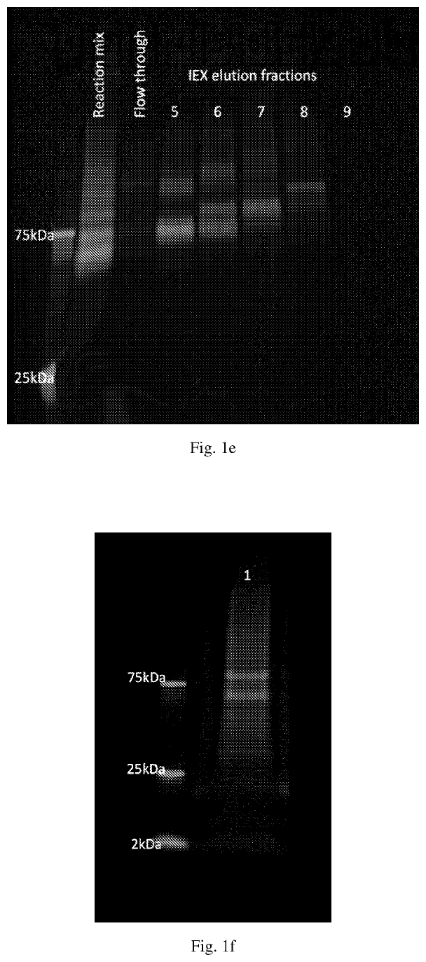

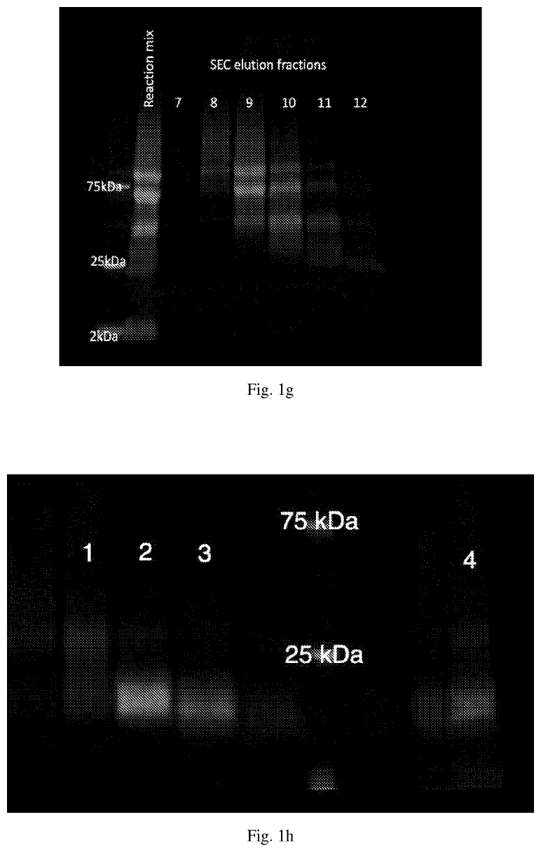

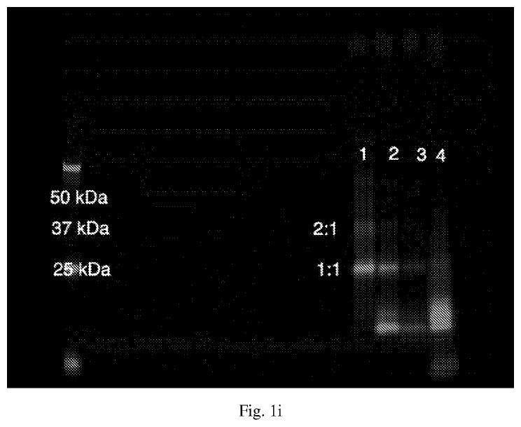

Purification method same as method used for the purification of Compound 83 and 84. to give the nanobody-dendrimer conjugates Compound 85 (232 μg; 0.45 μg/μL in HEPES buffer at pH 8; as shown in FIG. 1a Lanes 6 and 7) and the nanobody-dendrimer conjugate Compound 86 (60 μg; 0.30 μg/μL in HEPES buffer at pH 8; as shown in FIG. 1a Lane 9).

2a.4.4 BHALys[Lys]₂[Lys]₄[Lys]₈[((α-NH-COPEG₂₄NH-COPEG₄(PhMeTz)/TCO-PEG₃NH-COPEG₂₄NH-Glu-DBCO/N₃-Nanobody)₁(α-Lys(α-NHCy5)(ε-NHDFO))₁(α-NH₂)₆)(ε-NH-COPEG₄₁₂)₈], G3, Compound 87 (and BHALys[Lys]₂[Lys]₄[Lys]₈[((α-NH-COPEG₂₄NH-COPEG₄(PhMeTz)/TCO-PEG₃NH-COPEG₂₄NH-Glu-DBCO/N₃-Nanobody)₂(α-Lys(α-NHCy5)(ε-NHDFO))₁(α-NH₂)₅)(ε-NH-COPEG₄₁₂)₈], G3, Compound 88

Prepared according to General Procedure G using BHALys[Lys]₂[Lys]₄[Lys]₈[((α-NH-COPEG₂₄NH-COPEG₄(PhMeTz))₁(α-Lys(α-NHCy5)(ε-NHDFO))₁(α-NH₂)s)(ε-NH-COPEG₄₁₂)₈], G3, Compound 26 except:
Step 1: The TCO linker Compound 51 solution was prepared in neat DMSO; and
Step 2: The dendrimer was dissolved in Tris buffer at pH 8 and, upon complete reaction of the TCO linker with the dendrimer (UPLC or LCMS), the reaction mixture was diluted with Tris buffer such that the final concentration of DMSO was <5% (v/v) and purified by centrifugal ultrafiltration (Amicon, 0.5 mL regenerated cellulose membrane, 10 kDa MWCO).

Purification method same as method used for the purification of Compound 83 and 84 to give the nanobody-dendrimer conjugates Compound 87 (140 μg; 1.48 μg/μL in HEPES buffer at pH 8; as shown in FIG. 1b Lanes 6 and 7)) and Compound 88 (not pure; as shown in FIG. 1b Lanes 8 and 9)

2a.4.5 BHALys[Lys]₂[Lys]₄[Lys]₈[((α-NH-COPEG₂₄NH-COPEG₄(PhMeTz)/TCO-PEG₃NH-COPEG₂₄NH-Glu-DBCO/N₃-Nanobody)₁(α-Lys(α-NHCy5)(ε-NHDFO))₁(α-NH₂)₆)(ε-NH-COPEG₁₀₀₀)₈], G3, Compound 89 and BHALys[Lys]₂[Lys]₄[Lys]₈[((α-NH-COPEG₂₄NH-COPEG₄(PhMeTz)/TCO-PEG₃NH-COPEG₂₄NH-Glu-DBCO/N₃-Nanobody)₂(α-Lys(α-NHCy5)(ε-NHDFO))₁(α-NH₂)s)(ε-NH-COPEG₁₀₀₀)₈], G3, Compound 90

Prepared according to General Procedure G using BHALys[Lys]₂[Lys]₄[Lys]₈[((α-NH-COPEG₂₄NH-COPEG₄(PhMeTz))₁(α-Lys(α-NHCy5)(ε-NHDFO))₁(α-NH₂)₆)(ε-NH-COPEG₁₀₀₀)₈], G3, Compound 27 except:
Step 1: The TCO linker (Compound 51) solution was prepared in neat DMSO; and
Step 2: The dendrimer was dissolved in Tris buffer at pH 8 and, upon complete reaction of the TCO linker with the dendrimer (UPLC or LCMS), the reaction mixture was diluted with Tris buffer such that the final concentration of DMSO was <5% (v/v) and purified by centrifugal ultrafiltration (Amicon, 0.5 mL regenerated cellulose membrane, 10 kDa MWCO).

Purification method same as method used for the purification of Compound 83 and 84 to give the nanobody-dendrimer conjugates Compound 89(249 μg; 2.68 μg/μL in HEPES buffer at pH 8; as shown in FIG. 1c Lanes 6 and 7) and Compound 90 (not pure, as shown in FIG. 1c lanes 9 and 10).

2a.4.6 BHA[Lys]₂[Lys]₄[Lys]₈[Lys]₁₆[((α-NH-COPEG₂₄NH-COPEG₄(PhMeTz)/TCO-PEG₃NH-COPEG₂₄NH-Glu-DBCO/N₃-Nanobody)₁(α-Lys(α-NHCy5)(ε-NHDFO))₁(α-NH₂)₁₄(ε-NH-COPEG₁₀₀₀)₁₆], G4, Compound 91 and BHA[Lys]₂[Lys]₄[Lys]₈[Lys]₁₆[((α-NH-COPEG₂₄NH-COPEG₄(PhMeTz)/TCO-PEG₃NH-COPEG₂₄NH-Glu-DBCO/N₃-Nanobody)₂(α-Lys(α-NHCy5)(ε-NHDFO))₁(α-NH₂)₁₃(ε-NHCOPEG₁₀₀₀)₁₆], G4, Compound 92

Prepared according to General Procedure G using BHALys[Lys]₂[Lys]₄[Lys]s [Lys]₁₆[((α-NH-COPEG₂₄NH-COPEG₄(PhMeTz)₁(α-Lys(α-NHCy5)(ε-NHDFO))₁(α-NH₂)₁₄(ε-NH-COPEG₁₀₀₀)₁₆], G4, Compound 28 except:
Step1: The TCO linker (Compound 51) solution was prepared in neat DMSO; and
Step 2: The dendrimer was dissolved in Tris buffer at pH 8 and, upon complete reaction of the TCO linker with the dendrimer (UPLC or LCMS), the reaction mixture was diluted with Tris buffer such that the final concentration of DMSO was <5% (v/v) and purified by centrifugal ultrafiltration (Amicon, 0.5 mL regenerated cellulose membrane, 10 kDa MWCO).

Purification method same as method used for the purification of Compound 83 and 84 to give the nanobody-dendrimer conjugates Compound 91; as shown in FIG. 1d: Lane 1 and 2) (203 μg; 0.975 μg/μL and 0.556 μg/μL in HEPES buffer at pH 8) and Compound 92; as shown in FIG. 1d: Lane 3 (101 μg; 0.34 μg/μL in HEPES buffer at pH 8).

2a.4.7 BHA[Lys]₂[Lys]₄[Lys]₈[Lys]₁₆[Lys]₃₂[((α-NH-COPEG₂₄NH-COPEG₄(PhMeTz) TCO-PEG₃NH-COPEG₂₄NH-Glu-DBCO N₃-Nanobody)₁(α-Lys(α-NHCy5)(ε-NHDFO))₁(α-NH₂)₃₀(ε-NH-COPEG₁₀₀₀)₃₂], G5, Compound 93 and BHA[Lys]₂[Lys]₄[Lys]₈[Lys]₁₆[Lys]₃₂[((α-NH-COPEG₂₄NH-COPEG₄(PhMeTz) TCO-PEG₃NH-COPEG₂₄NH-Glu-DBCO N₃-Nanobody)₂[α-Lys(α-NHCy5)(ε-NHDFO)]₁(α-NH₂)₂₉(ε-NH-COPEG₁₀₀₀)₃₂], G5, Compound 94

Prepared according to General Procedure G using BHALys[Lys]₂[Lys]₄[Lys]₈[Lys]₁₆ [Lys]₃₂[((α-NH-COPEG₂₄NH-COPEG₄(PhMeTz))₁(α-Lys(α-NHCy5)(ε-NHDFO))₁(α-NH₂)₃₀)(ε-NH-COPEG₁₀₀₀)₃₂], G5, Compound 29 except:
Step 1: The TCO linker Compound 51 solution was prepared in neat DMSO; and
Step 2: The dendrimer was dissolved in Tris buffer at pH 8 and, upon complete reaction of the TCO linker with the dendrimer (UPLC or LCMS), the reaction mixture was diluted with Tris buffer such that the final concentration of DMSO was <5% (v/v) and purified by centrifugal ultrafiltration (Amicon, 0.5 mL regenerated cellulose membrane, 10 kDa MWCO).

Purification method same as method used for the purification of Compound 83 and 84 to give the nanobody-dendrimer conjugates Compound 93; as shown in FIG. 1e Lane 5) (370 μg; 3.66 μg/μL in HEPES buffer at pH 8) and product Compound 94; as shown in FIG. 1e Lane 7)(177 μg; 1.72 μg/μL in HEPES buffer at pH 8).

2a.4.8 BHALys[Lys]₂[Lys]₄[(α-Lys(α-NHCy5)(ε-NHDFO))₁(α-NH₂)₃)(ε-NH-COPEG₂₄NH-COPEG₄(PhMeTz))₂)(ε-NH-COPEG₂₄NH-COPEG₄(PhMeTz)) TCO-PEG₃NH-COPEG₂₄NH-Glu-DBCO N₃-Nanobody)₂], G2, Compound 95, BHALys[Lys]₂[Lys]₄[(α-Lys(α-NHCy5)(ε-NHDFO))₁(α-NH₂)₃)(ε-NH-COPEG₂₄NH-COPEG₄(PhMeTz))₁(ε-NH-COPEG₂₄NH-COPEG₄(PhMeTz) TCO-PEG₃NH-COPEG₂₄NH-Glu-DBCO N₃-Nanobody)₃], G2, Compound 96, and BHALys[Lys]₂[Lys]₄[(α-Lys(α-NHCy5)(ε-NHDFO))₁(α-NH₂)₃)(ε-NH-COPEG₂₄NH-COPEG₄(PhMeTz) TCO-PEG₃NH-COPEG₂₄NH-Glu-DBCO N₃-Nanobody)₄], G2, Compound 97

Prepared according to General Procedure G using BHALys[Lys]₂[Lys]₄[(α-Lys(α-NHCy5)(ε-NHDFO))₁(α-NH₂)₃)(ε-NH-COPEG₂₄NH-COPEG₄(PhMeTz))₄], G2 dendrimer, Compound 24 except:
Step 1: The TCO linker Compound 51 solution was prepared in water; and
Step 2: The dendrimer was dissolved in MQ water.

Purification was carried out using the used for the purification of Compound 83 and 84 to give the nanobody-dendrimer conjugates Compound 97 and Compound 96 as an inseparable mixture (final concentration of 294 ug in HEPES buffer at pH 8; as shown in FIG. if Lane 1).

Also prepared by adding a solution of DBCO-Glu-NHPEG₂₄CO-NHPEG₃-TCO Compound 51 (57 μL of a 10 mg/mL solution in MQ water, 0.322 μmol) to a solution of the Nanobody-N₃ (“Nanobody-N3-C-terminal Tag”) (2.5 mg, 0.161 μmol, in 900 μL of Tris buffer at pH 8. The reaction mixture was left at RT for 2 d whereupon the reaction mixture was then purified by centrifugal ultrafiltration using Tris buffer pH 8 (Amicon, 0.5 mL regenerated cellulose membrane, 10 kDa MWCO; 10×450 μL) a. To this solution was added a solution of Compound 24 (230.0 μg in 500 μL MQ water; 0.026 μmol) and the reaction mixture was left to stand at RT overnight. Purification method same as method used for the purification of Compound 83 and 84 to give gave the nanobody-dendrimer Compound 97, Compound 95 and Compound 96 as an inseparable mixture (final concentration of 124 μg in HEPES buffer at pH 8; as shown in FIG. 1g, Lanes 9 and 10).

2a.4.9 BHALys[Lys]₂[Lys]₄[((α-NH-COPEG₂₄NH-COPEG₄(PhTzMe) TCO-PEGs-Nanobody)₁(α-Lys(α-NHCy5)₁(ε-NHDFO)₁)₁(α-NH₂)₂)(ε-NH-COPEG₁₀₀₀)₄], G2, Compound 98

A solution of the di-bromo maleimide linker Compound 57 was prepared by dissolving 2.0 mg in 1 mL DMSO (1.0 mL). A solution of TCEP (0.5 M in PBS pH 7) (2.3 μL, 1.174 μmmol) was added to a solution of the nanobody (“Nanobody-N₃—C-terminal Tag”) (Compound 118) (1.0 mg, 0.058 μmmol in 715 μL of Tris buffer at pH 8). The reaction mixture was heated at 37° C. for 1 h whereupon DMSO was added (673 μL) followed by a solution of the linker Compound 57 (93.0 μg, 0.117 μmmol) in DMSO (47.0 μL). After 1.5 h, the reaction mixture was cooled to RT and centrifuged whereupon a solution of Compound 25 (100 μg, 17 μL, 1.78 mg in 300 μL MQ water) was added to the precipitate and the solution left at 4° C. overnight. Purification method same as the method used for the purification of Compound 83 and 84 to give gave Compound 98 (29 μg; as shown in FIG. 1h, Lane 2 and 3) as a solution in Tris buffer at pH 8 (final concentration=1.78 mg/mL).

2a.4.10 BHALys[Lys]₂[Lys]₄[((α-NH-COPEG₂₄-NH-COPEG₄(PhTzMe) TCO-PEG₃-Nanobody)₁(α-Lys(α-NHCy5)₁(ε-NHDFO)i)₁(α-NH₂)₂)(ε-NH-COPEG₁₀₀₀)₄], G2, Compound 99

A solution of TCO-PEG₃-aldehyde (Conju-Probe) linker solution (0.7 mg, 1.46 μmol) in DMSO (50 μL) was added to a solution of the nanobody (“Nanobody-N3-C-terminal Tag”) (Compound 118) (931 μL of a 1.07 mg/mL stock solution in PBS buffer at pH 6.5) followed by the addition of solution of NaBH₃CN (0.09 mg, 1.5 μmol) in water (19 μL). The reaction mixture was cooled to 4° C. and the reaction monitored by UPLC analysis. After 16 h, the reaction mixture was diluted with PBS buffer (pH 6.5) to a total volume of 2.0 mL and then purified by centrifugal ultrafiltration using PBS buffer pH 6.5 (Amicon, 0.5 mL regenerated cellulose membrane, 10 kDa MWCO; 14×450 μL) UPLC: 5-20-30 MeCN % over 15 mins using 0.01% TFA buffer; Nanobody (Compound 118) Rt=8.46 mins with m/z 13802; Product:9.99 mins with m/z 14263. To the nanobody-PEG₃-TCO solution (500 μL PBS pH 6.5) was added a solution of the G2 dendrimer Compound 17 (179 μg, 0.020 μmol) in MQ water (15 μL). After standing at 4° C. overnight, the reaction mixture was purified using same method used for the purification of Compound 83 and 84 to give Compound 99 (12 μg; as shown in FIG. 1i, Lane 1) as a solution in Tris buffer at pH 8 (final concentration=575 μg/mL).

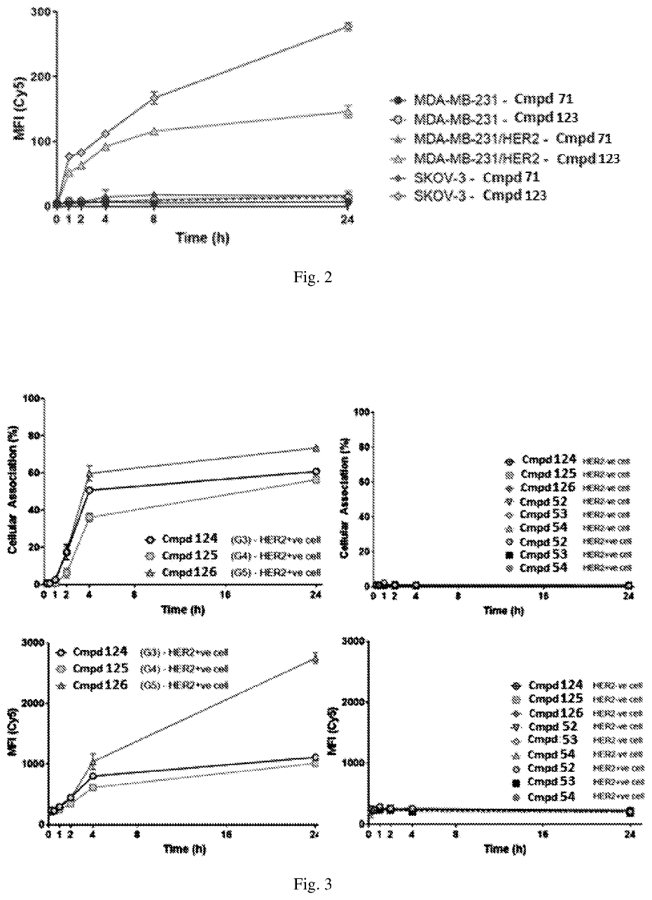

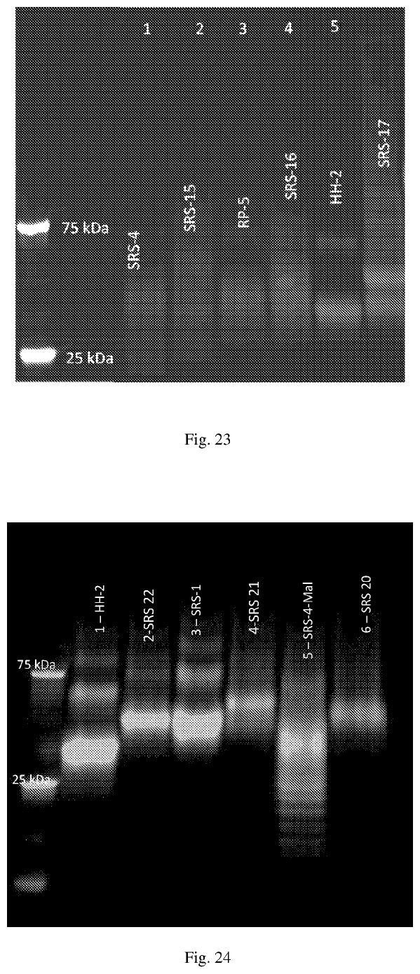

2a.4.11 [[(ε-NHCO-PEG₁₁₀₀)₈(α-NHDOTA)_{4.75}(α-NHCy5)_{0.5}(α-NH₂)_{2.75}][Lys]₈[Lys]₄[Lys]₂[Lys]-CONH—CH₂—CH₂—S-]₂MAL-PEG₃-NHCO-PEG₂₄-CONH-PEG₄-(PhTzMe)/TCO-PEG₃-NHCO-PEG₂₄-Glu-DBCO/N₃-Nanobody SRS-20
Step 1: Synthesis of [[(ε-NHCO-PEG₁₁₀₀)₈(α-NHDOTA)_{4.75}(α-NHCy5)_{0.5}(α-NH₂)_{2.75}][Lys]₈[Lys]₄[Lys]₂[Lys]-CONH—CH₂—CH₂—S-]₂MAL-PEG₃-NHCO-PEG₂₄-CONH-PEG₄-(PhTzMe)/TCO-PEG₃-NHCO-PEG₂₄-Glu-DBCO SRS-20a: To a solution of [[(F—NHCO-PEG₁₁₀₀)₈(α-NHDOTA)_{4.75}(α-NHCy5)_{0.5}(α-NH₂)_{2.75}][Lys]₈[Lys]₄[Lys]₂[Lys]-CONH—CH₂—CH₂—S-]₂MAL-PEG₃-NHCO-PEG₂₄-CONH-PEG₄-(PhTzMe) SRS-4-Mal (8.89 mg; 10 mg/mL in deionised water) was added TCO-PEG₃-NHCO-PEG₂₄-Glu-DBCO Compound 51 (1.05 mg; 2 eq; 10 mg/mL in deionised water) and the reaction mixture left at room temperaturert for 2 h. The Excess excess linker was removed from the reaction mixture by Amicon® Ultra centrifugal filters with 10 kDa MWCO Ultracel® regenerated cellulose membrane and concentrated by centrifugation at 4000 rpm for 10 min. The retentate was washed with deionised water (×5) by centrifugation at 4000 rpm. Removal of the excess linker was confirmed by analysis of purified product using LCMS (LCMS Method 3).
Step 2: Conjugation reaction: A solution of [[(ε-NHCO-PEG₁₁₀₀)₈(α-NHDOTA)_{4.75}(α-NHCy5)_{0.5}(α-NH₂)_{2.75}][Lys][Lys]₄[Lys]₂[Lys]-CONH—CH₂—CH₂—S-]₂MAL-PEG₃-NHCO-PEG₂₄-CONH-PEG₄-(PhTzMe)/TCO-PEG₃-NHCO-PEG₂₄-Glu-DBCO SRS-20a (9.41 mg; 10 mg/mL; 1 eq) in deionised water was added to a solution of Nanobody-N₃ (8.91 mg; 10 mg/mL in pH 8 Tris buffer; 2 eq) at rt. After 18 h, the Nanobody-dendrimer construct was first purified by Amicon® Ultra centrifugal filters with 30 kDa MWCO Ultracel® regenerated cellulose membrane and concentrated by centrifugation at 4000 rpm for 10 min. The retentate was washed with pH 8 Tris, buffer (×5) by centrifugation at 4000 rpm. The product obtained was then purified by nNickel affinity column chromatography followed by SEC to give [[(F—NHCO-PEG₁₁₀₀)₈(α-NHDOTA)_{4.75}(α-NHCy5)_{0.5}(α-NH₂)_{2.75}][Lys]₈[Lys]₄[Lys]₂[Lys]-CONH—CH₂—CH₂—S-]₂MAL-PEG₃-NHCO-PEG₂₄-CONH-PEG₄-(PhTzMe)/TCO-PEG₃-NHCO-PEG₂₄-Glu-DBCO/N₃-Nanobody SRS-20 (0.42 mg). See FIG. 2a, (Lane 6) for SDS Page gel analysis of the purified product. reaction mixture.
   2a.4.12 [(ε-NHCO-PEG₁₁₀₀)₁₆(α-DOTA)₇(α-NHCy5)₁(α-NH₂)₈][Lys]₁₆[Lys]₈[Lys]₄[Lys]₂[Lys]-(PN)NCO-PEG₂₄-CONH-PEG₄-(PhTzMe) TCO-PEG₃-NHCO-PEG₂₄-Glu-DBCO N₃-Nanobody SRS-22
Step 1: Synthesis of [(ε-NHCO-PEG₁₁₀₀)₁₆(α-DOTA)₇(α-NHCy5)₁(α-NH₂)₈][Lys]₁₆[Lys]₈[Lys]₄[Lys]₂[Lys]-(PN)NCO-PEG₂₄-CONH-PEG₄-(PhTzMe)/TCO-PEG₃-NHCO-PEG₂₄-Glu-DBCO SRS-22a: To a solution of [(ε-NHCO-PEG₁₁₀₀)₁₆(α-DOTA)₇(α-NHCy5)₁(α-NH₂)₈][Lys]₁₆[Lys]₈[Lys]₄[Lys]₂[Lys]-(PN)NCO-PEG₂₄-CONH-PEG₄-(PhTzMe) HH-2 (1.50 mg; 10 mg/mL in deionised water) was added TCO-PEG₃-NHCO-PEG₂₄-Glu-DBCO Compound 51 (0.20 mg; 2 eq; 10 mg/mL in deionised water) and the reaction mixture left at rt for 2 h. The excess linker was removed by Amicon® Ultra centrifugal filters with 10 kDa MWCO Ultracel® regenerated cellulose membrane and concentrated by centrifugation at 4000 rpm for 10 min. The retentate was washed with deionised water (×5) by centrifugation at 4000 rpm. Removal of the excess linker was confirmed by analysis of purified product using LCMS (LCMS Method 20-90, 8 min, TFA).
Step 2: Conjugation reaction: A solution of [(ε-NHCO-PEG₁₁₀₀)₁₆(α-DOTA)₇(α-NHCy5)₁(α-NH₂)₈][Lys]₁₆[Lys]₈[Lys]₄[Lys]₂[Lys]-(PN)NCO-PEG₂₄-CONH-PEG₄-(PhTzMe)/TCO-PEG₃-NHCO-PEG₂₄-Glu-DBCO (1.59 mg; 10 mg/mL; 1 eq) in deionised water was added to a solution of Nanobody-N3 (1.64 mg; 10 mg/mL in pH 8 Tris buffer; 2 eq) at rt. After 18 h, the reaction mixture was first purified by Amicon® Ultra centrifugal filters with 30 kDa MWCO Ultracel® regenerated cellulose membrane and concentrated by centrifugation at 4000 rpm for 10 min. The retentate was washed with pH 8 Tris, buffer (×5) by centrifugation at 4000 rpm. The product obtained then purified by nickel affinity column chromatography followed by SEC to give [(ε-NHCO-PEG₁₁₀₀)₁₆(α-DOTA)₇(α-NHCy5)₁(α-NH₂)₈][Lys]₁₆[Lys]₈[Lys]₄[Lys]₂[Lys]-(PN)NCO-PEG₂₄-CONH-PEG₄-(PhTzMe)/TCO-PEG₃-NHCO-PEG₂₄-Glu-DBCO/N₃-Nanobody SRS-22 (0.65 mg). See FIG. 23, (Lane 2) for SDS Page gel analysis of the purified product.
   2.4a.12 BHALys[Lys]₂[Lys]₄[Lys]₈[Lys]₁₆[((α-NH-COPEG₂₄NH-COPEG₄(PhTzMe) TCO-PEG₃NH-COPEG₂₄NH-Glu-DBCO N₃-Nanobody)₁(α-NHCy5)₁(α-NHDOTA))₁₀(α-NH₂)₄)(ε-NH-COPEG₁₀₀₀)₁₆], G4, Compound SRS-2-304
Step 1: Preparation of TCO-PEG₃NH-COPEG₂₄NH-Glu-DBCO/N₃-Nanobody (using “Nanobody-N₃—C-terminal Tag”) Compound SRS-2a: A solution of TCO-PEG₃NH-COPEG₂₄NH-Glu-DBCO, Compound 51 (4.69 mg, 2.6 μmol) in MQ water (469 μL) was added to a solution of the Nanobody-N₃ (“Nanobody-N₃—C-terminal tag”) (3.5 mL of a 5.84 mg/mL solution in pH 8 Tris buffer; 1.3 μmol). The reaction mixture was left to stand at rt for 18 h and then concentrated by spin column (Amicon Ultra-15, 10 kDa MWCO). The retentate was washed repeatedly with 20 mM pH 8 Tris Buffer (6×15 mL) and finally concentrated to give Compound SRS-2a (11.3 mg/mL). UPLC-ToF (Method 2): Rₜ=4.74 mins. ESI MS (+ve) 17242 [M]⁺; calc m/z [M]⁺: 17242
Step 2: To a solution of BHALys[Lys]₂[Lys]₄[Lys]₈[Lys]₁₆[((α-NH-COPEG₂₄NH-COPEG₄(PhTzMe)₁(α-NHCy5)₁(α-NHDOTA))₁₀(α-NH₂)₄)(ε-NH-COPEG₁₀₀₀)₁₆], G4 Compound SRS-1 (36.0 mg. 1.09 μmol) in MQ water (4.0 mL) was added TCO-PEG₃NH-COPEG₂₄NH-Glu-DBCO/N₃-Nanobody (using “Nanobody-N₃—C-terminal Tag”) Compound SRS-2a (1.2 eq, 2.0 mL of a 11.3 mg/mL solution in pH8 Tris buffer). The resulting solution was left at rt for 18 h and then purified by metal affinity chromatography followed by SEC to give Compound SRS-2-304 (32.28 mg, 60%; confirmed by SDS-gel, not shown.) See Example 7 Table for Her2+ cell binding data.

### 2a.4 Nanobody-Cys SRS-13 Expression Plasmid

The coding sequence of anti-HER2 nanobody clone 2D3 (US20110028695A1 SEQ ID NO: 1986) was inserted into the expression plasmid pET-His6-TEV-1B (Addgene plasmid 29653) using standard molecular biology techniques. E. coli K12 codon optimised DNA sequences were synthesised and then cloned into plasmid pET-His6-TEV-1B.

For incorporation of C-terminal unpaired cysteine residue into the recombinant protein, the cysteine codon (TGC) as part of a sulfatase peptide motif (LCTPSR) (Carrico et. al, Nat Chem Biol, 3, 2007, p321-322) was inserted in-frame after the nanobody coding sequence, followed then by the sequence encoding the TEV protease cleavage site, a 6his tag and an opal (TGA) stop codon for translational termination. It will be appreciated that these additional motifs are optional beyond simply the cysteine codon

### 2a.5 Expression and Purification of Nanobody-Cys SRS-13

Anti-HER2 nanobody 2D3 expression was performed in E. coli strain BL21(DE3) (Invitrogen) transformed with nanobody expression plasmid. Cells were cultured in Terrific broth (25 g/L tryptone, 30 g/L yeast and 5 g/L glycerol, 0.017 M KH₂PO₄, 0.072 M K₂HPO₄, 50 μg/mL kanamycin sulphate) contained in baffled shake flasks at 37° C. until a cell density of OD₆₀₀ 0.7-1.0 was reached. Recombinant protein expression was induced by addition of 1.5 mM IPTG and allowed to proceed for 20 hours at 25° C. Bacteria were harvested by centrifugation and a cell lysate generated by high pressure homogenisation followed by addition of a protease inhibitor cocktail, lysozyme and DNAse treatment. The lysate was incubated in refolding buffer (6 M Guanidine HCl, 50 mM NaH2PO4, 300 mM NaCl, 20 mM Imidazole, pH 8) overnight. Following clarification by high speed centrifugation and filtration through a 0.45 μM membrane filter, his6-tagged nanobody was purified by immobilised metal affinity chromatography (IMAC) using nickel-charged nitrilotriacetic acid-agarose. On-column refolding was achieved by first washing the bound protein with a column volume of 6 M Guanidine HCl, 50 mM NaH₂PO₄, 300 mM NaCl, 20 mM Imidazole, pH 8, followed by subsequent washes with the same buffer but at 4 M, 3 M, 2 M, 1 M and 0.5 M Guanidine HCl concentrations. This was followed by two column volumes of 50 mM NaH₂PO₄, 300 mM NaCl, 20 mM Imidazole, pH 8. Refolded proteins were eluted with 2 column volumes of 50 mM NaH₂PO₄, 300 mM NaCl, 250 mM Imidazole, pH 8. Following IMAC, Nanobody-Cys SRS-13 was further purified by anion exchange chromatography using a HiTrap Q HP column (GE Healthcare, catalogue 17115301). Binding and washing steps were performed using Tris buffer (20 mM, pH 8) and elution performed using a gradient of 0% to 50% of 1 M NaCl buffer (1M NaCl, 20 mM Tris, pH 8). Relevant nanobody fractions obtained were buffer exchanged into 20 mM Tris, pH 8 using Amicon 10 k MWCO filter units (Merck, catalogue UFC901008). Note: Nanobody-Cys SRS-13 is predominantly in a dimerized form (via-S—S— bond) and must be reduced before conjugation).

### 2a.6 Conjugation of Nanobody-Cys SRS-13 to Dendrimers

Nanobody-Cys SRS-13 was conjugated to the dendrimers SRS-4-Mal, RP-5, HH-2 and SRS-1 using maleimide/Cys conjugation chemistry to generate Compounds SRS-15-17 and 21 according to the procedure described in General Procedure K.

Gels were run for the conjugates SRS-15-17 and 21 (FIG. 23), which showed bands at the appropriate MW for the nanobody-dendrimer conjugates. The purity of each nanobody-dendrimer conjugate was confirmed using non-reducing sodium dodecyl sulfate-polyacrylamide gel electrophoresis (SDS-PAGE). Following electrophoretic separation on a 4-15% polyacrylamide gel (Bio-rad, catalogue 4561086), the nanobody-dendrimer conjugates appear as fluorescent bands corresponding approximately to the size of the dendrimer plus the additional mass of the nanobodies when imaged using a Typhoon Biomolecular Imager (GE Healthcare). Subsequent staining with Coomassie Brilliant Blue (CBB) revealed the presence of nanobody at the same locations. No other bands were revealed by CBB indicating the absence of unreacted nanobody and the purity of the preparation.

2a.6.1 [[(ε-NHCO-PEG₁₁₀₀)₈(α-NHDOTA)_{4.75}(α-NHCy5)_{0.5}(α-NH₂)_{2.75}][Lys]₈[Lys]₄[Lys]₂[Lys]-CONH—CH₂—CH₂—S-]₂MAL-PEG₃-NHCO-PEG₂₄-CONH-PEG₄-(PhTzMe)/TCO—NHCO-PEG₂₄-(MAL)Me Cys-Nanobody SRS-15

SRS-15 was synthesised using the General Procedure K using Nanobody-Cys/Me(MAL)-PEG₂₄-CONH-PEG₃-TCO SRS-14 (6.50 mg) and [[(ε-NHCO-PEG₁₁₀₀)₈(α-NHDOTA)_{4.75}(α-NHCy5)_{0.5}((α-NH₂)_{2.75}][Lys]₈[Lys]₄[Lys]₂[Lys]-CONH—CH₂—CH₂—S-]₂MAL-PEG₃-NHCO-PEG₂₄-CONH-PEG₄-(PhTzMe) SRS-4-Mal (9.0 mg) to give [[(ε-NHCO-PEG₁₁₀₀)₈(α-NHDOTA)_{4.75}(α-NHCy5)_{0.5}(α-NH₂)_{2.75}][Lys]₈[Lys]₄[Lys]₂[Lys]-CONH—CH₂—CH₂—S-]₂MAL-PEG₃-NHCO-PEG₂₄-CONH-PEG₄-(PhTzMe)/TCO—NHCO-PEG₂₄-(MAL)Me/Cys-Nanobody SRS-15 (0.80 mg). See FIG. 22, (Lane 2) for SDS Page gel analysis of the reaction mixture.

2a.6.2 [(ε-NHCO-PEG₁₁₀₀)₁₆(α-NHCy5)₁(α-NHDOTA)₈(α-NH₂)₇][Lys]₁₆[Lys]₈[Lys]₄[Lys]₂[Lys]-CONH—CH₂—CH₂—S-(Me)MAL-PEG₂₄-CONH-Bn-Tz(Me)/TCO—NHCO-PEG₂₄-(MAL)Me Cys-Nanobody SRS-16

SRS-16 was synthesised using the General Procedure K using Nanobody-Cys/Me(MAL)-PEG₂₄-CONH-PEG₃-TCO SRS-14 (6.66 mg) and [(ε-NHCO-PEG₁₁₀₀)₁₆(α-NHCy5)₁(α-NHDOTA)₈(α-NH₂)₇][Lys]₁₆[Lys]₈[Lys]₄[Lys]₂ [Lys]-CONH—CH₂—CH₂—S-(Me)MAL-PEG₂₄-CONH-Bn-Tz(Me) RP-5 (9.5 mg) to give [(ε-NHCO-PEG₁₁₀₀)₁₆(α-NHCy5)₁(α-NHDOTA)₈(α-NH₂)₇][Lys]₁₆[Lys]₈[Lys]₄[Lys]₂[Lys]-CONH—CH₂—CH₂—S-(Me)MAL-PEG₂₄-CONH-Bn-Tz(Me)/TCO—NHCO-PEG₂₄-(MAL)Me/Cys-Nanobody SRS-16 (0.81 mg). See FIG. 22, (Lane 4) for SDS Page gel analysis of the reaction mixture.

2a.6.3 [(ε-NHCO-PEG₁₁₀₀)₁₆(α-DOTA)₇(α-NHCy5)₁(α-NH₂)₈][Lys]₁₆[Lys]₈[Lys]₄[Lys]₂[Lys]-(PN)NCO-PEG₂₄-CONH-PEG₄-(PhTzMe)/TCO—NHCO-PEG₂₄-(MAL)Me/Cys-Nb SRS-17

SRS-17 was synthesised using the General Procedure K using Nanobody-Cys/Me(MAL)-PEG₂₄-CONH-PEG₃-TCO SRS-14 (9.0 mg) and [(ε-NHCO-PEG₁₁₀₀)₁₆(α-DOTA)₇(α-NHCy5)₁(α-NH₂)₈][Lys]₁₆[Lys]₈[Lys]₄[Lys]₂[Lys]-(PN)NCO-PEG₂₄-CONH-PEG₄-(PhTzMe) HH-2 (14.2 mg) to give [(ε-NHCO-PEG₁₁₀₀)₁₆(α-DOTA)₇(α-NHCy5)₁(α-NH₂)₈][Lys]₁₆[Lys]₈[Lys]₄[Lys]₂[Lys]-(PN)NCO-PEG₂₄-CONH-PEG₄-(PhTzMe)/TCO—NHCO-PEG₂₄-(MAL)Me/Cys-Nanobody SRS-17 (3.26 mg). See FIG. 22, (Lane 6) for SDS Page gel analysis of the reaction mixture.

2a.6.4 BHALys[Lys]₂[Lys]₄[Lys]₈[Lys]₁₆[((α-NH-COPEG₂₄NH-COPEG₄(PhTzMe) TCO-PEG₃-NHCO-PEG₂₄-(MAL)Me/Cys-Nanobody)₁(α-NHCy5)₁(α-NHDOTA))₁₀(α-NH₂)₄)(ε-NH-COPEG₁₀₀₀)₁₆SRS-21
Step 1: Synthesis of BHALys[Lys]₂[Lys]₄[Lys]₈[Lys]₁₆[((α-NH-COPEG₂₄NH-COPEG₄(PhTzMe)/TCO-PEG₃-NHCO-PEG₂₄-(MAL)Me)₁(α-NHCy5)₁(α-NHDOTA))₁₀(α-NH₂)₄)(F—NH-COPEG₁₀₀₀)₁₆] SRS-21a: To a solution of BHALys[Lys]₂[Lys]₄[Lys]₈[Lys]₁₆[((α-NH-COPEG₂₄NH-COPEG₄(PhTzMe)₁(α-NHCy5)₁(α-NHDOTA))₁₀(α-NH₂)₄)(F—NH-COPEG₁₀₀₀)₁₆] SRS-1 (13.0 mg; 10 mg/mL in pH 6.8 water) was added TCO-PEG₃-NHCO-PEG₂₄-(MAL)Me SRS-12 (2 eq.; 1.40 mg; 10 mg/mL in deionised water) and the reaction mixture left at rt for 2 h. The excess linker was removed from the reaction mixture by Amicon® Ultra centrifugal filters with 10 kDa MWCO Ultracel® regenerated cellulose membrane and concentrated by centrifugation at 4000 rpm for 10 min. The retentate was washed with pH 6.8 water (×5) by centrifugation at 4000 rpm.
Step 2: Reduction of Nanobody-Cys dimer to Nanobody-Cys SRS-13: To a solution of the Nanobody-Cys dimer (3.33 mg/mL in 10 mM PBS, pH 7.2; 1 eq) was added aq 0.5 M TCEP (10 eq). The reaction mixture was incubated at 37° C. for 2 h and used in subsequent steps without further purification.
Step 3: Conjugation reaction: A solution of BHALys[Lys]₂[Lys]₄[Lys]₈[Lys]₁₆[((α-NH-COPEG₂₄NH-COPEG₄(PhTzMe)/TCO-PEG₃-NHCO-PEG₂₄-(MAL)Me)₁(α-NHCy5)₁(α-NHDOTA))₁₀(α-NH₂)₄)(ε-NH-COPEG₁₀₀₀)₁₆] SRS-21a (13.68 mg; 10 mg/mL; 1 eq) in pH 6.8 water was added to a solution of Nanobody-Cys SRS-13 (7.0 mg; 3.33 mg/mL in 10 mM PBS, pH 7.2; 1.1 eq) at rt. After 18 h, the reaction mixture was first purified by Amicon® Ultra centrifugal filters with 30 kDa MWCO Ultracel® regenerated cellulose membrane and concentrated by centrifugation at 4000 rpm for 10 min. The retentate was washed with pH 7.2 PBS, buffer (×5) by centrifugation at 4000 rpm. The semi-pure product obtained was then purified by nickel affinity column chromatography followed by SEC to give BHALys[Lys]₂[Lys]₄[Lys]₈[Lys]₁₆[((α-NH-COPEG₂₄NH-COPEG₄(PhTzMe)/TCO-PEG₃-NHCO-PEG₂₄-(MAL)Me/Cys-Nanobody)₁(α-NHCy5)₁(α-NHDOTA))₁₀(α-NH₂)₄)(ε-NH-COPEG₁₀₀₀)₁₆] SRS-21 (0.65 mg). See FIG. 23, (Lane 4) for SDS Page gel analysis of the purified product.

### 2b HER-2 Affibody-Dendrimer Conjugates

2b.1 Affibody-BCN/N₃-PEG₂₄CO—[N(PN)₂][Lys]₂[Lys]₄[Lys]₈[(α-NHGlu-vc-PAB-MMAE)₈(ε-NH-COPEG₁₁₀₀)₈], G3, Compound 77

Prepared according to General Procedure F, using affibody-BCN (2.0 mg, 286 nmol at 1.0 mg/mL PBS) and azido-PEG₂₄CO—[N(PN)₂][Lys]₂[Lys]₄[Lys]₈[(α-NHGlu-vc-PAB-MMAE)₈(ε-NH-COPEG₁₁₀₀)₈] Compound 65 (1.0 mL of a 240 μM solution). Lyophilisation of the purified material gave a white fluffy powder (2.19 mg, 31%). SDS-PAGE analysis showed band corresponding to affibody-dendrimer conjugate around 30 kDa (700 nm).

2b.2 Affibody-BCN N₃-PEG₂₄CO—[N(PN)₂][Lys]₂[Lys]₄[Lys]₈[(α-NHGlu-vc-PAB-MMAE)₈(ε-NH-COPEG₂₀₀₀)₈], G3, Compound 78

Prepared according to General Procedure F, using affibody-BCN (1.0 mg, 143 nmol at 1.0 mg/mL PBS) and azido-PEG₂₄CO—[N(PN)₂][Lys]₂[Lys]₄[Lys]₈[(α-NHGlu-vc-PAB-MMAE)₈(ε-NH-COPEG₂₀₀₀)₈] Compound 66 (250 μL of a 233 μM solution). SDS-PAGE analysis showed band corresponding to affibody-dendrimer conjugate around 40 kDa (700 nm).

2b.3 Affibody-BCN/N₃-PEG₂₄CO—[N(PN)₂][Lys]₂[Lys]₄[Lys]₈[(α-NHDGA-MMAF(OMe)₈(ε-NH-COPEG₁₁₀₀)₈], G3, Compound 81

Prepared according to General Procedure F, using affibody-BCN (2.0 mg, 286 nmol at 1.0 mg/mL PBS) and azido-PEG₂₄CO—[N(PN)₂][Lys]₂[Lys]₄[Lys]₈[(α-NHDGA-MMAF(OMe)₈(ε-NH-COPEG₁₁₀₀)₈] Compound 67 (600 μL of a 365 μM solution). Lyophilisation of the purified material gave a white fluffy powder (2.06 mg, 33%). SDS-PAGE analysis showed a band corresponding to affibody-dendrimer conjugate around 30 kDa (700 nm).

2c. FAPI-Targeted Dendrimers and Controls
2c.1 BHALys[Lys]₂[Lys]₄[Lys]₈[Lys]₁₆[Lys]₃₂[(α-NHCO-Lys[(α-NHCy5)(ε-NH-Ph-DFO)])_{1.5}(α-NH₂)_{30.5}(ε-NHCO-dPEG₁₁₀₀-C(O)N-FAPI-04)₁(ε-NHCO-mPEG₁₀₀₀)₃₁] RHa-26

To a solution of BHALys[Lys]₂[Lys]₄[Lys]₈[Lys]₁₆[Lys]₃₂[(α-NH₂.TFA)₃₂(ε-NHCO-dPEG₁₁₀₀-C(O)N-FAPI-04)₁(ε-NHCO-mPEG₁₀₀₀)₃₁] RHa-20 (1.7 μmol) in DMF (1.0 mL) at rt was added NMM in DMF (10 μL/mL, 37 μL, 34 μmol), a solution of HO-Lys[(α-NHCy5)(ε-NH-Ph-DFO)] RHa-9 in DMF (20 mg/mL, 185 μL, 2.5 μmol) and a solution of PyBOP in DMF (100 mg/mL, 13 μL, 2.5 μmol). The progress of the reaction mixture was followed by HPLC (HPLC-Method 1) by monitoring the disappearance of HO-Lys[(α-NHCy5)(ε-NH-Ph-DFO)] RHa-9 (Rₜ=5.32 min) After 18 h, NMM in DMF was added (70 μL, 54 μmol) followed by freshly prepared PyBOP in DMF (50 mg/mL, 30 μL, 2.9 μmol). The reaction mixture was stirred for 18 h whereupon additional portions of NMM in DMF (100 μL, 109 μmol) and PyBOP (6 mg, 11.5 μmol) were added. After stirring for an additional 16 h, the reaction mixture was concentrated in vacuo and the residue dissolved in deionised water (10 mL). The resulting solution was transferred into an Amicon® Ultra 1,5 10 kDa MWCO spin column and concentrated by centrifugation (4000 rpm for 20 min). The retentate was washed with water (10×5 mL @ 4000 rpm for 15 min) and the final retentate then lyophilised to give a dark blue gum (108 mg). Since complete consumption of HO-Lys[(α-NHCy5)(F—NH-Ph-DFO)] RHa-9 was observed by HPLC, the product of the reaction was assigned as BHALys[Lys]₂[Lys]₄[Lys]₈[Lys]₁[Lys]₃₂[(α-NHCO-Lys[(α-NHCy5)(F—NH-Ph-DFO)])_{1.5}(α-NH₂)_{30.5}(ε-NHCO-dPEG₁₁₀₀-C(O)N-FAPI-04)₁(ε-NHCO-mPEG₁₀₀₀)₃₁] RHa-26. HPLC (HPLC-Method 5-80, 8 min, TFA) Rₜ=4.99 min.

2c.2 BHALys[Lys]₂[Lys]₄[Lys]₈[Lys]₁₆[Lys]₃₂[(α-NHCO-Lys[(α-NHCy5)(ε-NH-Ph-DFO)])_{1.5}(α-NH₂)_{30.5}(ε-NHCO-dPEG₁₁₀₀-C(O)N-FAPI-04)₁₀(ε-NHCO-mPEG₁₀₀₀)₁(ε-NH₂)₃] RHa-25

Synthesised according to the method described above for BHALys[Lys]₂[Lys]₄[Lys]₈[Lys]₁₆[Lys]₃₂[(α-NHCO-Lys[(α-NHCy5)(F—NH-Ph-DFO)])_{1.5}(α-NH₂)_{30.5}(ε-NHCO-dPEG₁₁₀₀-C(O)N-FAPI-04)₁(ε-NHCO-mPEG₁₀₀₀)₃₁] RHa-26 from BHALys[Lys]₂[Lys]₄[Lys]₈[Lys]₁₆[Lys]₃₂[(α-NHBoc)₃(ε-NHCO-dPEG₁₁₀₀-C(O)N-FAPI-04)₁₀(ε-NHCO-mPEG₁₀₀₀)₁₉(ε-NH₂)₃] RHa-13 (1.6 μmol). A total of 2.4 μmol of PyBOP, 90 μmol of NMM in DMF (1.5 mL) were added portionwise over 3 d at rt to ensure all HO-Lys[(α-NHCy5)(ε-NH-Ph-DFO)] RHa-9 (2.2 μmol) was consumed (reaction monitored by HPLC). After purification, BHALys[Lys]₂[Lys]₄[Lys]₈[Lys]₁₆[Lys]₃₂[(α-NHCO-Lys[(α-NHCy5)(ε-NH-Ph-DFO)])_{1.5}(α-NH₂)_{30.5}(ε-NHCO-dPEG₁₁₀₀-C(O)N-FAPI-04)₁₀(ε-NHCO-mPEG₁₀₀₀)₁₉(ε-NH₂)₃] RHa-25 was obtained as a thick dark-blue oil (81 mg). IPLC (HPLC-Method 5-80, 8 min, TFA) Rₜ=4.73 min.

2c.3 BHALys[Lys]₂[Lys]₄[Lys]₈[Lys]₁₆[Lys]₃₂[(α-NHCO-Lys[(α-NHCy5)(ε-NH-Ph-DFO)])_{1.5}(α-NH₂)_{30.5}(ε-NHCO-dPEG₁₁₀₀-C(O)N-FAPI-04)₄(ε-NHCO-dPEG₁₀₀₀)₂₈(ε-NH₂)₂] RHa-27

Synthesised according to the method described above for BHALys[Lys]₂[Lys]₄[Lys]₈[Lys]₁₆[Lys]₃₂[(α-NHCO-Lys[(α-NHCy5)(F—NH-Ph-DFO)])_{1.5}(α-NH₂)_{30.5}(ε-NHCO-dPEG₁₁₀₀-C(O)N-FAPI-04)₁(ε-NHCO-mPEG₁₀₀₀)₃₁] RHa-26 from BHALys[Lys]₂[Lys]₄[Lys]₈[Lys]₁[Lys]₃₂[(α-NHBoc)₃₂(ε-NHCO-dPEG₁₁₀₀-C(O)N-FAPI-04)₄(ε-NHCO-dPEG₁₁₀₀)₂₈(ε-NH₂)₂] RHa-14 (82 mg, 1.7 μmol). A total of 16.9 μmol of PyBOP, 1.58 mmol of NMM in DMF (1.5 mL) were added portionwise over 3 d at rt to ensure all HO-Lys[(α-NHCy5)(F—NH-Ph-DFO)] RHa-9 (2.6 μmol) was consumed (reaction monitored by HPLC). After purification, BHALys[Lys]₂[Lys]₄[Lys]₈[Lys]₁₆[Lys]₃₂[(α-NHCO-Lys[(α-NHCy5)(ε-NH-Ph-DFO)])_{1.5}(α-NH₂)_{30.5}(ε-NHCO-dPEG₁₁₀₀-C(O)N-FAPI-04)₄(ε-NHCO-dPEG₁₁₀₀)₂₈(ε-NH₂)₂] RHa-27 was obtained as a blue gum (83 mg). HPLC (HPLC-Method 5-80, 8 min, TFA) Rₜ=4.78 min.

2c.4 BHALys[Lys]₂[Lys]₄[Lys]₈[(α-NHCO-Lys[(α-NHCy5)(ε-NH-Ph-DFO)])_{1.5}(α-NH₂)_{6.5}(ε-NHCO-dPEG₁₁₀₀-C(O)N-FAPI-04)₁(ε-NHCO-mPEG₁₀₀₀)₈(ε-NH₂)₁] RHa-28

Synthesised according to the method described above for BHALys[Lys]₂[Lys]₄[Lys]₈[Lys]₁₆[Lys]₃₂[(α-NHCO-Lys[(α-NHCy5)(F—NH-Ph-DFO)])_{1.5}(α-NH₂)_{30.5}(ε-NHCO-dPEG₁₁₀₀-C(O)N-FAPI-04)₁(ε-NHCO-mPEG₁₀₀₀)₃₁] RHa-26 from BHALys[Lys]₂[Lys]₄[Lys]₈[(α-NHBoc)₈(ε-NHCO-dPEG₁₁₀₀-C(O)N-FAPI-04)₁(ε-NHCO-mPEG₁₀₀₀)₈(ε-NH₂)₂] RHa-16 (85 mg, 6.9 μmol). A total of 20.9 μmol of PyBOP, 1.23 mmol of NMM in DMF (1.5 mL) were added portionwise over 2 d at rt to ensure all HO-Lys[(α-NHCy5)(F—NH-Ph-DFO)] RHa-9 (10.3 μmol) was consumed (reaction monitored by HPLC). After purification, BHALys[Lys]₂[Lys]₄[Lys]₈[(α-NHCO-Lys[(α-NHCy5)(ε-NH-Ph-DFO)])_{1.5}(α-NH₂)_{6.5}(ε-NHCO-dPEG₁₁₀₀-C(O)N-FAPI-04)₁(ε-NHCO-mPEG₁₀₀₀)₅(ε-NH₂)₂] RHa-28 was obtained as a blue gum (96 mg). HPLC (HPLC-Method 5-80, 8 min, TFA) Rₜ=4.89 min.

2c.5 BHALys[Lys]₂[Lys]₄[Lys]₈[(α-NHCO-Lys[(α-NHCy5)(ε-NH-Ph-DFO)])_{1.5}(α-NH₂)_{6.5}(ε-NHCO-dPEG₁₁₀₀-C(O)N-FAPI-04)₃(ε-NHCO-mPEG₁₀₀₀)₅]RHa-29

Synthesised according to the method described above for BHALys[Lys]₂[Lys]₄[Lys]₈[Lys]₁₆[Lys]₃₂[(α-NHCO-Lys[(α-NHCy5)(F—NH-Ph-DFO)])_{1.5}(α-NH₂)_{30.5}(ε-NHCO-dPEG₁₁₀₀-C(O)N-FAPI-04)₁(ε-NHCO-mPEG₁₀₀₀)₃₁] RHa-26 from BHALys[Lys]₂[Lys]₄[Lys]₈[(α-NHBoc)₈(ε-NHCO-dPEG₁₁₀₀-C(O)N-FAPI-04)₃(ε-NHCO-mPEG₁₀₀₀)₅] RHa-17 (85 mg, 6.9 μmol). A total of 19.7 μmol of PyBOP, 1.22 mmol of NMM in DMF (1.5 mL) were added portionwise over 2 d at rt to ensure all HO-Lys[(α-NHCy5)(ε-NH-Ph-DFO)] RHa-9 (9.5 μmol) was consumed (reaction monitored by HPLC). After purification, BHALys[Lys]₂[Lys]₄[Lys]₈[(α-NHCO-Lys[(α-NHCy5)(ε-NH-Ph-DFO)])_{1.5}(α-NH₂)_{6.5}(ε-NHCO-dPEG₁₁₀₀-C(O)N-FAPI-04)₃(ε-NHCO-mPEG₁₀₀₀)₅] RHa-29 was obtained as a blue gum (94 mg). HPLC (HPLC-Method 5-80, 8 min, TFA) Rₜ=5.23 min.

2c.6 BHALys[Lys]₂[Lys]₄[Lys]₈[(α-NHCO-Lys[(α-NHCy5)(ε-NH-Ph-DFO)])_{1.5}(α-NH₂)_{6.5}(ε-NHCO-dPEG₁₁₀₀-C(O)N-FAPI-04)_{7.4}(ε-NH₂)_{0.6}] RHa-30

Synthesised according to the method described above for BHALys[Lys]₂[Lys]₄[Lys]₈[Lys]₁₆[Lys]₃₂[(α-NHCO-Lys[(α-NHCy5)(F—NH-Ph-DFO)])_{1.5}(α-NH₂)_{30.5}(ε-NHCO-dPEG₁₁₀₀-C(O)N-FAPI-04)₁(ε-NHCO-mPEG₁₀₀₀)₃₁] RHa-26 from BHALys[Lys]₂[Lys]₄[Lys]₈[(α-NHBoc)₈(ε-NHCO-dPEG₁₁₀₀-C(O)N-FAPI-04)_{7.4}(ε-NH₂)_{0.6}]RHa-18 (40 mg, 2.5 μmol). A total of 3.9 μmol of PyBOP, 0.1 mmol of NMM in DMF (1.0 mL) were added to ensure all HO-Lys[(α-NHCy5)(F—NH-Ph-DFO)] RHa-9 (3.9 μmol) was consumed (reaction monitored by HPLC). After purification, BHALys[Lys]₂[Lys]₄[Lys]₈[(α-NHCO-Lys[(α-NHCy5)(ε-NH-Ph-DFO)])_{1.5}(α-NH₂)_{6.5}(ε-NHCO-dPEG₁₁₀₀-C(O)N-FAPI-04)_{7.4}(ε-NH₂)_{0.6}] RHa-30 was obtained as a blue gum (45 mg). HPLC (HPLC-Method 5-80, 8 min, TFA) Rₜ=4.61 min. ¹H NMR (300 MHz, CD₃OD): δ (ppm): 1.10-2.02 (m, 186H), 2.08 (s, 6H), 2.14-2.37 (m, 18H), 2.37-2.59 (m, 25H), 2.59-3.27 (m, 120H), 3.36-403 (m, 906H), 4.03-4.52 (m, 54H), 5.03-5.23 (m, 5H), 6.07-6.44 (m, 4H), 6.44-6.78 (m, 1H), 7.12-7.43 (m, 28H), 7.43-7.69 (m, 21H), 7.85-7.92 (m, 1H), 7.92-8.07 (m, FAPI-04-DFO 15H), 8.15-8.30 (m, 4H) and 8.70-8.88 (m, 8H).

2c.7 BHALys[Lys]₂[Lys]₄[Lys]₈[(α-NH—C(S)—NH-Bn-DOTA)₄(α-NH₂)₄(ε-NHCO-dPEG₁₁₀₀-C(O)N-FAPI-04)₃(ε-NHCO-mPEG₁₀₀₀)₅] RHa-34

To BHALys[Lys]₂[Lys]₄[Lys]₈[(α-NH₂.TFA)₈(ε-NHCO-dPEG₁₁₀₀-C(O)N-FAPI-04)₃(ε-NHCO-mPEG₁₀₀₀)s] RHa-23 was added a solution of p-SCN-Bn-DOTA RHa-33 in DMF (10.3 mg/mL, 1.2 mL, 18 μmol). The reaction mixture diluted with DMF (1.2 mL) and DIPEA (100 μL, 0.17 mmol) added. After stirring for 1.5 h, the reaction mixture was concentrated in vacuo, dissolved in water (6 mL) and the resulting solution was transferred into 1× Amicon® Ultra 15 3 k MWCO spin column and centrifuged at 4000 rpm for 25 min at rt. The concentrated retentate was washed with water (10×2.5 mL, 4000 rpm, 15 min) and lyophilised to give BHALys[Lys]₂[Lys]₄[Lys]₈[(α-NH—C(S)-Bn-DOTA)₄(α-NH₂)₄(ε-NHCO-dPEG₁₁₀₀-C(O)N-FAPI-04)₃(ε-NHCO-mPEG₁₀₀₀)₅] RHa-34 as a pale-yellow solid (59 mg, quant). HPLC (HPLC-Method 5-80, 8 min, TFA) Rₜ=4.45 min. ¹H NMR (300 MHz, CD₃OD): δ (ppm) 1.00-2.10 (m, 104H), 2.10-2.37 (m, 8H), 2.37-2.54 (m, 8H), 2.54-2.78 (m, 13H), 2.78-3.28 (m, 74H), 3.35 (s, 21H), 3.37-3.92 (m, 914H), 3.92-4.60 (m, 52H), 5.07-5.23 (m, 3H), 6.12-6.23 (m, 1H), 7.09-7.40 (m, 19H), 7.40-7.72 (m, 14H), 7.92-8.06 (m, 6H), 8.70-8.74 (m, 3H).

2c.8 BHALys[Lys]₂[Lys]₄[Lys]₈[Lys]₁₆[Lys]₃₂[(α-NH—C(S)—NH-Bn-DOTA)₈(α-NH₂)₂₄(ε-NHCO-dPEG₁₁₀₀-C(O)N-FAPI-04)₁₀(ε-NHCO-mPEG₁₀₀₀)₁₉(ε-NH₂)₃] RHa-35

To BHALys[Lys]₂[Lys]₄[Lys]₈[Lys]₁₆[Lys]₃₂[(α-NH₂.TFA)₃₂(ε-NHCO-dPEG₁₁₀₀-C(O)N-FAPI-04)₁₀(ε-NHCO-mPEG₁₀₀₀)₁₉(ε-NH₂)₃] RHa-19 was added a solution of p-SCN-Bn-DOTA RHa-33 in DMF (10.3 mg/mL, 0.55 mL, 8 μmol). The reaction mixture diluted with DMF (1.45 mL) and DIPEA (100 μL, 0.17 mmol) added. After stirring for 1.5 h, the reaction mixture was concentrated in vacuo, dissolved in water (6 mL) and the resulting solution was transferred into 1 x Amicon® Ultra 15 10 k MWCO spin column and centrifuged at 4000 rpm for 25 min at rt. The concentrated retentate was washed with water (10×2.5 mL, 4000 rpm, 15 min) and lyophilised to give BHALys[Lys]₂[Lys]₄[Lys]₈[Lys]₁₆[Lys]₃₂[(α-NH—C(S)—NH-Bn-DOTA)₈(α-NH₂)₂₄(ε-NHCO-dPEG₁₁₀₀-C(O)N-FAPI-04)₁₀(ε-NHCO-mPEG₁₀₀₀)₁₉(ε-NH₂)₃] RHa-35 as a pale-yellow solid (51 mg, 98%). HPLC (HPLC-Method 5-80, 8 min, TFA) Rₜ=4.61 min. ¹H NMR (300 MHz, CD₃OD): δ (ppm) 0.95-2.34 (m, 401H), 2.34-2.61 (m, 35H), 2.61-3.28 (m, 235H), 3.36H (s, 76H), 3.38-3.81 (m, 3358H), 3.81-3.91 (m, 46H), 3.91-4.55 (m, 203H), 5.04-5.21 (m, 16H), 7.03-7.42 (m, 28H), 7.55-7.72 (m, 17H), 7.93-8.06 (m, 20H), 8.72-8.84 (m, 10H).

### 2c.9 FAPI-04-DFO RHa-31 (FAPI-DFO Control)

A solution of p-SCN-Ph-DFO RHa-8 (24.7 mg, 33 μmol) in DMSO (0.5 mL) was added to FAPI-04-NH (TsOH salt) RHa-2 (25.9 mg, 39 μmol) at rt followed by DIPEA (51 μL, 293 μmol). After 1.5 h, another portion of FAPI-04-NH (TsOH salt) RHa-2 (15 mg, 23 μmol) was added and stirring continued. After 2 d, the reaction mixture was purified directly by automated flash column chromatography (Autoflash-Method 2, R_{CV}=10-12 CV) to give FAPI-04-DFO RHa-31 as an off-white solid (29 mg, 71%). LCMS (LCMS Method 5-80, 8 min, TFA) Rₜ=4.56 min. ESI MS (+ve) 1240 [M+H]⁺; calc. m/z for C₅₇H₈₁F₂N₁₄O₁₁S₂ [M+H]⁺=1240. ¹H NMR (300 MHz, CD₃OD): 1.24-1.71 (m, 20H), 2.09-2.15 (m, 5H), 2.41-2.50 (m, 4H), 2.66-3.00 (m, 13H), 3.12-3.19 (m, 4H), 3.54-3.63 (m, 8H), 3.95-4.40 (m, 7H), 5.05-5.20 (m, 1H), 7.19-7.34 (m, 4H), 7.48 (dd, J=3.0, 9.0 Hz, 1H), 7.58 (d, J=3.0 Hz, 1H), 7.86-8.05 (m, 2H) and 8.76 (d, J=6.0 Hz, 1H).

2d. DUPA-Targeted Dendrimers and Controls
2d.1 BHALys[Lys]₂[Lys]₄[Lys]₈[(α-NH₂.TFA)₈(ε-NH-COPEG₂₄NH-DUPA(OH)₃)₈], G3, Compound 37

Prepared according to General Procedure A using BHALys[Lys]₂[Lys]₄[Lys]₈[(α-NHBoc)₈(ε-NH-COPEG₂₄NH-DUPA(OtBu)₃)s], Compound 36 (36 mg, 2.29 μmol). The reaction was stirred overnight at rt. The reaction was concentrated under reduced pressure and purified using SEC (400 drops/tube, MeCN sephadex LH20, 35 drops/min). Fractions were checked using TLC analysis (5% BaCl₂ solution followed by iodine stain; dark brown spot) and those deemed to contain the product were combined and concentrated under reduced pressure. The title compound, Compound 37 was collected as a colourless film (24 mg, 72%). ¹H NMR (300 MHz, CD₃OD) δ (ppm): 0.60-1.99 (m, 138H), 2.08-2.22 (m, 16H), 2.24-2.52 (m, 48H), 3.07-4.06 (m, 845H), 4.27-4.35 (m, 16H), 6.18 (s, 1H), 7.25-7.39 (m, 10H). HPLC (C8 XBridge, 3×100 mm) gradient: 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=6.80 min (broad peak, low intensity).

2d.2 BHALys[Lys]₂[Lys]₄[Lys]₈[((α-(NHDOTA))₁(α-NHAc)₇)(ε-NH-COPEG₂₄NH-DUPA)₈], G3, Compound 38

Prepared according to General Procedure I, Step 2 using BHALys[Lys]₂[Lys]₄[Lys]₈[((α-(NHDOTA))₁(α-NH₂)₇)(ε-NH-COPEG₂₄NH-DUPA)₈]Compound 106 (23 mg, 1.66 μmol) The reaction was concentrated under a stream of N₂ and purified by SEC (400 drops/tube, MeOH sephadex LH20, 35 drops/min). Fractions were checked by TLC analysis (5% BaCl₂ solution followed by iodine stain; dark brown spot) followed by HPLC, and those containing the product were combined and concentrated under reduced pressure. The residue was taken up in MQ water filtered (0.45 μm acrodisc filter) and freeze dried to yield the title compound as a brown waxy solid (22 mg, 91%). HPLC (C₈ XBridge, 3×100 mm) gradient: 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=7.22 min (broad peak).

2d.3 BHALys[Lys]₂[Lys]₄[Lys]₈[Lys]₁₆[Lys]₃₂[(α-NHCy5)₁(α-NHCO-PEG₂₄-NH-DUPA)₁₅(α-NHCO-PEG₂₄NH-GADOTA)₄(α-NH₂.TFA)n₁(ε-NHCOPEG₁₀₀₀)₃₂] RL-22

To a stirred solution of BHALys[Lys]₂[Lys]₄[Lys]₈[Lys]₁₆[Lys]₃₂[(α-NH₂.TFA)₃₂(ε-NHCOPEG₁₀₀₀)₃₂] (100 mg, 1.97 μmol) and Cy5-NHS ester (2.6 mg, 3.94 μmol) in DMF (3 mL) was added NMM (20 μL, 189 μmol) and the ensuing reaction was left to stir for overnight at rt whereupon the volatiles were removed in vacuo to give a blue oil. The residue was dissolved in DMF (3 mL) and DUPA(OtBu)₃-NHPEG₂₄CO₂H, Compound 40 (30 mg, 18.6 μmol), PyBOP (10 mg, 18.6 μmol) and NMM (10 μL, 92.9 μmol) were added sequentially. The ensuing reaction was left to stir overnight at rt. The reaction mixture was divided in half and to one portion was added DOTA(OtBu)₄GA-NHCO-PEG₂₄-COOH RL-21 (34 mg, 19.9 μmol) and PyBOP (10 mg, 18.9 μmol) and the ensuing reaction was left to stir overnight at rt. The volatiles were removed in vacuo and the resulting blue residue was dissolved in deionised water. The solution was concentrated by spin column (Amicon Ultra, 15 mL, 30 kDa MW cut off, 4000 rpm for 20 min) and the retentate was washed repeatedly with water (10×5 mL @4000 rpm for 20 min) and the final retentate was dried by lyophilisation to give a dark blue oil. A portion of the resulting oil (20 mg) was dissolved in dichloromethane (0.5 mL) and TFA (0.5 mL) was added in one portion at rt. The ensuing reaction was left to stir overnight at rt. The reaction mixture was concentrated under a stream of N₂ and the resulting blue residue was dissolved in water, filtered (0.45 μm acrodisc syringe filter) and then dried by lyophilisation to give the title compound RL-22 as a blue solid (15 mg).

The number of DUPA and DOTA were determined by comparing the changes in relative integration for the resonances between 0.0-2.0 ppm and 3.4-4.0 ppm region on the ¹H NMR spectra of isolated intermediate compounds. The terminal methoxy group of the PEG₁₀₀₀ (3.83 ppm, 96H) was used as the reference.

¹H NMR (300 MHz, CD₃OD): δ (ppm) 0.77-2.03 (m, 383H), 2.08-2.27 (m, 36H), 2.27-2.37 (m, 28H), 2.36-2.47 (m, 28), 2.48-2.83 (m, 63H), 3.83 (s, 96H), 3.39-3.93 (m, 4520), 4.01 (m, 62H) and 6.0-8.59 (m, 24H). HPLC (HPLC-Method 5-80, 8 min, TFA) Rₜ=7.08 min.

2d.4BHALys[Lys]₂[Lys]₄[Lys]₈[Lys]₁₆[Lys]₃₂[(α-NHCy5)₁(α-NHCO-PEG₂₄NH-DUPA)₈(α-NHDGA-SN38)₁₆(α-NH₂.TFA)₁₀(ε-NHCOPEG₁₀₀₀)₃₂] RL-23

To a stirred solution of BHALys[Lys]₂[Lys]₄[Lys]₈[Lys]₁₆[Lys]₃₂[(α-NH₂.TFA)₃₂(F-NHCOPEG₁₀₀₀)₃₂] (200 mg, 3.94 μmol) and Cy5-NHS ester (3 mg, 4.73 μmol) in DMF (5 mL) was added NMM (42 μL, 378 μmol) and the ensuing reaction was left to stir overnight at rt. The volatiles were removed in vacuo to give a blue residue, which was dissolved in deionised water and the solution was concentrated by spin column (Amicon Ultra, 15 mL, 30 kDa MW cut off, 4000 rpm for 15 min) and the retentate was washed repeatedly with water (6×5 mL @ 4000 rpm for 15 min) and the final retentate then lyophilised to give as a dark-blue solid (171 mg). A portion of the resulting solid (46 mg, 0.968 μmol) was dissolved in DMF (2 mL) and a solution of DGA-(C-20) SN38 RL-24 (9.4 mg, 18.6 μmol) in DMF (1 mL) was added to the dark blue solution, followed by PyBOP (9.6 mg, 18.6 μmol) and NMM (10 μL, 92.2 μmol) and the ensuing reaction was left to stir overnight at room temperature. The volatiles were removed in vacuo to give a blue residue, which was purified by size exclusion chromatography (stationary phase=Sephadex LH-20@, mobile phase=MeCN, flow rate ˜35 drops/min, collecting 400 drops/fraction). The blue fractions were collected, combined, and concentrated in vacuo to give a dark blue oil. The resulting oil (33 mg, 0.593 μmol) was redissolved in DMF (2 mL) to which was added DUPA(OtBu)₃-NHPEG₂₄CO₂H Compound 40, (18 mg, 11.4 μmol), PyBOP (6 mg, 11.4 μmol) followed by NMM (6.25 μL, 56.9 μL). The ensuing reaction was left to stir overnight at rt. The volatiles were removed in vacuo to give a blue residue, which was purified size exclusion chromatography (stationary phase=Sephadex LH-20@, mobile phase=MeCN, flow rate ˜35 drops/min, collecting 400 drops/fraction). Fractions containing blue material were checked by LCMS analysis and those deemed to contain pure product were concentrated in vacuo to give a dark-blue residue. To a solution of the resulting residue in dichloromethane (1 mL) was added TFA (1 mL) in one portion at rt and the resulting bright green solution was left to stir overnight. The reaction was concentrated under a stream of N₂ and then under high vacuum and the resulting blue/green residue was purified using size exclusion chromatography (stationary phase=Sephadex LH-20@, mobile phase=MeCN, flow rate ˜35 drops/min, collecting 400 drops/fraction). Fractions containing blue material were checked using HPLC analysis and those deemed to contain the product were combined and concentrated in vacuo. The resulting blue residue was dissolved in water, filtered (0.45 μm acrodisc syringe filter) and then dried by lyophilisation to give the title compound RL-23 as a blue solid (9 mg).

The number of DUPA and SN38 were determined by analysis of the ¹H NMR spectrum using the terminal methoxy group of the PEG₁₀₀₀ (3.83 ppm, 96H) was used as the reference.

¹H NMR (300 MHz, CD₃CN): δ (ppm) 0.5-1.9 (m, 582H), 2.51-3.00 (m, 76H), 3.33 (s, 96H), 3.40-5.0 (m, 3282H), 4.45 (m, 16H), 5.3 (m, 16H) and 6.0-8.59 (m, 102H). HPLC (HPLC-Method 5-80, 8 min, TFA) Rₜ=8.60 min.

2d.5 BHALys[Lys]₂[Lys]₄[Lys]₈[Lys]₁₆[Lys]₃₂[(α-NHCy5)₁(α-NHCO-PEG₂₄NH-DUPA)₈(α-NHDOTA)₁₄(α-NH₂.TFA)₉(ε-NHCOPEG₁₀₀₀)₃₂] RL-25

To a stirred solution of BHALys[Lys]₂[Lys]₄[Lys][Lys]₁₆[Lys]₃₂[(α-NH₂.TFA)₃₂(F-NHCOPEG₁₀₀₀)₃₂] (300 mg, 5.92 μmol) and Cy5-NHS ester (6 mg, 7.10 μmol) in DMF (3 mL) was added NMM (62 μL, 568 μmol) and the ensuing reaction was left to stir overnight at rt. The reaction was checked for the consumption of Cy5-NHS ester using LCMS analysis and the volatiles were removed in vacuo to give a blue oil. A portion of the resulting oil (46 mg, 0.968 μmol) was dissolved in DMF (2 mL) and DOTAGA-tetra-(tert-butyl ester) RL-19 (12 mg, 17.4 μmol), PyBOP (9 mg, 17.4 μmol) and NMM (10 μL, 92.9 mmol) were added sequentially. Upon complete consumption of DOTAGA-tetra-(tert-butyl ester) RL-19 (as monitored by LCMS analysis of the reaction mixture) the volatiles were removed in vacuo. The resulting blue residue was dissolved in deionised water and the solution was concentrated by spin column (Amicon Ultra, 15 mL, 30 kDa MW cut off, 4000 rpm for 15 min) and the retentate was washed repeatedly with water (6×5 mL @ 4000 rpm for 15 min) and the final retentate concentrated in vacuo. The residue was dissolved in DMF (2 mL) and DUPA(OtBu)₃-NHPEG₂₄CO₂H, Compound 40 (30 mg, 18.6 μmol), PyBOP (10 mg, 18.6 μmol) and NMM (10 μL, 92.9 μmol) were added sequentially. The ensuing reaction was left to stir overnight at rt. The volatiles were removed in vacuo the resulting blue residue was dissolved in deionised water and the solution was concentrated by spin column (Amicon Ultra, 15 mL, 30 kDa MW cut off, 4000 rpm for 15 min). The retentate was washed repeatedly with MQ water (6×5 mL @ 4000 rpm for 15 min) and the final retentate was dried by lyophilisation to give a dark blue solid. The resulting solid was dissolved in dichloromethane (2 mL) and TFA (1 mL) was added to the solution in one portion at rt. The reaction ensuing reaction was left to stir overnight at rt and then concentrated under a stream of N₂, dissolved in deionised water and the solution was concentrated by spin column (Amicon Ultra, 15 mL, 30 kDa MW cut off, 4000 rpm for 15 min). The retentate was washed repeatedly with MQ water (5×5 mL @ 4000 rpm for 15 min) and the final retentate was dried by lyophilisation to give a blue oil (11 mg).

The number of DUPA and DOTA were determined by comparing the between the 0.0-2.0 ppm region on the ¹H NMR spectra of the isolated intermediates. The terminal methoxy group of the PEG₁₀₀₀ (3.83 ppm, 96H) was used as the reference.

¹H NMR (300 MHz, CD₃OD): δ (ppm) 0.97-1.99 (m, 412H), 2.06-2.93 (m, 182H), 2.93-3.29 (m, 144H), 3.37 (s, 96H), 3.33-4.72 (m, 4228H), 7.32 (m, 10H) and 6.0-8.59 (m, 24H). HPLC (HPLC-Method 5-80, 8 min, TFA) Rₜ=8.29 min.

2d.6BHALys[Lys]₂[Lys]₄[Lys]₈[(α/ε-NHCy5)₁(α/ε-NHCO-PEG₂₄NH-DUPA)₈(α/ε-NH-DOTAGA)₇(α/ε-NH₂.TFA)₃] RL-26

To a solution of BHALys[Lys]₂[Lys]₄[Lys]₈[(ca-NH₂.TFA)₈(ε-NH₂.TFA)₈] (50 mg, 25.1 μmol) in DMF (3 mL) was added Cy5-NHS ester (20 mg, 30.1 μmol) and NMM (52 μL, 0.481 mmol) and the ensuing reaction was stirred overnight at rt. The volatiles were removed in vacuo and a portion of the resulting blue residue (25 mg, 16.2 μmol) was redissolved in DMF (2 mL). To the resulting blue solution was added DOTAGA-tetra-(tert-butyl ester) RL-19 (95 mg, 0.136 mmol), PyBOP (70 mg, 0.136 mmol) and NMM (85 μL, 0.778 mmol) sequentially and the reaction was stirred overnight at rt. The volatiles were removed in vacuo and the residue was purified using size exclusion chromatography (stationary phase=Sephadex LH-20@, mobile phase=MeCN, flow rate ˜35 drops/min, collecting 400 drops/fraction). Fractions containing dark blue material were combined and concentrated in vacuo and the residue was re-dissolved in water and then dried by lyophilisation. To a solution of the resulting blue solid (14 mg, 2.31 μmol) was added DUPA(OtBu)₃-NHPEG₂₄CO₂H, Compound 40 (35 mg, 22.2 μmol), PyBOP (11 mg, 22.2 μmol) followed by NMM (12 μL, 110 μmol) and the ensuing reaction was stirred overnight at rt. The volatiles were removed in vacuo and the residue was purified using size exclusion chromatography (stationary phase=Sephadex LH-20@, mobile phase=MeCN, flow rate ˜35 drops/min, collecting 400 drops/fraction). Fractions containing dark blue material were checked using LCMS analysis, and those deemed to contain the product were combined and concentrated in vacuo. The resulting blue oil was redissolved in dichloromethane (1 mL) and TFA (1 mL) was added in one portion at room temperature and the ensuing green solution was stirred overnight. The reaction was concentrated under a stream of N₂ and then dissolved in deionised water and the solution was concentrated by spin column (Amicon Ultra, 15 mL, 30 kDa MW cut off, 4000 rpm for 15 min). The retentate was washed repeatedly with water (4 x 5 mL @ 4000 rpm for 15 min) and the final retentate was dried by lyophilisation to give a blue oil (11 mg).

The number of DUPA and DOTA were determined by comparing the changes in relative integration for the resonances between 0.0-2.0 ppm, 3.4-4.0 ppm, and 4.0-4.5 ppm and 6.0-8.5 ppm regions on the ¹H NMR spectra of the isolated intermediate compounds.

¹H NMR (300 MHz, CD₃OD): δ (ppm) 0.67-2.03 (m, 112H), 2.05-2.23 (m, 19H), 2.24-2.84 (m, 67H), 2.90-3.20 (m, 58H), 3.33-3.48 (m, 30H), 3.48-4.09 (m, 565H), 4.10-4.52 (m, 33H) and 6.0-8.59 (m, 24H). HPLC (HPLC-Method 5-80, 8 min, TFA) Rₜ=9.92 min (broad).

2d.7BHALys[Lys]₂[Lys]₄[Lys]₈[Lys]₁₆[Lys]₃₂[((α-NHCy5)₁(α-NHAc)₃₁)(ε-NH-COPEG₅₇₀N₃)₁₂(ε-NH-COPEG₅₇₀N₃/BCN-DUPA)₂₀], Compound 100

Prepared according to General Procedure J using BHALys[Lys]₂[Lys]₄[Lys]₈[Lys]₁₆-[Lys]₃₂[((α-NHCy5)₁(α-NHAc)₃₁)(ε-NH-COPEG₅₇₀N₃)₃₂], Compound 34 (10.0 mg, 0.4 μmol) and DUPA-BCN, Compound 61 (10.9 mg, 17.4 μmol). Compound 100 was obtained as a blue solid (11.6 mg, 80%) after purification by SEC (Sephadex LH-20). ¹HNMR (300 MHz, D₂O) δ (ppm): 0.54-2.54 (m, 797H); 2.54-2.86 (m, 44H); 2.88-3.18 (m, 123H); 3.19-4.32 (m, 1409H); 4.34-4.53 (m, 40H); 6.92-7.53 (m, 21H); 7.76-8.25 (m, 14H). LC (LCMS Method 5-80, 15 min, TFA, no MS) Rₜ=9.15 min. ¹HNMR analysis shows 20 DUPA/dendrimer. MW of Compound 100 is ˜40.2 kDa.

2d.8 BHALys[Lys]₂[Lys]₄[Lys]₈[Lys]₁₆[Lys]₃₂[((α-NHCy5)₁(α-NHAc)₃₁)((ε-NH-COPEG₅₇₀N₃)₂₀(ε-NH-COPEG₅₇₀N₃ BCN-DUPA)₁₂)], Compound 101

Prepared according to General Procedure J using BHALys[Lys]₂[Lys]₄[Lys]₈-[Lys]₁₆[Lys]₃₂[((α-NHCy5)₁(α-NHAc)₃₁)(ε-NH-COPEG₅₇₀N₃)₃₂], Compound 34 (10.0 mg, 0.4 μmol) and DUPA-BCN, Compound 61 (4.2 mg, 6.7 μmol). Compound 101 was obtained as a blue solid (15.4 mg, 121%) after purification by SEC (Sephadex LH-20). ¹HNMR (300 MHz, D₂O) δ (ppm): 0.40-2.53 (m, 1430H); 2.55-2.83 (m, 36H); 2.91-3.17 (m, 123H); 3.18-4.32 (m, 1392H); 4.35-4.55 (m, 24H); 6.98-7.50 (m, 26H); 7.76-8.17 (m, 20H). LC (LCMS Method 5-80, 15 min, TFA, no MS) Rₜ=9.15 min. ¹HNMR analysis shows 12 DUPA/dendrimer. MW of Compound 101 is ˜ 35.2 kDa.

2d.9 BHALys[Lys]₂[Lys]₄[Lys]₈[Lys]₁₆[Lys]₃₂[((α-NHCy5)₁(α-NHAc)₃₁)(ε-NH-COPEG₁₁₀₀N₃)₂₅(ε-NH-COPEG₁₁₀₀N₃/BCN-DUPA)₇], Compound 102

Prepared according to General Procedure J using BHALys[Lys]₂[Lys]₄[Lys]₈[Lys]₁₆[Lys]₃₂[(α-NHCy5)₁(α-NHAc)₃₁(ε-NH-COPEG₁₁₀₀N₃)₃₂], Compound 35 (25.2 mg, 0.5 μmol) and DUPA-BCN, Compound 61 (4.2 mg, 6.7 μmol). Compound 102 was obtained as a blue solid (21.9 mg, 79%) after purification by ultrafiltration and lyophilisation. ¹H NMR (300 MHz, CD₃OD) δ (ppm): 0.50-2.56 (m, 668H); 2.76-2.92 (m, 125H); 3.04-3.62 (m, 2982H); 3.74-3.88 (m, 28H); 3.91-4.21 (m, 94H); 6.85-7.24 (m, 26H); 7.57-7.69 (m, 47H). LC (LCMS Method 5-80, 15 min, TFA, no MS) Rₜ=10.11 min. ¹H NMR analysis shows 7 DUPA/dendrimer. MW of Compound 102 is ˜54.6 kDa.

2d.10 BHALys[Lys]₂[Lys]₄[Lys]₈[Lys]₁₆[Lys]₃₂[((α-NHCy5)₁(α-NHAc)₃₁)(ε-NH-COPEG₁₁₀₀N₃)₂₂(ε-NH-COPEG₁₁₀₀N₃/BCN-DUPA)₁₀], Compound 103

Prepared according to General Procedure J using BHALys[Lys]₂[Lys]₄[Lys]₈[Lys]₁₆-[Lys]₃₂[((α-Cy5)₁(α-NHAc)₃₁)(ε-NH-COPEG₁₁₀₀N₃)₃₂](25.0 mg, 0.5 μmol) and DUPA-BCN, Compound 61 (4.2 mg, 6.7 μmol). Compound 103 was obtained as a blue solid (19.7 mg, 70%) after purification by ultrafiltration and lyophilisation. ¹H NMR (300 MHz, D₂O) δ (ppm): 0.82-2.19 (m, 707H); 2.45 (bs, 64H); 3.05-3.21 (m, 135H); 3.38-3.92 (m, 3053H); 4.04-4.30 (m, 101H); 6.93-7.54 (m, 26H). LC (LCMS Method 5-80, 15 min, TFA, no MS) Rₜ=10.44 min. ¹H NMR analysis shows 10 DUPA/dendrimer. MW of Compound 103 is ˜54.6 kDa.

2d.11 BHALys[Lys]₂[Lys]₄[Lys]₈[Lys]₁₆[Lys]₃₂[((α-Cy5)₁(α-NHAc)₃₁(ε-NH-COPEG₁₁₀₀N₃)₁₉(ε-NH-COPEG₁₁₀₀N₃/BCN-DUPA)₁₃], Compound 104

Prepared according to General Procedure J using BHALys[Lys]₂[Lys]₄[Lys]₈[Lys]₁₆-[Lys]₃₂[((α-Cy5)₁(α-NHAc)₃₁(ε-NH-COPEG₁₁₀₀N₃)₃₂], Compound 35 (26.3 mg, 0.5 μmol) and DUPA-BCN, Compound 61 (12.6 mg, 20.1 μmol). The product was obtained as a blue solid (28.8 mg, 94%) after purification by ultrafiltration and lyophilisation. ¹H NMR (300 MHz, CD₃OD) δ (ppm): 0.73-2.90 (m, 823H); 2.96-3.27 (m, 133H); 3.34-3.99 (m, 2894H); 4.05-4.22 (m, 40H); 4.24-4.44 (m, 78H); 4.44-4.58 (m, 34H); 7.16-7.57 (m, 22H); 7.89-8.05 (m, 38H). LC (LCMS Method 5-80, 15 min, TFA, no MS) Rₜ=9.62 min. ¹H NMR analysis shows 13 DUPA/dendrimer. MW of Compound 104 is ˜58.4 kDa.

2d.12 [[(ε-NHCO-PEG₁₁₀₀)₈(α-NHDOTA)_{4.75}(α-NHCy5)_{0.5}(α-1.90 BHALys[Lys]₂[Lys]₄[Lys]₈[((α-(NHDOTA))(α-NH₂)₇)(ε-NH-COPEG₂₄-N₃/BCN-DUPA)₈], G3, Compound 105

To a solution of BHALys[Lys]₂[Lys]₄[Lys]₈[(ca-NH₂.TFA)₈(ε-NH-COPEG₂₄N₃/BCN-DUPA(OH)₃)₈] Compound 37 (23 mg, 1.66 μmol) in DMF (4 mL) was added a solution of p-SCN-Bn-DOTA (2.6 mg, 3.8 μmol) in DMF and the reaction was stirred overnight at rt. HPLC analysis indicated consumption of the starting material and the reaction was concentrated under reduced pressure. Compound 105 was used without further purification. HPLC (C8 XBridge, 3×100 mm) gradient: 5% MeCN/H₂O (0-1 min), 5-80% MeCN (1-7 min), 80% MeCN (7-12 min), 80-5% MeCN (12-13 min), 5% MeCN (13-15 min), 214 nm, 0.4 mL/min, Rₜ=6.15-6.97 min (broad peak).

### Example 3. SPR Binding Studies of Affibody-MMAE Dendrimers with Erb2 ECD

### Direct Immobilization of ErbB2-ECD

Using a ProteOn XPR36 instrument, ErbB2-ECD was immobilized at 25° C. onto a GLC sensor chip surface (Bio-Rad), with HBS-P+ running buffer (10 mM HEPES, pH 7.4, 150 mM NaCl, 0.05% Tween 20). Standard amine coupling methodology described in Bio-Rad's amine coupling kit was utilized for this immobilization. Lane 1 was activated with a 50:50 mixture of EDC (0.5 mM) and sulfo-NHS (0.125 mM). ErbB2 protein was diluted to 2 mg/mL in 10 mM sodium acetate, pH 5.0 and injected over the activated surface channel. Any remaining activated sites were blocked with 1 M ethanolamine-HCl, pH 8.5. Protein coupling at average response levels of approximately 590 RU (1 RU=1 μg of protein/mm²) was observed.

### SPR Experimental Analysis

All SPR binding experiments were performed at 25° C. using HBS-EP+/BSA (10 mM HEPES, pH 7.4, 150 mM NaCl, 3 mM EDTA, 0.05% Tween20, 0.1 mg/ml BSA) as the instrument running buffer (RB). Following immobilization procedures, various concentrations of anti-ErbB2 affibodies and their MMAE-dendrimer conjugates were injected at 60 μl/min over immobilized ErbB2 proteins and their association monitored for 90 sec. Running buffer was subsequently injected over bound Ab-ErbB2 complexes and dissociation monitored for up to 60 min. Herceptin and Affibody controls failed to dissociate from the chip surface in the running buffer in a reasonable timeframe (≤60 min). All binding measurements were performed in triplicate.

### SPR Data Processing and Analysis

Collected experimental data were processed using Scrubber-Pro software (www.biologic.com.au). Kinetic parameters (ka=association and kd=dissociation rate constants) and equilibrium dissociation constant (K_{D}=k_{d}/kₐ) were derived by fitting each set of experimental data to a Langmuir 1:1 binding model. All binding parameters were reported as averaged values +/−standard deviation.

### SPR Results

Affibody-MMAE-dendrimer conjugates bound specifically to ErbB2 proteins in a process yielding classic kinetic binding profiles. Kinetic binding analyses of Affibody-MMAE-dendrimer conjugates were subsequently performed using dose response methodology. Kinetic rate and affinity parameters derived from SPR sensorgrams are summarized below. Note that the affibody is dimeric and so its binding constant is lower than would be observed for the monomeric affibody used in the dendrimer conjugate. No binding was observed for Compounds 65 and 66. The results clearly indicate nanomolar specific binding for the affibody-dendrimer targets albeit at a reduced amount compared to native affibody.

TABLE
SPR Binding Studies

| | kₐ | k_{d} | |
|---|---|---|---|
| | (M⁻¹s⁻¹) × 10⁴ | (s⁻¹) × 10⁻⁴ | K_{D} |
| Herceptin | 230 ± 10 | 0.1 ± 0.0 | 4.3 ± 0.1 (pM) |
| Affibody | 190 ± 30 | 5.1 ± 0.2 | 266 ± 46 (pM) |
| Compound 77 | 2.5 ± 0.2 | 13.0 ± 00.1 | 50 ± 5 (nM) |
| (Affibody-MMAE- | | | |
| PEG 1100) | | | |
| Compound 78 | 3.2 ± 0.2 | 11.4 ± 00.3 | 36 ± 3 (nM) |
| (Affibody-MMAE- | | | |
| PEG 2000) | | | |
| Compound 65 | — | — | — |
| (G3-MMAE-PEG | | | |
| 1100) | | | |
| Compound 66 | — | — | — |
| (G3-MMAE-PEG | | | |
| 2000) | | | |

### Example 4. Cell Growth Inhibition (SRB Assay) by Affibody-MMAE Dendrimers

Cell growth inhibition in HER2-(ES2) and HER2+(SKOV3) cell lines was determined using the Sulforhodamine B (SRB) assay [Voigt W. “Sulforhodamine B assay and chemosensitivity” Methods Mol. Med. 2005, 110, 39-48.] against various cancer cell lines after 72 h with each experiment run in duplicate. GI50 is the concentration required to inhibit total cell growth by 50%, as per NCI standard protocols.

All compounds were tested based on equivalent drug loading. Results show that the PEG 1100 targeted dendrimer conjugate was more effective at inhibiting cell growth than the dendrimer with a PEG 2000 surface. Affibody alone was ineffective in this assay in either HER2+ or HER2− cell lines.

TABLE
Cell Growth Inhibition in HER2− (ES2) and HER2+
(SKOV3) Cells Cell

| | | ES2 Cell | SKOV3 Cell |
|---|---|---|---|
| | Solvent | line | line |
| Compound tested | used | GI₅₀ (nM) | GI₅₀ (nM) |
| MMAE | DMSO | <5, 2 | <5, 0.6 |
| MMAE-Cit-Val-PAB | DMSO | 320, >500 | 201, 330 |
| Affibody | PBS | >500, >500 | >500, >500 |
| Compound 77 | PBS | 248, 250 | 4, 5 |
| Compound 78 | PBS | >100 | >500 |

### Example 5. Tolerability of Affibody-MMAE Dendrimers In Vivo

Groups of mice were administered an intravenous injection of dendrimer (0.1-0.3 ml solution in PBS) once weekly for 3 weeks (day 1, 8 and 15). Mice were weighed daily and watched for signs of toxicity. Animals were monitored for up to 10 days following the final drug dose. Any mice exceeding ethical endpoints (≥20% body weight loss, poor general health) were immediately sacrificed and observations were noted.

Compound 77 at 1 mg/kg was well tolerated in both nude SCID and balb/c mice with no animal needing to be sacrificed for poor health.

### Example 6. In Vitro Efficacy of Conjugates

TABLE
Description of cell lines used

| | | | Reported | | |
|---|---|---|---|---|---|
| | | | HER2 | | |
| | | | Receptor | EGFR | |
| | Cancer | HER2 | Density | (HER1) | HER 3 |
| Cell Line | Type | Status | per Cell | Density | Status |
| MDA-MB-231 | Breast | 1+ | | 200,000 | Low/Mid |
| SKOV-3 | Ovarian | 2+/3+ | 3-700,000 | 50,000 | Low |
| SK-BR-3 | Breast | 3+ | ~1,500,000 | | Mid |
| NCI-N87 | Gastric | 3+ | ~1,300,000 | | Low |
| OE19 | Oesophageal | 3+ | >1,000,000 | | |

### Part 1: GI₅₀ Panel:

The cytotoxicity of Herceptin®, Kadcyla®, Lapatinib, Compound 71 (control) and 2D3 nanobody targeted dendrimer (Compound 123) toward MDA-MB-231, SKOV-3, SK-Br-3, NCI-N87 and OE-19 cells were evaluated using an MTT assay. Cells were seeded at a density of 5×103, in 96-well plates and incubated overnight. Cells were then treated with 1 log unit serial dilutions of test compositions for 72 h (or six days for Herceptin). 10% media volume of MTT (Thiazolyl Blue Tetrazolium Bromide (Merck, Cat #M5655, 5 mg/ml sterile solution) was added during the final 2 h of incubation. Reduction of MTT in living cells yields an insoluble purple formazan metabolite. After incubation for 2 h, all media was removed from the assay plate, 100 μl of DMSO added and the plate immediately read for absorbance at 570 nm. GI₅₀ is defined as the concentration inhibiting the growth/proliferation of cells by 50%. Cell viability and subsequent GI₅₀ values were was determined from the blank corrected dose-response curves, with 4-parameter nonlinear curve fit, in GraphPad Prism 7.02. The results demonstrate that example conjugates of the present disclosure have potent cytotoxic effects, particularly against cell lines which overexpress HER2, such as SK-BR-3, NCI-N87, and OE19.

TABLE
GI₅₀ results for Compound 71 and Compound 123

| | | | | Com- | Com- |
|---|---|---|---|---|---|
| | | | | pound | pound |
| | Herceptin | Kadcyla* | Lapatinib | 123 | 71 |
| | (n = 3) | (n = 3) | (n = 2) | (n = 2) | (n = 2) |
| | % growth | GI₅₀ | GI₅₀ | GI₅₀ | GI₅₀ |
| | inhibition | nM | nM | nM | nM |
| MDA-MB-231 | 1.7 | 22.9 | 112,000 | 172 | 12,470 |
| SKOV-3 | 10 | 50.7 | 7,200 | 13.1 | 1600 |
| SK-BR-3 | 51 | 0.04 | 138 | 0.83 | 948 |
| NCI-N87 | 52 | 0.10 | 8.1 | 0.61 | 2040 |
| OE19 | 62 | 0.06 | 200 | 1.67 | 6120 |

*Based on estimated weight ADC;
# Based on MMAE drug equivalents

### Part 2: IC₅₀ in Paired Hi/Lo HER2 Cell Lines

### Generation of HER2+ Knock in MDA-MB-231 Breast Cancer Cell Line for Paired Hi/Lo HER2 Cell Lines

MDA-MB-231 breast cancer cells were transfected with plasmid HER2 WT Addgene, 16257) using Lipofectamine 3000 according to manufacturer's instructions. After passaging in the presence of 500 μg/ml Geneticin (Thermo Fisher, catalogue 10131035), cells with stable overexpression of HER2 WT were isolated by fluorescence activated single cell sorting using a MoFlo Astrios (Beckman Coulter). A clonal cell population isolated from this process was further passaged in Geneticin before a second round of cell sorting and screening for transgene expression. A selected clone was then further expanded and stocks of this cell line, referred to as MDA-MB-231/HER2, were cryopreserved to generate a master stock of stably transduced cells.

### IC₅₀ in Lo/Hi Expressing MDA-MB-231 Model:

The cytotoxicity of Compound 71 (control) and Compound 123 (targeted) toward MDA-MB-231, and MDA-MB-231/HER2 transfected cells were evaluated via alamarBlue assays. Cells were seeded at a density of 5×10³, in 96-well plates and incubated overnight. Cells were then treated with indicated concentrations of test compositions for 48 h. AlamarBlue (Thermo Fisher, DAL1025) was added during the final 4 h of incubation. Reduction of alamarBlue in living cells yields a red fluorescent metabolite that can be read on a plate reader (560 nm excitation/610 nm emission). Cell viability and subsequent IC₅₀ was determined from the blank corrected dose-response curves, with 4-parameter nonlinear nonlinear curve fit, variable slope (four parameters) in GraphPad Prism 7.02. The table below shows the IC₅₀ values of Compound 71 (control) and Compound 123 (targeted) with MDA-MB-231 and MDA-MB-231/HER2 cells. The IC₅₀ values of MDA-MB-231/HER2 with Compound 71 (control) and Compound 123 (targeted) were 2.842 μM and 13.55 nM, respectively. Compound 123 (targeted) is approximately 140-fold increase in MDA-MB-231/HER2 cell growth inhibition compared to Compound 71 (control).

The dose response curve and IC₅₀ value for MDA-MB-231 and MDA-MB-231/HER2 of Compound 71 (control) and Compound 123 (targeted) at different MMAE concentrations is provided below. Values are mean±standard deviation (sd; n=4):

TABLE
IC₅₀ results for Compound 71 and Compound 123
in MDA-MB-231 HER2 lo/hi model

| | Compound 71 | | Compound 123 | |
|---|---|---|---|---|
| MMAE | (Cell viability %) | | (Cell viability %) | |
| Concentration | MDA- | MDA-MB- | MDA- | MDA-MB- |
| (M) | MB-231 | 231/HER2 | MB-231 | 231/HER2 |
| 5 × 10⁻⁵ | 81.1 ± 5.4 | 49.9 ± 15.0 | — | |
| 1 × 10⁻⁵ | 94.2 ± 12.1 | 61.6 ± 3.1 | | 5.25 ± 1.24 |
| 1 × 10⁻⁶ | 94.4 ± 8.2 | 78.2 ± 9.1 | | 10.9 ± 2.4 |
| 1 × 10⁻⁷ | 94.2 ± 12.5 | 96.1 ± 3.6 | 49.7 ± 2.7 | 27.4 ± 1.3 |
| 1 × 10⁻⁸ | 96.6 ± 11.7 | 95.4 ± 7.6 | 67.2 ± 3.0 | 57.8 ± 2.3 |
| 1 × 10⁻⁹ | | | 81.0 ± 1.8 | 80.0 ± 3.3 |
| 1 × 10⁻¹⁰ | | | 87.6 ± 4.2 | 91.1 ± 2.5 |
| 1 × 10⁻¹¹ | | | 96.1 ± 5.6 | 90.0 ± 3.0 |
| IC₅₀ | Ambiguous | 2.842 μM | Ambiguous | 13.55 nM |

### Example 7. Binding of 2D3-Dendrimer Conjugates to HER2+ Cells

Different sized nanobody-dendrimer conjugates labelled with Cyanine 5 (Cy5), Compounds 124, 125, and 126 were used to demonstrate binding to HER2+ human cell lines, against untargeted Compounds 52, 53, and 54 (G3, G4, and G5).

The HER2-expressing human metastatic carcinoma cell line MDA-MB-453 (ATCC HTB-131) and the HER2-lo epithelial adenocarcinoma cell line MDA-MB-231 (ATCC HTB-26) were maintained in Dulbecco's Modified Eagle's Medium (DMEM) containing 10% FBS and penicillin-streptomycin (100 U/mL) at 37° C. under a 5% CO₂ humidified atmosphere and subcultured prior to confluence using trypsin. Human adenocarcinoma ovary cell line SKOV-3 (ATCC® HTB-77) cells were maintained in RPMI media with the addition of 10% (v/v) FBS and penicillin-streptomycin (100 U/mL) at 37° C. in a 5% CO₂ humidified atmosphere and subcultured prior to confluence.

For measurement and imaging of cell association by fluorescence microscopy, cells were seeded at 1×10⁵ cells per well in an 8-well chamber slide and allowed to adhere overnight. The following day, unconjugated dendrimer Compounds 52, 53, and 54 (control) or nanobody dendrimer Compounds 124, 125, and 126 were added to the media to a final concentration of 0.5 μg/ml and incubated on ice for 1 hour. Cells were stained to visualise plasma membrane (wheat germ agglutinin-alexa fluor 488) and nucleus (Hoechst 33342). Cells were washed three times with 400 μl of chilled Fluorobrite media supplemented with 10% FBS. Cells were imaged using an Olympus IX83 microscope with a 40×0.9 NA air objective with a standard “Pinkel” DAPI/FITC/CY5 filter set.

### In Vitro Association Studies

MDA-MB-231, MDA-MB-231/HER2 (HER2 knock in) (as described in Ex 6 above), or SKOV-3 cells were seeded in a 24-well plate (1×10⁵ cells per well) in 500 μL of appropriate growth media with the addition of 10% (v/v) fetal bovine serum (FBS) and incubated with Compound 36 or Compound 41 at 3.33 nM, with incubation time varied from 1 to 24 h at 37° C. in a 5% CO₂ humidified atmosphere. After incubation, non-binding/non-associating particles were removed from adherent cells by gently washing with DPBS three times (300 μL/well). Cells were removed from plates by treatment with TrypLE™ Express Enzyme (1×), no phenol red (150 μL/well) for 5-10 min at room temperature. The plates were then placed on ice. Cellular binding and association of sample were then determined through flow cytometry by the acquisition of the signal from Cy5.

To measure internalisation kinetics, nanobody-dendrimer was incubated with cells for 24, 4, 1, 0.5, and 0.1 hours at 37° C. before unbound nanobody-dendrimer was removed by washing and analysed by flow cytometry.

### Flow Cytometry Results: Binding of G3 to G5 Dendrimers to HER Hi/Lo Cell Line:

Results show that the nanobody-dendrimer Compounds 124, 125, and 126 bind to HER2-positive MDA-MB-453 cells. Unconjugated dendrimer Compounds 52, 53, and 54 did not bind to MDA-MB-453 cells. Data shown is the mean MFI of cells treated in triplicate wells, with standard deviation. An ANOVA with Dunnett's multiple comparisons test was used for statistical analysis.

TABLE
Mean fluorescence intensity (Cy5)
of cells incubated with dendrimers

| Compound tested | MFI | ANOVA |
|---|---|---|
| Cells only control | 3.047 ± 0.02 | — |
| 52 | 3.097 ± 0.01 | NS |
| 53 | 6.827 ± 0.07 | NS |
| 54 | 4.349 ± 0.08 | NS |
| 124 | 50.83 ± 14.98 | <0.0001 |
| 125 | 158.4 ± 6.611 | <0.0001 |
| 44126 | 176.9 ± 4.041 | <0.0001 |

### Flow Cytometry Results: Compound 71 and Compound 123 Binding to HER2 Lo and HER2+ Cell Lines

The results clearly indicate minimal binding of Compound 71 (control) towards all three cell lines over 24 h. Compound 123 (targeted) showed increasing binding in relation to the level of HER2 receptor expression of cell line. By 24 h, flow cytometry revealed that MDA-MB-231/HER2 cells treated with Compound 123 (146.1±9.6) displayed approximately 9-fold stronger fluorescence compared to the cells treated with Compound 71 (16.05±1.89). Also, SKOV-3 cells treated with Compound 123 (277.5±5.9) displayed approximately 16-fold stronger fluorescence compared to the cells treated with Compound 71 (16.4±6.86). The results are shown in FIG. 2.

### Cellular Association: Dendrimer Size G3 to G5 Binding to HER2 Lo and HER+ Cell Lines:

Additional dendrimer generations were tested: Compounds 52 and 124 (G3), Compounds 53 and 125 (G4), and Compounds 54 and 126 (G5), unconjugated and conjugated to 2D3 respectively. Unconjugated control dendrimers Compounds 52, 53, and 54 had significantly lower association with MDA-MB-231 (HER2 lo) and MDA-MB-231/HER2 (HER2 positive). 2D3-conjugated dendrimers Compounds 124, 125, and 126 have significant association with MDA-MB-231/HER2 over 24 h, with Compound 126 (G5) having the highest percent of cellular association (73.47%±0.92), second being Compound 124 (G3, 60.73±0.21), and Compound 125 (G4, 56.27±1.02). Regardless of dendrimer generation, 2D3 HER2-nanobody conjugated with dendrimer substantially improves binding towards cells which overexpress HER2 receptors. Flow cytometry results for the conjugates are shown in FIG. 3.

The table below shows percent of cellular association value of various sized G2, G3, G4, and G5 2D3 conjugated dendrimers with MDA-MB-231 and MDA-MB-2311/5ER2 cells over 24 h. Values are mean±standard deviation (SD; n=3):

TABLE
Percent of cellular association value of various dendrimers.
MDA-MB-231/HER2

| Time | Compound | Compound | Compound | Compound | Compound | Compound | Compound |
|---|---|---|---|---|---|---|---|
| (h) | 124 | 125 | 126 | 85 | 89 | 91 | 93 |
| 0.25 | 0.67 ± 0.12 | 0.57 ± 0.12 | 0.43 ± 0.40 | — | — | — | — |
| 0.5 | 0.60 ± 0.0 | 0.67 ± 0.15 | 0.70 ± 0.10 | 189.7 ± 9.074 | 218.3 ± 16.5 | 157.3 ± 7.02 | 165.3 ± 31.5 |
| 0.75 | — | — | — | — | — | — | — |
| 1 | 2.57 ± 0.64 | 0.77 ± 0.15 | 2.80 ± 0.61 | — | — | — | — |
| 2 | 17.43 ± 4.15 | 6.13 ± 2.60 | 17.97 ± 2.20 | — | — | — | — |
| 4 | 50.63 ± 0.15 | 36.03 ± 2.36 | 59.77 ± 4.13 | — | — | — | — |
| 24 | 60.73 ± 0.21 | 56.27 ± 1.02 | 73.47 ± 0.92 | — | — | — | — |

MDA-MB-231/HER2

| Time | Compound | Compound | Compound | Compound | Compound | Compound |
|---|---|---|---|---|---|---|
| (h) | 98 | 99 | 129 | 130 | 131 | 132 |
| 0.25 | — | — | — | — | — | — |
| 0.5 | — | — | 14.83 ± 1.60 | 43.23 ± 3.48 | 16.7 ± 2.48 | 25.6 ± 0.43 |
| 0.75 | 14.97 ± 0.7 | 82.5 | — | — | — | — |
| 1 | — | — | — | — | — | — |
| 2 | — | — | — | — | — | — |
| 4 | — | — | — | — | — | — |
| 24 | — | — | — | — | — | — |

MDA-MB-231

| Time | Compound | Compound | Compound | Compound | Compound | Compound | Compound |
|---|---|---|---|---|---|---|---|
| (h) | 124 | 125 | 126 | 85 | 89 | 91 | 93 |
| 0.25 | 1.07 ± 0.38 | 0.87 ± 0.25 | 0.53 ± 0.50 | — | — | — | — |
| 0.5 | 0.87 ± 0.12 | 0.53 ± 0.15 | 1.03 ± 0.32 | 30.77 ± 0.50 | 25.07 ± 3.91 | 20.03 ± 2.73 | 17.47 ± 1.62 |
| 0.75 | — | — | — | — | — | — | — |
| 1 | 0.80 ± 0.20 | 1.00 ± 0.44 | 1.37 ± 0.59 | — | — | — | — |
| 2 | 1.13 ± 0.21 | 0.77 ± 0.31 | 0.73 ± 0.49 | — | — | — | — |
| 4 | 0.93 ± 0.45 | 0.83 ± 0.21 | 0.67 ± 0.25 | — | — | — | — |
| 24 | 0.83 ± 0.06 | 0.87 ± 0.06 | 0.80 ± 0.10 | — | — | — | — |

MDA-MB-231

| Time | Compound | Compound | Compound | Compound | Compound | Compound |
|---|---|---|---|---|---|---|
| (h) | 98 | 99 | 129 | 130 | 131 | 132 |
| 0.25 | — | — | — | — | — | — |
| 0.5 | — | — | 23.3 ± 3.98 | 42.8 ± 2.78 | 20.23 ± 0.63 | 33.23 ± 2.2 |
| 0.75 | 13.36 ± 2.82 | 0.15 ± 0.07 | | | | |
| 1 | — | — | — | — | — | — |
| 2 | — | — | — | — | — | — |
| 4 | — | — | — | — | — | — |
| 24 | — | — | — | — | — | — |

The table below shows percent of cellular association value of Compound 52 (G3), Compound 53 (G4), and Compound 54 (G5) with MDA-MB-231 and MDA-MB-23 1/HER2 cells over 24 h. Values are mean±standard deviation (SD; n=3):

TABLE
Percent cellular association of G3, G4,
and G5 unconjugated dendrimers.

| Time (h) | Compound 52 | Compound 53 | Compound 54 |
|---|---|---|---|
| | MDA-MB-231/HER2 | | |
| 1 | 2.47 ± 0.81 | 0.90 ± 0.35 | 1.07 ± 0.35 |
| 2 | 1.00 ± 0.26 | 0.50 ± 0.10 | 0.83 ± 0.31 |
| 4 | 1.07 ± 0.64 | 0.50 ± 0.35 | 0.47 ± 0.21 |
| 24 | 0.60 ± 0.17 | 0.37 ± 0.15 | 0.53 ± 0.49 |
| | MDA-MB-231 | | |
| 1 | 1.10 ± 0.35 | 0.80 ± 0.10 | 0.97 ± 0.12 |
| 2 | 1.23 ± 0.47 | 0.77 ± 0.06 | 1.07 ± 0.21 |
| 4 | 0.83 ± 0.15 | 0.60 ± 0.10 | 0.87 ± 0.15 |
| 24 | 0.73 ± 0.06 | 0.60 ± 0.17 | 0.57 ± 0.06 |

Mean fluorescence intensity value of Compound 124 (G3), Compound 125 (G4), and Compound 126 (G5) with MDA-MB-231 and MDA-MB-231/HER2 cells over 24 h. Values are mean±standard deviation (SD; n=3):

TABLE
MFI of G3, G4 and G5 2D3 conjugated dendrimers

| Time (h) | Compound 124 | Compound 125 | Compound 126 |
|---|---|---|---|
| | MDA-MB-231 | | |
| 0.25 | 245.7 ± 17.5 | 227.3 ± 14.0 | 194.7 ± 95.8 |
| 0.5 | 228.0 ± 4.6 | 222.7 ± 2.1 | 245.0 ± 4.0 |
| 1 | 237.0 ± 9.2 | 229.7 ± 6.4 | 253.7 ± 12.9 |
| 2 | 241.0 ± 18.2 | 235.7 ± 4.0 | 233.3 ± 9.7 |
| 4 | 250.0 ± 5.8 | 246.3 ± 5.5 | 247.0 ± 10.6 |
| 24 | 224.3 ± 5.1 | 219.0 ± 4.6 | 223.0 ± 4.4 |
| | MDA-MB-231/HER2 | | |
| 0.25 | 235.0 ± 7.0 | 215.3 ± 14.0 | 207.0 ± 51.6 |
| 0.5 | 226.3 ± 8.7 | 224.3 ± 2.9 | 237.0 ± 9.5 |
| 1 | 294.3 ± 15.6 | 241.0 ± 11.5 | 295.0 ± 4.0 |
| 2 | 445.0 ± 33.2 | 342.3 ± 22.2 | 435.7 ± 16.3 |
| 4 | 799.7 ± 18.0 | 611.3 ± 35.2 | 1,038 ± 127.6 |
| 24 | 1,110 ± 27.8 | 1,012 ± 45.6 | 2,749 ± 88.1 |

Mean fluorescence intensity value of Compound 52 (G3), Compound 53 (G4), and Compound 54 (G5) with MDA-MB-231 and MDA-MB-231/HER2 cells over 24 h.
Values are mean ± standard deviation (SD; n = 3):

TABLE
MFI of G3, G4, and G5 unconjugated dendrimers.

| Time (h) | Compound 52 | Compound 53 | Compound 54 |
|---|---|---|---|
| | MDA-MB-231/HER2 | | |
| 1 | 293.0 ± 8.5 | 269.0 ± 19.8 | 257.5 ± 12.0 |
| 2 | 268.0 ± 11.3 | 225.0 ± 5.7 | 249.0 ± 15.6 |
| 4 | 241.5 ± 17.7 | 198.0 ± 2.8 | 207.0 ± 2.8 |
| 24 | 206.0 ± 2.8 | 175.5 ± 0.7 | 207.5 ± 40.3 |
| | MDA-MB-231 | | |
| 1 | 258.3 ± 19.6 | 239.3 ± 1.2 | 252.0 ± 6.0 |
| 2 | 256.7 ± 24.8 | 230.7 ± 2.3 | 249.0 ± 8.7 |
| 4 | 221.3 ± 3.5 | 217.3 ± 3.2 | 247.3 ± 7.2 |
| 24 | 215.3 ± 4.9 | 195.7 ± 0.6 | 196.3 ± 3.1 |

Mean fluorescence intensity value of Compound SRS-2-304 with MDA-MB-231 and MDA-MB-231/HER2 cells over 24 h.
Values are mean ± standard deviation (SD; n = 3):

TABLE
MFI of SRS-2-304 (Her2 nanobody dendimer)
compared to KY-2a (Trastuzumab).

| Compound tested | Cell line | MFI |
|---|---|---|
| — | HER2 + ve cells alone | 6.2 ± 0.8 |
| — | HER2 − ve cells alone | 6.2 ± 0.1 |
| SRS-2-304 | HER2 + ve | 403.0 ± 9.8 |
| SRS-2-304 | HER2 − ve | 28.1 ± 0.7 |
| KY-2a | HER + ve | 555.0 ± 132.3 |
| KY-2a | HER − ve | 23.6 ± 4.1 |

HER2-nanobody conjugated with dendrimer shows binding to cells which overexpress ITER2 receptors at levels comparable with Trastuzumab.

The above binding assay was also carried out using BT-474 cell line. Mean fluorescence intensity value of Compounds at a range of concentrations after 1 hour incubation is shown in the table below. Values are mean±standard deviation (SD; n=3). The data shows the Her2 targeted dendrimers binding to Her2+ cells.

TABLE
Mean Flourescent Intensity of compounds at different concentrations, performed on BT-474 cell line.

| | (SRS- | | | | | | | | |
|---|---|---|---|---|---|---|---|---|---|
| Concentration | 4-Mal) | (RP-5) | (HH-2) | (SRS-15) | (SRS-16) | (SRS-17) | (SRS-20) | (SRS-21) | (SRS-22) |
| 3 mM | 15.3 ± | 26.2 ± | 28.9 ± | 915 ± | 715.5 ± | 1021 ± | 1505 ± | 3104.5 ± | 2638.5 ± |
| | 0.7 | 8.4 | 3.8 | 141.4 | 217 | 459.6 | 202.2 | 2.1 | 511.2 |
| 6 mM | 23.8 ± | 25.6 ± | 33.8 ± | 1463 ± | 731.5 ± | 1532 ± | 2168 ± | 4644.5 ± | 4911.5 ± |
| | 7 | 3.3 | 5.3 | 291.3 | 70 | 152.7 | 60.8 | 1044.3 | 24.7 |
| 12 mM | 21.9 ± | 23.4 ± | 34.6 ± | 2269.5 ± | 1369 ± | 2271.5 ± | 3135 ± | 5264 ± | 6552 ± |
| | 5.1 | 2.8 | 5.3 | 53 | 86.2 | 366.9 | 33.9 | 838.6 | 436.9 |
| 25 mM | 18.6 ± | 25.6 ± | 49.3 ± | 2152.5 ± | 1449 ± | 2551.5 ± | 3401 ± | 4755.5 ± | 6492 ± |
| | 0.6 | 2.7 | 2.2 | 92.6 | 0 | 246.7 | 73.5 | 217 | 196.5 |

### Example 8. Studies Demonstrating Internalisation of Conjugates into HER2-Overexpressing Cells

### Confocal Cellular Uptake

MDA-MB-231, MDA-MB-231/HER2 and SKOV-3 cells were plated at 1.0×10⁴ cells per well into μ-slide 8-well chambered coverslip (ibidi) and allowed to adhere overnight at 37° C., 5% CO₂. Compound 71 and Compound 123 (3.33 nM) were then added and allowed to incubate for 1, 3, 6, and 24 h. The cells were washed and then fixed with 1% paraformaldehyde for 20 min at room temperature. The cell membrane was stained with Alexa Fluor 488-wheat germ agglutinin (AF488-WGA, 5 μg mL⁻¹) in DPBS at room temperature for 10 min. The cell nuclei were counterstained with 4′,6-diamidino-2-phenylindole (DAPI, 2 μg mL⁻¹) in DPBS at room temperature for 10 min. Fluorescence images and optical sections were collected using a confocal microscope (Leica SP8). Images were processed with Fiji (Image J 1.52n).

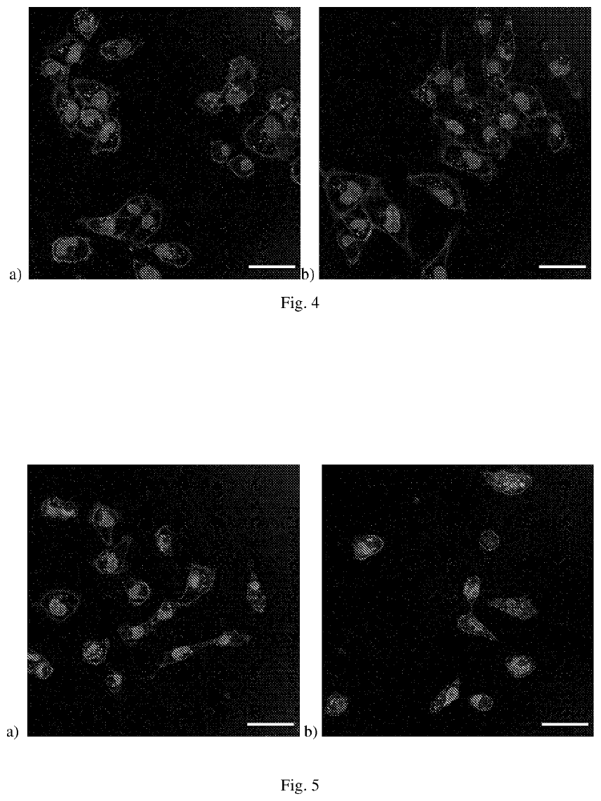

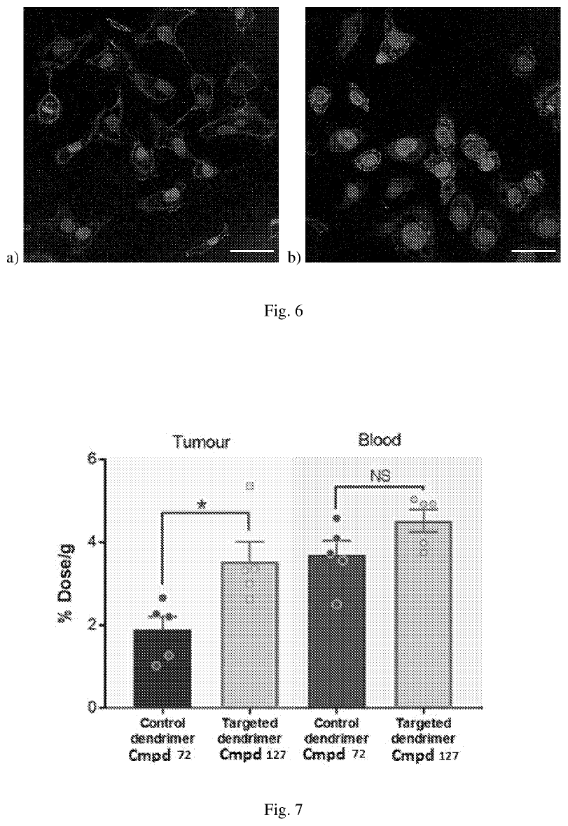

Confocal microscopy images are shown in FIGS. 4 to 6. The confocal images show little of either FIG. 4 a) Compound 71 and FIG. 4 b) Compound 123 associated with MDA-MB-231 after 24 h of incubation. Whereas targeted Compound 123 was internalised by MDA-MB-231/HER2 and SKOV-3 cells after 24 h of incubation, (FIGS. 5a and 6a), Compound 71 was not (FIGS. 5b and 6b).

### Example 9. Tumour Distribution of Tritium-Labelled MMAE-Conjugated Dendrimers (Compounds 72 and 127)

MDA-MB-231/HER2 cells (5×10⁶ cells in 50 μL of PBS:Matrigel) were transplanted into the 4^{th} mammary fat pad of female NOD/SCID mice (5-7 weeks old) subcutaneously. Solid tumours were allowed to grow to 100 mm³ (approximately 3-4 weeks). The mice were divided into two different groups, consisting of six mice in each group. Compound 72 (control group) or Compound 127 (targeted group) (0.5 μCi in 100 μL, PBS pH 7) was then injected via tail vein under isoflurane sedation. The mice were anaesthetised with isoflurane 48 h later, and blood collected via cardiac puncture immediately prior to cervical dislocation. Selected organs (including tumour, liver, spleen, kidney, pancreas, lung, heart and brain) were subsequently removed, weighed and processed for tritium (³H) biodistribution.

The results are shown in FIG. 7. Targeted dendrimer Compound 127 accumulated in HER2 positive tumours (3.53% dose/g±0.43) to a greater extent than the unconjugated control Compound 72 (1.88% dose/g±0.28), which equates to an increase in tumour uptake by approximately 80% (p<0.05). The differences in blood concentration were insignificant between the targeted and control dendrimer, 4.52% dose/g±0.24 and 3.69% dose/g±0.31, respectively. As the differences in pharmacokinetics are unlikely to be affecting tumour retention, 2D3 HER2-nanobody targeting improved dendrimer retention.

### Example 10. Efficacy and Confocal Imaging of Tumour Uptake of Cy5 Labelled—MMAE-Conjugated Dendrimers (Compound 71 and Compound 123) in SKOV3 Tumour Model

Female NOD SCID mice (age 8 weeks) were inoculated subcutaneously on the flank with 3×10⁶ SKOV3 cells in PBS:Matrigel (1:1). Solid tumours were allowed to grow to 200 mm³ (approximately 3 weeks). The mice were divided into five different groups, Compound 71 (control group) or Compound 123 (targeted group) (0.5 mg/kg MMAE equivalents in 250 μL, PBS pH 7), Kadcyla® 40 mg/kg and Herceptin® (40 mg/kg), was then injected via tail vein under isoflurane sedation.

### (a) Confocal Imaging

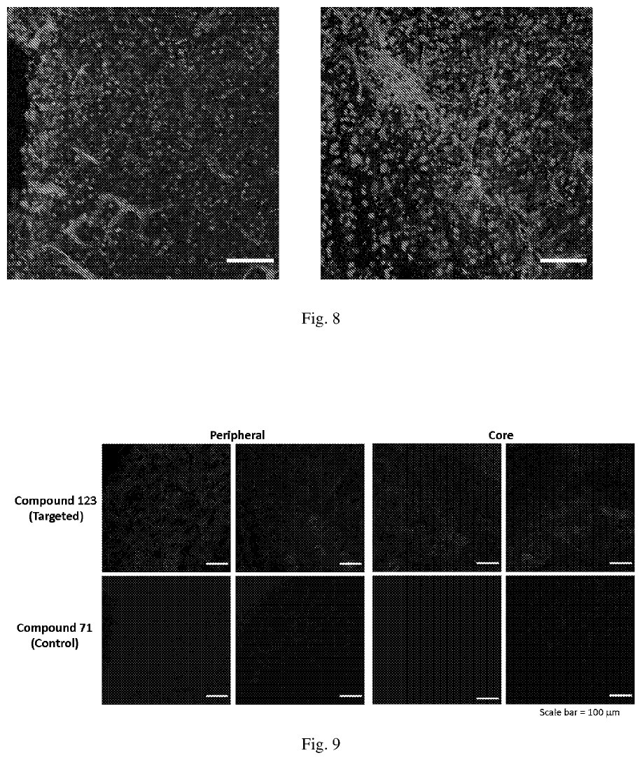

The mice were anaesthetised with isoflurane 48 h later, and blood collected via cardiac puncture immediately prior to cervical dislocation (n=2/group). Tumours were subsequently removed, fixed in 4% paraformaldehyde overnight, washed in PBS and embedded in agarose. Tumours were then vibratome sectioned to 100 μm, with sections stained for nuclei (DAPI, conc, time, supplier) and blood vessels (CD31, conc, time, supplier). Fluorescence images and optical sections were collected using a confocal microscope (Leica SP8). The results are shown in FIGS. 8 and 9. Targeted Dendrimer (Compound 123) showed uptake in core and peripheral regions of the tumour, Compound 71 did not.

### (b) Efficacy of Conjugate

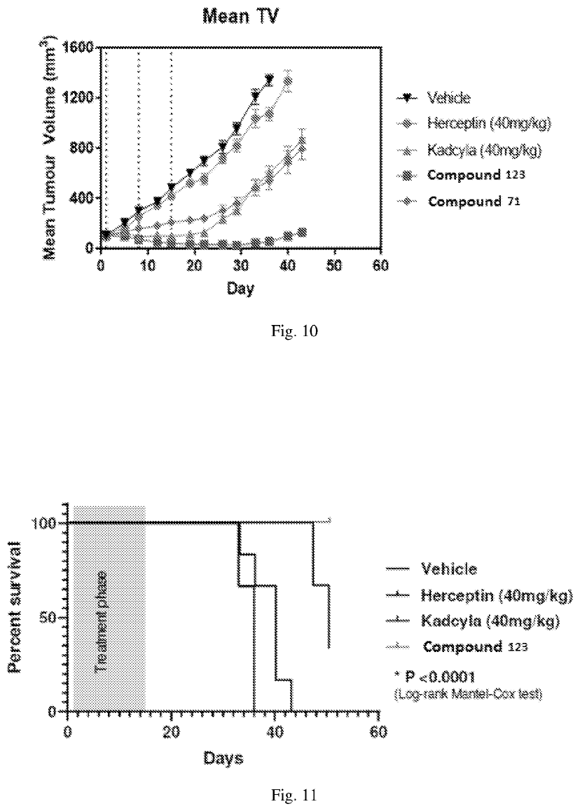

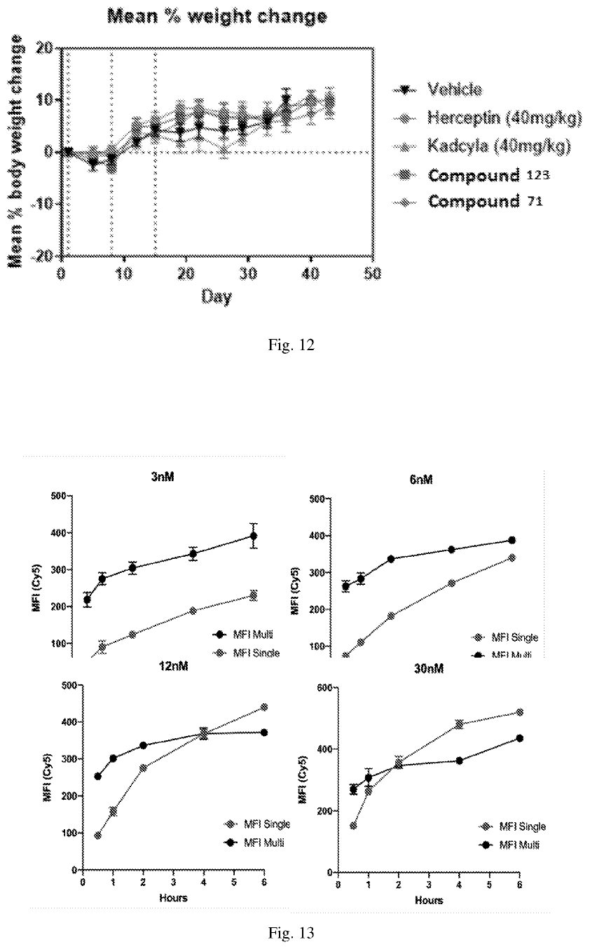

Tumour measurements were taken at regular intervals (n=6/group), until ethical endpoints were met. The results are shown in FIG. 10 (a plot of mean tumour volume over time), FIG. 11 (a plot of survival over time) and FIG. 12 (a plot of mean weight change over time). Targeted dendrimer Compound 123 showed total tumour regression compared to Compound 71, Herceptin®, and Kadcyla®.

### Example 11. Kinetics of Generation 4 Multi-Nanobody Dendrimer Internalisation in MDA-MB-231/HER2+ Cell Line

Generation 4, Cy5-labelled nanobody-dendrimer conjugates with siderophore-derived chelator desferrioxamine (DFO) that were conjugated to either 1 (single nanobody) or from 2 to 4 (multiple nanobody) anti-HER2 2D3 nanobodies, Compound 91 and Compound 92, separated as described above, were used to compare binding to HER2+ human cell lines where different numbers of nanobody are attached per dendrimer.

The epithelial adenocarcinoma cell line with HER2 knock-in, MDA-MB-23 1/HER2 (as described in Example 6) were maintained in Dulbecco's Modified Eagle's Medium (DMEM) containing 10% FBS and penicillin-streptomycin (100 UmL⁻¹) at 37° C. under a 5% CO₂ humidified atmosphere.

For measurement of cell binding by flow cytometry, 20,000 cells per well were plated overnight into 96-well culture plates in DMEM media supplemented with 10% FBS. Single or multiple nanobody dendrimers were added to cells at 3, 6, 12, and 30 nM and incubated for 0.5, 1, 2, 4, and 6 h at 37° C. Control cells prepared in the same manner were pre-chilled and incubated with 30 nM single and multiple nanobody dendrimers for 6 h on ice.

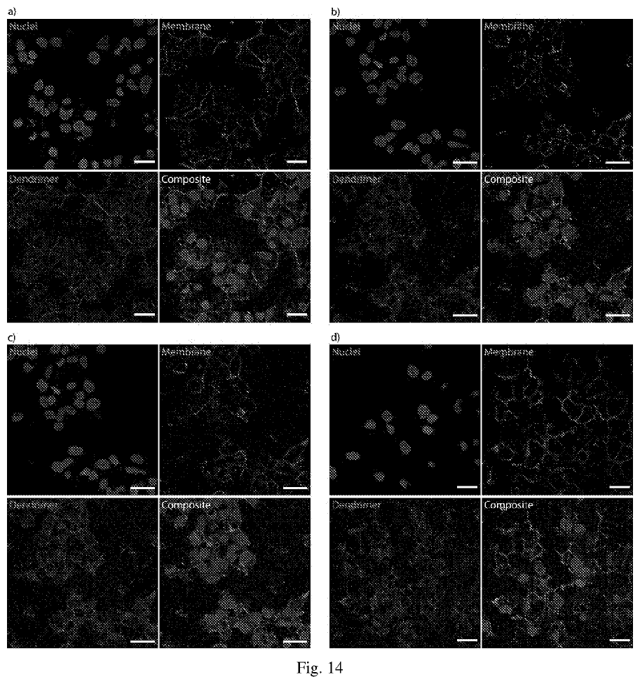

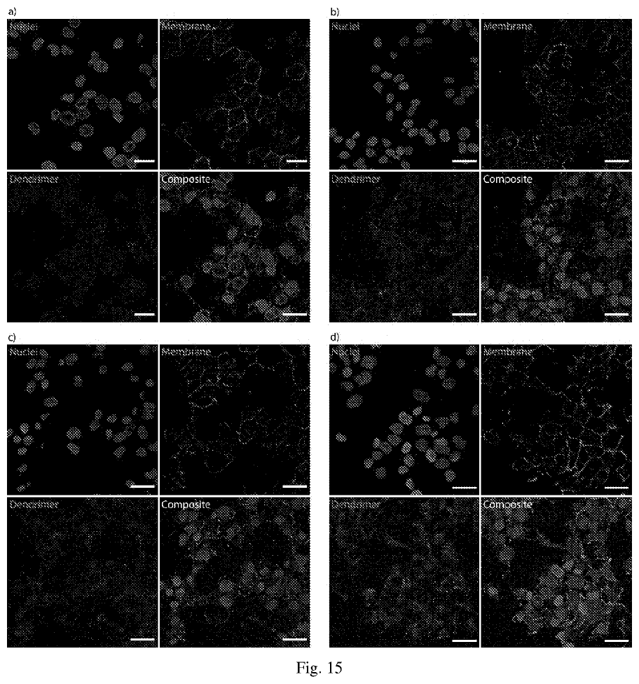

Following incubation with nanobody-conjugated dendrimers cells were washed three times with ice cold PBS supplemented with 1% Bovine serum albumin to remove unbound dendrimer and liberated from plates using TrypLE™ Express Enzyme (1×), no phenol red (Gibco, 12604013). Cells were resuspended in chilled DPBS for analysis by flow cytometry (Stratedigm S1000EON, California), with the presence of dendrimer measured by the acquisition of the signal from Cy5. To estimate the amount of internalised dendrimer at each concentration and time point, the Cy5 signal of cells incubated with dendrimer on ice was taken as the maximal surface bound dendrimer where no internalisation is assumed. This signal was subtracted from the signal measured in the cells incubated at 37° C., which represents surface bound and internalised dendrimer, to isolate the signal from internalised dendrimer only (see FIG. 13).

Multiple (Compound 92) conjugated anti-HER2 nanobody dendrimers internalise faster at low concentrations than with single (Compound 91). At higher concentration, Compound 91 shows greater levels of internalisation than Compound 92 after about 2 hours.

### Example 12. Confocal Imaging of Conjugates with Single and Multiple Nanobody in HER2 Hi Cell Lines

HER2 hi, SKOV-3 cells were plated at 1.0×10⁴ cells per well into μ-slide 8-well chambered coverslip (ibidi) and allowed to adhere overnight at 37° C., 5% CO₂. Compound 92 (multiple 2D3-dendrimer conjugate) and Compound 91 (single 2D3-dendrimer conjugate) (3.33 nM) were then added and allowed to incubate for 1, 3, 6, and 24 h. The cells were washed with DPBS and then fixed with 1% paraformaldehyde for 20 min at room temperature. The cell membrane was stained with Alexa Fluor 488® conjugate of wheat germ agglutinin (AF488-WGA, 5 μg mL⁻¹) in DPBS at room temperature for 10 min. The cell nuclei were counterstained with Hoechst 33342 (2 μg mL⁻¹) in PBS at room temperature for 10 min. Fluorescence images and optical sections were collected using a confocal microscope (Leica SP8). Images were processed with Fiji (ImageJ 1.52p).

Confocal microscopy images are shown in FIGS. 14 and 15. Both Compound 91 and Compound 92 bind and internalise with SKOV-3 cells after 24 h of incubation. Noticeably, Compound 92 (FIG. 14b, c) displays a greater extent of binding and internalisation at the 3 and 6 h time point compared to Compound 91 (FIG. 15b, c).

Multiple (Compound 92) conjugated anti-HER2 nanobody G4 dendrimers internalise faster than single nanobody G4 dendrimers (Compound 91) at this concentration and a difference can be seen up to 6 hours, but by 24 hours, they do not appear different.

### Example 13. Imaging Study with Targeted Zr Radionuclide-Containing Dendrimers—SKOV3 Breast Cancer Xenograft

The accumulation of ⁸⁹Zr-labelled HER2 targeted and untargeted dendrimer constructs in a SKOV3 murine xenograft model of ovarian cancer was investigated. The biodistribution was measured by PET-CT out to 9 days post-injection and validated by ex vivo gamma scintillation of excised organs at day 2 and 9 (where available). The study was conducted in three parts.

### Tumour Initiation and Growth

5×10⁶ SKOV3 cells (in 50 μL of 50:50 matrigel:PBS) were injected SC into the right flank of healthy female NOD-SCID (˜20 g) 8 week old mice. Tumours were allowed to grow for 4 weeks prior to injection of imaging compounds.

All tumours were palpable at the time of imaging, with sizes ˜3-5 mm at the time of the imaging experiment.

### Study Compounds

The compounds in the table below were labelled as described below.

TABLE
Study Compounds

| Com- | | Com- | |
|---|---|---|---|
| pound | | pound | |
| No. | Untargeted | No. | Targeted |
| | | 114 | DFO-NB |
| 129 | BHA-G2-PEG₁₀₀₀ | 85 | BHA-G2-PEG₁₀₀₀ + NB |
| | | 85 | BHA-G3-PEG₁₀₀₀ + NB + |
| | | | lysine preload control |
| 128 | BHA-G3-PEG₄₁₂ | 87 | BHA-G3-PEG₄₁₂ + NB |
| 130 | BHA-G3-PEG₁₀₀₀ | 89 | BHA-G3-PEG₁₀₀₀ + NB |
| 131 | BHA-G4-PEG₁₀₀₀ | 91 | BHA-G4-PEG₁₀₀₀ + NB |
| 132 | BHA-G5-PEG₁₀₀₀ | 93 | BHA-G5-PEG₁₀₀₀ + NB |

# = repeat

Radiolabeling of Study Compounds with ⁸⁹Zr and RadioTLC Analysis

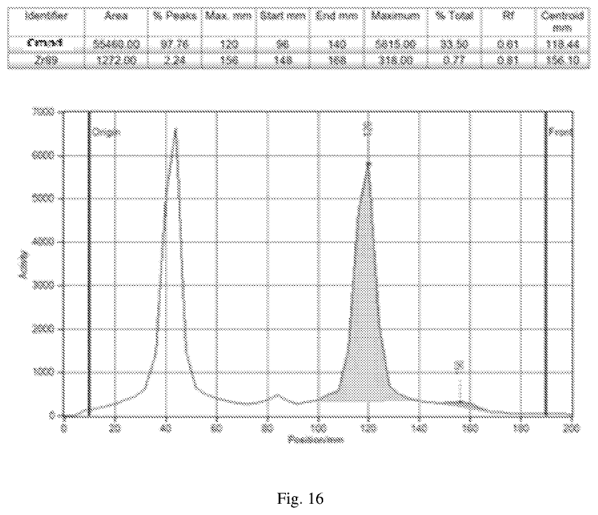

All constructs (pre-treated for Iron removal as needed: http://jnm.snmjournals.org/content/44/8/1271.long) were incubated with ⁸⁹Zr at an excess of dendrimer (see above Table for excess) in 0.1 M pH 7.4 HEPES buffer for 45 min at 37° C. Samples of each solution were taken and mixed 1:1 with 50 mM DTPA. 5 μL of each solution was spotted on TLC paper (Agilent iTLC-SG Glass microfiber chromatography paper impregnated with silica gel) and run with 50:50 H₂O:Ethanol. Plates were then imaged on a Eckert & Ziegler Mini-Scan and Flow-Count iTLC Reader. Where necessary, unbound zirconium was removed by purification using 7 K MWCO Zeba Spin Columns (Thermo Scientific) as per manufacturers protocols. All samples showed >95% labelling. Control experiments were conducted to monitor the elution behaviour of free ⁸⁹Zr and ⁸⁹Zr bound to DTPA for quality control, as well as each sample also run with and without DTPA to check for any unbound chelator labelled with ⁸⁹Zr. A representative RadioTLC image is shown in FIG. 16 showing that all ⁸⁹Zr was bound to the dendrimer and there was no free ⁸⁹Zr.

### Study Injection Details

For the in vivo imaging experiments, 100 μL study compounds were injected via the tail vein (29G needle; approx. 1.5 to 3.5 MBq) into two mice to monitor tumour accumulation and biodistribution at various timepoints. Quantitation was performed ex vivo by gamma counting to determine organ distribution 9 days post-injection. For the biodistribution experiments, constructs were injected into two mice to monitor tumour accumulation and biodistribution ex vivo at 48 h post-injection by gamma counting.

### Results

### PET-CT Imaging

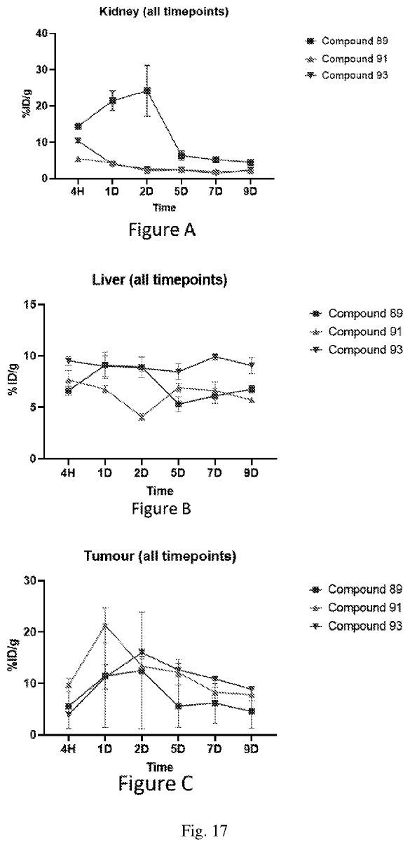

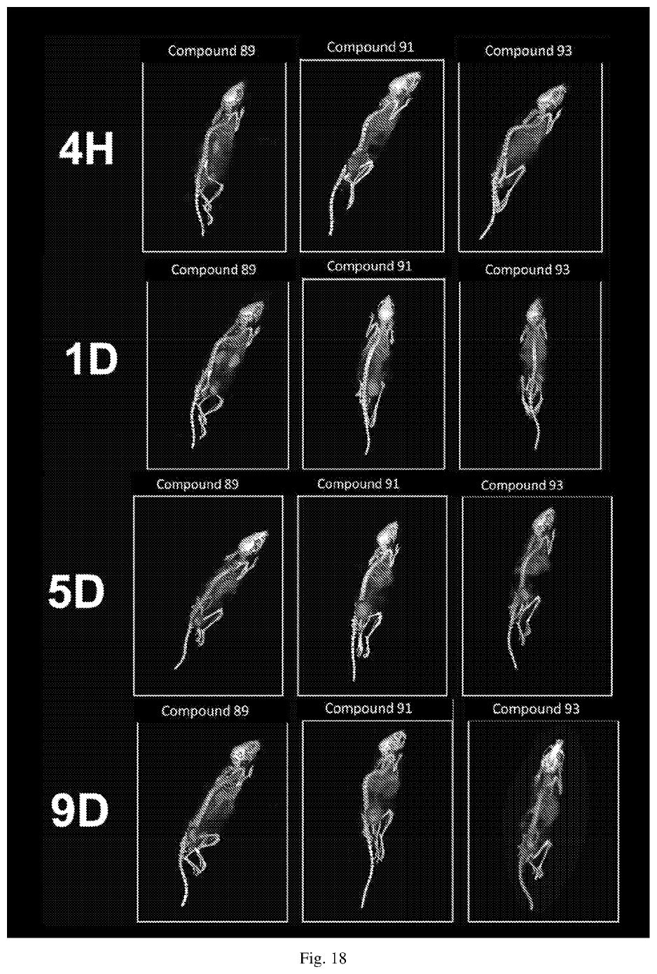

Images were taken at 4 h, 24 h, 48 h, 5 d, 7 d, and 9 d post-injection using 30-90 minute static acquisitions. The PET Images were reconstructed using an ordered-subset expectation maximization (OSEM2D) algorithm and analysed using the Inveon Research Workplace software (IRW 4.1) (Siemens) which allows fusion of CT and PET images and definition of regions of interest (ROIs). CT and PET datasets of each individual animal were aligned using IRW software (Siemens) to ensure good overlap of the organs of interest. Activity per voxel was converted to nci/cc using a conversion factor obtained by scanning a cylindrical phantom filled with a known activity of ⁸⁹Zr to account for PET scanner efficiency. Activity concentrations were then expressed as percent of the decay-corrected injected activity per cm³ of tissue that can be approximate as percentage injected dose/g (% ID/g). Results are shown in FIG. 17 as graphs of percentage injected Zirconium dose per gram in (a) kidney, (b) liver and (c) tumour over 9 days for Compounds 89, 91, and 93. Representative PET images are shown in FIG. 18. Representative maximum intensity projections of radiolabelled conjugates. All PET data is represented in Becquerel per voxel (cm³) and have been thresholded to highlight tumour uptake.

At 2- and 9-days post-injection, the organs were removed and signal intensity quantified by ex vivo gamma analysis and imaged. In addition, n=2 per cohort were culled at 48 h and evaluated for biodistribution by gamma analysis. Ex Vivo biodistribution results are shown in the tables below.

TABLE
Tumour:organ ratio of ex vivo signal of injected dose/g at days 2 and 9.

| | | Organs | | | | | | | |
|---|---|---|---|---|---|---|---|---|---|
| | | Tumour:Blood | | Tumour:Liver | | Tumour:Spleen | | Tumour:Kidneys | |
| Compound | Time/days | 2 | 9 | 2 | 9 | 2 | 9 | 2 | 9 |
| 25 | G2 | 1.02 | 6.57 | 0.20 | 0.11 | 0.29 | 0.11 | 0.73 | 0.82 |
| 85 | G2 + NB | 13.04 | 10.58 | 0.94 | 0.28 | 1.09 | 0.23 | 0.10 | 0.06 |
| 26 | G3 | 1.70 | 0.74 | 0.04 | 0.01 | 0.13 | 0.01 | 0.06 | 0.05 |
| 87 | G3 + NB | 24.30 | 1.67 | 0.31 | 0.05 | 1.16 | 0.05 | 0.03 | 0.01 |
| 27 | G3 − P1K | 0.91 | 7.76 | 0.28 | 0.15 | 0.33 | 0.04 | 0.69 | 0.51 |
| 89 | G3 − | 4.87 | 25.86 | 0.96 | 0.71 | 1.12 | 0.25 | 0.24 | 1.15 |
| | P1K + NB | | | | | | | | |
| 28 | G4 | 1.66 | 21.92 | 0.67 | 0.29 | 0.35 | 0.05 | 2.05 | 2.03 |
| 91 | G4 + NB | 4.33 | 98.30 | 2.24 | 0.98 | 1.54 | 0.29 | 5.83 | 4.00 |
| 29 | G5 | 0.69 | 13.68 | 0.75 | 0.37 | 0.29 | 0.07 | 2.22 | 2.29 |
| 93 | G5 + NB | 7.14 | 325.01 | 1.63 | 1.09 | 1.12 | 0.09 | 7.15 | 6.36 |
| 114 | NB | 17.17 | 1.24 | 4.51 | 1.36 | 2.56 | 0.10 | 0.01 | 0.00 |

The results show higher tumour:blood ratio for the targeted compared to non targeted in particular the at day 2 in Compound 87 (small G3 dendrimers). By day 9, tumour:blood ratio for the targeted compared to non targeted is better, and in particular for Compound 91 and Compound 93 (the larger G4 and G5 dendrimers).

TABLE
Percentage injected ⁸⁹Zr dose/gram in ex vivo tumour and organs at days 2 and 9.

Organ
Tumour (average % ID/g)

| Compound | Time (days) | 2 | 9 |
|---|---|---|---|
| 129 | G2 | 1.74 | 0.78 |
| 85 | G2 + NB | 2.70 | 1.06 |
| 128 | G3 | 0.71 | 0.25 |
| 87 | G3 − NB | 2.28 | 0.31 |
| 130 | G3 − P1K | 2.67 | 1.53 |
| 89 | G3 − P1K − NB | 4.78 | 4.56 |
| 131 | G4 | 3.96 | 1.72 |
| 91 | G4 − NB | 12.30 | 5.11 |
| 132 | G5 | 4.04 | 3.31 |
| 93 | G5 − NB | 11.96 | 8.18 |
| 114 | NB | 1.05 | 0.31 |

| | | Organ | | | | | | | |
|---|---|---|---|---|---|---|---|---|---|
| | | Liver | | Spleen | | Kidneys | | Heart | |
| Compound | Time (days) | 2 | 9 | 2 | 9 | 2 | 9 | 2 | 9 |
| 129 | G2 | 8.70 | 7.18 | 5.83 | 7.38 | 2.36 | 0.93 | 1.42 | 0.64 |
| 85 | G2 NB | 3.06 | 3.71 | 2.96 | 4.47 | 28.56 | 17.03 | 0.61 | 0.52 |
| 128 | G3 | 18.25 | 20.85 | 5.51 | 35.24 | 11.23 | 5.36 | 1.39 | 0.92 |
| 87 | G3 − NB | 7.82 | 6.02 | 2.14 | 6.88 | 79.08 | 55.61 | 0.49 | 0.28 |
| 130 | G3 − P1K | 9.62 | 10.10 | 8.12 | 47.73 | 4.05 | 3.11 | 1.91 | 1.23 |
| 89 | G3 − | 5.22 | 6.44 | 4.27 | 18.02 | 20.07 | 4.06 | 1.29 | 0.81 |
| | P1K − NB | | | | | | | | |
| 131 | G4 | 5.85 | 5.85 | 11.13 | 34.23 | 1.93 | 0.85 | 1.53 | 1.11 |
| 91 | G4 − NB | 5.54 | 5.23 | 8.03 | 17.53 | 2.15 | 1.26 | 2.51 | 1.24 |
| 132 | G5 | 5.48 | 9.59 | 16.45 | 45.59 | 1.87 | 1.43 | 3.08 | 1.83 |
| 93 | G5 − NB | 7.29 | 7.56 | 10.65 | 87.39 | 1.66 | 1.29 | 3.33 | 1.99 |
| 114 | NB | 0.23 | 0.23 | 0.53 | 0.00 | 109.01 | 62.22 | 0.10 | 0.00 |

| | | Organ | | | | | | | |
|---|---|---|---|---|---|---|---|---|---|
| | | Lungs | | Bone | | Blood | | GI Tract | |
| Compound | Time (days) | 2 | 9 | 2 | 9 | 2 | 9 | 2 | 9 |
| 129 | G2 | 1.25 | 0.44 | 1.04 | 0.28 | 1.67 | 0.00 | 0.87 | 0.31 |
| 85 | G2 − NB | 0.54 | 0.38 | 0.63 | 0.34 | 0.22 | 0.00 | 0.37 | 0.32 |
| 128 | G3 | 4.95 | 2.78 | 0.98 | 0.58 | 0.42 | 0.00 | 1.17 | 0.80 |
| 87 | G3 − NB | 0.58 | 0.55 | 0.63 | 0.00 | 0.09 | 0.00 | 0.34 | 0.29 |
| 130 | G3 − P1K | 4.36 | 3.55 | 0.93 | 0.79 | 3.05 | 0.00 | 1.01 | 0.77 |
| 89 | G3 − | 2.31 | 1.33 | 1.75 | 0.54 | 1.03 | 0.00 | 0.67 | 0.52 |
| | P1K − NB | | | | | | | | |
| 131 | G4 | 1.39 | 1.06 | 1.31 | 1.54 | 2.37 | 0.08 | 0.68 | 0.37 |
| 91 | G4 − NB | 2.69 | 1.10 | 2.05 | 1.18 | 3.07 | 0.00 | 1.01 | 0.41 |
| 132 | G5 | 2.71 | 1.12 | 1.70 | 1.30 | 5.97 | 0.04 | 0.85 | 0.63 |
| 93 | G5 − NB | 2.86 | 1.43 | 8.07 | 2.72 | 2.75 | 0.00 | 0.89 | 0.62 |
| 114 | NB | 0.11 | 0.00 | 0.26 | 0.00 | 0.08 | 0.00 | 0.04 | 0.11 |

The results show higher signal in the tumour for the targeted compared to non-targeted in particular for Compound 91 and Compound 93 (the larger G4 and G5 dendrimers) at day 2, and also at day 9 for Compound 89 (G3 dendrimer).

### CONCLUSION

1. The larger the dendrimer, the better tumour accumulation. Compound 49 (2D3) showed rapid clearance and low accumulation.
2. Targeted therapies lead to higher tumour accumulation than untargeted dendrimers.
3. Compound 87 (G3 1K-nanobody) shows a significantly enhanced signal in tumour compared to the other small dendrimers.
4. Compound 91 and Compound 93 (G4 and G5 nanobody) showed >12% ID/g in tumour at 48 h, and >8% at 9 days.
5. Small nanobody containing dendrimers and the nanobody alone have a higher kidney retention. No unusual accumulation in other clearance organs was observed, with the liver and spleen signal showing expected concentration ranges as typically observed for polymeric nanomaterials.

### Human Dosimetry Modeling Based on the PET-CT Imaging

Dosimetry calculations were performed following the MIRD schema.
A monoexponential was fitted to the ⁸⁹Zr mouse data supplied. Λ_{phys} was then modified for the isotopes ¹⁷⁷Lu and ⁶⁸Ga to calculate the total number of disintegrations for each isotope.

A₁e^{-(λ}^{₁}^{+λ}^{_{phys}}^{)t}

Tumour dose was modelled on the size of the mouse tumour.

### General Observations;

The dose to organs not listed from cross irradiation is minimal and multiple orders of magnitude less than that of organs with notable uptake.
The kidney mGy/MBq for ¹⁷⁷Lu appears to be of similar order of magnitude to other ⁷⁷Lu radiopharmaceuticals such as ¹Lutathera and ¹⁷⁷LuPSMA-617.
Tumour dose is relatively high in comparison to normal organs uptake

| G2 + NB | | | | G2 | | | |
|---|---|---|---|---|---|---|---|
| (Compound 85) | Isotope (mGy/MBq) | | | (Compound 129) | Isotope (mGy/MBq) | | |
| Organ | Zr-89 | Lu-177 | Ga-68 | Organ | Zr-89 | Lu-177 | Ga-68 |
| Heart | 0.048 | 0.067 | 0.004 | Heart | 0.112 | 0.141 | 0.013 |
| Lung | 0.025 | 0.038 | 0.002 | Lung | 0.035 | 0.058 | 0.003 |
| Liver | 0.528 | 0.534 | 0.027 | Liver | 0.837 | 0.922 | 0.039 |
| Kidney | 1.514 | 1.859 | 0.151 | Kidney | 0.160 | 0.211 | 0.014 |
| Tumour | 224 | 527 | 26 | Tumour | 88 | 215 | 10 |

| G3 − P1K + NB | | | | G3 − P1K | | | |
|---|---|---|---|---|---|---|---|
| (Compound 89) | Isotope (mGy/MBq) | | | (Compound 130) | Isotope (mGy/MBq) | | |
| Organ | Zr-89 | Lu-177 | Ga-68 | Organ | Zr-89 | Lu-177 | Ga-68 |
| Heart | 0.136 | 0.165 | 0.017 | Heart | 0.256 | 0.300 | 0.036 |
| Lung | 0.054 | 0.074 | 0.007 | Lung | 0.077 | 0.110 | 0.009 |
| Liver | 0.722 | 0.715 | 0.038 | Liver | 1.411 | 1.480 | 0.069 |
| Kidney | 1.870 | 2.136 | 0.221 | Kidney | 0.488 | 0.605 | 0.048 |
| Tumour | 529 | 1511 | 60 | Tumour | 171 | 498 | 20 |

| | |
|---|---|
| G4 + NB | G4 |

| | | | |
|---|---|---|---|
| (Compound 91) | Isotope (mGy/MBq) | (Compound 131) | Isotope (mGy/MBq) |

| Organ | Zr-89 | Lu-177 | Ga-68 | Organ | Zr-89 | Lu-177 | Ga-68 |
|---|---|---|---|---|---|---|---|
| Heart | 0.153 | 0.191 | 0.018 | Heart | 0.174 | 0.203 | 0.024 |
| Lung | 0.065 | 0.095 | 0.007 | Lung | 0.059 | 0.080 | 0.007 |
| Liver | 0.807 | 0.910 | 0.037 | Liver | 0.741 | 0.743 | 0.038 |
| Kidney | 0.313 | 0.393 | 0.030 | Kidney | 0.369 | 0.472 | 0.034 |
| Tumour | 532 | 1404 | 54 | Tumour | 101 | 325 | 10 |
| G5 + NB | | | | G5 | | | |

| | | | |
|---|---|---|---|
| (Compound 93) | Isotope (mGy/MBq) | (Compound 132) | Isotope (mGy/MBq) |

| Organ | Zr-89 | Lu-177 | Ga-68 | Organ | Zr-89 | Lu-177 | Ga-68 |
|---|---|---|---|---|---|---|---|
| Heart | 0.191 | 0.234 | 0.024 | Heart | 0.215 | 0.259 | 0.028 |
| Lung | 0.077 | 0.108 | 0.009 | Lung | 0.071 | 0.106 | 0.008 |
| Liver | 1.022 | 1.153 | 0.046 | Liver | 0.957 | 1.063 | 0.044 |
| Kidney | 0.327 | 0.395 | 0.034 | Kidney | 0.403 | 0.512 | 0.037 |
| Tumour | 732 | 2008 | 82 | Tumour | 250 | 992 | 18 |

| NB-DFO | | | |
|---|---|---|---|
| (Compound 114) | Isotope (mGy/MBq) | | |
| Organ | Zr-89 | Lu-177 | Ga-68 |
| Heart | 0.009 | 0.010 | 0.002 |
| Lung | 0.008 | 0.010 | 0.001 |
| Liver | 0.097 | 0.083 | 0.006 |
| Kidney | 3.125 | 4.081 | 0.277 |
| Tumour | 98 | 195 | 25 |

### Example 14. Radiotherapeutic Efficacy in BT474 Xenograft Model

### Animal Model

Healthy in female Balb/c nude mice (aged 9 weeks) were obtained from the Animal Resource Centre, Western Australia. A total of four mice per dosing group were initially implanted with estrogen pellets (17Beta-estradiol 0.72 mg/pellet 60 Day Release; Innovative Research of America) and then on day 2 were innoculated subcutaneously in the breast mammary fat pad with 10×10⁶ BT474 cells in PBS:Matrigel (1:1). Mice were weighed, and tumours measured 2-3 times weekly using electronic callipers. Tumour volume (mm³) were calculated as ((length (mm)×((width (mm))²)/2. Mice were randomised into eight dosing groups outlined in the Table below as tumours reached mean volumes of approximately 100 mm³ (approximately 5 weeks from implantation of cells).

### Radiolabelling and TLC Analysis.

Either dendrimer or modified Trastuzumab were incubated with ¹⁷⁷Lu at a 100-fold excess of dendrimer/Trastuzumab in 0.1 M pH 5.5 ammonium acetate buffer for 45 min at 37° C. Samples of each solution were taken and mixed 1:1 with 50 mM DTPA. 5 μL of each DTPA incubated sample or neat solution was spotted on TLC paper (Agilent iTLC-SG Glass microfiber chromatography paper impregnated with silica gel) and run with 50:50 H2O:ethanol. Detection of radiolabelled species migration was then achieved using an Eckert and Ziegler Mini-Scan and Flow-Count system. All samples showed >90% labelling. Control experiments were conducted to monitor the elution behaviour of free ¹⁷⁷Lu and ¹⁷⁷Lu bound to DTPA for quality control.

Where necessary, unbound copper was removed by purification using 7 K MWCO Zeba Spin Columns (Thermo Scientific) as per manufacturers protocols.

For each of the analyses discussed, radioisotopic TLCs were obtained by mixing samples with an excess of DTPA (50 mM) to scavenge any unbound ¹⁷⁷Lu. Test compounds were radiolabeled with ¹⁷⁷Lu to deliver a radiometric dose of ˜9 or 15 MBq by dilution of hot material with cold material to achieve the desired specific activity and drug loading.

### Treatment Protocol

Test articles were administered at approximately 0.1 ml/10 g body weight IV by tail vein injection (29G needle) on the schedule described below. Mice were culled if the tumours reached significant size (>1 cm³), or in accordance with ethical requirements.

TABLE
Dosing groups

| Test Article | ¹⁷⁷Lu dose | Dose Regimen |
|---|---|---|
| Saline | 0 | |
| KY-3-310 | 1 × 15 MBq | Day 1 |
| (Trastuzumab) | | |
| RH-3-160 | 1 × 9 MBq | Day 1 |
| (untargeted) | | |
| RH-3-160 | 2 × 9 MBq | day 1 and 15 |
| (untargeted) | | |
| RH-3-160 | 1 × 15 MBq | Day 1 |
| (untargeted) | | |
| SRS-2-304 | 1 × 9 MBq | Day 1 |
| (nanobody) | | |
| SRS-2-304 | 2 × 9 MBq | day 1 and 15 |
| (nanobody) | | |
| SRS-2-304 | 1 × 15 MBq | Day 1 |
| (nanobody) | | |

### Results

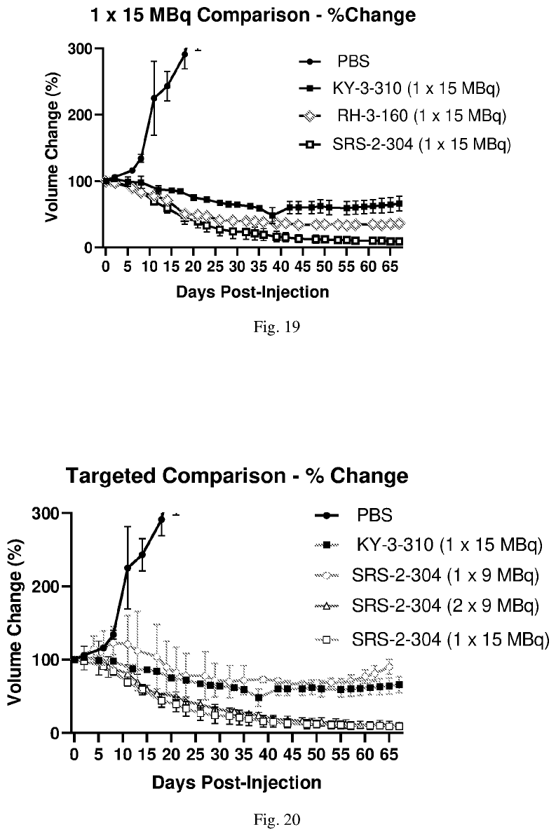

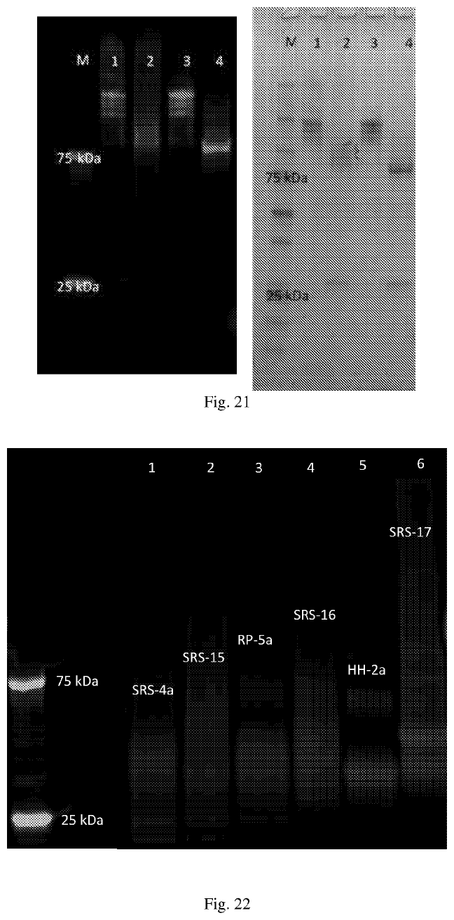

As shown in FIG. 19, and the table below, targeted radionuclide dendrimer (Compound SRS-2-304) and the untargeted radionuclide dendrimer (Compound RH-3-160) were both effective in suppressing tumour growth, with the targeted dendimer being the most effective. FIG. 20 shows no tumour regrowth at day 67 for the targeted (Compound SRS-2-304) at 15 mBq and 2×9 MBq ¹⁷⁷Lu doses.

TABLE
Statistical comparison of % volume change of
HER2 targeted and untargeted dendrimers at day
67 (unpaired T-Test with Welch's correction).

| | Compared to | Compared to |
|---|---|---|
| | Compound | Compound |
| | KY-3-310 | RH-3-160 |
| Test Article | (Trastuzumab) | (Untargeted) at |
| (MBq dose) | 1 × 15 MBq | equivalent MBq dose |
| SRS-2-304 | NS | P = 0.0155 |
| (1 × 9 MBq) | | |
| SRS-2-304 | P = 0.0007 | P = 0.0025 |
| (1 × 15 MBq) | | |
| SRS-2-304 | P = 0.0009 | P = 0.0126 |
| (2 × 9 MBq) | | |
| RH-3-160 | P = 0.0107 | |
| (1 × 15 MBq) | | |

1. A dendrimer-targeting agent conjugate comprising:
a) a dendrimer comprising
i) a core unit (C); and
ii) building units (BU),
wherein the dendrimer has from two to six generations of building units; and
wherein the core unit is covalently attached to at least two building units;
b) a targeting agent which is covalently linked to the dendrimer by a spacer group;
c) one or more first terminal groups attached to an outermost building unit of the dendrimer, wherein the first terminal group comprises a complexation group for complexing a radionuclide; and
d) one or more second terminal groups attached to an outermost building unit of the dendrimer, wherein the second terminal group comprises a pharmacokinetic-modifying moiety;
or a salt thereof.

2. A conjugate as claimed in claim 1, wherein the targeting agent is a peptidic moiety having a molecular weight of up to about 150 kDa, or up to about 110 KDa, or up to about 80 KDa, or up to about 55 KDa, or up to about 16 kDa, and comprising an antigen-binding site.

3. (canceled)

4. A conjugate as claimed in claim 1, wherein the targeting agent is selected from: an antibody, a heavy chain antibody, ScFV-Fc, Fab, Fab₂, Fv, scFv or a single domain antibody, or wherein the targeting agent comprises or consists of a heavy chain variable (V_{H}) domain, or wherein the targeting agent comprises or consists of a light chain variable (V_{L}) domain.

5-6. (canceled)

7. A conjugate as claimed in claim 1, wherein the targeting agent has a molecular weight of about 5 kDa to about 30 kDa, or about 3 kDa to about 20 kDa.

8. (canceled)

9. A conjugate as claimed in claim 1, wherein the targeting agent comprises fewer than 120 amino acid residues.

10. A conjugate as claimed in claim 1, wherein the targeting agent is a HER2 targeting agent, optionally a HER2 nanobody.

11-12. (canceled)

13. A conjugate as claimed in claim 1, wherein the targeting agent is a small molecule, optionally a small molecule that binds PSMA, optionally a DUPA analogue, or the targeting agent is a FAP binding group.

14-16. (canceled)

17. A conjugate as claimed in claim 1, wherein a covalent linkage between the targeting agent and the spacer group has been formed by reaction between complementary reactive functional groups present on an intermediate comprising the targeting agent and an intermediate comprising the dendrimer,
optionally wherein the intermediate comprising the targeting agent comprises an unnatural amino acid residue, the unnatural amino acid residue having a side-chain including a reactive functional group, optionally wherein the unnatural amino acid residue is a 4-azidophenylalanine residue, or
optionally wherein the intermediate comprising the targeting agent comprises a reactive cysteine residue.

18-20. (canceled)

21. A conjugate as claimed in claim 1, wherein the spacer group comprises a PEG group.

22. A conjugate as claimed in claim 1, wherein the targeting agent is covalently linked to the spacer group at or near the C-terminus of the targeting agent.

23. A conjugate as claimed in claim 17, wherein the intermediate comprising the dendrimer comprises a reactive functional group which is an alkyne group, optionally wherein the alkyne group is a dibenzocyclooctyne group.

24. (canceled)

25. A conjugate as claimed in claim 1, wherein the first terminal group further comprises a radionuclide complexed with the complexation group.

26. A conjugate as claimed in claim 1, wherein the complexation group is a DOTA, benzyl-DOTA, NOTA, DTPA, sarcophagine, macropa, DFO, PEPA or EDTA group.

27. A conjugate as claimed in claim 1, wherein the radionuclide in the radionuclide-containing moiety is a lutetium, gallium, zirconium, actinium, bismuth, astatine, technetium, lead, yttrium or copper radionuclide.

28-30. (canceled)

31. A conjugate as claimed in claim 1, wherein the pharmacokinetic-modifying moiety is a polyethylene glycol (PEG) group, or a polyethyloxazoline (PEOX) group, or a poly-(2) methyl-(2)-oxazolamine (POZ), or a polysarcosine, or a poly(2-hydroxypropyl)methacrylamide (pHPMA) group.

32-33. (canceled)

34. A conjugate as claimed in claim 1, wherein the dendrimer has between two to five generations of building units.

35. A conjugate as claimed in claim 1, wherein the core unit comprises or is selected from:

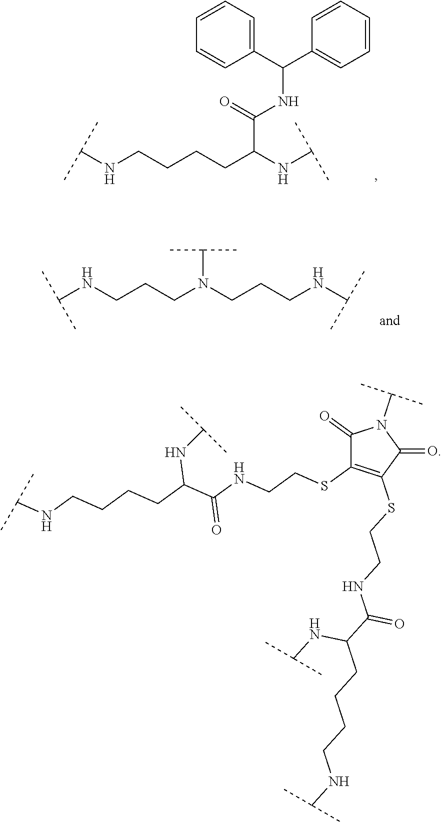

36-37. (canceled)

38. A conjugate as claimed in claim 1, wherein the conjugate is any of the example conjugates.

39. (canceled)

40. A pharmaceutical composition, comprising:
i) a conjugate as claimed in claim 1; and
ii) a pharmaceutically acceptable excipient.

41-46. (canceled)

47. A kit for producing a therapeutic conjugate as defined in claim 1, comprising:
a) a conjugate as claimed in claim 1; and
b) a radionuclide.
